(19) United States
(12) Patent Application Publication
(10) Pub. No.: US 2016/0102317 A1
(43) Pub. Date: Apr. 14, 2016

(54) PLANTS HAVING INCREASED TOLERANCE TO HERBICIDES

(71) Applicants: BASF SE, Ludwigshafen (DE); BASF (CHINA) COMPANY LIMITED, Shanghai (CN)

(72) Inventors: Maciej Pasternak, Ludwigshafen (DE); Stefan Tresch, Kirchheim (DE); Helmut Kraus, Wissembourg (FR); Johannes Hutzler, Waldsee (DE); Jens Lerchl, Potsdam OT Golm (DE); Thomas Mietzner, Annweiler (DE); Liliana Parra Rapado, Offenburg (DE); Jill Marie Paulik, Cary, NC (US)

(73) Assignee: BASF SE, Ludwigshafen (DE)

(21) Appl. No.: 14/888,025

(22) PCT Filed: Apr. 28, 2014
(86) PCT No.: PCT/IB2014/061052

§ 371 Date: Oct. 29, 2015

Related U.S. Application Data
(60) Provisional application No. 61/817,326, filed on Apr. 30, 2013.
(60) Provisional application No. 61/817,327, filed on Apr. 30, 2013.

(51) Int. Cl. C12N 15/82; A01N 43/82; A01N 43/713; C12N 9/02

(52) U.S. Cl. CPC C12N 15/8274; C12N 9/0069; A01N 43/82; A01N 43/713; C12Y 113/11027

(57) ABSTRACT

The present invention refers to a method for controlling undesired vegetation at a plant cultivation site, the method comprising the steps of providing, at said site, a plant that comprises at least one nucleic acid comprising a nucleotide sequence encoding a wild-type hydroxyphenyl pyruvate dioxygenase or a mutated hydroxyphenyl pyruvate dioxygenase (mut-HPPD) which is resistant or tolerant to a HPPD-inhibiting herbicide and/or a nucleotide sequence encoding a wild-type homogentisate solanesyl transferase or a mutated homogentisate solanesyl transferase (mut-HST) which is resistant or tolerant to a HPPD-inhibiting herbicide, preferably a bicycloarylcarboxamide, applying to said site an effective amount of said herbicide. The invention further refers to plants comprising mut-HPPD, and methods of obtaining such plants.

This application claims priority to US provisional applications U.S. 61/817,326 and U.S. 61/817,327, both filed on Apr. 30, 2013, all of which are incorporated herein by reference in their entirety.

### FIELD OF THE INVENTION

The present invention relates in general to methods for conferring on plants agricultural levels of tolerance towards an herbicide. Particularly, the invention refers to plants having an increased tolerance to “HPPD-inhibiting” herbicides. More specifically, the present invention relates to methods and plants obtained by mutagenesis and cross-breeding and transformation that have an increased tolerance to “HPPD-inhibiting” herbicides.

### BACKGROUND OF THE INVENTION

Herbicides that inhibit 4-hydroxyphenylpyruvate dioxygenase (4-HPPD; EC 1.13.11.27), a key enzyme in the biosynthesis of the prenylquinones plastoquinone and tocopherols, have been used for selective weed control since the early 1990s. They block the conversion of 4-hydroxyphenylpyruvate to homogentisate in the biosynthetic pathway (Matringe et al., 2005, Pest Manag Sci., vol. 61:269-276; Mitchell et al., 2001, Pest Manag Sci. vol 57:120-128). Plastoquinone is thought to be a necessary cofactor of the enzyme phytoene desaturase in carotenoid biosynthesis (Boeger and Sandmann, 1998, Pestic Outlook, vol 9:29-35). Its inhibition results in the depletion of the plant plastoquinone and vitamin E pools, leading to bleaching symptoms. The loss of carotenoids, particularly in their function as protectors of the photosystems against photooxidation, leads to oxidative degradation of chlorophyll and photosynthetic membranes in growing shoot tissues. Consequently, chloroplast synthesis and function are disturbed (Boeger and Sandmann, 1998). The enzyme homogentisate solanesyl transferase (HST) catalyses the step following HPPD in the plastoquinone biosynthetic pathway. HST is a prenyl transferase that both decarboxylates homogentisate and also transfers to it the solanesyl group from solanesyl diphosphate and thus forms 2-methyl-6-solanesyl-1,4-benzoquinol (MSBQ), an intermediate along the biosynthetic pathway to plastoquinone. HST enzymes are membrane bound and the genes that encode them include a plastid targeting sequence.

The most important chemical classes of commercial 4-HPPD-inhibiting herbicides include pyrazolones, triketones and isoxazoles. The inhibitors mimic the binding of the substrate 4-hydroxyphenylpyruvate to an enzyme-bound ferrous ion in the active site by forming a stable iondipole charge transfer complex. Among 4-HPPD-inhibiting herbicides, the triketone sulcotrione was the first example of this herbicide group to be used in agriculture and identified in its mechanism of action (Schulz et al., 1993, FEBS Lett. Vol 318:162-166) The triketones have been reported to be derivatives of leptospermone, a herbicidal component from the bottlebrush plant, Callistemon spp (Lee et al. 1997, Weed Sci. Vol 45, 162-166).

Some of these molecules have been used as herbicides since inhibition of the reaction in plants leads to whitening of the leaves of the treated plants and to the death of the said plants (Pallett, K. E. et al. 1997 Pestic. Sci. 50 83-84). The herbicides for which HPPD is the target, and which are described in the state of the art, are, in particular, isoxazoles (EP418175, EP470856, EP487352, EP527036, EP560482, EP682659, U.S. Pat. No. 5,424,276), in particular isoxaflutole, which is a selective herbicide for maize, diketonitriles (EP496630, EP496631), in particular 2-cyano-3-cyclopropyl-1-(2-SO₂CH₃-4-CF₃ phenyl)propane-1,3-dione and 2-cyano-3-cyclopropyl-1-(2-SO₂CH₃-4-2,3Cl₂phenyl)propane-1,3-dione, triketones such as described in EP625505, EP625508, U.S. Pat. No. 5,506,195, in particular sulcotrione, or else pyrazolinates. Furthermore, the well-known herbicide topramezone elicits the same type of phytotoxic symptoms, with chlorophyll loss and necrosis in the growing shoot tissues, as 4-HPPD inhibiting, bleaching herbicides described supra in susceptible plant species. Topramezone belongs to the chemical class of pyrazolones or benzoyl pyrazoles and was commercially introduced in 2006. When applied post-emergence, the compound selectively controls a wide spectrum of annual grass and broadleaf weeds in corn.

Three main strategies are available for making plants tolerant to herbicides, i.e. (1) detoxifying the herbicide with an enzyme which transforms the herbicide, or its active metabolite, into non-toxic products, such as, for example, the enzymes for tolerance to bromoxynil or to Basta (EP242236, EP337899); (2) mutating the target enzyme into a functional enzyme which is less sensitive to the herbicide, or to its active metabolite, such as, for example, the enzymes for tolerance to glyphosate (EP293356, Padgette S. R. et al., J. Biol. Chem., 266, 33, 1991); or (3) overexpressing the sensitive enzyme so as to produce quantities of the target enzyme in the plant which are sufficient in relation to the herbicide, in view of the kinetic constants of this enzyme, so as to have enough of the functional enzyme available despite the presence of its inhibitor. The third strategy was described for successfully obtaining plants which were tolerant to HPPD inhibitors (WO96/38567). US2009/0172831 discloses nucleotide sequences encoding amino acid sequences having enzymatic activity such that the amino acid sequences are resistant to HPPD inhibitor herbicidal chemicals, in particular triketone inhibitor specific HPPD mutants.

To date, the prior art has not described HPPD-inhibiting herbicide tolerant plants containing at least one mutated HPPD nucleic acid according to the present invention. What are needed in the art are crop plants and crop plants having increased tolerance to herbicides such as HPPD-inhibiting herbicide and containing at least one mutated HPPD nucleic acid according to the present invention. Also needed are methods for controlling weed growth in the vicinity of such crop plants or crop plants. These compositions and methods would allow for the use of spray over techniques when applying herbicides to areas containing crop plant or crop plants.

### SUMMARY OF THE INVENTION

The problem is solved by the present invention which refers to a method for controlling undesired vegetation at a plant cultivation site, the method comprising the steps of:

a) providing, at said site, a plant that comprises at least one nucleic acid comprising
(i) a nucleotide sequence encoding a wild-type hydroxyphenyl pyruvate dioxygenase or a mutated hydroxyphenyl pyruvate dioxygenase (mut-HPPD) which is resistant or tolerant to a HPPD-inhibiting herbicide and/or
(ii) a nucleotide sequence encoding a wild-type homogentisate solanesyl transferase or a mutated homogentisate solanesyl transferase (mut-HST) which is resistant or tolerant to a HPPD-inhibiting herbicide
   b) applying to said site an effective amount of said herbicide.

In addition, the present invention refers to a method for identifying a HPPD-inhibiting herbicide by using a mut-HPPD encoded by a nucleic acid which comprises the nucleotide sequence of SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 52, 54, 56, or a variant thereof, and/or by using a mut-HST encoded by a nucleic acid which comprises the nucleotide sequence of SEQ ID NO: 47 or 49 or a variant thereof.

Said method comprises the steps of:
a) generating a transgenic cell or plant comprising a nucleic acid encoding a wild-type or mut-HPPD, wherein the wild-type or mut-HPPD is expressed;
b) applying a HPPD-inhibiting herbicide to the transgenic cell or plant of a) and to a control cell or plant of the same variety;
c) determining the growth or the viability of the transgenic cell or plant and the control cell or plant after application of said test compound, and
d) selecting test compounds which confer reduced growth to the control cell or plant as compared to the growth of the transgenic cell or plant.

Another object refers to a method of identifying a nucleotide sequence encoding a mut-HPPD which is resistant or tolerant to a HPPD-inhibiting herbicide, the method comprising:
a) generating a library of mut-HPPD-encoding nucleic acids,
b) screening a population of the resulting mut-HPPD-encoding nucleic acids by expressing each of said nucleic acids in a cell or plant and treating said cell or plant with a HPPD-inhibiting herbicide,
c) comparing the HPPD-inhibiting herbicide-tolerance levels provided by said population of mut-HPPD encoding nucleic acids with the HPPD-inhibiting herbicide-tolerance level provided by a control HPPD-encoding nucleic acid,
d) selecting at least one mut-HPPD-encoding nucleic acid that provides a significantly increased level of tolerance to a HPPD-inhibiting herbicide as compared to that provided by the control HPPD-encoding nucleic acid.

In a preferred embodiment, the mut-HPPD-encoding nucleic acid selected in step d) provides at least 2-fold as much or tolerance to a HPPD-inhibiting herbicide as compared to that provided by the control HPPD-encoding nucleic acid.

The resistance or tolerance can be determined by generating a transgenic plant comprising a nucleic acid sequence of the library of step a) and comparing said transgenic plant with a control plant.

Another object refers to a method of identifying a plant or algae containing a nucleic acid encoding a mut-HPPD or mut-HST which is resistant or tolerant to a HPPD-inhibiting herbicide, the method comprising:
a) identifying an effective amount of a HPPD-inhibiting herbicide in a culture of plant cells or green algae.
b) treating said plant cells or green algae with a mutagenizing agent,
c) contacting said mutagenized cells population with an effective amount of HPPD-inhibiting herbicide, identified in a),
d) selecting at least one cell surviving these test conditions,
e) PCR-amplification and sequencing of HPPD and/or HST genes from cells selected in d) and comparing such sequences to wild-type HPPD or HST gene sequences, respectively.

In a preferred embodiment, the mutagenizing agent is ethylmethanesulfonate.

Another object refers to an isolated nucleic acid encoding a mut-HPPD, the nucleic acid being identifiable by a method as defined above.

In another embodiment, the invention refers to a plant cell transformed by a wild-type or a mut-HPPD nucleic acid or or a plant which has been mutated to obtain a plant expressing, preferably over-expressing, a wild-type or a mut-HPPD nucleic acid, wherein expression of the nucleic acid in the plant cell results in increased resistance or tolerance to a HPPD-inhibiting herbicide as compared to a wild-type variety of the plant cell.

In a preferred embodiment, the plant cell of the present is transformed by a wild-type or a mut-HPPD nucleic acid comprising a sequence of SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69 or a variant or derivative thereof.

In another embodiment, the invention refers to a transgenic plant comprising a plant cell according to the present invention, wherein expression of the nucleic acid in the plant results in the plant's increased resistance to HPPD-inhibiting herbicide as compared to a wild-type variety of the plant.

The plants of the present invention can be transgenic or non-transgenic.

Preferably, the expression of the nucleic acid in the plant results in the plant's increased resistance to HPPD-inhibiting herbicide as compared to a wild-type variety of the plant.

In another embodiment, the invention refers to a seed produced by a transgenic plant comprising a plant cell of the present invention, wherein the seed is true breeding for an increased resistance to a HPPD-inhibiting herbicide as compared to a wild-type variety of the seed.

In another embodiment, the invention refers to a method of producing a transgenic plant cell with an increased resistance to a HPPD-inhibiting herbicide as compared to a wild-type variety of the plant cell comprising, transforming the plant cell with an expression cassette comprising a wild-type or a mut-HPPD nucleic acid.

In another embodiment, the invention refers to a method of producing a transgenic plant comprising, (a) transforming a plant cell with an expression cassette comprising a wild-type or a mut-HPPD nucleic acid, and (b) generating a plant with an increased resistance to HPPD-inhibiting herbicide from the plant cell.

Preferably, the expression cassette further comprises a transcription initiation regulatory region and a translation initiation regulatory region that are functional in the plant.

In another embodiment, the invention relates to using the mut-HPPD of the invention as selectable marker. The invention provides a method of identifying or selecting a transformed plant cell, plant tissue, plant or part thereof comprising a) providing a transformed plant cell, plant tissue, plant or part thereof, wherein said transformed plant cell, plant tissue, plant or part thereof comprises an isolated nucleic acid encoding a mut-HPPD polypeptide of the invention as described hereinafter, wherein the polypeptide is used as a selection marker, and wherein said transformed plant cell, plant tissue, plant or part thereof may optionally comprise a further isolated nucleic acid of interest; b) contacting the transformed plant cell, plant tissue, plant or part thereof with at least one HPPD-inhibiting inhibiting compound; c) determining whether the plant cell, plant tissue, plant or part thereof is affected by the inhibitor or inhibiting compound; and d) identifying or selecting the transformed plant cell, plant tissue, plant or part thereof.

The invention is also embodied in purified mut-HPPD proteins that contain the mutations described herein, which are useful in molecular modeling studies to design further improvements to herbicide tolerance. Methods of protein purification are well known, and can be readily accomplished using commercially available products or specially designed methods, as set forth for example, in Protein Biotechnology, Walsh and Headon (Wiley, 1994).

### BRIEF DESCRIPTION OF THE DRAWINGS

FIG. 1 Amino acid sequence alignment and conserved regions of HPPD enzymes from Chlamydomonas reinhardtii (Cr_HPPD1a, Cr_H PP D1b), Physcomitrella patens (Pp_HPPD1), Oryza sativa (Osj_HPPD1), Triticum aestivum (Ta_HPPD1), Zea mays (Zm_HPPD1), Arabidopsis thaliana (At_HPPD), Glycine max(Gm_HPPD), Vitis vinifera (Vv_HPPD) and Hordeum vulgare (Hv_HPPD).

* Sequence derived from genome sequencing project. Locus ID: GRMZM2G088396

** Amino acid sequence based on NCBI GenPept accession CAG25475

FIG. 2 shows a vector map of a plant transformation vector which is used for soybean transformation with HPPD/HST sequences.

FIG. 3 shows a germination assay with transgenic Arabidopsis seedlings expressing Arabidopsis wild-type HPPD (AtHPPD). Rows A-F are individual events. Non-transformed control plants are marked as wild-type (WT). The HPPD-inhibiting herbicide used refers to (NE)-8-chloro-N-(4-methoxy-1,2,5-oxadiazol-3-ylidene)-4,4-dimethyl-1,1-dioxo-2,3-dihydrothiochromene-7-carboxamide

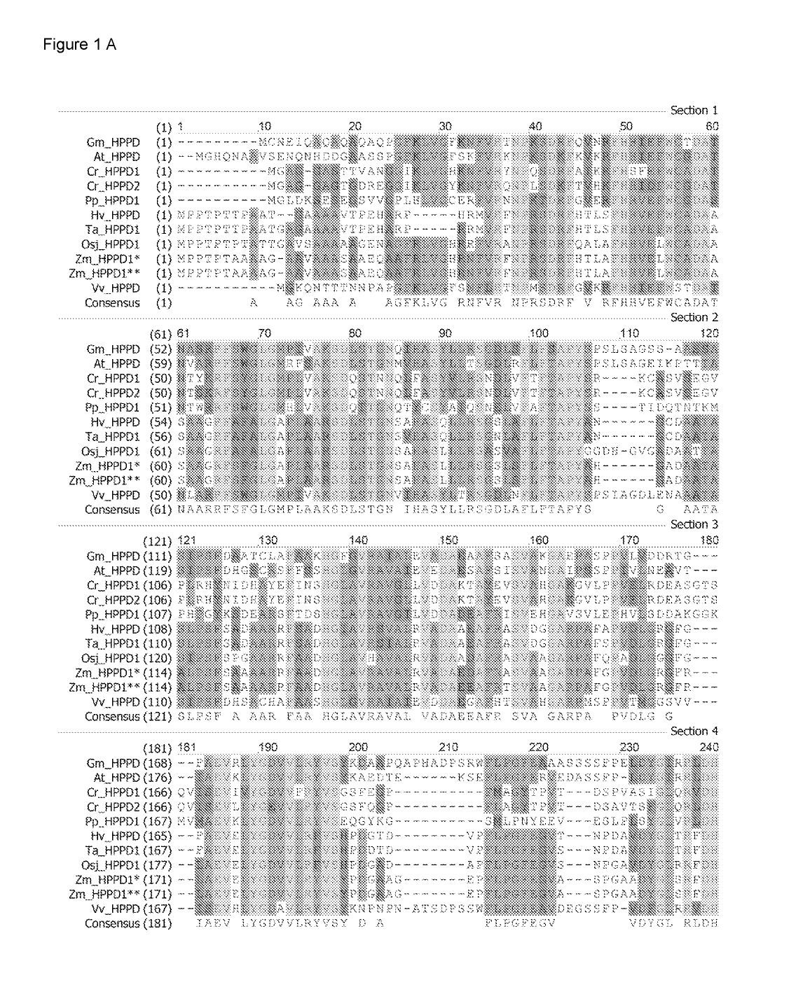

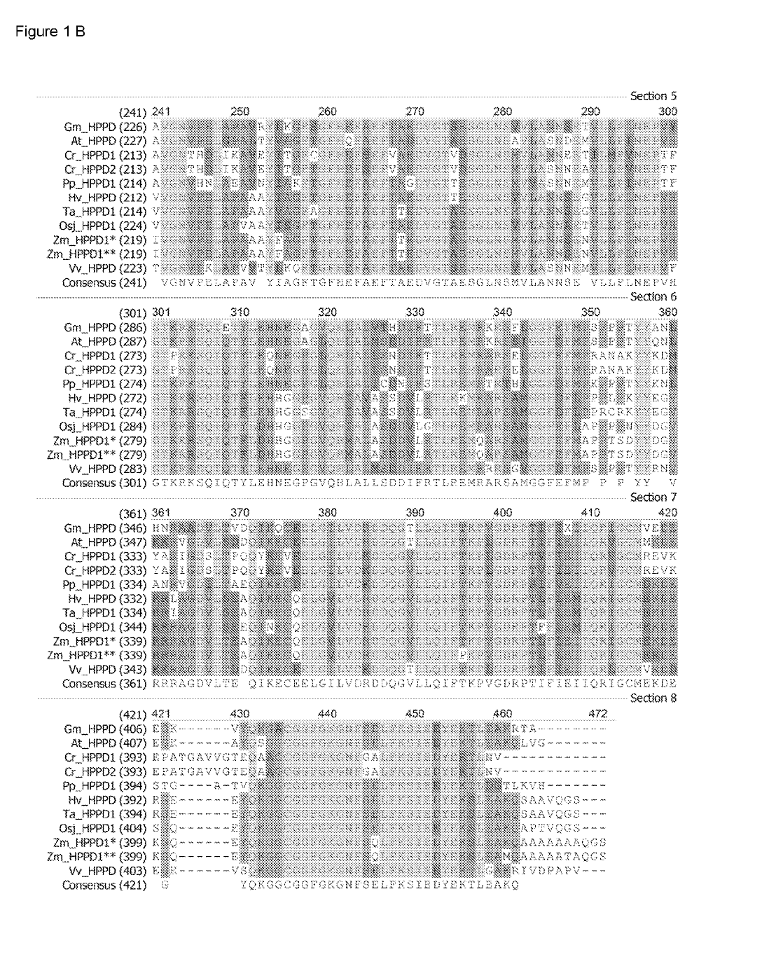

### SEQUENCE LISTING

TABLE 1

| SEQ | | | | |
|---|---|---|---|---|
| ID | | | | Accession |
| NO: | Description | Organism | Locus | number |
| 1 | HPPD nucleic acid | Hordeum | | |
| 51 | HPPD nucl acid opt | Hordeum | | |
| 2 | HPPD amino acid | Hordeum | | |
| 3 | HPPD nucleic acid | Fragilariopsis | | |
| 4 | HPPD nucl acid opt | Fragilariopsis | | |
| 5 | HPPD amino acid | Fragilariopsis | | |
| 6 | HPPD nucleic acid | Chlorella | | |
| 7 | HPPD nucl acid opt | Chlorella | | |
| 8 | HPPD amino acid | Chlorella | | |
| 9 | HPPD nucleic acid | Thalassiosira | | |
| 10 | HPPD nucl acid opt | Thalassiosira | | |
| 11 | HPPD amino acid | Thalassiosira | | |
| 12 | HPPD nucleic acid | Cyanothece | | |
| 13 | HPPD nucl acid opt | Cyanothece | | |
| 14 | HPPD amino acid | Cyanothece | | |
| 15 | HPPD nucleic acid | Acaryochloris | | |
| 16 | HPPD nucl acid opt | Acaryochloris | | |
| 17 | HPPD amino acid | Acaryochloris | | |
| 18 | HPPD nucleic acid | Synechocystis | | |
| 19 | HPPD nucl acid opt | Synechocystis | | |
| 20 | HPPD amino acid | Synechocystis | | |
| 21 | HPPD nucleic acid1 | Alopecurus | | |
| 22 | HPPD amino acid1 | Alopecurus | | |
| 23 | HPPD nucleic acid2 | Alopecurus | | |
| 24 | HPPD amino acid2 | Alopecurus | | |
| 25 | HPPD nucleic acid1 | Sorghum | | |
| 26 | HPPD amino acid1 | Sorghum | | |
| 27 | HPPD nucleic acid2 | Sorghum | | |
| 28 | HPPD amino acid2 | Sorghum | | |
| 29 | HPPD nucleic acid1 | Poa | | |
| 30 | HPPD amino acid1 | Poa | | |
| 31 | HPPD nucleic acid2 | Poa | | |
| 32 | HPPD amino acid2 | Poa | | |
| 33 | HPPD nucleic acid | Lolium | | |
| 34 | HPPD amino acid | Lolium | | |
| 35 | HPPD nucleic acid | Synechococcus | | |
| 36 | HPPD amino acid | Synechococcus | | |
| 37 | HPPD nucleic acid | Blepharisma | | |
| 38 | HPPD amino acid | Blepharisma | | |
| 39 | HPPD nucleic acid | Picrophilus | | |
| 40 | HPPD amino acid | Picrophilus | | |
| 41 | HPPD nucleic acid | Kordia | | |
| 42 | HPPD amino acid | Kordia | | |
| 43 | HPPD nucleic acid1 | Rhodococcus | | |
| 44 | HPPD amino acid1 | Rhodococcus | | |
| 45 | HPPD nucleic acid2 | Rhodococcus | | |
| 46 | HPPD amino acid2 | Rhodococcus | | |
| 47 | HST nucleic acid | Arabidopsis | At3g11945 | DQ231060 |
| 48 | HST amino acid | Arabidopsis | At3g11945 | Q1ACB3 |
| 49 | HST nucleic acid | Chlamydomonas | | AM285678 |
| 50 | HST amino acid | Chlamydomonas | | A1JHN0 |
| 52 | HPPD nucleic acid | Arabidopsis | At1g06570 | AF047834 |
| 53 | HPPD amino acid | Arabidopsis | At1g06570 | AAC15697 |
| 54 | HPPD nucleic acid1 | Chlamydomonas | | |
| 55 | HPPD amino acid1 | Chlamydomonas | | |
| 56 | HPPD nucleic acid2 | Chlamydomonas | | XM_ |
| | | | | 001694671.1 |
| 57 | HPPD amino acid2 | Chlamydomonas | | Q70ZL8 |
| 58 | HPPD amino acid | Physcomitrella | | A9RPY0 |
| 59 | HPPD amino acid | Oryza | Os02g07160 | |
| 60 | HPPD amino acid | Triticum | | Q45FE8 |
| 61 | HPPD amino acid | Zea | | CAG25475 |
| 62 | HPPD amino acid | Glycine | | A5Z1N7 |
| 63 | HPPD amino acid | Vitis | | A5ADC8 |
| 64 | HPPD amino acid | Pseudomonas | | AXW96633 |
| | | fluorescens | | |
| | | strain | | |
| | | 87-79 | | |
| 65 | HPPD amino acid | Pseudomonas | | ADR00548 |
| | | fluorescens | | |
| 66 | HPPD amino acid | Avena sativa | | AXW96634 |
| 67 | HPPD amino acid | Zea mays variant | | |
| 68 | HPPD nucleic acid | Zea mays mut | | codon- |
| | | 10 | | optimised |
| 69 | HPPD nucleic acid | Zea mays mut | | codon- |
| | | 406 | | optimised |

### DETAILED DESCRIPTION

The articles “a” and “an” are used herein to refer to one or more than one (i.e., to at least one) of the grammatical object of the article. By way of example, “an element” means one or more elements.

As used herein, the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The inventors of the present invention have found, that the tolerance or resistance of a plant to a HPPD-inhibiting herbicide herbicide could be remarkably increased by overexpressing wild-type or mutated HPPD enzymes comprising SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67.

Consequently, the present invention refers to a method for controlling undesired vegetation at a plant cultivation site, the method comprising the steps of:
a) providing, at said site, a plant that comprises at least one nucleic acid comprising
   (i) a nucleotide sequence encoding a wild-type hydroxyphenyl pyruvate dioxygenase (HPPD) or a mutated hydroxyphenyl pyruvate dioxygenase (mut-HPPD) which is resistant or tolerant to a “HPPD-inhibiting herbicide” and/or
   (ii) a nucleotide sequence encoding a wild-type homogentisate solanesyl transferase (HST) or a mutated homogentisate solanesyl transferase (mut-HST) which is resistant or tolerant to a “HPPD-inhibiting herbicide”
b) applying to said site an effective amount of said herbicide.

The term “control of undesired vegetation” is to be understood as meaning the killing of weeds and/or otherwise retarding or inhibiting the normal growth of the weeds. Weeds, in the broadest sense, are understood as meaning all those plants which grow in locations where they are undesired. The weeds of the present invention include, for example, dicotyledonous and monocotyledonous weeds. Dicotyledonous weeds include, but are not limited to, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera. In addition, the weeds of the present invention can include, for example, crop plants that are growing in an undesired location. For example, a volunteer maize plant that is in a field that predominantly comprises soybean plants can be considered a weed, if the maize plant is undesired in the field of soybean plants.

The term “plant” is used in its broadest sense as it pertains to organic material and is intended to encompass eukaryotic organisms that are members of the Kingdom Plantae, examples of which include but are not limited to vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, under-ground stems, clumps, crowns, bulbs, corms, tubers, rhizomes, plants/tissues produced in tissue culture, etc.). The term “plant” further encompasses whole plants, ancestors and progeny of the plants and plant parts, including seeds, shoots, stems, leaves, roots (including tubers), flowers, florets, fruits, pedicles, peduncles, stamen, anther, stigma, style, ovary, petal, sepal, carpel, root tip, root cap, root hair, leaf hair, seed hair, pollen grain, microspore, cotyledon, hypocotyl, epicotyl, xylem, phloem, parenchyma, endosperm, a companion cell, a guard cell, and any other known organs, tissues, and cells of a plant, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites austrails, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco. Further preferably, the plant is a monocotyledonous plant, such as sugarcane. Further preferably, the plant is a cereal, such as rice, maize, wheat, barley, millet, rye, sorghum or oats.

In a preferred embodiment, the plant has been previously produced by a process comprising recombinantly preparing a plant by introducing and over-expressing a wild-type or mut-HPPD and/or wild-type or mut-HST transgene, as described in greater detail hereinafter.

In another preferred embodiment, the plant has been previously produced by a process comprising in situ mutagenizing plant cells, to obtain plant cells which express a mut-HPPD and/or mut-HST.

As disclosed herein, the nucleic acids of the invention find use in enhancing the herbicide tolerance of plants that comprise in their genomes a gene encoding a herbicide-tolerant wild-type or mut-HPPD and/or wild-type or mut-HST protein. Such a gene may be an endogenous gene or a transgene, as described hereinafter.

Therefore, in a other embodiment the present invention refers to a method of increasing or enhancing the HPPD-inhibiting herbicide tolerance or resistance of a plant, the method comprising overexpressing a nucleic acid encoding a wild-type or mut HPPD enzymes comprising SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67.

In one embodiment, the wild-type HPPD enzyme comprises SEQ ID NO: 40, 44, or 46.

Additionally, in certain embodiments, the nucleic acids of the present invention can be stacked with any combination of polynucleotide sequences of interest in order to create plants with a desired phenotype. For example, the nucleic acids of the present invention may be stacked with any other polynucleotides encoding polypeptides having pesticidal and/or insecticidal activity, such as, for example, the Bacillus thuringiensis toxin proteins (described in U.S. Pat. Nos. 5,366,892; 5,747,450; 5,737,514; 5,723,756; 5,593,881; and Geiser et al (1986) Gene 48: 109). The combinations generated can also include multiple copies of any one of the polynucleotides of interest.

By way of example, polynucleotides that may be stacked with the nucleic acids of the present invention include nucleic acids encoding polypeptides conferring resistance to pests/pathogens such as viruses, nematodes, insects or fungi, and the like. Exemplary polynucleotides that may be stacked with nucleic acids of the invention include polynucleotides encoding: polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al., (1986) Gene 48:109), lectins (Van Damme et al. (1994) Plant Mol. Biol. 24:825, pentin (described in U.S. Pat. No. 5,981,722), and the like; traits desirable for disease or herbicide resistance (e.g., fumonisin detoxification genes (U.S. Pat. No. 5,792,931); avirulence and disease resistance genes (Jones et al. (1994) Science 266:789; Martin et al., (1993) Science 262:1432; Mindrinos et al. (1994) Cell 78:1089); acetolactate synthase (ALS) mutants that lead to herbicide resistance such as the S4 and/or Hra mutations; glyphosate resistance (e.g., 5-enol-pyrovyl-shikimate-3-phosphate-synthase (EPSPS) gene, described in U.S. Pat. Nos. 4,940,935 and 5,188,642; or the glyphosate N-acetyltransferase (GAT) gene, described in Castle et al. (2004) Science, 304:1151-1154; and in U.S. Patent App. Pub. Nos. 20070004912, 20050246798, and 20050060767)); glufosinate resistance (e.g, phosphinothricin acetyl transferase genes PAT and BAR, described in U.S. Pat. Nos. 5,561,236 and 5,276,268); resistance to herbicides including sulfonyl urea, DHT (2,4D), and PPO herbicides (e.g., glyphosate acetyl transferase, aryloxy alkanoate dioxygenase, acetolactate synthase, and protoporphyrinogen oxidase); a cytochrome P450 or variant thereof that confers herbicide resistance or tolerance to, inter alia, HPPD herbicides (U.S. patent application Ser. No. 12/156,247; U.S. Pat. Nos. 6,380,465; 6,121,512; 5,349,127; 6,649,814; and 6,300,544; and PCT Patent App. Pub. No. WO2007000077); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)); the disclosures of which are herein incorporated by reference.

In a particularly preferred embodiment, the plant comprises at least one additional heterologous nucleic acid comprising (iii) a nucleotide sequence encoding a herbicide tolerance enzyme selected, for example, from the group consisting of 5-enolpyruvylshikimate-3-phosphate synthase (EPSPS), Glyphosate acetyl transferase (GAT), Cytochrome P450, phosphinothricin acetyltransferase (PAT), Acetohydroxyacid synthase (AHAS; EC 4.1.3.18, also known as acetolactate synthase or ALS), Protoporphyrinogen oxidase (PPGO), Phytoene desaturase (PD) and dicamba degrading enzymes as disclosed in WO 02/068607.

Generally, the term “herbicide” is used herein to mean an active ingredient that kills, controls or otherwise adversely modifies the growth of plants. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present invention. Typically, the effective amount of a herbicide is an amount that is routinely used in agricultural production systems to kill weeds of interest. Such an amount is known to those of ordinary skill in the art. Herbicidal activity is exhibited by HPPD-inhibiting herbicide useful for the present invention when they are applied directly to the plant or to the locus of the plant at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, it is preferred to apply the HPPD-inhibiting herbicide postemergence to relatively immature undesirable vegetation to achieve the maximum control of weeds.

By a “herbicide-tolerant” or “herbicide-resistant” plant, it is intended that a plant that is tolerant or resistant to at least one herbicide at a level that would normally kill, or inhibit the growth of, a normal or wild-type plant. By “herbicide-tolerant mut-HPPD protein” or “herbicide-resistant mut-HPPD protein”, it is intended that such a mut-HPPD protein displays higher HPPD activity, relative to the HPPD activity of a wild-type mut-HPPD protein, when in the presence of at least one herbicide that is known to interfere with HPPD activity and at a concentration or level of the herbicide that is known to inhibit the HPPD activity of the wild-type mut-HPPD protein. Furthermore, the HPPD activity of such a herbicide-tolerant or herbicide-resistant mut-HPPD protein may be referred to herein as “herbicide-tolerant” or “herbicide-resistant” HPPD activity.

The HPPD-inhibiting herbicides which are particularly useful for the present invention encompasses the compounds as depicted in the following Table 2.

TABLE 2

| | | Possible Substituents as | |
|---|---|---|---|
| | | defined in: | |
| | | Application number and | Publication |
| No: | General Structure | reference | Number |
| 1 | 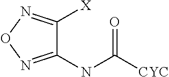 | PCT/EP2012/072692 PF72975 | WO 2013/072402 |
| | I | | |
| 2 | 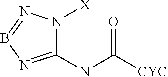 | US 61/639081 PF73636 | WO 2013/076315 |
| | II | | |

The above referenced application, in particular the disclosures referring to the compounds of Table 2 and their possible substitutents are entirely incorporated by reference.

In one embodiment of the present invention, the HPPD-inhibiting herbicide refers to a bicycloarylcarboxamide shown in Number 1 of Table 2 above having the above formula I:
wherein
X is selected from the group consisting of hydrogen, cyano, nitro, halogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, C₁-C₄-haloalkoxy-C₁-C₄-alkyl, O—R^{a}, Z—S(O)ₙ—R^{b}, Z—C(═O)—R^{c}, Z—C(═O)—OR^{d}, Z—C(═O)—NR^{e}R^{f}, Z—NR^{g}R^{h}, Z-phenyl and Z-heterocyclyl, where heterocyclyl is a 5- or 6-membered monocyclic or 8-, 9- or 10-membered bicyclic saturated, partially unsaturated or aromatic heterocycle, which contains 1, 2, 3 or 4 heteroatoms as ring members, which are selected from the group consisting of O, N and S, where phenyl and heterocyclyl are unsubstituted or substituted by 1, 2, 3 or 4 groups R′, which are identical or different;

In one embodiment of the present invention, the HPPD-inhibiting herbicide refers to a bicycloarylcarboxamide shown in Number 2 of Table 2 above having the above formula II:
wherein
B is N or CH;
Y is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, C₁-C₄-haloalkoxy-C₁-C₄-alkyl, R^{b}—S(O)ₙ—C₁-C₃-alkyl, R^{b}—C(═O)—C₁-C₃-alkyl, R^{d}O—C(═O)—C₁-C₃-alkyl, R^{e}R^{f}N—C(═O)—C₁-C₃-alkyl, R^{g}R^{h}N—C₁-C₃-alkyl, phenyl-Z and heterocyclyl-Z, where heterocyclyl is a 5- or 6-membered monocyclic or 8-, 9- or 10-membered bicyclic saturated, partially unsaturated or aromatic heterocycle, which contains 1, 2, 3 or 4 heteroatoms as ring members, which are selected from the group consisting of O, N and S, where phenyl and heterocyclyl are unsubstituted or substituted by 1, 2, 3 or 4 groups R′, which are identical or different;

For both formula I and II, CYC indicates a bi- or tricyclic radical of the following formulae Cyc-1 or Cyc-2

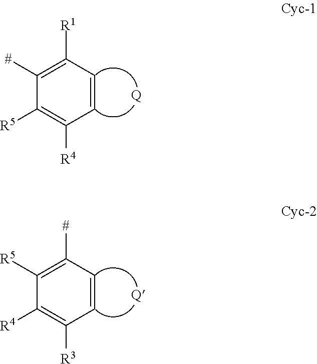

where
# indicates the point of attachment of the bicyclic radical to the carbonyl group,
Q, Q′ independently of each other indicate a fused 5-, 6-, 7-, 8-, 9- or 10-membered carbocycle or a fused 5-, 6-, 7-, 8-, 9- or 10-membered heterocycle, where the fused heterocycle has 1, 2, 3 or 4 heteroatoms selected from O, S and N as ring members, where the fused carbocycle and the fused heterocycle are monocyclic or bicyclic and where the fused carbocycle and the fused heterocycle are unsubstituted or carry 1, 2, 3, 4, 5, 6, 7, 8, 9 or 10 radicals R²;
R¹ in formula Cyc-1 is selected from the group consisting of Z¹-cyano, halogen, nitro, C₁-C₈-alkyl, C₂-C₈-alkenyl, C₂-C₈-alkynyl, C₁-C₈-haloalkyl, C₁-C₈-alkoxy, C₁-C₄-alkoxy, C₁-C₄-alkyl, Z¹—C₁-C₄-alkoxy-C₁-C₄-alkoxy, C₁-C₄-alkylthio-C₁-C₄-alkyl, Z¹—C₁-C₄-alkylthio-C₁-C₄-alkylthio, C₂-C₆-alkenyloxy, C₂-C₆-alkynyloxy, C₁-C₆-haloalkoxy, C₁-C₄-haloalkoxy-C₁-C₄-alkyl, Z¹—C₁-C₄-haloalkoxy-C₁-C₄-alkoxy, Z¹—S(O)ₖ—R^{1b}, Z¹-phenoxy and Z¹-heterocyclyloxy, where heterocyclyloxy is an oxygen bound 5- or 6-membered monocyclic or 8-, 9- or 10-membered bicyclic saturated, partially unsaturated or aromatic heterocycle, which contains 1, 2, 3 or 4 heteroatoms as ring members, which are selected from the group consisting of O, N and S, where the cyclic groups in phenoxy and heterocyclyloxy are unsubstituted or substituted by 1, 2, 3 or 4 groups R¹¹, which are identical or different;
R² is selected from the group consisting of halogen, Z²—OH, Z²—NO₂, Z²-cyano, oxo (═O), ═N—R²², C₁-C₄-alkyl, C₁-C₄-haloalkyl, C₂-C₄-alkenyl, C₂-C₄-alkynyl, Z²—C₁-C₄-alkoxy, C₁-C₄-alkoxy-C₁-C₄-alkoxy-C₁-C₄-alkyl, C₁-C₄-alkylthio, C₁-C₄-haloalkylthio, Z²—C₁-C₄-haloalkoxy, Z²—C₃-C₁₀-cycloalkyl, O—Z²—C₃-C₁₀-cycloalkyl, Z²-(tri-C₁-C₄-alkyl)silyl, Z²—S(O)ₖ—R^{2b}, Z²—C(═O)—R^{2b}, Z²—NR^{2g}R^{2h} and Z²-phenyl, where phenyl in Z²-phenyl is unsubstituted or substituted by 1, 2, 3 or 4 groups R²¹, which are identical or different;
R³ in formula Cyc-2 is selected from the group consisting of hydrogen, halogen, Z³—OH, Z³—NO₂, Z³-cyano, C₁-C₆-alkyl, C₂-C₈-alkenyl, C₂-C₈-alkynyl, Z³—C₃-C₁₀-cycloalkyl, Z³—C₃-C₁₀-cycloalkoxy, where the C₃-C₁₀-cycloalkyl groups in the two aforementioned radicals are unsubstituted or partially or completely halogenated, C₁-C₈-haloalkyl, Z³—C₁-C₈-alkoxy, Z³—C₁-C₈-haloalkoxy, Z³—C₁-C₄-alkoxy-C₁-C₄-alkoxy, Z³—C₁-C₄-alkylthio-C₁-C₄-alkylthio, Z³—C₂-C₈-alkenyloxy, Z³—C₂-C₈-alkynyloxy, Z³—C₁-C₈-haloalkoxy, Z³—C₁-C₄-haloalkoxy-C₁-C₄-alkoxy, Z³-(tri-C₁-C₄-alkyl)silyl, Z³—S(O)ₖ—R^{3b}, Z³—C(═O)—R^{3c}, Z³—C(═O)—OR^{3d}, Z³—C(═O)—NR^{3e}R^{3f}, Z³—NR^{3g}R^{3b}, Z^{3a}-phenyl and Z^{3a}-heterocyclyl, where heterocyclyl is a 3-, 4-, 5- or 6-membered monocyclic or 8-, 9- or 10-membered bicyclic saturated, partially unsaturated or aromatic heterocycle, which contains 1, 2, 3 or 4 heteroatoms as ring members, which are selected from the group consisting of O, N and S, where the cyclic groups in Z^{3a}-phenyl and Z^{3a}-heterocyclyl are unsubstituted or substituted by 1, 2, 3 or 4 groups R³¹, which are identical or different;
R⁴ is selected from the group consisting of hydrogen, halogen, cyano, nitro, C₁-C₄-alkyl and C₁-C₄-haloalkyl;
R⁵ is selected from the group consisting of hydrogen, halogen, C₁-C₄-alkyl and C₁-C₄-haloalkyl;
n is 0, 1 or 2;
k is 0, 1 or 2;
R′, R¹¹, R²¹, R³¹ independently of each other are selected from the group consisting of halogen, NO₂, CN, C₁-C₆-alkyl, C₃-C₇-cycloalkyl, C₃-C₇-halocycloalkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₄-alkoxy-C₁-C₄-alkyl, C₁-C₄-haloalkoxy-C₁-C₄-alkyl, C₃-C₇-cycloalkoxy and C₁-C₆-haloalkyloxy;
R²² is selected from the group consisting of C₁-C₄-alkoxy, C₁-C₄-haloalkoxy and C₃-C₇-cycloalkoxy, which is unsubstituted or partially or completely halogenated;
Z, Z¹, Z², Z³ independently of each other are selected from the group consisting of a covalent bond and C₁-C₄-alkanediyl;
Z^{3a} is selected from the group consisting of a covalent bond, C₁-C₄-alkanediyl, O—C₁-C₄-alkanediyl, C₁-C₄-alkanediyl-O and C₁-C₄-alkanediyl-O—C₁-C₄-alkanediyl;
R^{a} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{b}, R^{1b}, R^{2b}, R^{3b} independently of each other are selected from the group consisting of C₁-C₆-alkyl, C₃-C₇-cycloalkyl, C₁-C₆-haloalkyl, C₂-C₆-alkenyl, C₂-C₆-haloalkenyl, C₂-C₆-alkynyl, C₂-C₆-haloalkynyl and phenyl, where phenyl is unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{c}, R^{2c}, R^{3c} independently of each other are selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl, benzyl and heterocyclyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl, benzyl and heterocyclyl are unsubstituted or substituted by 1, 2, 3 or 4 groups selected from the group consisting of halogen, C₁-C₄-alkyl, C₁-C₄-haloalkyl, C₁-C₄-alkoxy and C₁-C₄-haloalkoxy;
R^{d}, R^{3d} independently of each other are selected from the group consisting of C₁-C₆-alkyl, C₃-C₇-cycloalkyl, C₃-C₇-cycloalkyl-C₁-C₄-alkyl, where the C₃-C₁-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{e}, R^{f} independently of each other are selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy, or
R^{e}, R^{f} together with the nitrogen atom, to which they are bound may form a 5-, 6 or 7-membered, saturated or unsaturated N-bound heterocyclic radical, which may carry as a ring member a further heteroatom selected from O, S and N and which is unsubstituted or may carry 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{3e}, R^{3f} independently of each other have the meanings given for R^{e}, R^{f};
R^{g} is from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{h} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, a radical C(═O)—R^{k}, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy, or
R^{g}, R^{h} together with the nitrogen atom, to which they are bound may form a 5-, 6 or 7-membered, saturated or unsaturated N-bound heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl, C₁-C₄-haloalkyl, C₁-C₄-alkoxy and C₁-C₄-haloalkoxy;
R^{2g}, R^{2h} independently of each other have the meanings given for R^{g}, R^{h};
R^{3g}, R^{3h} independently of each other have the meanings given for R^{g}, R^{h};
R^{k} has the meanings given for R^{c};
or an N-oxide or an agriculturally suitable salt thereof.

Depending on the substitution pattern, the compounds of the formula I or formula II may have one or more centers of chirality, in which case they are present as mixtures of enantiomers or diastereomers. Useful are both the pure enantiomers or pure diastereomers of the compounds of formula I or formula II, and their mixtures and the use according to the invention of the pure enantiomers or pure diastereomers of the compound of formula I or formula II or its mixtures. Suitable compounds of the formula I or formula II also include all possible geometrical stereoisomers (cis/trans isomers) and mixtures thereof. Cis/trans isomers may be present with respect to an alkene, carbon-nitrogen double-bond, nitrogen-sulfur double bond or amide group. The term “stereoisomer(s)” encompasses both optical isomers, such as enantiomers or diastereomers, the latter existing due to more than one center of chirality in the molecule, as well as geometrical isomers (cis/trans isomers).

Depending on the substitution pattern, the compounds of the formula I or formula II may be present in the form of their tautomers. Hence the invention also relates to the tautomers of the formula I or formula II and the stereoisomers, salts and N-oxides of said tautomers.

The term “N-oxide” includes any compound of the present invention which has at least one tertiary nitrogen atom that is oxidized to an N-oxide moiety. N-oxides in compounds I or II can in particular be prepared by oxidizing the ring nitrogen atom(s) of the oxadiazole ring with a suitable oxidizing agent, such as peroxo carboxylic acids or other peroxides, or the ring nitrogen atom(s) of a heterocyclic substituent X and Y, respectively, R¹, R² or R³.

The HPPD-inhibiting herbicide, in particular the bicycloarylcarboxamide, as described herein relates to compounds as defined herein, wherein one or more of the atoms depicted in formula I or formula II have been replaced by its stable, preferably non-radioactive isotope (e.g., hydrogen by deuterium, ¹²C by ¹³C, ¹⁴N by ¹⁵N, ¹⁶O by ¹⁸O) and in particular wherein at least one hydrogen atom has been replaced by a deuterium atom. Of course, the compounds according to the invention contain more of the respective isotope than this naturally occurs and thus is anyway present in the compounds I.

The HPPD-inhibiting herbicide, in particular the bicycloarylcarboxamide, as described herein may be amorphous or may exist in one ore more different crystalline states (polymorphs) which may have different macroscopic properties such as stability or show different biological properties such as activities. The present invention includes both amorphous and crystalline compounds of formula I or formula II, their enantiomers or diastereomers, mixtures of different crystalline states of the respective compound of formula I or formula II, its enantiomers or diastereomers, as well as amorphous or crystalline salts thereof.

Salts of the HPPD-inhibiting herbicide, in particular the bicycloarylcarboxamide, as described herein, are preferably agriculturally suitable salts. They can be formed in a customary method, e.g. by reacting the compound with an acid if the compound of the present invention has a basic functionality or by reacting the compound with a suitable base if the compound of the present invention has an acidic functionality.

Useful agriculturally suitable salts are especially the salts of those cations or the acid addition salts of those acids whose cations and anions, respectively, do not have any adverse effect on the herbicidal action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting compounds of the present invention with an acid of the corresponding anion, preferably with hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine or bromine.

The term “partially or completely halogenated” will be taken to mean that 1 or more, e.g. 1, 2, 3, 4 or 5 or all of the hydrogen atoms of a given radical have been replaced by a halogen atom, in particular by fluorine or chlorine. A partially or completely halogenated radical is termed below also “halo-radical”. For example, partially or completely halogenated alkyl is also termed haloalkyl.

The term “alkyl” as used herein (and in the alkyl moieties of other groups comprising an alkyl group, e.g. alkoxy, alkylcarbonyl, alkoxycarbonyl, alkylthio, alkylsulfonyl and alkoxyalkyl) denotes in each case a straight-chain or branched alkyl group having usually from 1 to 10 carbon atoms, frequently from 1 to 6 carbon atoms, preferably 1 to 4 carbon atoms and in particular from 1 to 3 carbon atoms. Examples of C₁-C₄-alkyl are methyl, ethyl, n-propyl, iso-propyl, n-butyl, 2-butyl (sec-butyl), isobutyl and tert-butyl. Examples for C₁-C₆-alkyl are, apart those mentioned for C₁-C₄-alkyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethyl butyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Examples for C₁-C₁₀-alkyl are, apart those mentioned for C₁-C₆-alkyl, n-heptyl, 1-methylhexyl, 2-methylhexyl, 3-methylhexyl, 4-methylhexyl, 5-methyl hexyl, 1-ethylpentyl, 2-ethylpentyl, 3-ethylpentyl, n-octyl, 1-methyloctyl, 2-methylheptyl, 1-ethylhexyl, 2-ethylhexyl, 1,2-dimethylhexyl, 1-propylpentyl, 2-propylpentyl, nonyl, decyl, 2-propylheptyl and 3-propylheptyl.

The term “alkylene” (or alkanediyl) as used herein in each case denotes an alkyl radical as defined above, wherein one hydrogen atom at any position of the carbon backbone is replaced by one further binding site, thus forming a bivalent moiety.

The term “haloalkyl” as used herein (and in the haloalkyl moieties of other groups comprising a haloalkyl group, e.g. haloalkoxy, haloalkylthio, haloalkylcarbonyl, haloalkylsulfonyl and haloalkylsulfinyl) denotes in each case a straight-chain or branched alkyl group having usually from 1 to 8 carbon atoms (“C₁-C₈-haloalkyl”), frequently from 1 to 6 carbon atoms (“C₁-C₆-haloalkyl”), more frequently 1 to 4 carbon atoms (“C₁-C₄-haloalkyl”), wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl. Halomethyl is methyl in which 1, 2 or 3 of the hydrogen atoms are replaced by halogen atoms. Examples are bromomethyl, chloromethyl, dichloromethyl, trichloromethyl, fluoromethyl, difluoromethyl, trifluoromethyl, chlorofluoromethyl, dichlorofluoromethyl, chlorodifluoromethyl and the like. Examples for C₁-C₂-fluoroalkyl are fluoromethyl, difluoromethyl, trifluoromethyl, 1-fluoroethyl, 2-fluoroethyl, 2,2-difluoroethyl, 2,2,2-trifluoroethyl, pentafluoroethyl, and the like. Examples for C₁-C₂-haloalkyl are, apart those mentioned for C₁-C₂-fluoroalkyl, chloromethyl, dichloromethyl, trichloromethyl, bromomethyl, chlorofluoromethyl, dichlorofluoromethyl, chlorodifluoromethyl, 1-chloroethyl, 2-chloroethyl, 2,2,-dichloroethyl, 2,2,2-trichloroethyl, 2-chloro-2-fluoroethyl, 2-chloro-2,2-difluoroethyl, 2,2-dichloro-2-fluoroethyl, 1-bromoethyl, and the like. Examples for C₁-C₄-haloalkyl are, apart those mentioned for C₁-C₂-haloalkyl, 1-fluoropropyl, 2-fluoropropyl, 3-fluoropropyl, 3,3-difluoropropyl, 3,3,3-trifluoropropyl, heptafluoropropyl, 1,1,1-trifluoroprop-2-yl, 3-chloropropyl, 4-chlorobutyl and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms (“03-C₁₀-cycloalkyl”), preferably 3 to 7 carbon atoms (“C₃-C₇-cycloalkyl”) or in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl. Examples of monocyclic radicals having 3 to 7 carbon atoms comprise cyclopropyl, cyclobutyl, cyclopentyl, cyclohexyl and cycloheptyl. Examples of bicyclic radicals having 7 or 8 carbon atoms comprise bicyclo[2.1.1]hexyl, bicyclo[2.2.1]heptyl, bicyclo[3.1.1]heptyl, bicyclo[2.2.1]heptyl, bicyclo[2.2.2]octyl and bicyclo[3.2.1]octyl.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, preferably 3 to 7 carbon atoms or in particular 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “cycloalkyl-alkyl” used herein denotes a cycloalkyl group, as defined above, which is bound to the remainder of the molecule via an alkylene group. The term “C₃-C₇-cycloalkyl-C₁-C₄-alkyl” refers to a C₃-C₇-cycloalkyl group as defined above which is bound to the remainder of the molecule via a C₁-C₄-alkyl group, as defined above. Examples are cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, and the like.

The term “alkenyl” as used herein denotes in each case a monounsaturated straight-chain or branched hydrocarbon radical having usually 2 to 8 (“C₂-C₈-alkenyl”), preferably 2 to 6 carbon atoms (“C₂-C₆-alkenyl”), in particular 2 to 4 carbon atoms (“C₂-C₄-alkenyl”), and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₈-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl and the positional isomers thereof.

The term “haloalkenyl” as used herein, which may also be expressed as “alkenyl which may be substituted by halogen”, and the haloalkenyl moieties in haloalkenyloxy and the like refers to unsaturated straight-chain or branched hydrocarbon radicals having 2 to 8 (“C₂-C₈-haloalkenyl”) or 2 to 6 (“C₂-C₆-haloalkenyl”) or 2 to 4 (“C₂-C₄-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like.

The term “alkynyl” as used herein denotes unsaturated straight-chain or branched hydrocarbon radicals having usually 2 to 8 (“C₂-C₈-alkynyl”), frequently 2 to 6 (“C₂-C₆-alkynyl”), preferably 2 to 4 carbon atoms (“C₂-C₄-alkynyl”) and one or two triple bonds in any position, for example C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-di methyl-1-butynyl, 1-ethyl-2-butynyl, 1-ethyl-3-butynyl, 2-ethyl-3-butynyl, 1-ethyl-1-methyl-2-propynyl and the like.

The term “haloalkynyl” as used herein, which is also expressed as “alkynyl which may be substituted by halogen”, refers to unsaturated straight-chain or branched hydrocarbon radicals having usually 3 to 8 carbon atoms (“C₂-C₈-haloalkynyl”), frequently 2 to 6 (“C₂-C₆-haloalkynyl”), preferably 2 to 4 carbon atoms (“C₂-C₄-haloalkynyl”), and one or two triple bonds in any position (as mentioned above), where some or all of the hydrogen atoms in these groups are replaced by halogen atoms as mentioned above, in particular fluorine, chlorine and bromine.

The term “alkoxy” as used herein denotes in each case a straight-chain or branched alkyl group usually having from 1 to 8 carbon atoms (“C₁-C₈-alkoxy”), frequently from 1 to 6 carbon atoms (“C₁-C₆-alkoxy”), preferably 1 to 4 carbon atoms (“C₁-C₄-alkoxy”), which is bound to the remainder of the molecule via an oxygen atom. C₁-C₂-Alkoxy is methoxy or ethoxy. C₁-C₄-Alkoxy is additionally, for example, n-propoxy, 1-methylethoxy (isopropoxy), butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof.

The term “haloalkoxy” as used herein denotes in each case a straight-chain or branched alkoxy group, as defined above, having from 1 to 8 carbon atoms (“C₁-C₈-haloalkoxy”), frequently from 1 to 6 carbon atoms (“C₁-C₆-haloalkoxy”), preferably 1 to 4 carbon atoms (“C₁-C₄-haloalkoxy”), more preferably 1 to 3 carbon atoms (“C₁-C₃-haloalkoxy”), wherein the hydrogen atoms of this group are partially or totally replaced with halogen atoms, in particular fluorine atoms. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “alkoxyalkyl” as used herein denotes in each case alkyl usually comprising 1 to 6 carbon atoms, preferably 1 to 4 carbon atoms, wherein 1 carbon atom carries an alkoxy radical usually comprising 1 to 8, frequently 1 to 6, in particular 1 to 4, carbon atoms as defined above. “C₁-C₆-Alkoxy-C₁-C₆-alkyl” is a C₁-C₆-alkyl group, as defined above, in which one hydrogen atom is replaced by a C₁-C₆-alkoxy group, as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)-ethyl, 2-(2-methyl propoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)-propyl, 2-(1,1-dimethylethoxy)-propyl, 3-(methoxy)-propyl, 3-(ethoxy)-propyl, 3-(n-propoxy)-propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methyl-propoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “haloalkoxy-alkyl” as used herein denotes in each case alkyl as defined above, usually comprising 1 to 6 carbon atoms, preferably 1 to 4 carbon atoms, wherein 1 carbon atom carries an haloalkoxy radical as defined above, usually comprising 1 to 8, frequently 1 to 6, in particular 1 to 4, carbon atoms as defined above. Examples are fluoromethoxymethyl, difluoro-methoxymethyl, trifluoromethoxymethyl, 1-fluoroethoxymethyl, 2-fluoroethoxymethyl, 1,1-difluoroethoxymethyl, 1,2-difluoroethoxymethyl, 2,2-difluoroethoxymethyl, 1,1,2-trifluoroethoxymethyl, 1,2,2-trifluoroethoxymethyl, 2,2,2-trifluoroethoxymethyl, pentafluoroethoxymethyl, 1-fluoroethoxy-1-ethyl, 2-fluoroethoxy-1-ethyl, 1,1-difluoroethoxy-1-ethyl, 1,2-difluoroethoxy-1-ethyl, 2,2-difluoroethoxy-1-ethyl, 1,1,2-trifluoroethoxy-1-ethyl, 1,2,2-trifluoroethoxy-1-ethyl, 2,2,2-trifluoroethoxy-1-ethyl, pentafluoroethoxy-1-ethyl, 1-fluoroethoxy-2-ethyl, 2-fluoroethoxy-2-ethyl, 1,1-difluoroethoxy-2-ethyl, 1,2-difluoroethoxy-2-ethyl, 2,2-difluoroethoxy-2-ethyl, 1,1,2-trifluoroethoxy-2-ethyl, 1,2,2-trifluoroethoxy-2-ethyl, 2,2,2-trifluoroethoxy-2-ethyl, pentafluoroethoxy-2-ethyl, and the like.

The term “alkylthio” (also alkylsulfanyl or S-alkyl)” as used herein denotes in each case a straight-chain or branched saturated alkyl group as defined above, usually comprising 1 to 8 carbon atoms (“C₁-C₃-alkylthio”), frequently comprising 1 to 6 carbon atoms (“C₁-C₆-alkylthio”), preferably 1 to 4 carbon atoms (“C₁-C₄-alkylthio”), which is attached via a sulfur atom at any position in the alkyl group. C₁-C₂-Alkylthio is methylthio or ethylthio. C₁-C₄-Alkylthio is additionally, for example, n-propylthio, 1-methylethylthio (isopropylthio), butylthio, 1-methylpropylthio (sec-butylthio), 2-methylpropylthio (isobutylthio) or 1,1-dimethylethylthio (tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof.

The term “haloalkylthio” as used herein refers to an alkylthio group as defined above wherein the hydrogen atoms are partially or completely substituted by fluorine, chlorine, bromine and/or iodine. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The terms “alkylsulfinyl” and “S(O)ₙ-alkyl” (wherein n is 1) are equivalent and, as used herein, denote an alkyl group, as defined above, attached via a sulfinyl [S(O)] group. For example, the term “C₁-C₂-alkylsulfinyl” refers to a C₁-C₂-alkyl group, as defined above, attached via a sulfinyl [S(O)] group. The term “C₁-C₄-alkylsulfinyl” refers to a C₁-C₄-alkyl group, as defined above, attached via a sulfinyl [S(O)] group. The term “C₁-C₆-alkylsulfinyl” refers to a C₁-C₆-alkyl group, as defined above, attached via a sulfinyl [S(O)] group. C₁-C₂-alkylsulfinyl is methylsulfinyl or ethylsulfinyl. C₁-C₄-alkylsulfinyl is additionally, for example, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl) or 1,1-dimethylethylsulfinyl (tert-butylsulfinyl). C₁-C₆-alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-di methylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The terms “alkylsulfonyl” and “S(O)ₙ-alkyl” (wherein n is 2) are equivalent and, as used herein, denote an alkyl group, as defined above, attached via a sulfonyl [S(O)₂] group. The term “C₁-C₂-alkylsulfonyl” refers to a C₁-C₂-alkyl group, as defined above, attached via a sulfonyl [S(O)₂] group. The term “C₁-C₄-alkylsulfonyl” refers to a C₁-C₄-alkyl group, as defined above, attached via a sulfonyl [S(O)₂] group. The term “C₁-C₆-alkylsulfonyl” refers to a C₁-C₆-alkyl group, as defined above, attached via a sulfonyl [S(O)₂] group. C₁-C₂-alkylsulfonyl is methylsulfonyl or ethylsulfonyl. C₁-C₄-alkylsulfonyl is additionally, for example, n-propylsulfonyl, 1-methylethylsulfonyl (isopropylsulfonyl), butylsulfonyl, 1-methylpropylsulfonyl (sec-butylsulfonyl), 2-methylpropylsulfonyl (isobutylsulfonyl) or 1,1-dimethylethylsulfonyl (tert-butylsulfonyl). C₁-C₆-alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.

The term “alkylamino” as used herein denotes in each case a group —NHR*, wherein R* is a straight-chain or branched alkyl group usually having from 1 to 6 carbon atoms (“C₁-C₆-alkylamino”), preferably 1 to 4 carbon atoms (“C₁-C₄-alkylamino”). Examples of C₁-C₆-alkylamino are methylamino, ethylamino, n-propylamino, isopropylamino, n-butylamino, 2-butylamino, iso-butylamino, tert-butylamino, and the like.

The term “dialkylamino” as used herein denotes in each case a group-NR*R^{∘}, wherein R* and R^{∘}, independently of each other, are a straight-chain or branched alkyl group each usually having from 1 to 6 carbon atoms (“di-(C₁-C₆-alkyl)-amino”), preferably 1 to 4 carbon atoms (“di-(C₁-C₄-alkyl)-amino”). Examples of a di-(C₁-C₆-alkyl)-amino group are dimethylamino, diethylamino, dipropylamino, dibutylamino, methyl-ethyl-amino, methyl-propyl-amino, methyl-isopropylamino, methyl-butyl-amino, methyl-isobutyl-amino, ethyl-propyl-amino, ethyl-isopropylamino, ethyl-butyl-amino, ethyl-isobutyl-amino, and the like.

The suffix “-carbonyl” in a group denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxycarbonyl.

The term “aryl” as used herein refers to a mono-, bi- or tricyclic aromatic hydrocarbon radical such as phenyl or naphthyl, in particular phenyl.

The term “het(ero)aryl” as used herein refers to a mono-, bi- or tricyclic heteroaromatic hydrocarbon radical, preferably to a monocyclic heteroaromatic radical, such as pyridyl, pyrimidyl and the like.

The term “5- or 6-membered monocyclic or 8-, 9- or 10-membered bicyclic saturated, unsaturated or aromatic heterocycle containing 1, 2, 3 or 4 heteroatoms as ring members selected from the groups consisting of N, O and S” as used herein denotes monocyclic or bicyclic heterocyclic radicals, the monocyclic or bicyclic heterocyclic radicals being saturated, unsaturated or aromatic. An unsaturated heterocyclic radical contains at least one C—C and/or C—N and/or N—N double bond(s). A partially unsaturated heterocyclic radical contains less conjugated C—C and/or C—N and/or N—N double bonds than maximally allowed by the size(s) of the ring(s). A fully unsaturated heterocyclic radical contains as many conjugated C—C and/or C—N and/or N—N double bonds as allowed by the size(s) of the ring(s). An aromatic monocyclic heterocyclic radical is a fully unsaturated 5- or 6-membered monocyclic heterocyclic radical. An aromatic bicyclic heterocyclic radical is an 8-, 9- or 10-membered bicyclic heterocyclic radical consisting of a 5- or 6-membered heteroaromatic ring which is fused to a phenyl ring or to another 5- or 6-membered heteroaromatic ring. The heterocyclic radical may be attached to the remainder of the molecule via a carbon ring member or via a nitrogen ring member. As a matter of course, the heterocyclic ring contains at least one carbon ring atom. If the ring contains more than one 0 ring atom, these are not adjacent.

Examples of a 3-, 4-, 5- or 6-membered monocyclic saturated heterocycle include: oxirane-2-yl, aziridine-1-yl, aziridine-2-yl, oxetan-2-yl, azetidine-1-yl, azetidine-2-yl, azetidine-3-yl, thietane-1-yl, thietan-2-yl, thietane-3-yl, tetrahydrofuran-2-yl, tetrahydrofuran-3-yl, tetrahydrothien-2-yl, tetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2-tetrahydropyranyl, 4-tetrahydropyranyl, 1,3-dioxan-5-yl, 1,4-dioxan-2-yl, piperidin-1-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl and 1,2,4-hexahydrotriazin-3-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl and the like.

Examples of a 5- or 6-membered monocyclic partially unsaturated heterocycle include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl and 1,2,4-di- or tetrahydrotriazin-3-yl.

A 5- or 6-membered monocyclic fully unsaturated (including aromatic) heterocyclic ring is e.g. a 5- or 6-membered monocyclic fully unsaturated (including aromatic) heterocyclic ring. Examples are: 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl and 2-pyrazinyl.

Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like.

If two radicals bound on the same nitrogen atom (for example R^{e} and R^{f} or R^{2e} and R^{2f} or R^{g} and R^{h} or R^{2g} and R^{2h}) together with the nitrogen atom, to which they are bound, form a 5-, 6 or 7-membered, saturated or unsaturated N-bound heterocyclic radical, which may carry as a ring member a further heteroatom selected from O, S and N, this is for example pyrrolidine-1-yl, pyrazolidin-1-yl, imidazolidin-1-yl, oxazolidin-3-yl, thiazolidin-3-yl, isoxazolidin-2-yl, isothiazolin-2-yl, [1,2,3]-triazolidin-1-yl, [1,2,3]-triazolidin-2-yl, [1,2,4]-triazolidin-1-yl, [1,2,4]-triazolidin-4-yl, [1,2,3]-oxadiazolidin-2-yl, [1,2,3]-oxadiazolidin-3-yl, [1,2,5]oxadiazolidin-2-yl, [1,2,4]-oxadiazolidin-2-yl, [1,2,4]-oxadiazolidin-4-yl, [1,3,4]-oxadiazolidin-3-yl, [1,2,3]-thiadiazolidin-2-yl, [1,2,3]-thiadiazolidin-3-yl, [1,2,5]-thiadiazolidin-2-yl, [1,2,4]-thiadiazolidin-2-yl, [1,2,4]-thiadiazolidin-4-yl, [1,3,4]-thiadiazolidin-3-yl, piperdin-1-yl, piperazine-1-yl, morpholin-1-yl, thiomorpholin-1-yl, 1-oxothiomorpholin-1-yl, 1,1-dioxothiomorpholin-1-yl, azepan-1-yl, 1,4-diazepan-1-yl, pyrrolin-1-yl, pyrazolin-1-yl, imidazolin-1-yl, oxazolin-3-yl, isoxazolin-2-yl, thiazolin-3-yl, isothiazolin-1-yl, 1,2-dihydropyridin-1-yl, 1,2,3,4-tetrahydropyridin-1-yl, 1,2,5,6-tetrahydropyridin-1-yl, 1,2-dihydropyridazin, 1,6-dihydropyridazin, 1,2,3,4-tetrahydropyridazin-1-yl, 1,2,5,6-tetrahydropyridazin-1-yl, 1,2-dihydropyrimidin, 1,6-dihydropyrimidin, 1,2,3,4-tetrahydropyrimidin-1-yl, 1,2,5,6-tetrahydropyrimidin-1-yl, 1,2-dihydropyrazin-1-yl, 1,2,3,4-tetrahydropyrazin-1-yl, 1,2,5,6-tetrahydropyrazin-1-yl, pyrrol-1-yl, pyrazol-1-yl, imidazol-1-yl, [1,2,3]-1H-triazol-1-yl, [1,2,3]-2H-triazol-2-yl, [1,2,4]-1H-triazol-1-yl and [1,2,4]-4H-triazol-4-yl.

The term “fused 5-, 6-, 7-, 8-, 9- and 10-membered carbocycle or 5-, 6-, 7-, 8-, 9- and 10-membered heterocycle” refers to a carbo- or heterocycle that is adjoined at two consecutive positions with the phenyl group of the radical Cyc-1 or Cyc-1 in such a way that both rings share the ring atoms at said two positions. The fused carbo- and heterocycles may be saturated, partially unsaturated or fully unsaturated and in addition may be mono-, bi- or tricyclic, where each one of the two or three rings of the bi- and tricyclic fused carbo- and heterocycles is either fused to one or two of the other rings, i.e. two rings share two ring atoms, or spiro-linked, i.e. two rings share 1 ring atom. Examples of 5-, 6-, 7-, 8-, 9- and 10-membered fused carbocycles are cyclopentane, cyclohexane, cycloheptane, cyclo[3.3.0]octane, cyclo[4.3.0]nonane, cyclo[4.4.0]decane cyclopentene, cyclohexene and benzene. Examples of 5-, 6-, 7-, 8-, 9- and 10-membered fused carbocycles are pyrrolidine, tetrahydrofuran, tetrahydrothiophen, dihydrofuran, dihydrothiophen, pyrrole, furan, thiopene, thiazole, thiazine, piperidine, tetrahydropyran, tetrahydrothiopyrane, dioxane, piperazine, morpholine, pyridine, azepane, oxepane, thiepane, azepine, oxepine, thiepine, pyrazole, pyrazoline, imidazole, benzimidazole, imidazoline, indole, indoline, chinoline, isochinoline, pyrimidine, oxazole, isoxazole, oxazoline, isoxazoline and the like.

The remarks made below as to preferred embodiments of the variables (substituents) of the compounds of formula I or II are valid on their own as well as preferably in combination with each other, as well as in combination with the stereoisomers, salts, tautomers or N-oxides thereof.

The remarks made below concerning preferred embodiments of the variables further are valid on their own as well as preferably in combination with each other concerning the compounds of formula I or II, where applicable, as well as concerning the uses and methods according to the invention and the composition according to the invention.

Preferred compounds according to the invention are compounds of formula I or II or a stereoisomer, salt or N-oxide thereof, wherein the salt is an agriculturally suitable salt. Further preferred compounds according to the invention are compounds of formula I or II or an N-oxide or salt thereof, especially an agriculturally suitable salt. Particularly preferred compounds according to the invention are compounds of formula I or II or a salt thereof, especially an agriculturally suitable salt thereof.

According to a preferred embodiment of the invention the variable X in the compounds of formula I is selected from the group consisting of halogen, cyano, nitro, C₁-C₆-alkyl, C₂-C₆-alkenyl, C₂-C₆-haloalkenyl, C₁-C₄-alkoxy-C₁-C₄-alkyl, C₃-C₇-cycloalkyl, C₁-C₆-haloalkyl, C(═O)—R^{c}, C(═O)—OR^{d}, C(═O)—NR^{e}R^{f}, NH—C(═O)R^{k} and NR^{g}R^{h}, where R^{c}, R^{d}, R^{e}, R^{f}, R^{k}, R^{g} and R^{h} are as defined above and which preferably have on their own or in particular in combination the following meanings:
R^{c} is hydrogen, C₁-C₆-alkyl C₃-C₇-cycloalkyl, C₂-C₆-alkenyl, C₂-C₆-haloalkenyl, C₁-C₆-haloalkyl or phenyl, in particular C₁-C₄-alkyl or C₁-C₄-haloalkyl;
R^{d} is C₁-C₆-alkyl or C₁-C₆-haloalkyl, in particular C₁-C₄-alkyl;
R^{e}, R^{f} are independently of each other selected from hydrogen, C₁-C₆-alkyl, C₁-C₆-haloalkyl and benzyl, and in particular from the group consisting of hydrogen and C₁-C₄-alkyl; or
R^{e}, R^{f} together with the nitrogen atom, to which they are bound form a 5-, 6- or 7-membered, saturated or unsaturated N-bound heterocyclic radical, which may carry as a ring member a further heteroatom selected from O, S and N and which is unsubstituted or may carry 1, 2, 3 or 4 groups, which are identical or different and selected from the group consisting of halogen, C₁-C₄-alkyl and C₁-C₄-haloalkyl, and in particular R^{e}, R^{f} together with the nitrogen atom, to which they are bound may form a
5-, 6- or 7-membered, saturated N-bound heterocyclic radical, which may carry as a ring member a further heteroatom selected from O, S and N and which is unsubstituted or may carry 1, 2, 3 or 4 methyl groups;
R^{g}, R^{h} are independently of each other selected from hydrogen, C₁-C₆-alkyl, C₁-C₆-haloalkyl and benzyl, and in particular from the group consisting of hydrogen and C₁-C₄-alkyl, or
R^{g}, R^{h} together with the nitrogen atom, to which they are bound form a 5-, 6 or 7-membered, saturated or unsaturated N-bound heterocyclic radical, which may carry as a ring member a further heteroatom selected from O, S and N and which is unsubstituted or may carry 1, 2, 3 or 4 groups, which are identical or different and selected from the group consisting of halogen, C₁-C₄-alkyl and C₁-C₄-haloalkyl, and in particular R^{g}, R^{h} together with the nitrogen atom, to which they are bound may form a 5-, 6- or 7-membered, saturated N-bound heterocyclic radical, which may carry as a ring member a further heteroatom selected from O, S and N and which is unsubstituted or may carry 1, 2, 3 or 4 methyl groups; and
R^{k} is hydrogen, C₁-C₄-alkyl, C₁-C₄-haloalkyl or phenyl, in particular C₁-C₄-alkyl.

According to a more preferred embodiment the variable X of the compounds of the formula I is selected from the group consisting of halogen, cyano, nitro, NH₂, C₁-C₄-alkyl, C₁-C₄-alkoxy-C₁-C₄-alkyl, C₃-C₇-cycloalkyl, C₁-C₄-haloalkyl, C(═O)—R^{c}, C(═O)—OR^{d}, C(═O)—NR^{e}R^{f} and NH—C(═O)R^{k}, where R^{c}, R^{d}, R^{e}, R^{f} and R^{k} are as defined above and which preferably have on their own or in particular in combination the following meanings:
R^{c} is C₁-C₄-alkyl or C₁-C₄-haloalkyl,
R^{d} is C₁-C₄-alkyl,
R^{e} is hydrogen or C₁-C₄-alkyl,
R^{f} is hydrogen or C₁-C₄-alkyl, or
R^{e}, R^{f} together with the nitrogen atom, to which they are bound may form a 5-, 6 or 7-membered, saturated N-bound heterocyclic radical, which may carry as a ring member a further heteroatom selected from O, S and N and which is unsubstituted or may carry 1, 2, 3 or 4 methyl groups, and
R^{k} is C₁-C₄-alkyl.

According to a particularly preferred embodiment of the invention the variable X in the compounds of formula I is selected from halogen, cyano, nitro, C₁-C₄-alkyl, C₃-C₇-cycloalkyl, C₁-C₄-haloalkyl, acetylamino, methoxycarbonyl, ethoxycarbonyl, methylcarbonyl, piperidinylcarbonyl, trifluoromethylcarbonyl, amino, aminocarbonyl, methylaminocarbonyl, dimethylaminocarbonyl and methoxymethyl, in particular from Cl, Br, F, methyl, ethyl, isopropyl, tert-butyl, cyclopropyl, cyclopentyl, cyclohexyl, CF₃, CHF₂, CClF₂, CH₂CF₃, CF₂CF₃, CH₂Cl, CHCl₂, cyano, nitro, acetylamino, methoxycarbonyl, ethoxycarbonyl, methylcarbonyl, piperidinylcarbonyl, trifluoromethylcarbonyl, amino, aminocarbonyl, methylaminocarbonyl, dimethylaminocarbonyl and methoxymethyl.

According to a further preferred embodiment of the invention the variable X in the cornpounds of formula I is a radical OR^{a}, where R^{a} is as defined above and in particular selected from the group consisting of hydrogen, C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl and benzyl, preferably from hydrogen, C₁-C₆-alkyl, C₁-C₆ haloalkyl, C₂-C₆-alkenyl, C₂-C₆-haloalkenyl, C₂-C₆-alkynyl, C₁-C₄-alkoxy-C₁-C₄-alkyl and C₃-C₇-cycloalkyl, which is unsubstituted or partly or completely halogenated, and in particular from hydrogen, C₁-C₄-alkyl, C₁-C₄-haloalkyl, C₂-C₄-alkenyl, C₂-C₄-alkynyl, C₁-C₄-alkoxy-C₁-C₄-alkyl and C₃-C₆-cycloalkyl. In this context R^{a} specifically is hydrogen, CH₃, CH₂H₃, CH(CH₃)₂, CH₂CH₂CH₃, cylcopropyl, cyclobutyl, cyclopentyl, cyclohexyl, CH₂Cl, C(CH₃)₃, CHF₂, CF₃, CH₂CH═CH₂, CH₂C≡CH, CH₂OCH₃, CH₂CH₂OCH₃ and CH₂CH₂OCH₂CH₃.

According to another preferred embodiment of the invention the variable X in the compounds of formula I is phenyl or heterocyclyl, where heterocyclyl is a 5- or 6-membered monocyclic or 8-, 9- or 10-membered bicyclic saturated, partially unsaturated or aromatic heterocycle, which contains 1, 2, 3 or 4 heteroatoms as ring members, which are selected from the group consisting of O, N and S, where phenyl and heterocyclyl are unsubstituted or substituted by 1, 2, 3 or 4 groups R′ which are as defined above and which are independently from one another preferably selected from the group consisting of halogen, C₁-C₄-alkyl, C₃-C₆-cycloalkyl, C₃-C₆-halocycloalkyl, C₁-C₄-haloalkyl, C₁-C₄-alkoxy, C₁-C₄-alkoxy-C₁-C₄-alkyl and C₁-C₆-haloalkyloxy, more preferably from halogen, C₁-C₄-alkyl, C₃-C₆-cycloalkyl, C₁-C₄-haloalkyl and C₁-C₄-alkoxy, in particular from halogen, methyl, ethyl, methoxy and trifluoromethyl, and specifically from Cl, F, Br, methyl, methoxy and trifluoromethyl.

According to a more preferred embodiment of the invention the variable X in the compounds of formula I is phenyl or heterocyclyl, where heterocyclyl is a partially unsaturated or aromatic 5- or 6-membered monocyclic or 9- or 10-membered bicyclic heterocycle containing 1, 2, 3 or 4 heteroatoms as ring members, which are selected from the group consisting of O, N and S, where the bicyclic heterocycle consists of a 5- or 6-membered heteroaromatic ring which is fused to a phenyl ring, and where phenyl and heterocyclyl are unsubstituted or substituted by 1, 2, 3 or 4 groups R′ which independently from one another have the aforementioned preferred meanings.

According to particular preferred embodiments the variable X in the compounds of the formula I is phenyl or heterocyclyl selected from pyridin-2-yl, pyridin-3-yl, pyridin-4-yl, piperidin-2-yl, piperidin-3-yl, piperidin-4-yl, benzisoxazole-2-yl, 1,2,4-oxadiazol-3-yl, 1,2,4-triazol-3-yl, 1-ethylbenzimidazol-2-yl, 4-methylthiazol-2-yl, thiophen-2-yl, furan-2-yl, furan-3-yl, tetrahydrofuran-2-yl, tetrahydrofuran-3-yl, isoxazol-2-yl, isoxazol-3-yl, isoxazol-4-yl, isoxazol-5-yl, oxazol-2-yl, oxazol-3-yl, oxazol-4-yl, oxazol-5-yl, pyrrol-2-yl, pyrrol-3-yl, imidazol-2-yl, imidazol-4-yl, imidazol-5-yl, pyrazol-3-yl, pyrazol-4-yl, pyrazol-5-yl, isothiazol-3-yl, isothiazol-4-yl, isothiazol-5-yl, thiazol-2-yl, thiazol-4-yl, thiazol-5-yl, 1,2,3-triazol-4-yl, 1,2,3-triazol-5-yl, 1,2,5-triazol-3-yl, 1,3,4-triazol-2-yl, 1,2,4-triazol-3-yl, 1,2,4-triazol-5-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, 1,2,4-thiadiazol-3-yl, 1,2,4-thiadiazol-5-yl, 1,3,4-thiadiazol-2-yl, 1,2,3-thiadiazol-4-yl, 1,2,3-thiadiazol-5-yl, 1,2,5-thiadiazol-3-yl, 2H-1,2,3,4-tetrazol-5-yl, 1H-1,2,3,4-tetrazol-1-yl, 1,2,3,4-oxatriazol-5-yl, 1,2,3,5-oxatriazol-4-yl, 1,2,3,4-thiatriazol-5-yl, 1,2,3,5-thiatriazol-4-yl, pyrazin-2-yl, pyrazin-3-yl, pyrimidin-2-yl, pyrimidin-4-yl, pyrimidin-5-yl, pyridazin-3-yl and pyridazin-4-yl, where phenyl and heterocyclyl are unsubstituted or carry 1, 2, or 3 groups R′ which independently from one another have the aforementioned preferred meanings.

According to a preferred embodiment of the invention the variable X in the compounds of formula I is S(O)ₙ—R^{b}, where R^{b} is as defined above and in particular selected from the group consisting of C₁-C₆-alkyl, C₃-C₇-cycloalkyl, C₁-C₆-haloalkyl, C₂-C₆-alkenyl, C₂-C₆-haloalkenyl, C₂-C₆-alkynyl, C₂-C₆-haloalkynyl, phenyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S, where phenyl and heterocyclyl are unsubstituted or substituted by 1, 2 or 3 groups, which are identical or different and preferably selected from the group consisting of halogen, C₁-C₄-alkyl, C₁-C₂-haloalkyl and C₁-C₂-alkoxy.

According to a more preferred embodiment of the invention the variable X in the compounds of formula I is S(O)ₙ-R^{b}, where R^{b} is selected from the group consisting of C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₇-cycloalkyl, phenyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S.

According to an even more preferred embodiment of the invention the variable X in the compounds of formula I is S(O)ₙ—R^{b}, where R^{b} is selected from C₁-C₆-alkyl, C₁-C₆-haloalkyl, C₂-C₆-alkenyl, C₂-C₆-haloalkenyl, C₂-C₆-alkynyl, C₃-C₇-cycloalkyl, phenyl and heterocyclyl, where heterocyclyl is a 6-membered aromatic heterocyclic radical having 1 or 2 nitrogen atoms as ring members.

According to a particularly preferred embodiment of the invention the variable X in the compounds of formula I is S(O)₂—R^{b}, where R^{b} is CH₃, CH₂H₃, CH(CH₃)₂, CH₂CH₂CH₃, CH₂CH═CH₂, CH₂C≡CH or phenyl.

According to specifically preferred embodiments of the invention the variable X in the compounds of formula I is selected from the group consisting of Cl, Br, F, methyl, ethyl, isopropyl, tert-butyl, cyclopropyl, cyclopentyl, cyclohexyl, CF₃, CClF₂, CH₂CF₃, CF₂CF₃, CH₂Cl, CHF₂, CHCl₂, cyano, nitro, acetylamino, benzoylamino, methoxycarbonyl, ethoxycarbonyl, benzoyl, methylcarbonyl, piperidinylcarbonyl, trifluoromethylcarbonyl, amino, aminocarbonyl, methylaminocarbonyl, dimethylaminocarbonyl, methoxymethyl, OH, OCH₃, OCH₂H₃, OCH(CH₃)₂, OCH₂CH₂CH₃, O-cylcopropyl, O-cyclobutyl, O-cyclopentyl, O-cyclohexyl, O—CH₂Cl, O—C(CH₃)₃, O—CHF₂, O—CF₃, O—CH₂CH═CH₂, O—CH₂C≡CH, O—CH₂OCH₃, O—CH₂CH₂OCH₃, O—CH₂CH₂OCH₂CH₃, S(O)₂—CH₃, S(O)₂—CH₂H₃, S(O)₂—CH(CH₃)₂, S(O)₂—CH₂CH₂CH₃, S(O)₂—CH₂CH═CH₂, S(O)₂—CH₂C≡CH and S(O)₂-phenyl, and in particular from methyl, ethyl and methoxy.

According to one embodiment of the invention the variable B in the compounds of formula II is N.

According to another embodiment of the invention the variable B in the compounds of formula II is CH.

According to a preferred embodiment of the invention the variable Y in the compounds of formula II is selected from the group consisting of C₁-C₆-alkyl, C₂-C₆-alkenyl, C₂-C₆-haloalkenyl, C₁-C₄-alkoxy-C₁-C₄-alkyl, C₃-C₇-cycloalkyl, C₁-C₆-haloalkyl, R^{c}—C(═O)—C₁-C₂-alkyl, R^{d}O—C(═O)—C₁-C₂-alkyl, R^{e}R^{f}N—C(═O)—C₁-C₂-alkyl and R^{k}—C(═O)NH—C₁-C₂-alkyl; where R^{c}, R^{d}, R^{e}, R^{f}, R^{k}, R^{g} and R^{h} are as defined above and which preferably have on their own or in particular in combination the following meanings:
R^{c} is hydrogen, C₁-C₆-alkyl, C₃-C₇-cycloalkyl, C₂-C₆-alkenyl, C₂-C₆-haloalkenyl, C₁-C₆-haloalkyl or phenyl, in particular C₁-C₄-alkyl or C₁-C₄-haloalkyl;
R^{d} is C₁-C₆-alkyl or C₁-C₆-haloalkyl, in particular C₁-C₄-alkyl,
R^{e}, R^{f} are independently of each other selected from hydrogen, C₁-C₆-alkyl, C₁-C₆-haloalkyl and benzyl, and in particular from the group consisting of hydrogen and C₁-C₄-alkyl, or
R^{e}, R^{f} together with the nitrogen atom, to which they are bound form a 5-, 6- or 7-membered, saturated or unsaturated N-bound heterocyclic radical, which may carry as a ring member a further heteroatom selected from O, S and N and which is unsubstituted or may carry 1, 2, 3 or 4 groups, which are identical or different and selected from the group consisting of halogen, C₁-C₄-alkyl and C₁-C₄-haloalkyl, and in particular R^{e}, R^{f} together with the nitrogen atom, to which they are bound may form a 5-, 6- or 7-membered, saturated N-bound heterocyclic radical, which may carry as a ring member a further heteroatom selected from O, S and N and which is unsubstituted or may carry 1, 2, 3 or 4 methyl groups;
R^{g}, R^{h} are independently of each other selected from hydrogen, C₁-C₆-alkyl, C₁-C₆-haloalkyl and benzyl and in particular from the group consisting of hydrogen or C₁-C₄-alkyl, or
R^{g}, R^{h} together with the nitrogen atom, to which they are bound form a 5-, 6 or 7-membered, saturated or unsaturated N-bound heterocyclic radical, which may carry as a ring member a further heteroatom selected from O, S and N and which is unsubstituted or may carry 1, 2, 3 or 4 groups, which are identical or different and selected from the group consisting of halogen, C₁-C₄-alkyl and C₁-C₄-haloalkyl, and in particular R^{g}, R^{h} together with the nitrogen atom, to which they are bound may form a 5-, 6- or 7-membered, saturated N-bound heterocyclic radical, which may carry as a ring member a further heteroatom selected from O, S and N and which is unsubstituted or may carry 1, 2, 3 or 4 methyl groups; and
R^{k} is H, C₁-C₄-alkyl, C₁-C₄-haloalkyl or phenyl, in particular C₁-C₄-alkyl.

According to a more preferred embodiment the variable Y of the compounds of formula II is selected from the group consisting of C₁-C₄-alkyl, C₁-C₄-alkoxy-C₁-C₄-alkyl, C₃-C₇-cycloalkyl, C₁-C₄-haloalkyl, R^{c}—C(═O)—C₁-C₂-alkyl, R^{d}O—C(═O)—C₁-C₂-alkyl, R^{e}R^{f}N—C(═O)—C₁-C₂-alkyl and R^{k}—C(═O)NH—C₁-C₂-alkyl, where R^{c}, R^{d}, R^{e}, R^{f} and R^{k} are as defined above and which preferably have on their own or in particular in combination the following meanings:
R^{c} is C₁-C₄-alkyl or C₁-C₄-haloalkyl,
R^{d} is C₁-C₄-alkyl,
R^{e} is hydrogen or C₁-C₄-alkyl,
R^{f} is hydrogen or C₁-C₄-alkyl, or
R^{e}, R^{f} together with the nitrogen atom, to which they are bound may form a 5-, 6 or 7-membered, saturated N-bound heterocyclic radical, which may carry as a ring member a further heteroatom selected from O, S and N and which is unsubstituted or may carry 1, 2, 3 or 4 methyl groups, and
   R^{k} is C₁-C₄-alkyl.

According to a particular preferred embodiment of the invention the variable Y in the compounds of formula II is selected from C₁-C₄-alkyl, C₃-C₇-cycloalkyl, C₁-C₄-haloalkyl and C₁-C₄-alkoxy-C₁-C₄-alkyl, in particular from methyl, ethyl, isopropyl, tert-butyl, cyclopropyl, cyclopentyl, cyclohexyl, CF₃, CHF₂, CClF₂, CH₂CF₃, CF₂CF₃, CH₂Cl, CHCl₂, ethoxyethyl, ethoxymethyl, methoxyethyl and methoxymethyl.

According to another particular preferred embodiment of the invention the variable Y in the compounds of formula II is selected from C₁-C₄-alkyl, C₃-C₇-cycloalkyl, C₁-C₄-haloalkyl, methoxyethyl and methoxymethyl, in particular from methyl, ethyl, isopropyl, tert-butyl, cyclopropyl, cyclopentyl, cyclohexyl, CF₃, CHF₂, CClF₂, CH₂CF₃, CF₂CF₃, CH₂Cl, CHCl₂, methoxyethyl and methoxymethyl.

According to another preferred embodiment of the invention the variable Y in the compounds of formula II is phenyl or heterocyclyl, where heterocyclyl is a 5- or 6-membered monocyclic or 8-, 9- or 10-membered bicyclic saturated, partially unsaturated or aromatic heterocycle, which contains 1, 2, 3 or 4 heteroatoms as ring members, which are selected from the group consisting of O, N and S, where phenyl and heterocyclyl are unsubstituted or substituted by 1, 2, 3 or 4 groups R′ which are as defined above and which are independently from one another preferably selected from the group consisting of halogen, C₁-C₄-alkyl, C₃-C₆-cycloalkyl, C₃-C₆-halocycloalkyl, C₁-C₄-haloalkyl, C₁-C₄-alkoxy, C₁-C₄-alkoxy-C₁-C₄-alkyl and C₁-C₆-haloalkyloxy, more preferably from halogen, C₁-C₄-alkyl, C₃-C₆-cycloalkyl, C₁-C₄-haloalkyl and C₁-C₄-alkoxy, in particular from halogen, methyl, ethyl, methoxy and trifluoromethyl, and specifically from Cl, F, Br, methyl, methoxy and trifluoromethyl.

According to a more preferred embodiment of the invention the variable Y in the cornpounds of formula II is phenyl or heterocyclyl, where heterocyclyl is a partially unsaturated or aromatic 5- or 6-membered monocyclic or 9- or 10-membered bicyclic heterocycle containing 1, 2, 3 or 4 heteroatoms as ring members, which are selected from the group consisting of O, N and S, where the bicyclic heterocycle consists of a 5- or 6-membered heteroaromatic ring which is fused to a phenyl ring, and where phenyl and heterocyclyl are unsubstituted or substituted by 1, 2, 3 or 4 groups R′ which independently from one another have the aforementioned preferred meanings.

According to particular preferred embodiments the variable Y in the compounds of formula II is phenyl or heterocyclyl selected from pyridin-2-yl, pyridin-3-yl, pyridin-4-yl, piperidin-2-yl, piperidin-3-yl, piperidin-4-yl, benzisoxazole-2-yl, 1,2,4-oxadiazol-3-yl, 1,2,4-triazol-3-yl, 1-ethylbenzimidazol-2-yl, 4-methylthiazol-2-yl, thiophen-2-yl, furan-2-yl, furan-3-yl, tetrahydrofuran-2-yl, tetrahydrofuran-3-yl, isoxazol-2-yl, isoxazol-3-yl, isoxazol-4-yl, isoxazol-5-yl, oxazol-2-yl, oxazol-3-yl, oxazol-4-yl, oxazol-5-yl, pyrrol-2-yl, pyrrol-3-yl, imidazol-2-yl, imidazol-4-yl, imidazol-5-yl, pyrazol-3-yl, pyrazol-4-yl, pyrazol-5-yl, isothiazol-3-yl, isothiazol-4-yl, isothiazol-5-yl, thiazol-2-yl, thiazol-4-yl, thiazol-5-yl, 1,2,3-triazol-4-yl, 1,2,3-triazol-5-yl, 1,2,5-triazol-3-yl, 1,3,4-triazol-2-yl, 1,2,4-triazol-3-yl, 1,2,4-triazol-5-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, 1,2,4-thiadiazol-3-yl, 1,2,4-thiadiazol-5-yl, 1,3,4-thiadiazol-2-yl, 1,2,3-thiadiazol-4-yl, 1,2,3-thiadiazol-5-yl, 1,2,5-thiadiazol-3-yl, 2H-1,2,3,4-tetrazol-5-yl, 1H-1,2,3,4-tetrazol-1-yl, 1,2,3,4-oxatriazol-5-yl, 1,2,3,5-oxatriazol-4-yl, 1,2,3,4-thiatriazol-5-yl, 1,2,3,5-thiatriazol-4-yl, pyrazin-2-yl, pyrazin-3-yl, pyrimidin-2-yl, pyrimidin-4-yl, pyrimidin-5-yl, pyridazin-3-yl and pyridazin-4-yl, where phenyl and heterocyclyl are unsubstituted or carry 1, 2, or 3 groups R′ which independently from one another have the aforementioned preferred meanings.

According to a preferred embodiment of the invention the variable Y in the compounds of formula II is R^{b}—S(O)ₙ—C₁-C₃-alkyl, where R^{b} is as defined above and in particular selected from the group consisting of C₁-C₆-alkyl, C₃-C₇-cycloalkyl, C₁-C₆-haloalkyl, C₂-C₆-alkenyl, C₂-C₆-haloalkenyl, C₂-C₆-alkynyl, C₂-C₆-haloalkynyl, phenyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S, where phenyl and heterocyclyl are unsubstituted or substituted by 1, 2 or 3 groups, which are identical or different and preferably selected from the group consisting of halogen, C₁-C₄-alkyl, C₁-C₂-haloalkyl and C₁-C₂-alkoxy.

According to a more preferred embodiment of the invention the variable Y in the compounds of formula II is R^{b}—S(O)ₙ—C₁-C₃-alkyl, where R^{b} is selected from the group consisting of C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₃-C₇-cycloalkyl, phenyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S.

According to an even more preferred embodiment of the invention the variable Y in the compounds of formula II is R^{b}—S(O)ₙ—C₁-C₂-alkyl, where R^{b} is selected from C₁-C₆-alkyl, C₁-C₆-haloalkyl, C₂-C₆-alkenyl, C₂-C₆-haloalkenyl, C₂-C₆-alkynyl, C₃-C₇-cycloalkyl, phenyl and heterocyclyl, where heterocyclyl is a 6-membered aromatic heterocyclic radical having 1 or 2 nitrogen atoms as ring members.

According to a particularly preferred embodiment of the invention the variable Y in the compounds of formula II is R^{b}—S(O)₂—C₁-C₂-alkyl, where R^{b} is CH₃, CH₂H₃, CH(CH₃)₂, CH₂CH₂CH₃, CH₂CH═CH₂, CH₂C≡CH or phenyl.

According to specifically preferred embodiments of the invention the variable Y in the compounds of formula II is selected from the group consisting of methyl, ethyl, isopropyl, tert-butyl, cyclopropyl, cyclopentyl, cyclohexyl, CF₃, CHF₂, CClF₂, CH₂CF₃, CF₂CF₃, CH₂Cl, CHCl₂, methoxyethyl, methoxymethyl, and in particular from methyl and ethyl.

Preferred compounds according to the invention are compounds of formula I or II, wherein R², if present, is selected from the group consisting of halogen, NO₂, cyano, oxo (═O), ═N—R²², where R²² is as defined above and in particular is C₁-C₄-alkoxy or C₁-C₄-haloalkoxy, C₁-C₄-alkyl, C₁-C₄haloalkyl, C₂-C₄-alkenyl, C₂-C₄-alkynyl, C₁-C₄-alkoxy, C₁-C₄-alkoxy-C₁-C₄-alkyl, C₁-C₄-alkoxy-C₁-C₄-alkoxy-C₁-C₄-alkyl, C₁-C₄-alkylthio, C₁-C₄-haloalkylthio, C₁-C₄haloalkoxy, C₃-C₁₀-cycloalkyl, O—C₃-C₁₀-cycloalkyl, C₁-C₄-alkylsulfonyl, C₁-C₄-alkylcarbonyl, C₁-C₄-alkylamino, di-(C₁-C₄-alkyl)-amino and Z²-phenyl, where Z² is as defined herein, and where phenyl is unsubstituted or carries 1, 2 or 3 radicals R²¹ which are as defined above and preferably are indepentendly of one another selected from halogen, C₁-C₄-alkyl, C₁-C₄-alkoxy, C₁-C₄-haloalkyl, C₁-C₄-alkoxy-C₁-C₄-alkyl and C₁-C₄-alkoxy-C₁-C₄-alkoxy, and more preferably from halogen, C₁-C₂-alkyl, C₁-C₂-alkoxy, C₁-C₂-haloalkyl and C₁-C₂-alkoxy-C₁-C₂-alkoxy.

More preferably R², if present, is selected from halogen, NO₂, cyano, oxo, ═N—R²², where R²² is C₁-C₄-alkoxy or C₁-C₄-haloalkoxy, C₁-C₄-alkyl, C₁-C₄-haloalkyl, C₂-C₄-alkenyl, C₂-C₄-alkynyl, C₁-C₄-alkoxy, C₁-C₄-alkoxy-C₁-C₄-alkyl, C₁-C₄-alkoxy-C₁-C₄-alkoxy-C₁-C₄-alkyl, C₁-C₄-alkylthio, C₁-C₄haloalkylthio, C₁-C₄-haloalkoxy, C₃-C₁₀-cycloalkyl, C₁-C₄-alkylsulfonyl, C₁-C₄-alkylcarbonyl, phenyl and benzyl, where phenyl in the last two mentioned radicals is unsubstituted or carries 1, 2 or 3 radicals R²¹ which are as defined above and in particular are indepentendly of one another selected from halogen, C₁-C₄-alkyl, C₁-C₄-alkoxy, C₁-C₄haloalkyl and C₁-C₄-alkoxy-C₁-C₄-alkoxy.

Even more preferably R², if present, is selected from halogen, oxo, C₁-C₄-alkyl, C₁-C₄-haloalkyl, C₂-C₄-alkenyl, C₂-C₄-alkynyl, C₁-C₄-alkoxy, C₁-C₄-alkoxy-C₁-C₄-alkyl, C₁-C₄-alkoxy-C₁-C₄-alkoxy-C₁-C₄-alkyl, C₁-C₄-alkylthio, C₁-C₄-haloalkylthio, C₁-C₄-haloalkoxy, C₁-C₄-alkylsulfonyl, ═N—R²², where R²² is C₁-C₄-alkoxy, and phenyl, where phenyl is unsubstituted or carries 1, 2 or 3 radicals R²¹ which are identical or different and are selected from halogen, C₁-C₄-alkyl and C₁-C₄-alkoxy.

Particularly preferred R², if present, is selected from halogen, oxo, C₁-C₄-alkyl, C₁-C₄-alkoxy, C₁-C₄-alkoxy-C₁-C₄-alkyl, C₁-C₄-alkoxy-C₁-C₄-alkoxy-C₁-C₄-alkyl, C₁-C₄-alkylthio, C₁-C₄-haloalkyl, C₁-C₄-haloalkoxy, C₁-C₄-haloalkylthio, C₃-C₄-alkenyl, C₃-C₄-alkynyl and ═N—R²², where R²² is C₁-C₄-alkoxy.

In particular, R², if present, is selected from halogen, oxo, C₁-C₄-alkyl, C₁-C₄-alkoxy, C₁-C₄-haloalkyl, C₁-C₄-haloalkoxy, C₃-C₄-alkenyl and ═N—R²², where R²² is C₁-C₄-alkoxy.

Specifically, R², if present, is halogen, oxo, C₁-C₄-alkyl, C₁-C₄-alkoxy, C₁-C₄-haloalkoxy, C₃-C₄-alkenyl or ═N—R²², where R²² is C₁-C₄-alkoxy, and more specifically F, Cl, ═O, CH₃, CH₂CH₃, CH₂CH₂CH₃, CH(CH₃)₂, CH₂CH═CH₂, OCF₃, OCHF₂, OCH₂F, OCH₂Cl, OCH₂CH₂F, OCF₂CF₃, OCH₃, OCH₂CH₃, ═N—OCH₃ or ═N—OCH₂CH₃.

Preferred compounds according to the invention are compounds of formula I or II, wherein R⁴ is selected from the group consisting of hydrogen, cyano, halogen, nitro, C₁-C₂-alkyl and C₁-C₂-haloalkyl, in particular from the group consisting of hydrogen, CHF₂, CF₃, CN, NO₂, CH₃ and halogen, which is preferably from Cl, Br and F. Specifically R⁴ is hydrogen.

Preferred compounds according to the invention are compounds of formula I or II, wherein R⁵ is selected from the group consisting of hydrogen, halogen, C₁-C₂-alkyl and C₁-C₂-haloalkyl, and in particular from the group consisting of hydrogen, CHF₂, CF₃ and halogen.

According to a particular embodiment of the invention R⁴ and R⁵ are both hydrogen.

According to a preferred embodiment of the invention the variable CYC in the compound of formula I or II is a radical Cyc-1, as defined above.

According to a more preferred embodiment of the invention the variable Q of the radical Cyc-1 indicates a fused 5- or 6-membered monocyclic hetorcycle or a fused 7-, 8-, 9- or 10-membered spiro-bicyclic heterocycle, where the fused monocyclic heterocycle has 1 or 2 heteroatoms selected from O, S and N as ring members and is unsubstituted or carries 1, 2, 3, 4, 5, 6, 7 or 8 radicals R², where the fused spiro-bicyclic heterocycle has 1, 2, 3 or 4 heteroatoms selected from O, S and N as ring members and is unsubstituted or carries 1, 2, 3, 4, 5, 6, 7, 8, 9 or 10 radicals R², where R² has the herein defined meanings and in particular those mentioned as preferred.

According to an even more preferred embodiment of the invention the variable Q of the radical Cyc-1 indicates a fused 5- or 6-membered monocyclic hetorcycle or a fused 8-, 9- or 10-membered spiro-bicyclic heterocycle which are both either saturated or partially unsaturated, where the fused monocyclic heterocycle has 1 or 2 and the fused spiro-bicyclic heterocycle has 1, 2, 3 or 4 heteroatoms selected from O, S and N as ring members, where S as ring member is unsubstituted or is part of a S(O)₂ group or a S(O) group, and where one carbon atom that is a ring member of the fused monocyclic or spiro-bicyclic heterocycle may be part of a carbonyl group. In addition, according to this embodiment the fused monocyclic heterocycle carries 0, 1, 2, 3, 4 or 5 and the fused spiro-bicyclic heterocycle carries 0, 1, 2, 3, 4, 5, 6 or 7 radicals R², which have the herein defined meanings and in particular are indepentendly of one another selected from halogen, C₁-C₄-alkyl, C₁-C₄-alkoxy, C₁-C₄-haloalkyl, C₁-C₄-haloalkoxy, C₃-C₄-alkenyl and ═N—R²², where R²² is C₁-C₄-alkoxy.

According to a particularly preferred embodiment of the invention the variable Q of the radical Cyc-1 indicates a fused 5- or 6-membered monocyclic hetorcycle or a fused 8- or 9-membered spiro-bicyclic heterocycle which are both either saturated or partially unsaturated, where the fused monocyclic heterocycle has 1 or 2 and the fused spiro-bicyclic heterocycle has 1, 2 or 3 heteroatoms selected from O, S and N as ring members, where S as ring member is unsubstituted or is part of a S(O)₂ group and where one carbon atom that is a ring member of the fused monocyclic or spiro-bicyclic heterocycle may be part of a carbonyl group, where said heterocycle includes one or two S(O)₂ groups and/or one carbonyl group. In addition, according to this embodiment the fused monocyclic heterocycle carries 0, 1, 2 or 3 and the fused spiro-bicyclic heterocycle carries 0, 1, 2, 3 or 4 radicals R², which have the herein defined meanings and in particular are indepentendly of one another are selected from halogen, C₁-C₄-alkyl, C₁-C₄-alkoxy, C₁-C₄-haloalkoxy, C₃-C₄-alkenyl and ═N—R²², where R²² is C₁-C₄-alkoxy.

According to a particular embodiment of the invention the radical CYC of the bicycloarylcarboxamide compound of the formula I or II is a radical Cyc-1 that is selected from the following groups Cyc-1a to Cyc-1h:

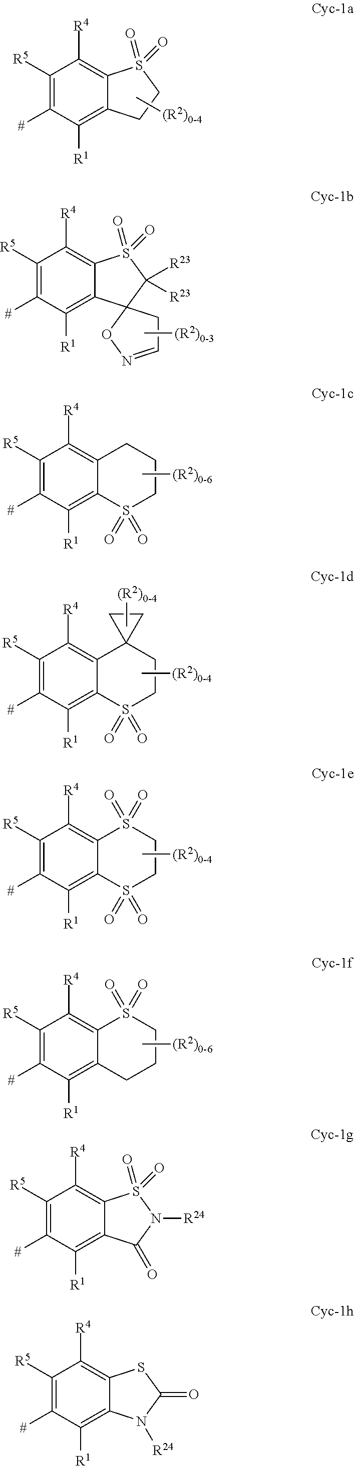

where # indicates the point of attachment of the bi- or tricyclic radical to the carbonyl group of the compound of formula I, R¹, R², R⁴ and R⁵ have the herein defined meanings, in particular those mentioned as preferred, R⁵ is in particular hydrogen or halogen, especially hydrogen, F, Cl or Br, and R²³ and R²⁴ are hydrogen or have one of the meanings given for R² in particular those mentioned as preferred.

According to a specific embodiment of the invention the radical Cyc-1 is selected from the following groups Cyc-1a′ to Cyc-1h′ and Cyc-1f″:

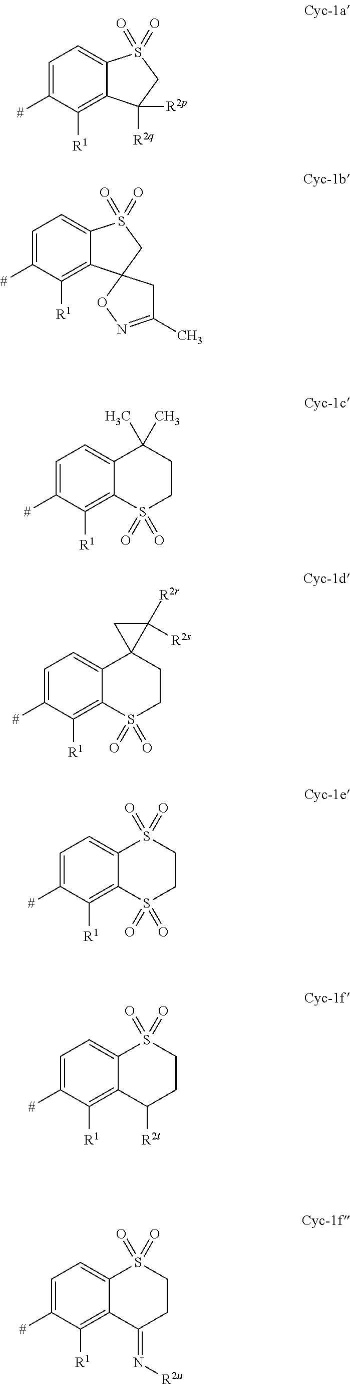

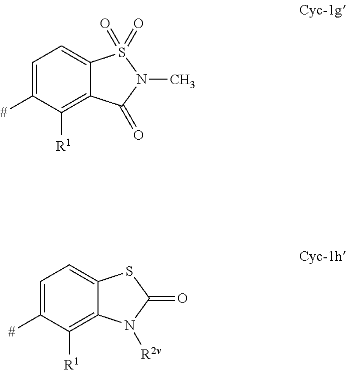

where
# indicates the point of attachment of the bi- or tricyclic radical to the carbonyl group of the compound of formula I;
R¹ has the herein defined meanings, in particular those mentioned herein below as preferred;
R^{2p}, R^{2q} are independently of each other hydrogen, C₁-C₄-alkyl or C₁-C₄-alkoxy, preferably R^{2p} is hydrogen, CH₃, CH₂CH₃ or CH₂(CH₃)₂ and R^{2q} is hydrogen, CH₃, CH₂CH₃, CH₂(CH₃)₂, OCH₃ or OCH₂CH₃, and in particular R^{2p} is hydrogen or CH₃ and R^{2q} is hydrogen, CH₃ or OCH₃;
R^{2r}, R^{2s} are independently of each other hydrogen, halogen or C₁-C₄-alkyl, preferably hydrogen or halogen, and in particular hydrogen, fluorine or chlorine;
R^{2t} is C₁-C₄-alkoxy or C₁-C₄-haloalkoxy, preferably C₁-C₄-haloalkoxy, and in particular OCH₂CH₂F;
R^{2u} is C₁-C₄-alkoxy or C₁-C₄-haloalkoxy, preferably C₁-C₄-alkoxy, and in particular OCH₃ or OCH₂CH₃;
R^{2v} is C₁-C₄-alkyl or C₃-C₄-alkenyl, preferably C₁-C₃-alkyl or C₃-C₄-alkenyl, and in particular CH₃, CH₂CH₃, CH₂CH₂CH₃, CH(CH₃)₂ or CH₂CH═CH₂.

According to an even more specific embodiment of the invention the radical Cyc-1 is selected from the following groups Cyc-1a′-1 to Cyc-1a′-6, Cyc-1 b′, Cyc-1c′, Cyc-1 d′-1 to Cyc-1d′-9, Cyc-1e′, Cyc-1f′, Cyc-1f″-1 and Cyc-1f″-2, Cyc-1fg′, and Cyc-1h′-1 to Cyc-1h′-5:

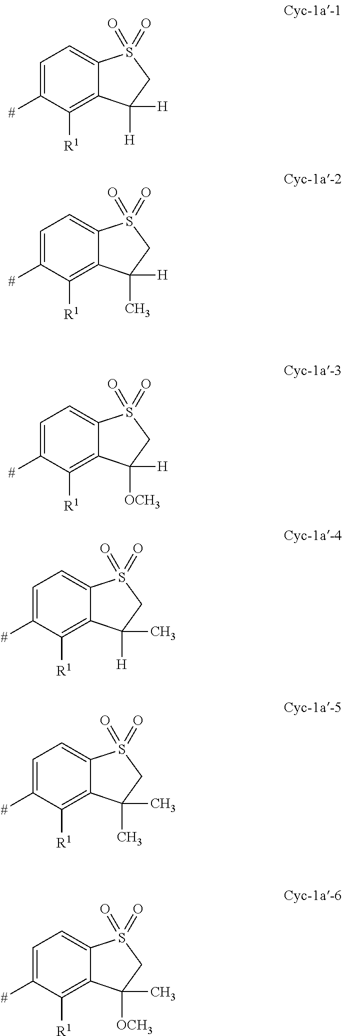

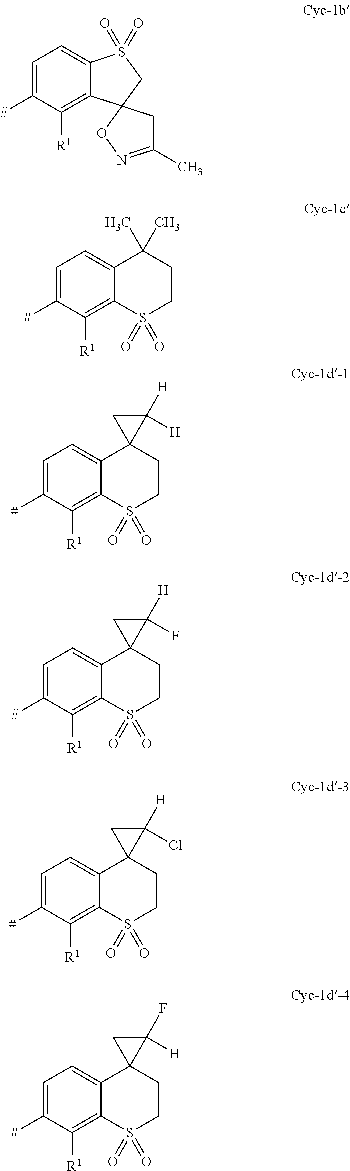

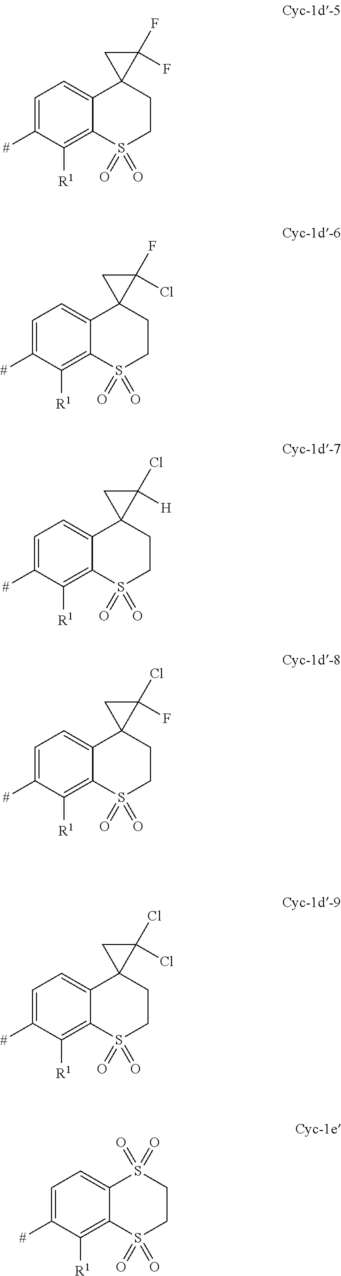

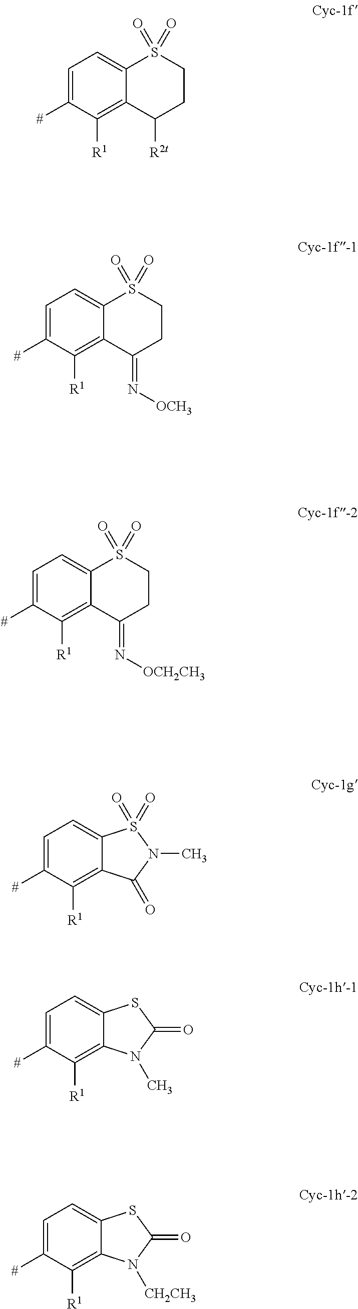

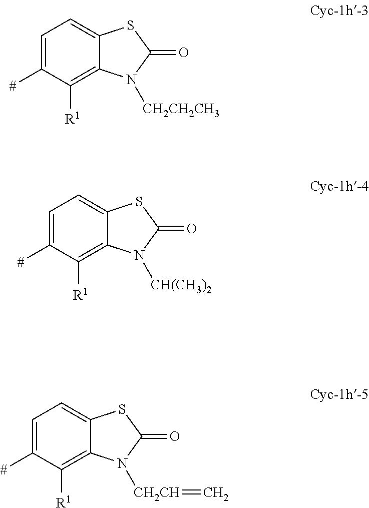

where # indicates the point of attachment of the bi- or tricyclic radical to the carbonyl group of the compound of formula I and R¹ has the herein defined meanings, in particular those mentioned herein below as preferred.

Among the compounds of formula I or II wherein CYC is Cyc-1, preference is given to those compounds, wherein R¹ is selected from the group consisting of CN, halogen, nitro, C₁-C₆-alkyl, C₂-C₆-alkenyl, C₂-C₆-alkynyl, C₁-C₆-haloalkyl, C₁-C₆-alkoxy, C₁-C₄alkoxy, C₁-C₄alkyl, C₁-C₄-haloalkoxy-C₁-C₄-alkyl, Z¹—C₁-C₄-alkoxy-C₁-C₄-alkoxy, C₁-C₄-alkylthio-C₁-C₄-alkyl, Z¹—C₁-C₄-alkylthio-C₁-C₄-alkylthio, C₂-C₆-alkenyloxy, C₂-C₆-alkynyloxy, C₁-C₆-haloalkoxy, C₁-C₄ haloalkoxy-C₁-C₄-alkoxy and S(O)ₖR^{1b}, where k and Z¹ are as defined herein and where R^{1b} is as defined above and in particular selected from the group consisting of C₁-C₄-alkyl and C₁-C₄-haloalkyl. In this context Z¹ is in particular a covalent bond.

More preferably, R¹ is selected from halogen, CN, nitro, C₁-C₄-alkyl, C₁-C₄-haloalkyl, C₁-C₄-haloalkoxy-C₁-C₄-alkyl, C₁-C₄-alkoxy-C₁-C₄-alkoxy-C₁-C₄-alkyl, C₁-C₄-alkylthio-C₁-C₄-alkyl, C₁-C₄-alkoxy, C₁-C₄-haloalkoxy, C₃-C₄-alkenyloxy, C₃-C₄-alkynyloxy, C₁-C₄-alkoxy-C₁-C₄-alkoxy, C₁-C₄-haloalkoxy-C₁-C₄-alkoxy, S(O)ₖ—C₁-C₄-alkyl and S(O)ₖ—C₁-C₄-haloalkyl, where k is 0 or 2.

In particular, R¹ is selected from the group consisting of halogen, C₁-C₄-alkyl, C₁-C₄-haloalkyl, C₁-C₄-alkoxy-C₁-C₄-alkyl, C₁-C₄-alkoxy-C₁-C₄-alkoxy-C₁-C₄-alkyl, C₁-C₄-alkoxy, C₁-C₄-haloalkoxy, C₁-C₄-alkylthio, C₁-C₄-haloalkylthio and C₁-C₄-alkylsufonyl, specifically R¹ is F, Cl, Br, CH₃, CF₃, OCH₃, OCF₃, SCF₃, SO₂CH₃ or CH₂OCH₂CH₂OCH₃, and more specifically R¹ is Cl, CH₃, CF₃ or SO₂CH₃.

According to a preferred embodiment of the invention the variable CYC in the compound of formula I or II is a radical Cyc-2, as defined above.

According to a more preferred embodiment of the invention the variable Q′ of the radical Cyc-1 indicates a fused 5- or 6-membered monocyclic heterocycle or a fused 7-, 8-, 9- or 10-membered bicyclic heterocycle, where the fused monocyclic heterocycle has 1 or 2 heteroatoms selected from O, S and N as ring members and is unsubstituted or carries 1, 2, 3, 4, 5, 6, 7 or 8 radicals R², where the fused bicyclic heterocycle has 1, 2, 3 or 4 heteroatoms selected from O, S and N as ring members and is unsubstituted or carries 1, 2, 3, 4, 5, 6, 7, 8, 9 or 10 radicals R², where R² has the herein defined meanings in particular those mentioned as preferred.

According to an even more preferred embodiment of the invention the variable Q′ of the radical Cyc-2 indicates a fused 5- or 6-membered monocyclic heterocycle or a fused 8-, 9- or 10-membered bicyclic heterocycle which are both either partially unsaturated or fully unsaturated, where the fused monocyclic heterocycle has 1 or 2 and the fused bicyclic heterocycle has 1, 2, 3 or 4 heteroatoms selected from O, S and N as ring members, and where the fused monocyclic heterocycle is unsubstituted or carries 1, 2, 3, 4, 5 or 6 and the fused bicyclic heterocycle is unsubstituted or carries 1, 2, 3, 4, 5, 6, 7 or 8 radicals R², which are as defined herein and in particular are indepentendly of one another selected from halogen, C₁-C₄-alkyl, C₁-C₄-alkoxy, C₁-C₄-haloalkyl, C₁-C₄-haloalkoxy, C₃-C₄-alkenyl and ═N—R²², where R²² is C₁-C₄-alkoxy.

According to a particularly preferred embodiment of the invention the variable Q′ of the radical Cyc-2 indicates a fused aromatic 5- or 6-membered monocyclic heterocycle or a fused aromatic 8-, 9- or 10-membered bicyclic heterocycle, where the fused monocyclic heterocycle has 1 or 2 and the fused bicyclic heterocycle has 1, 2 or 3 heteroatoms selected from O and N as ring members, and where the fused monocyclic heterocycle is unsubstituted or carries 1, 2, 3 or 4 and the fused bicyclic heterocycle is unsubstituted or carries 1, 2, 3, 4, 5 or 6 radicals R², which are as defined herein and in particular are indepentendly of one another selected from halogen, C₁-C₄-alkyl, C₁-C₄-alkoxy and C₁-C₄-haloalkyl.

According to a particular embodiment of the invention the radical CYC of the bicycloarylcarboxamide of the formula I or II is a radical Cyc-2 that is selected from the following groups Cyc-2a to Cyc-2d:

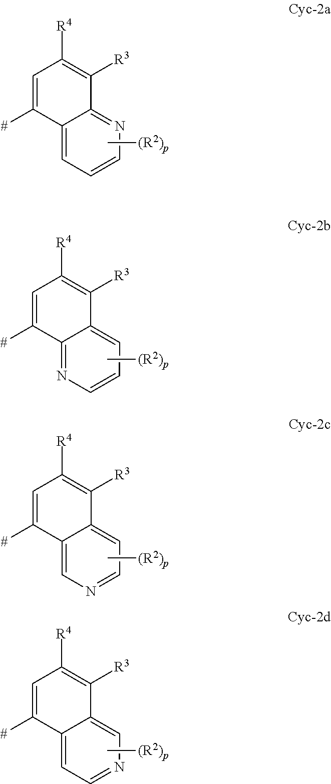

where # indicates the point of attachment of the bicyclic radical to the carbonyl group of the compound of formula I, R², R³ and R⁴ have the herein defined meanings, in particular those mentioned as preferred, and p is 0, 1, 2 or 3, preferably is 0 or 1 and in particular is 0.

According to a specific embodiment of the invention the radical Cyc-1 is selected from the following groups Cyc-2a′ to Cyc-2d′:

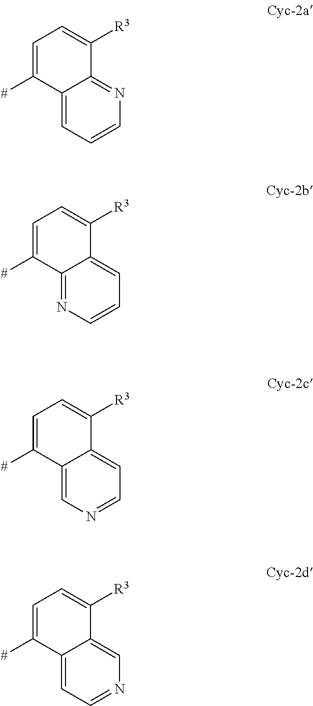

where # indicates the point of attachment of the bicyclic radical to the carbonyl group of the compound of formula I or II and R³ has the herein defined meanings, in particular those mentioned herein below as preferred.

Among the compounds of formula I or II wherein CYC is Cyc-2, preference is given to those compounds, wherein R³ is selected from the group consisting of hydrogen, cyano, halogen, nitro, C₁-C₄-alkyl, C₁-C₄-haloalkyl, C₁-C₄-alkoxy, C₁-C₄-haloalkoxy, C₂-C₄-alkenyl, C₂-C₄-alkynyl, C₂-C₄-alkenyloxy, C₂-C₄-alkynyloxy and S(O)ₖR^{2b}, where the variables k and R^{2b} have one of the herein defined meanings.

More preferably, R³ is selected from the group consisting of hydrogen, halogen, CN, NO₂, C₁-C₄-alkyl, C₁-C₄-haloalkyl, C₁-C₄-alkoxy, C₁-C₄-haloalkoxy, C₁-C₄-alkylthio, C₁-C₄-haloalkylthio, S(O)₂—C₁-C₄-alkyl and S(O)₂—C₁-C₄-haloalkyl.

In particular, R³ is selected from the group consisting of hydrogen, halogen, CN, NO₂, C₁-C₂-alkyl, C₁-C₂-haloalkyl, C₁-C₂-alkoxy, C₁-C₂-haloalkoxy, C₁-C₂-alkylthio, C₁-C₂-haloalkylthio, S(O)₂—C₁-C₂-alkyl and S(O)₂—C₁-C₂-haloalkyl, specifically from hydrogen, Cl, F, ON, NO₂, CH₃, CF₃, CHF₂, OCH₃, OCF₃, OCHF₂, SCH₃, SCF₃, SCHF₂, S(O)₂CH₃ and S(O)₂CH₂CH₃, and more specifically from Cl, F, CN, CF₃ and S(O)₂CH₃.

The variables R′, R¹¹, R²¹, R³¹, Z, Z¹, Z², Z³, Z^{3a}, R^{a}, R^{b}, R^{1b}, R^{2b}, R^{3b}, R^{c}, R^{2c}, R^{3c}, R^{d}, R^{3d}, R^{e}, R^{f}, R^{3e}, R^{3f}, R^{g}, R^{h}, R^{2g}, R^{2h}, R^{3g}, R^{3h}, R^{k}, n and k, independently of each other, preferably have one of the following meanings:
R′, R¹¹, R²¹, R³¹ independently of each other are selected from halogen, C₁-C₄-alkyl, C₃-C₆-cycloalkyl, C₃-C₆-halocycloalkyl, C₁-C₄-haloalkyl, C₁-C₄-alkoxy, C₁-C₄-alkoxy-C₁-C₄-alkyl and C₁-C₆-haloalkyloxy, more preferably from halogen, C₁-C₄-alkyl, C₃-C₆-cycloalkyl, C₁-C₄-haloalkyl and C₁-C₄-alkoxy.

More preferably R′, R¹¹, R²¹, R³¹ independently of each other are selected from the group consisting of halogen, C₁-C₄-alkyl, C₃-C₆-cycloalkyl, C₁-C₄-haloalkyl, C₁-C₄-alkoxy and C₁-C₄-alkoxy-C₁-C₄-alkyl; in particular selected from halogen, C₁-C₄-alkyl, C₁-C₄-alkoxy, C₁-C₄-haloalkyl and C₁-C₄-alkoxy-C₁-C₄-alkyl; and specifically from Cl, F, Br, methyl, ethyl, methoxy and trifluoromethyl.
R²² is selected from C₁-C₄-alkoxy, C₁-C₄-haloalkoxy and C₃-C₇-cycloalkoxy; more preferably from C₁-C₄-alkoxy and C₁-C₄-haloalkoxy, particularly from C₁-C₄-alkoxy, and specifically is OCH₃ or OCH₂CH₃.

Z, Z¹, Z², Z³ independently of each other are selected from a covalent bond, methanediyl and ethanediyl, and in particular are a covalent bond.

Z^{3a} is selected from a covalent bond, C₁-C₂-alkanediyl, O—C₁-C₂-alkanediyl, C₁-C₂-alkanediyl-O and C₁-C₂-alkanediyl-O—C₁-C₂-alkanediyl; more preferably from a covalent bond, methanediyl, ethanediyl, O-methanediyl, O-ethanediyl, methanediyl-O, and ethanediyl-O; and in particular from a covalent bond, methanediyl and ethanediyl.
R^{a} is selected from hydrogen, C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl and benzyl.

More preferably R^{a} is selected from hydrogen, C₁-C₆-alkyl, C₁-C₆-haloalkyl, C₂-C₆-alkenyl, C₂-C₆-haloalkenyl, C₂-C₆-alkynyl, C₁-C₄-alkoxy-C₁-C₄-alkyl and C₃-C₇-cycloalkyl, which is unsubstituted or partly or completely halogenated, and in particular selected from hydrogen, C₁-C₄-alkyl, C₁-C₄-haloalkyl, C₂-C₄-alkenyl, C₂-C₄-alkynyl, C₁-C₄-alkoxy-C₁-C₄-alkyl and C₃-C₆-cycloalkyl.
R^{b}, R^{1b}, R^{2b}, R^{3b} independently of each other are selected from C₁-C₆-alkyl, C₃-C₇-cycloalkyl, C₁-C₆-haloalkyl, C₂-C₆-alkenyl, C₂-C₆-haloalkenyl, C₂-C₆-alkynyl, C₂-C₆-haloalkynyl and phenyl, where phenyl is unsubstituted or substituted by 1, 2 or 3 groups, which are identical or different and selected from the group consisting of halogen, C₁-C₄-alkyl, C₁-C₂-haloalkyl and C₁-C₂-alkoxy.

More preferably R^{b}, R^{1b}, R^{2b}, R^{3b} independently of each other are selected from the group consisting of C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl and phenyl.

In particular, R^{b}, R^{1b}, R^{2b}, R^{3b} independently of each other are selected from C₁-C₄-alkyl, C₁-C₄-haloalkyl, C₂-C₄-alkenyl, C₂-C₄-haloalkenyl, C₂-C₄-alkynyl, C₃-C₆-cycloalkyl and phenyl.
R^{c}, R^{2c}, R^{3c}, R^{k} independently of each other are selected from hydrogen, C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl, benzyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S, where phenyl, benzyl and heterocyclyl are unsubstituted or substituted by 1, 2 or 3 groups, which are identical or different and selected from the group consisting of halogen, C₁-C₄-alkyl, C₁-C₄-haloalkyl and C₁-C₄-alkoxy.

More preferably R^{c}, R^{2c}, R^{3c}, R^{k} independently of each other are selected from hydrogen, C₁-C₄-alkyl, C₁-C₄-haloalkyl, C₂—C-alkenyl, C₂—C-haloalkenyl, C₂—C-alkynyl, C₃-C₆-cycloalkyl, phenyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S.

In particular, R^{c}, R^{2c}, R^{3c}, R^{k} independently of each other are selected from hydrogen, C₁-C₄-alkyl, C₁-C₄-haloalkyl, C₂-C₄-alkenyl, C₂-C₄-haloalkenyl, C₃-C₆-cycloalkyl, phenyl and heterocyclyl, where heterocyclyl is a 5- or 6-membered aromatic heterocyclic radical having 1 or 2 nitrogen atoms as ring members.
R^{d}, R^{3d} independently of each other are selected from C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl and benzyl. More preferably R^{d}, R^{3d} independently of each other are selected from C₁-C₆-alkyl, C₁-C₆-haloalkyl, C₂-C₆-alkenyl, C₂-C₆-haloalkenyl, C₂-C₆-alkynyl, C₁-C₄-alkoxy-C₁-C₄-alkyl and C₃-C₇-cycloalkyl, which is unsubstituted or partly or completely halogenated, and in particular selected from C₁-C₄-alkyl, C₁-C₄-haloalkyl, C₂-C₄-alkenyl, C₂-C₄-haloalkenyl, C₂-C₄-alkynyl and C₃-C₆-cycloalkyl.
R^{e}, R^{f}, R^{3e}, R^{3f} independently of each other are selected from the group consisting of hydrogen, C₁-C₆-alkyl, C₃-C₇-cycloalkyl, which is unsubstituted or partially or completely halogenated, C₁-C₆-haloalkyl, C₂-C₆-alkenyl, C₂-C₆-haloalkenyl, C₁-C₄-alkoxy-C₁-C₄-alkyl, phenyl and benzyl, where phenyl and benzyl are unsubstituted or substituted by 1, 2 or 3 groups, which are identical or different and selected from the group consisting of halogen, C₁-C₄-alkyl, C₁-C₄-haloalkyl and C₁-C₄-alkoxy, or R^{e} and R^{f} or R^{3e} and R^{3f} together with the nitrogen atom, to which they are bound may form a 5-, 6 or 7-membered, saturated or unsaturated N-bound heterocyclic radical, which may carry as a ring member a further heteroatom selected from O, S and N and which is unsubstituted or may carry 1, 2, 3 or 4 groups, which are identical or different and selected from the group consisting of halogen, C₁-C₄-alkyl, C₁-C₄-haloalkyl and C₁-C₄-alkoxy.

More preferably R^{e}, R^{f}, R^{3e}, R^{3f} independently of each other are selected from hydrogen, C₁-C₆-alkyl, C₁-C₆-haloalkyl and benzyl, or R^{e} and R^{f} or R^{3e} and R^{3f} together with the nitrogen atom, to which they are bound may form a 5- or 6-membered, saturated or unsaturated N-bound heterocyclic radical, which may carry as a ring member a further heteroatom selected from O, S and N and which is unsubstituted or may carry 1, 2 or 3 groups, which are identical or different and selected from the group consisting of halogen, C₁-C₄-alkyl and C₁-C₄-haloalkyl.

In particular, R^{e}, R^{f}, R^{3e}, R^{3f} independently of each other are selected from hydrogen and C₁-C₄-alkyl, or R^{e} and R^{f} or R^{3e} and R^{3f} together with the nitrogen atom, to which they are bound may form a 5- or 6-membered, saturated N-bound heterocyclic radical, which may carry as a ring member a further heteroatom selected from O, S and N and which is unsubstituted or may carry 1, 2 or 3 methyl groups.
R^{g}, R^{2g}, R^{3g} independently of each other are selected from hydrogen, C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl and benzyl.

More preferably R^{g}, R^{2g}, R^{3g} independently of each other are selected from hydrogen, C₁-C₆-alkyl, C₁-C₆-haloalkyl, C₂-C₆-alkenyl, C₂-C₆-haloalkenyl, benzyl, C₁-C₄-alkoxy-C₁-C₄-alkyl and C₃-C₇-cycloalkyl, which is unsubstituted or partly or completely halogenated, and in particular selected from hydrogen, C₁-C₄-alkyl, C₁-C₄-haloalkyl, C₂-C₄-alkenyl, C₂-C₄-haloalkenyl, benzyl and C₃-C₆-cycloalkyl.
R^{h}, R^{2h}, R^{3h} independently of each other are selected from hydrogen, C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₂-C₆-haloalkynyl, C₁-C₄-alkoxy-C₁-C₄-alkyl, phenyl, benzyl and a radical C(═O)—R^{k}, where R^{k} is hydrogen, C₁-C₄-alkyl, C₁-C₄-haloalkyl or phenyl.

More preferably R^{h}, R^{2h}, R^{3h} independently of each other are selected from hydrogen, C₁-C₆-alkyl, C₁-C₆-haloalkyl, C₂-C₆-alkenyl, C₂-C₆-haloalkenyl, benzyl, C₁-C₄-alkoxy-C₁-C₄-alkyl and C₃-C₇-cycloalkyl, which is unsubstituted or partly or completely halogenated, and in particular selected from hydrogen, C₁-C₄-alkyl, C₁-C₄-haloalkyl, C₂-C₄-alkenyl, C₂-C₄-haloalkenyl, benzyl and C₃-C₆-cycloalkyl; or
R^{g} and R^{h} or R^{2g} and R^{2h} or R^{3g} and R^{3h} together with the nitrogen atom, to which they are bound may form a 5-, 6 or 7-membered, saturated or unsaturated N-bound heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl and C₁-C₄-haloalkyl and C₁-C₄-alkoxy;
more preferably R^{g} and R^{h} or R^{2g} and R^{2h} or R^{3g} and R^{3h} together with the nitrogen atom, to which they are bound may form a 5- or 6-membered, saturated or unsaturated N-bound heterocyclic radical, which may carry as a ring member a further heteroatom selected from O, S and N and which is unsubstituted or may carry 1, 2 or 3 groups, which are identical or different and selected from the group consisting of halogen, C₁-C₄-alkyl and C₁-C₄-haloalkyl;
and in particular, R^{g} and R^{h} or R^{2g} and R^{2h} or R^{3g} and R^{3h} together with the nitrogen atom, to which they are bound may form a 5- or 6-membered, saturated N-bound heterocyclic radical, which may carry as a ring member a further heteroatom selected from O, S and N and which is unsubstituted or may carry 1, 2 or 3 methyl groups.
n and k independently of each other are 0 or 2, and in particular 2.

The HPPD-inhibiting herbicides useful for the present invention are often best applied in conjunction with one or more other HPPD- and/or HST targeting herbicides to obtain control of a wider variety of undesirable vegetation. When used in conjunction with other HPPD- and/or HST targeting herbicides, the presently claimed compounds can be formulated with the other herbicide or herbicides, tank mixed with the other herbicide or herbicides, or applied sequentially with the other herbicide or herbicides.

Some of the herbicides that are useful in conjunction with the HPPD-inhibiting herbicides of the present invention include benzobicyclon, mesotrione, sulcotrione, tefuryltrione, tembotrione, 4-hydroxy-3-[[2-(2-methoxyethoxy)methyl]-6-(trifluoromethyl)-3-pyridinyl]carbonylFbicyclo[3.2.1]-oct-3-en-2-one (bicyclopyrone), ketospiradox or the free acid thereof, benzofenap, pyrasulfotole, pyrazolynate, pyrazoxyfen, topramezone, [2-chloro-3-(2-methoxyethoxy)-4-(methylsulfonyl)pheny]-(I-ethyl-5-hydroxy-1H-pyrazol-4-ylymethanone, (2,3-dihydro-3,3,4-trimethyl-1,1-dioxidobenzo[b]thien-5-yl)(5-hydroxy-1-methyl-1H-pyrazol-4-yl)-methanone, isoxachlortole, isoxaflutole, α-(cyclopropylcarbonyl)-2-(methylsulfonyl)-β-oxo-4-chloro-benzenepropanenitrile, and α-(cyclopropylcarbonyl)-2-(methylsulfonyl)-β-oxo-4-(trifluoromethyl)-benzenepropanenitrile.

In a preferred embodiment the additional herbicide is topramezone.

In a particularly preferred embodiment the additional herbicide is
(1-Ethyl-5-prop-2-ynyloxy-1H-pyrazol-4-yl)-[4-methansulfonyl-2-methyl-3-(3-methyl-4,5-dihydro-isoxazol-5-yl)-phenyl]-methanon

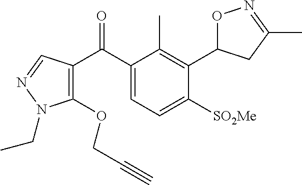 [SMILES: C#CCOc1c(C(=O)c2ccc(S(C)(=O)=O)c(C3CC(C)=NO3)c2C)cnn1CC]

or
(1-Ethyl-5-hydroxy-1H-pyrazol-4-yl)-[4-methansulfonyl-2-methyl-3-(3-methyl-4,5-dihydro-isoxazol-5-yl)-phenyl]methanon

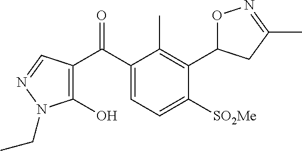 [SMILES: CCn1ncc(C(=O)c2ccc(S(C)(=O)=O)c(C3CC(C)=NO3)c2C)c1O]

The above described compounds are described in great detail in WO2011/067184 which is entirely incorporated herein by reference.

The herbicidal compounds useful for the present invention may further be used in conjunction with additional herbicides to which the crop plant is naturally tolerant, or to which it is resistant via expression of one or more additional transgenes as mentioned supra. Some of the herbicides that can be employed in conjunction with the compounds of the present invention include sulfonamides such as metosulam, flumetsulam, cloransulam-methyl, diclosulam, penoxsulam and florasulam, sulfonylureas such as chlorimuron, tribenuron, sulfometuron, nicosulfuron, chlorsulfuron, amidosulfuron, triasulfuron, prosulfuron, tritosulfuron, thifensulfuron, sulfosulfuron and metsulfuron, imidazolinones such as imazaquin, imazapic, ima-zethapyr, imzapyr, imazamethabenz and imazamox, phenoxyalkanoic acids such as 2,4-D, MCPA, dichlorprop and mecoprop, pyridinyloxyacetic acids such as triclopyr and fluroxypyr, carboxylic acids such as clopyralid, picloram, aminopyralid and dicamba, dinitroanilines such as trifluralin, benefin, benfluralin and pendimethalin, chloroacetanilides such as alachlor, acetochlor and metolachlor, semicarbazones (auxin transport inhibitors) such as chlorflurenol and diflufenzopyr, aryloxyphenoxypropionates such as fluazifop, haloxyfop, diclofop, clodinafop and fenoxaprop and other common herbicides including glyphosate, glufosinate, acifluorfen, bentazon, clomazone, fumiclorac, fluometuron, fomesafen, lactofen, linuron, isoproturon, simazine, norflurazon, paraquat, diuron, diflufenican, picolinafen, cinidon, sethoxydim, tralkoxydim, quinmerac, isoxaben, bromoxynil, metribuzin and mesotrione.

The HPPD-inhibiting herbicides employed for the present invention can, further, be used in conjunction with glyphosate and glufosinate on glyphosate-tolerant or glufosinate-tolerant crops.

Unless already included in the disclosure above, the HPPD-inhibiting herbicides of the present invention can, further, be used in conjunction with compounds:

a) from the group of Lipid Biosynthesis Inhibitors:
Alloxydim, Alloxydim-natrium, Butroxydim, Clethodim, Clodinafop, Clodinafop-propargyl, Cycloxydim, Cyhalofop, Cyhalofop-butyl, Diclofop, Diclofop-methyl, Fenoxaprop, Fenoxapropethyl, Fenoxaprop-P, Fenoxaprop-P-ethyl, Fluazifop, Fluazifop-butyl, Fluazifop-P, Fluazifop-P-butyl, Haloxyfop, Haloxyfop-methyl, Haloxyfop-P, Haloxyfop-P-methyl, Metamifop, Pinoxaden, Profoxydim, Propaquizafop, Quizalofop, Quizalofop-ethyl, Quizalofop-tefuryl, Quizalofop-P, Quizalofop-P-ethyl, Quizalofop-P-tefuryl, Sethoxydim, Tepraloxydim, Tralkoxydim, Benfuresat, Butylat, Cycloat, Dalapon, Dimepiperat, EPIC, Esprocarb, Ethofumesat, Flupropanat, Molinat, Orbencarb, Pebulat, Prosulfocarb, TCA, Thiobencarb, Tiocarbazil, Triallat and Vernolat;
b) from the group of ALS-Inhibitors:
Amidosulfuron, Azimsulfuron, Bensulfuron, Bensulfuron-methyl, Bispyribac, Bispyribac-natrium, Chlorimuron, Chlorimuron-ethyl, Chlorsulfuron, Cinosulfuron, Cloransulam, Cloransulam-methyl, Cyclosulfamuron, Diclosulam, Ethametsulfuron, Ethametsulfuron-methyl, Ethoxysulfuron, Flazasulfuron, Florasulam, Flucarbazon, Flucarbazon-natrium, Flucetosulfuron, Flumetsulam, Flupyrsulfuron, Flupyrsulfuron-methyl-natrium, Foramsulfuron, Halosulfuron, Halosulfuron-methyl, Imazamethabenz, Imazamethabenz-methyl, Imazamox, Imazapic, Imazapyr, Imazaquin, Imazethapyr, Imazosulfuron, Iodosulfuron, Iodosulfuron-methyl-natrium, Mesosulfuron, Metosulam, Metsulfuron, Metsulfuron-methyl, Nicosulfuron, Orthosulfamuron, Oxasulfuron, Penoxsulam, Primisulfuron, Primisulfuron-methyl, Propoxycarbazon, Propoxycarbazon-natrium, Prosulfuron, Pyrazosulfuron, Pyrazosulfuron-ethyl, Pyribenzoxim, Pyrimisulfan, Pyriftalid, Pyriminobac, Pyriminobac-methyl, Pyrithiobac, Pyrithiobac-natrium, Pyroxsulam, Rimsulfuron, Sulfometuron, Sulfometuron-methyl, Sulfosulfuron, Thiencarbazon, Thiencarbazon-methyl, Thifensulfuron, Thifensulfuron-methyl, Triasulfuron, Tribenuron, Tribenuron-methyl, Trifloxysulfuron, Triflusulfuron, Triflusulfuron-methyl and Tritosulfuron;
c) from the group of Photosynthese-Inhibitors:
Ametryn, Amicarbazon, Atrazin, Bentazon, Bentazon-natrium, Bromacil, Bromofenoxim, Bromoxynil and its salts and esters, Chlorobromuron, Chloridazon, Chlorotoluron, Chloroxuron, Cyanazin, Desmedipham, Desmetryn, Dimefuron, Dimethametryn, Diquat, Diquat-dibromid, Diuron, Fluometuron, Hexazinon, loxynil and its salts and esters, Isoproturon, Isouron, Karbutilat, Lenacil, Linuron, Metamitron, Methabenzthiazuron, Metobenzuron, Metoxuron, Metribuzin, Monolinuron, Neburon, Paraquat, Paraquat-dichlorid, Paraquat-dimetilsulfat, Pentanochlor, Phenmedipham, Phenmedipham-ethyl, Prometon, Prometryn, Propanil, Propazin, Pyridafol, Pyridat, Siduron, Simazin, Simetryn, Tebuthiuron, Terbacil, Terbumeton, Terbuthylazin, Terbutryn, Thidiazuron and Trietazin;
d) from the group of Protoporphyrinogen-IX-Oxidase-lnhibitors:
Acifluorfen, Acifluorfen-natrium, Azafenidin, Bencarbazon, Benzfendizon, Benzoxazinone (as described in WO2010/145992), Bifenox, Butafenacil, Carfentrazon, Carfentrazon-ethyl, Chlomethoxyfen, Cinidon-ethyl, Fluazolat, Flufenpyr, Flufenpyr-ethyl, Flumiclorac, Flumicloracpentyl, Flumioxazin, Fluoroglycofen, Fluoroglycofen-ethyl, Fluthiacet, Fluthiacet-methyl, Fomesafen, Halosafen, Lactofen, Oxadiargyl, Oxadiazon, Oxyfluorfen, Pentoxazon, Profluazol, Pyraclonil, Pyraflufen, Pyraflufen-ethyl, Saflufenacil, Sulfentrazon, Thidiazimin, 2-Chlor-5-[3,6-dihydro-3-methyl-2,6-dioxo-4-(trifluormethyl)-1(2H)-pyrimidinyl]-4-fluor-N-[(isopropyl)methylsulfamoyl]benzamid (H-1; CAS 372137-35-4), [3-[2-Chlor-4-fluor-5-(1-methyl-6-trifluormethyl-2,4-dioxo-1,2,3,4,-tetrahydropyrimidin-3-yl)phenoxy]-2-pyridyloxy]acetic acid-ethylester (H-2; CAS 353292-31-6), N-Ethyl-3-(2,6-dichlor-4-trifluormethylphenoxy)-5-methyl-1H-pyrazol-1-carboxamid (H-3; CAS 452098-92-9), N-Tetrahydrofurfuryl-3-(2,6-dichlor-4-trifluormethylphenoxy)-5-methyl-1H-pyrazol-1-carboxamid (H-4; CAS 915396-43-9), N-Ethyl-3-(2-chlor-6-fluor-4-trifluormethylphenoxy)-5-methyl-1H-pyrazol-1-carboxamid (H-5; CAS 452099-05-7) and N-Tetrahydrofurfuryl-3-(2-chlor-6-fluor-4-trifluormethylphenoxy)-5-methyl-1H-pyrazol-1-carboxamid (H-6; CAS 45100-03-7);
e) from the group of Bleacher-Herbicides:
Aclonifen, Amitrol, Beflubutamid, Benzobicyclon, Benzofenap, Clomazon, Diflufenican, Fluridon, Flurochloridon, Flurtamon, Isoxaflutol, Mesotrion, Norflurazon, Picolinafen, Pyrasulfutol, Pyrazolynat, Pyrazoxyfen, Sulcotrion, Tefuryltrion, Tembotrion, Topramezon, 4-Hydroxy-3-[[2-[(2-methoxyethoxy)methyl]-6-(trifluormethyl)-3-pyridyl]carbonyl]bicyclo[3.2.1]oct-3-en-2-one (H-7; CAS 352010-68-5) and 4-(3-Trifluormethylphenoxy)-2-(4-trifluormethylphenyl)pyrimidin (H-8; CAS 180608-33-7);
f) from the group of EPSP-Synthase-Inhibitors:

### Glyphosat, Glyphosat-isopropylammonium and Glyphosat-trimesium (Sulfosat);

g) from the group of Glutamin-Synthase-Inhibitors:

### Bilanaphos (Bialaphos), Bilanaphos-natrium, Glufosinat and Glufosinat-ammonium;

h) from the group of DHP-Synthase-Inhibitors: Asulam;
i) from the group of Mitose-Inhibitors:
Amiprophos, Amiprophos-methyl, Benfluralin, Butamiphos, Butralin, Carbetamid, Chlorpropham, Chlorthal, Chlorthal-dimethyl, Dinitramin, Dithiopyr, Ethalfluralin, Fluchloralin, Oryzalin, Pendimethalin, Prodiamin, Propham, Propyzamid, Tebutam, Thiazopyr and Trifluralin;
j) from the group of VLCFA-Inhibitors:
Acetochlor, Alachlor, Anilofos, Butachlor, Cafenstrol, Dimethachlor, Dimethanamid, Dimethenamid-P, Diphenamid, Fentrazamid, Flufenacet, Mefenacet, Metazachlor, Metolachlor, Metolachlor-S, Naproanilid, Napropamid, Pethoxamid, Piperophos, Pretilachlor, Propachlor, Propisochlor, Pyroxasulfon (KIH-485) and Thenylchlor;
Compounds of the formula 2:

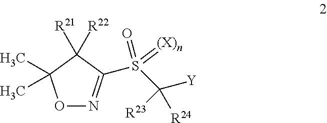

Particularly preferred Compounds of the formula 2 are:

3-[5-(2,2-Difluor-ethoxy)-1-methyl-3-trifluormethyl-1H-pyrazol-4-ylmethansulfonyl]-4-fluor-5,5-dimethyl-4,5-dihydro-isoxazol (2-1); 3-{[5-(2,2-Difluor-ethoxy)-1-methyl-3-trifluormethyl-1H-pyrazol-4-yl]-fluor-methansulfonyl}-5,5-dimethyl-4,5-dihydro-isoxazol (2-2); 4-(4-Fluor-5,5-dimethyl-4,5-dihydro-isoxazol-3-sulfonylmethyl)-2-methyl-5-trifluormethyl-2H-[1,2,3]triazol (2-3); 4-[(5,5-Dimethyl-4,5-dihydro-isoxazol-3-sulfonyl)-fluor-methyl]-2-methyl-5-trifluormethyl-2H-[1,2,3]triazol (2-4); 4-(5,5-Dimethyl-4,5-dihydro-isoxazol-3-sulfonylmethyl)-2-methyl-5-trifluormethyl-2H-[1,2,3]triazol (2-5); 3-{[5-(2,2-Difluor-ethoxy)-1-methyl-3-trifluormethyl-1H-pyrazol-4-yl]-difluor-methansulfonyl}-5,5-dimethyl-4,5-dihydro-isoxazol (2-6); 4-[(5,5-Dimethyl-4,5-dihydro-isoxazol-3-sulfonyl)-difluor-methyl]-2-methyl-5-trifluormethyl-2H-[1,2,3]triazol (2-7); 3-{[5-(2,2-Difluor-ethoxy)-1-methyl-3-trifluormethyl-1H-pyrazol-4-yl]-difluor-methansulfonyl}-4-fluor-5,5-dimethyl-4,5-dihydro-isoxazol (2-8); 4-[Difluor-(4-fluor-5,5-dimethyl-4,5-dihydro-isoxazol-3-sulfonyl)-methyl]-2-methyl-5-trifluormethyl-2H-[1,2,3]triazol (2-9);
k) from the group of Cellulose-Biosynthese-Inhibitors:

### Chlorthiamid, Dichlobenil, Flupoxam and Isoxaben;

l) from the group of Uncoupling-Herbicides:
Dinoseb, Dinoterb and DNOC and its salts;
m) from the group of Auxin-Herbicides:
2,4-D and its salts and esters, 2,4-DB and its salts and esters, Aminopyralid and its salts wie Aminopyralid-tris(2-hydroxypropyl)ammonium and its esters, Benazolin, Benazolin-ethyl, Chloramben and its salts and esters, Clomeprop, Clopyralid and its salts and esters, Dicamba and its salts and esters, Dichlorprop and its salts and esters, Dichlorprop-P and its salts and esters, Fluroxypyr, Fluroxypyr-butometyl, Fluroxypyr-meptyl, MCPA and its salts and esters, MCPA-thioethyl, MCPB and its salts and esters, Mecoprop and its salts and esters, Mecoprop-P and its salts and esters, Picloram and its salts and esters, Quinclorac, Quinmerac, TBA (2,3,6) and its salts and esters, Triclopyr and its salts and esters, and 5,6-Dichlor-2-cyclopropyl-4-pyrimidincarbonic acid (H-9; CAS 858956-08-8) and its salts and esters;
n) from the group of Auxin-Transport-Inhibitors: Diflufenzopyr, Diflufenzopyr-natrium, Naptalam and Naptalam-natrium;
o) from the group of other Herbicides: Bromobutid, Chlorflurenol, Chlorflurenol-methyl, Cinmethylin, Cumyluron, Dalapon, Dazomet, Difenzoquat, Difenzoquat-metilsulfate, Dimethipin, DSMA, Dymron, Endothal and its salts, Etobenzanid, Flamprop, Flamprop-isopropyl, Flampropmethyl Flamprop-M-isopropyl, Flamprop-M-methyl, Flurenol, Flurenol-butyl, Flurprimidol, Fosamin, Fosamine-ammonium, Indanofan, Maleinic acid-hydrazid, Mefluidid, Metam, Methylazid, Methylbromid, Methyl-dymron, Methyljodid. MSMA, oleic acid, Oxaziclomefon, Pelargonic acid, Pyributicarb, Quinoclamin, Triaziflam, Tridiphan and 6-Chlor-3-(2-cyclopropyl-6-methylphenoxy)-4-pyridazinol (H-10; CAS 499223-49-3) and its salts and esters.

Examples for preferred Safeners C are Benoxacor, Cloquintocet, Cyometrinil, Cyprosulfamid, Dichlormid, Dicyclonon, Dietholate, Fenchlorazol, Fenclorim, Flurazol, Fluxofenim, Furilazol, Isoxadifen, Mefenpyr, Mephenat, Naphthalic acid anhydrid, Oxabetrinil, 4-(Dichloracetyl)-1-oxa-4-azaspiro[4.5]decan (H-11; MON4660, CAS 71526-07-3) and 2,2,5-Trimethyl-3-(dichloracetyl)-1,3-oxazolidin (H-12; R-29148, CAS 52836-31-4).

The compounds of groups a) to o) and the Safeners C are known Herbicides and Safeners, see e.g. The Compendium of Pesticide Common Names (http://www.alanwood.net/pesticides/); B. Hock, C. Fedtke, R. R. Schmidt, Herbicides, Georg Thieme Verlag, Stuttgart 1995. Other herbicidal effectors are known from WO 96/26202, WO 97/41116, WO 97/41117, WO 97/41118, WO 01/83459 and WO 2008/074991 as well as from W. Kramer et al. (ed.) “Modern Crop Protection Compounds”, Vol. 1, Wiley VCH, 2007 and the literature cited therein.

It is generally preferred to use the compounds of the invention in combination with herbicides that are selective for the crop being treated and which complement the spectrum of weeds controlled by these compounds at the application rate employed. It is further generally preferred to apply the compounds of the invention and other complementary herbicides at the same time, either as a combination formulation or as a tank mix.

The term “mut-HPPD nucleic acid” refers to an HPPD nucleic acid having a sequence that is mutated from a wild-type HPPD nucleic acid and that confers increased “HPPD-inhibiting herbicide” tolerance to a plant in which it is expressed. Furthermore, the term “mutated hydroxyphenyl pyruvate dioxygenase (mut-HPPD)” refers to the replacement of an amino acid of the wild-type primary sequences SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, a variant, a derivative, a homologue, an orthologue, or paralogue thereof, with another amino acid. The expression “mutated amino acid” will be used below to designate the amino acid which is replaced by another amino acid, thereby designating the site of the mutation in the primary sequence of the protein.

The term “mut-HST nucleic acid” refers to an HST nucleic acid having a sequence that is mutated from a wild-type HST nucleic acid and that confers increased “HPPD-inhibiting herbicide” tolerance to a plant in which it is expressed. Furthermore, the term “mutated homogentisate solanesyl transferase (mut-HST)” refers to the replacement of an amino acid of the wild-type primary sequences SEQ ID NO: 48 or 50 with another amino acid. The expression “mutated amino acid” will be used below to designate the amino acid which is replaced by another amino acid, thereby designating the site of the mutation in the primary sequence of the protein.

Several HPPDs and their primary sequences have been described in the state of the art, in particular the HPPDs of bacteria such as Pseudomonas (Ruetschi et al., Eur. J. Biochem., 205, 459-466, 1992, WO96/38567), of plants such as Arabidopsis (WO96/38567, Genebank AF047834) or of carrot (WO96/38567, Genebank 87257), of Coccicoides (Genebank COITRP), HPPDs of Brassica, cotton, Synechocystis, and tomato (U.S. Pat. No. 7,297,541), of mammals such as the mouse or the pig. Furthermore, artificial HPPD sequences have been described, for example in U.S. Pat. No. 6,768,044; U.S. Pat. No. 6,268,549;

In a preferred embodiment, the nucleotide sequence of (i) comprises the sequence of SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69 or a variant or derivative thereof.

In a particularly preferred embodiment, the mut-HPPD nucleic acid useful for the present invention comprises a mutated nucleic acid sequence of SEQ ID NO: 1 or SEQ ID NO: 52, or a variant or derivative thereof.

In one embodiment, the nucleotide sequence of (ii) comprises the sequence of SEQ ID NO: 47 or 49, or a variant or derivative thereof.

Furthermore, it will be understood by the person skilled in the art that the nucleotide sequences of (i) or (ii) encompasse homologues, paralogues and and orthologues of SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, and respectively SEQ ID NO: 47 or 49, as defined hereinafter.

The term “variant” with respect to a sequence (e.g., a polypeptide or nucleic acid sequence such as—for example—a transcription regulating nucleotide sequence of the invention) is intended to mean substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide “sequence identity” to the nucleotide sequence of SEQ ID NO:1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, 47, or 49. By “variant” polypeptide is intended a polypeptide derived from the protein of SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

In a preferred embodiment, variants of the polynucleotides useful for the present invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide “sequence identity” to the nucleotide sequence of SEQ ID NO:1, or SEQ ID NO: 52.

It is recognized that the polynucleotide molecules and polypeptides of the invention encompass polynucleotide molecules and polypeptides comprising a nucleotide or an amino acid sequence that is sufficiently identical to nucleotide sequences set forth in SEQ ID Nos: 1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, 47, or 49, or to the amino acid sequences set forth in SEQ ID Nos: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, 48, or 50. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity.

“Sequence identity” refers to the extent to which two optimally aligned DNA or amino acid sequences are invariant throughout a window of alignment of components, e.g., nucleotides or amino acids. An “identity fraction” for aligned segments of a test sequence and a reference sequence is the number of identical components that are shared by the two aligned sequences divided by the total number of components in reference sequence segment, i.e., the entire reference sequence or a smaller defined part of the reference sequence. “Percent identity” is the identity fraction times 100. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG. Wisconsin Package. (Accelrys Inc. Burlington, Mass.)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

“Derivatives” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds).

TABLE 3
Examples of conserved amino acid substitutions

| Residue | Conservative Substitutions |
|---|---|
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuikChange Site Directed mutagenesis (Stratagene, San Diego, Calif.), PCR-mediated site-directed mutagenesis or other site-directed mutagenesis protocols.

“Derivatives” further include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

“Orthologues” and “paralogues” encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene. A nonlimiting list of examples of such orthologues is shown in Table 1.

It is well-known in the art that paralogues and orthologues may share distinct domains harboring suitable amino acid residues at given sites, such as binding pockets for particular substrates or binding motifs for interaction with other proteins.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788(2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

The inventors of the present invention have surprisingly found that by substituting one or more of the key amino acid residues the herbicide tolerance or resistance could be remarkably increased as compared to the activity of the wild-type HPPD enzymes with SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67. Preferred substitutions of mut-HPPD are those that increase the herbicide tolerance of the plant, but leave the biological activity of the dioxygenase activity substantially unaffected.

Accordingly, in another object of the present invention the key amino acid residues of a HPPD enzyme comprising SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, a variant, derivative, othologue, paralogue or homologue thereof, is substituted by any other amino acid.

In a preferred embodiment, the key amino acid residues of a HPPD enzyme, a variant, derivative, othologue, paralogue or homologue thereof, is substituted by a conserved amino acid as depicted in Table 3 above.

It will be understood by the person skilled in the art that amino acids located in a close proximity to the positions of amino acids mentioned below may also be substituted. Thus, in another embodiment the mut HPPD useful for the present invention comprises a sequence of SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, or a variant, derivative, orthologue, paralogue or homologue thereof, wherein an amino acid ±3, ±2 or ±1 amino acid positions from a key amino acid is substituted by any other amino acid.

Based on techniques well-known in the art, a highly characteristic sequence pattern can be developed, by means of which further of mut-HPPD candidates with the desired activity may be searched.

Searching for further mut-HPPD candidates by applying a suitable sequence pattern would also be encompassed by the present invention. It will be understood by a skilled reader that the present sequence pattern is not limited by the exact distances between two adjacent amino acid residues of said pattern. Each of the distances between two neighbours in the above patterns may, for example, vary independently of each other by up to ±10, ±5, ±3, ±2 or ±1 amino acid positions without substantially affecting the desired activity.

In line with said above functional and spatial analysis of individual amino acid residues based on the crystallographic data as obtained according to the present invention, unique partial amino acid sequences characteristic of potentially useful mut-HPPD candidates of the invention may be identified.

In a particularly preferred embodiment, the mut-HPPD refers to a variant or derivative of SEQ ID NO: 2 wherein the substitutions are selected from the following Table 4a.

TABLE 4a
(Sequence ID No: 2): single amino acid substitutions

| Key amino acid position | Substituents |
|---|---|
| Val212 | Ile, Leu |
| Val213 | Thr, Ala |
| Asn215 | Ala, His |
| Ala236 | Leu, Ser, Arg |
| Phe238 | Val, Ala |
| Leu250 | Val, Met |
| Ser252 | Thr |
| Pro265 | Ala |
| Asn267 | Tyr, Gln |
| Gln278 | His, Asn, Ser |
| Ile279 | Thr |
| Arg309 | Lys, Ala |
| Leu320 | Asn, Gln, His, Tyr, |
| Pro321 | Ala, Arg, Gly, Asn |
| Leu334 | Glu, Cys |
| Leu353 | Met, Tyr, Ala, Ser |
| Phe366 | Ile, Leu, Tyr |
| Gly371 | Ile, Phe |
| Thr375 | Pro |
| Phe377 | Ala, Leu, Ser |
| Gly403 | Arg |
| Phe404 | Leu, Pro |
| Lys406 | Thr |
| Gly407 | Cys, His |
| Phe409 | Ile, His |
| Glu411 | Thr |
| Leu412 | Met, Phe, Trp, Ala, Ser |
| Ile416 | Val, Phe |
| Ser410 | Gly |
| Val254 | Ala |

It is to be understood that any amino acid besides the ones mentioned in the above tables could be used as a substitutent. Assays to test for the functionality of such mutants are readily available in the art, and respectively, described in the Example section of the present invention.

In a preferred embodiment, the amino acid sequence of a mut-HPPD differs from an amino acid sequence of a wild-type HPPD at one or more of the following positions corresponding to or at positions of SEQ ID NO:2: 212, 213, 215, 236, 238, 250, 252, 254, 265, 267, 278, 279, 309, 320, 321, 334, 353, 366, 371, 375, 377, 403, 404, 406, 407, 409, 411, 410, 412 or 416.

Examples of differences at these amino acid positions include, but are not limited to, one or more of the following:

the amino acid corresponding to or at position 236 is other than alanine;
the amino acid corresponding to or at position 411 is other than glutamic acid;
the amino acid corresponding to or at position 320 is other than leucine;
the amino acid corresponding to or at position 403 is other than glycine;
the amino acid corresponding to or at position 334 is other than leucine;
the amino acid corresponding to or at position 353 is other than leucine;
the amino acid corresponding to or at position 321 is other than proline;
the amino acid corresponding to or at position 212 is other than valine;
the amino acid corresponding to or at position 407 is other than glycine;
the amino acid corresponding to or at position 377 is other than phenylalanine;
the amino acid corresponding to or at position 412 is other than leucine;
the amino acid corresponding to or at position 278 is other than glutamine;
the amino acid corresponding to or at position 406 is other than lysine;
the amino acid corresponding to or at position 404 is other than phenylalanine;
the amino acid corresponding to or at position 409 is other than phenylalanine;
the amino acid corresponding to or at position 416 is other than isoleucine;
the amino acid corresponding to or at position 250 is other than leucine;
the amino acid corresponding to or at position 267 is other than asparagine;
the amino acid corresponding to or at position 252 is other than serine;
the amino acid corresponding to or at position 265 is other than proline;
the amino acid corresponding to or at position 371 is other than glycine;
the amino acid corresponding to or at position 375 is other than threonine;
the amino acid corresponding to or at position 309 is other than arginine;
the amino acid corresponding to or at position 279 is other than isoleucine;
the amino acid corresponding to or at position 366 is other than phenylalanine;
the amino acid corresponding to or at position 238 is other than phenylalanine;
the amino acid corresponding to or at position 213 is other than valine;
the amino acid corresponding to or at position 215 is other than asparagine;
the amino acid corresponding to or at position 410 is other than serine;
the amino acid corresponding to or at position 254 is other than valine.

In some embodiments, the mut HPPD enzyme comprises one or more substitutions corresponding to or at the following positions of SEQ ID NO:2:

the amino acid corresponding to or at position 236 is leucine, serine or arginine; the amino acid corresponding to or at position 411 is threonine;
the amino acid corresponding to or at position 320 is asparagine, glutamine, histidine or tyrosine;
the amino acid corresponding to or at position 403 is arginine;
the amino acid corresponding to or at position 334 is glutamic acid or cysteine;
the amino acid corresponding to or at position 353 is methionine, tyrosine, alanine, or serine;
the amino acid corresponding to or at position 321 is alanine, arginine, glycine or asparagine;
the amino acid corresponding to or at position 212 is isoleucine or leucine;
the amino acid corresponding to or at position 407 is cysteine or histidine;
the amino acid corresponding to or at position 377 is alanine, leucine, serine;
the amino acid corresponding to or at position 412 is methionine, phenylalanine, tryptophan, alanine, serine;
the amino acid corresponding to or at position 278 is histidine, asparagine, serine;
the amino acid corresponding to or at position 406 is Threonine;
the amino acid corresponding to or at position 404 is leucine, Proline;
the amino acid corresponding to or at position 409 is isoleucine, histidine;
the amino acid corresponding to or at position 416 is valine, phenylalanine;
the amino acid corresponding to or at position 250 is valine, methionine;
the amino acid corresponding to or at position 267 is Tyrosin, Glutamine;
the amino acid corresponding to or at position 252 is Threonine;
the amino acid corresponding to or at position 265 is alanine;
the amino acid corresponding to or at position 371 is Isoleucine, phenylalanine;
the amino acid corresponding to or at position 375 is Proline;
the amino acid corresponding to or at position 309 is Lysine, alanine;
the amino acid corresponding to or at position 279 is Threonine;
the amino acid corresponding to or at position 366 is Isoleucine, leucine, Tyrosin;
the amino acid corresponding to or at position 238 is valine, alanine;
the amino acid corresponding to or at position 213 is Threonine, alanine;
the amino acid corresponding to or at position 215 is alanine, histidine;
the amino acid corresponding to or at position 410 is glycine:
the amino acid corresponding to or at position 254 is alanine.

Furthermore, the inventors of the present invention have surprisingly found that by substituting at least two of the key amino acid residues of SEQ ID NO: 2 with specific residues, the herbicide tolerance or resistance could be remarkably increased as compared to the activity of the wild-type HPPD enzymes or HPPD enzymes in which only one amino acid residue had been substituted. Therefore, in another preferred embodiment the present invention the variant or derivative of the mut-HPPD refers to a polypeptide of SEQ ID NO: 2, a homologue, paralogue, or orthologue thereof, in which two, three, four or five key amino acids are substituted by another amino acid residue. Particularly preferred double, triple, quadruple, or quintuple mutations are described in Table 4b.

TABLE 4b
(with reference to Sequence ID No: 2): combined amino acid
substitutions

| Combination | |
|---|---|
| No | Key amino acid position and and its substitutents |
| 1 | A236L, E411T |
| 2 | L320H, P321A |
| 3 | L320H, P321R |
| 4 | L320N, P321A |
| 5 | L320N, P321R |
| 6 | L320Q, P321A |
| 7 | L320Q, P321R |
| 8 | L320Y, P321A |
| 9 | L320Y, P321R |
| 10 | L353M, P321R |
| 11 | L353M, P321R, A236L |
| 12 | L353M, P321R, A236L, E411T |
| 13 | L353M, P321R, E411T |
| 14 | L353M, P321R, L320H |
| 15 | L353M, P321R, L320N |
| 16 | L353M, P321R, L320Q |
| 17 | L353M, P321R, L320Y |
| 18 | L353M, P321R, V212I |
| 19 | L353M, P321R, V212I, L334E |
| 20 | L353M, P321R, V212L, L334E |
| 21 | L353M, P321R, V212L, L334E, A236L |
| 22 | L353M, P321R, V212L, L334E, A236L, E411T |
| 23 | L353M, P321R, V212L, L334E, E411T |
| 24 | L353M, P321R, V212L, L334E, L320H |
| 25 | L353M, P321R, V212L, L334E, L320N |
| 26 | L353M, P321R, V212L, L334E, L320Q |
| 27 | L353M, P321R, V212L, L334E, L320Y |
| 28 | L353M, V212I |

In a particularly preferred embodiment, the mut HPPD enzyme useful for the present invention comprises one or more of the following substitutions referring to SEQ ID NO:2: the amino acid corresponding to or at position 320 is histidine, asparagine or glutamine; the amino acid corresponding to or at position 334 is glutamic acid; the amino acid corresponding to or at position 353 is methionine; the amino acid corresponding to or at position 321 is alanine or arginine; the amino acid corresponding to or at position 212 is isoleucine.

In an especially particularly preferred embodiment, the mut HPPD refers to a polypeptide comprising SEQ ID NO: 2, or a homologue, paralogue or orthologue thereof, wherein the Leucine corresponding to or at position 320 is substituted by a Histidine, and the Proline corresponding to or at position 321 is substituted by an Alanine.

Another especially particularly preferred embodiment, the mut HPPD refers to a polypeptide comprising SEQ ID NO: 2, or a homologue, paralogue or orthologue thereof, wherein Leucine corresponding to or at position 353 is substituted by a Methionine, the Proline corresponding to or at position 321 is substituted by an Arginine, and the Leucine corresponding to or at position 320 is substituted by an Asparagine.

In another especially particularly preferred embodiment, the mut HPPD refers to a polypeptide comprising SEQ ID NO: 2, or a homologue, paralogue or orthologue thereof, wherein leucine corresponding to or at position 353 is substituted by a methionine, the proline corresponding to or at position 321 is substituted by an arginine, and the Leucine corresponding to or at position 320 is substituted by a glutamine.

In another preferred embodiment, the mut-HPPD refers to a variant or derivative of SEQ ID NO: 53 wherein the substitutions are selected from the following Table 4c.

TABLE 4c
(reference to Sequence ID No: 53): single amino acid substitutions

| Key amino | | |
|---|---|---|
| acid position | Substituents | Preferred substituents |
| Val228 | Thr, Ala | Thr, Ala |
| Asn230 | Ala, His | Ala, His |
| Ala251 | Ser, Arg | Ser, Arg |
| Phe253 | Val, Ala | Val, Ala |
| Leu265 | Val, Met | Val, Met |
| Ser267 | Thr | Thr |
| Pro280 | Ala | Ala |
| Asn282 | Tyr, Gln | Tyr, Gln |
| Lys291 | Arg, Ala | Arg |
| Gln293 | Ala, Leu, Ile, Val, His, Asn, Ser | His, Asn, Ser |
| Ile294 | Thr | Thr |
| Arg324 | Lys, Ala | Lys, Ala |
| Met335 | Ala, Trp, Phe, Leu, Ile, Val, Asn, | Gln, Asn, His, Tyr |
| | Gln, His, Tyr, Ser, Thr, Cys | |
| Pro336 | Ala, Arg, Gly, Asn | Ala, Gly |
| Ser337 | Ala, Pro, Thr | Pro, Thr |
| Pro339 | Deletion | Deletion |
| Pro340 | Gly | Gly |
| Glu363 | Gln | Gln |
| Leu368 | Met, Tyr, | Met |
| Phe381 | Ile, Leu, Tyr | Ile, Leu |
| Leu385 | Ala, Val, Gln, Asp | Val, Asp |
| Gly386 | Ile, Phe | Ile, Phe |
| Thr390 | Pro | Pro |
| Phe392 | Ala, Leu, Ser | Ala |
| Ile393 | Ala, Leu, Phe, Val | Leu |
| Phe419 | Leu, Pro | Leu, Pro |
| Lys421 | Thr | Thr |
| Gly422 | His, Met, Phe, Cys | His, Cys |
| Phe424 | Ile, His | Ile, His |
| Leu427 | Phe, Trp, Ala, Ser, Met | Phe |
| Ile431 | Val, Phe | Val, Phe |
| Ser425 | Gly | Gly |
| Val269 | Ala | Ala |

It is to be understood that any amino acid besides the ones mentioned in the above tables could be used as a substitutent. Assays to test for the functionality of such mutants are readily available in the art, and respectively, described in the Example section of the present invention.

In another preferred embodiment, the mut-HPPD amino acid sequence differs from a wild-type amino acid sequence of an HPPD at one or more positions corresponding to or at the following positions of SEQ ID NO:53:

228, 230, 251, 253, 265, 267, 280, 282, 291, 293, 294, 324, 335, 336, 337, 339, 340, 363, 368, 381, 385, 386, 390, 392, 393, 419, 421, 422, 424, 427, 431, 425, 269.

Examples of differences at these amino acid positions include, but are not limited to, one or more of the following:

the amino acid corresponding to or at position 228 is other than valine;
the amino acid corresponding to or at position 230 is other than asparagine;
the amino acid corresponding to or at position 251 is other than alanine;
the amino acid corresponding to or at position 253 is other than phenylalanine;
the amino acid corresponding to or at position 265 is other than leucine;
the amino acid corresponding to or at position 267 is other than serine;
the amino acid corresponding to or at position 280 is other than proline;
the amino acid corresponding to or at position 282 is other than asparagine;
the amino acid corresponding to or at position 291 is other than lysine;
the amino acid corresponding to or at position 293 is other than glutamine;
the amino acid corresponding to or at position 294 is other than isoleucine;
the amino acid corresponding to or at position 324 is other than arginine;
the amino acid corresponding to or at position 335 is other than methionine;
the amino acid corresponding to or at position 336 is other than proline;
the amino acid corresponding to or at position 337 is other than serine;
the amino acid corresponding to or at position 339 is other than proline;
the amino acid corresponding to or at position 340 is other than proline;
the amino acid corresponding to or at position 363 is other than glutamic acid;
the amino acid corresponding to or at position 368 is other than leucine;
the amino acid corresponding to or at position 381 is other than phenylalanine;
the amino acid corresponding to or at position 385 is other than leucine;
the amino acid corresponding to or at position 386 is other than glycine;
the amino acid corresponding to or at position 390 is other than threonine;
the amino acid corresponding to or at position 392 is other than phenylalanine;
the amino acid corresponding to or at position 393 is other than an isoleucine;
the amino acid corresponding to or at position 419 is other than phenylalanine;
the amino acid corresponding to or at position 421 is other than lysine;
the amino acid corresponding to or at position 422 is other than glycine;
the amino acid corresponding to or at position 424 is other than phenylalanine;
the amino acid corresponding to or at position 427 is other than leucine;
the amino acid corresponding to or at position 431 is other than isoleucine;
the amino acid corresponding to or at position 425 is other than serine;
the amino acid corresponding to or at position 269 is other than valine.

In some embodiments, the mut-HPPD enzyme comprises one or more substitutions at positions corresponding to or at the following positions of SEQ ID NO: 53:

the amino acid corresponding to or at position 228 is Thr, or Ala;
the amino acid corresponding to or at position 230 is Ala, or His;
the amino acid corresponding to or at position 251 is Ser, or Arg;
the amino acid corresponding to or at position 253 is Val, or Ala;
the amino acid corresponding to or at position 265 is Val, or Met;
the amino acid corresponding to or at position 267 is threonine;
the amino acid corresponding to or at position 280 is Ala;
the amino acid corresponding to or at position 282 is Tyr, or Gln;
the amino acid corresponding to or at position 291 is Arg, or Ala;
the amino acid corresponding to or at position 293 is alanine, leucine, isoleucine, valine, histidine, asparagine or serine, preferably histidine, asparagine or serine;
the amino acid corresponding to or at position 294 is threonine;
the amino acid corresponding to or at position 324 is Lys, or Ala;
the amino acid corresponding to or at position 335 is alanine, tryptophane, phenylalanine, leucine, isoleucine, valine, asparagine, glutamine, histidine, tyrosine, serine, threonine or cysteine, preferably Gln, Asn, His, or Tyr;
the amino acid corresponding to or at position 336 is alanine, arginine, Gly, or Asn, preferably alanine or glycine;
the amino acid corresponding to or at position 337 is alanine, threonine or proline, preferably threonine or proline;
the amino acid corresponding to or at position 339 is deleted;
the amino acid corresponding to or at position 340 is glycine;
the amino acid corresponding to or at position 363 is glutamine;
the amino acid corresponding to or at position 368 is methionine or tyrosine, preferably methionine;
the amino acid corresponding to or at position 381 is Ile, Leu, or Tyr, preferably Isoleucine or leucine;
the amino acid corresponding to or at position 385 is valine, alanine, Gln, or Asp, preferably valine or aspartic acid;
the amino acid corresponding to or at position 386 is Ile, or Phe;
the amino acid corresponding to or at position 390 is Pro;
the amino acid corresponding to or at position 392 is alanine, leucine or serine, preferably alanine;
the amino acid corresponding to or at position 393 is Ala, Leu, Phe, Val, preferably leucine;
the amino acid corresponding to or at position 419 is Leu or Pro;
the amino acid corresponding to or at position 421 is threonine;
the amino acid corresponding to or at position 422 is histidine, methionine, phenylalanine, or cysteine, preferably histidine or cysteine;
the amino acid corresponding to or at position 424 is Ile or His;
the amino acid corresponding to or at position 427 is phenylalanine, tryptophan, Ala, Ser, or Met, preferably phenylalanine;
the amino acid corresponding to or at position 431 is Val or Phe;
the amino acid corresponding to or at position 425 is glycine;
the amino acid corresponding to or at position 269 is alanine.

Furthermore, the inventors of the present invention have found that by substituting at least two of the key amino acid residues of SEQ ID NO: 53 with specific residues, the herbicide tolerance or resistance could be remarkably increased as compared to the activity of the wild-type HPPD enzymes or HPPD enzymes in which only one amino acid residue had been substituted. Therefore, in another preferred embodiment the present invention the variant or derivative of the mut-HPPD refers to a polypeptide of SEQ ID NO: 53, a homologue, orthologue, or paralogue thereof, wherein two, three, four or five key amino acids are substituted by another amino acid residue. Particularly preferred double, triple, quadruple, or quintuple mutations are described in Table 4d.

TABLE 4d
(reference to Sequence ID No: 53): combined amino acid substitutions

| Combination | Key amino acid | | Preferred |
|---|---|---|---|
| No | position | Substituents | substituents |
| 1 | Pro336 | Ala, Arg | Ala |
| | Glu363 | Gln | Gln |
| 2 | Pro336 | Ala, Arg | Ala |
| | Glu363 | Gln | Gln |
| | Leu385 | Ala, Val | Val |
| 3 | Pro336 | Ala, Arg | Ala |
| | Glu363 | Gln | Gln |
| | Leu385 | Ala, Val | Val |
| | Ile393 | Ala, Leu | Leu |
| 4 | Leu385 | Ala, Val | Val |
| | Ile393 | Ala, Leu | Leu |
| 5 | Met335 | Ala, Trp, Phe, Leu, Ile, | Gln, Asn, |
| | | Val, Asn, Gln, His, Tyr, | His, Tyr |
| | | Ser, Thr, Cys | |
| | Pro336 | Ala, Arg, Gly | Ala, Gly |
| 6 | Met335 | Ala, Trp, Phe, Leu, Ile, | Gln, Asn, |
| | | Val, Asn, Gln, His, Tyr, | His, Tyr |
| | | Ser, Thr, Cys | |
| | Pro336 | Ala, Arg, Gly | Ala, Gly |
| | Glu363 | Gln | Gln |
| 7 | Met335 | Ala, Trp, Phe, Leu, Ile, | Gln, Asn, |
| | | Val, Asn, Gln, His, Tyr, | His, Tyr, |
| | | Ser, Thr, Cys | Leu |
| | Pro336 | Ala, Arg, Gly | Ala, Arg, Gly |
| | Ser337 | Ala, Pro, Thr | Pro, Thr |
| | Pro339 | Deletion | Deletion |
| | Pro340 | Gly | Gly |

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, or Arg, and the amino acid corresponding to or at position 363 of SEQ ID NO:53 is Gin.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, Arg, and the amino acid corresponding to or at position 363 of SEQ ID NO:53 is Gln, and the amino acid corresponding to or at position 385 of SEQ ID NO:53 is Ala, Val.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, and the amino acid corresponding to or at position 363 of SEQ ID NO:53 is Gln, and the amino acid corresponding to or at position 385 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, and the amino acid corresponding to or at position 363 of SEQ ID NO:53 is Gln, and the amino acid corresponding to or at position 385 of SEQ ID NO:53 is Val.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Arg, and the amino acid corresponding to or at position 363 of SEQ ID NO:53 is Gln, and the amino acid corresponding to or at position 385 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Arg, and the amino acid corresponding to or at position 363 of SEQ ID NO:53 is Gln, and the amino acid corresponding to or at position 385 of SEQ ID NO:53 is Val.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, Arg, and the amino acid corresponding to or at position 363 of SEQ ID NO:53 is Gln, and the amino acid corresponding to or at position 385 of SEQ ID NO:53 is Ala, Val, and the amino acid corresponding to or at position 393 of SEQ ID NO:53 is Ala, Leu.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, and the amino acid corresponding to or at position 363 of SEQ ID NO:53 is Gln, and the amino acid corresponding to or at position 385 of SEQ ID NO:53 is Ala, and the amino acid corresponding to or at position 393 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, and the amino acid corresponding to or at position 363 of SEQ ID NO:53 is Gln, and the amino acid corresponding to or at position 385 of SEQ ID NO:53 is Ala, and the amino acid corresponding to or at position 393 of SEQ ID NO:53 is Leu.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, and the amino acid corresponding to or at position 363 of SEQ ID NO:53 is Gln, and the amino acid corresponding to or at position 385 of SEQ ID NO:53 is Val, and the amino acid corresponding to or at position 393 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Ala, and the amino acid corresponding to or at position 363 of SEQ ID NO:53 is Gln, and the amino acid corresponding to or at position 385 of SEQ ID NO:53 is Val, and the amino acid corresponding to or at position 393 of SEQ ID NO:53 is Leu.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Arg, and the amino acid corresponding to or at position 363 of SEQ ID NO:53 is Gln, and the amino acid corresponding to or at position 385 of SEQ ID NO:53 is Ala, and the amino acid corresponding to or at position 393 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Arg, and the amino acid corresponding to or at position 363 of SEQ ID NO:53 is Gln, and the amino acid corresponding to or at position 385 of SEQ ID NO:53 is Ala, and the amino acid corresponding to or at position 393 of SEQ ID NO:53 is Leu.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Arg, and the amino acid corresponding to or at position 363 of SEQ ID NO:53 is Gln, and the amino acid corresponding to or at position 385 of SEQ ID NO:53 is Val, and the amino acid corresponding to or at position 393 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 336 of SEQ ID NO:53 is Arg, and the amino acid corresponding to or at position 363 of SEQ ID NO:53 is Gln, and the amino acid corresponding to or at position 385 of SEQ ID NO:53 is Val, and the amino acid corresponding to or at position 393 of SEQ ID NO:53 is Leu.

In another preferred embodiment, the mut-HPPD comprises a sequence of SEQ ID NO: 53 a variant, derivative, orthologue, paralogue or homologue thereof, in which:

the amino acid corresponding to or at position 385 of SEQ ID NO:53 is Ala, Val, and the amino acid corresponding to or at position 393 of SEQ ID NO:53 is Ala, Leu.

In another preferred embodiment, the mut-HPPD comprises a sequence of SEQ ID NO: 53 a variant, derivative, orthologue, paralogue or homologue thereof, in which:

the amino acid corresponding to or at position 385 of SEQ ID NO:53 is Ala, and the amino acid corresponding to or at position 393 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 385 of SEQ ID NO:53 is Ala, and the amino acid corresponding to or at position 393 of SEQ ID NO:53 is Leu.

In another preferred embodiment, the mut-HPPD comprises a sequence of SEQ ID NO: 53 a variant, derivative, orthologue, paralogue or homologue thereof, in which:

the amino acid corresponding to or at position 385 of SEQ ID NO:53 is Val, and the amino acid corresponding to or at position 393 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 385 of SEQ ID NO:53 is Val, and the amino acid corresponding to or at position 393 of SEQ ID NO:53 is Leu.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, Trp, Phe, Leu, Ile, Val, Asn, Gln, His, Tyr, Ser, Thr, Cys, and the amino acid corresponding to or at position 336 of SEQ ID NO:53 is Ala, Arg, Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Ala.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Arg.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, Trp, Phe, Leu, Ile, Val, Asn, Gln, His, Tyr, Ser, Thr, Cys, and the amino acid corresponding to or at position 336 of SEQ ID NO:53 is Ala, Arg, Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Gly, and the amino acid corresponding to or at position 363 of SEQ ID NO:53 is Gin.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Gly, and the amino acid corresponding to or at position 363 of SEQ ID NO:53 is Gin.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Gly, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Ala, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Arg, and the amino acid corresponding to or at position 363 of SEQ ID NO:53 is Gln.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Gly, and the amino acid corresponding to or at position 363 of SEQ ID NO:53 is Gin.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, Trp, Phe, Leu, Ile, Val, Asn, Gln, His, Tyr, Ser, Thr, Cys, and the amino acid corresponding to or at position 336 of SEQ ID NO:53 is Ala, Arg, Gly, and the amino acid corresponding to or at position 337 of SEQ ID NO:53 is Ala, Pro, Thr, and the amino acid corresponding to or at position 339 of SEQ ID N0:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ala, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Trp, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Phe, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Leu, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ile, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Val, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Asn, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Gln, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is His, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Tyr, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Ser, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Thr, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Ala, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Ala, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Ala, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Arg, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Arg, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Arg, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Gly, and the amino acid corresponding to or at position 337 of SEQ ID NO:53 is Ala, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Gly, and the amino acid corresponding to or at position 337 of SEQ ID NO:53 is Pro, and the amino acid corresponding to or at position 339 of SEQ ID NO:53 is deleted, and the amino acid corresponding to or at position 340 of SEQ ID NO:53 is Gly.

In another preferred embodiment, the mut-HPPD comprises a sequence of SEQ ID NO: 53 a variant, derivative, orthologue, paralogue or homologue thereof, in which:

the amino acid corresponding to or at position 335 of SEQ ID NO:53 is Cys, and the amino acid corresponding to or at position 336 of SEQ ID NO:53 is Gly, and the amino acid corresponding to or at position 337 of SEQ ID NO:53 is Thr, and the amino acid corresponding to or at position 339 of SEQ ID NO:53 is deleted, and the amino acid corresponding to or at position 340 of SEQ ID NO:53 is Gly.

In another embodiment, the variant or derivative of the HPPD enzyme of SEQ ID NO: 67 comprises one or more of the following substitutions:

the alanine at position 8 is substituted by threonine;
the glycine at position 68 is substituted by alanine;
the valine at position 261 is substituted by alanine;
the methionine at position 301 is substituted by isoleucine;
the methionine at position 327 is substituted by leucine;
the alanine at position 328 is substituted by proline;
the threonine at position 331 is substituted by proline;
the arginine at position 341 is substituted by glutamic acid;
the lysine at position 352 is substituted by asparagine;
the leucine at position 360 is substituted by mMethionine;

The leucine at position 383 is substituted by phenylalanine; glycine at position 414 is substituted by aspartic acid.

In another embodiment, the variant or derivative of the HPPD enzyme of SEQ ID NO: 67 comprises one or more of the following substitutions:

the alanine at position 8 is substituted by threonine;
the histidine at position 44 is substituted by glutamine;
the glycine at position 68 is substituted by alanine;
the alanine at position 71 is substituted by valine;
the phenylalanine at position 98 is substituted by leucine;
the phenylalanine at position 233 is substituted by methionine;
the alanine at position 253 is substituted by threonine;
the valine at position 261 is substituted by alanine;
the methionine at position 301 is substituted by isoleucine;
the glutamine at position 316 is substituted by arginine;
the methionine at position 327 is substituted by leucine;
the alanine at position 328 is substituted by proline;
the threonine at position 331 is substituted by proline;
the arginine at position 341 is substituted by cysteine;
the lysine at position 352 is substituted by asparagine;
the leucine at position 360 is substituted by methionine;
the leucine at position 383 is substituted by phenylalanine;
the serine at position 417 is substituted by glycine.

In a further preferred embodiment, the amino acid sequence differs from an amino acid sequence of an HPPD of SEQ ID NO: 57 at position 418. Preferably, the amino acid at position 418 is other than alanine. More preferably, the amino acid at position 418 is threonine.

In a further preferred embodiment, the amino acid sequence differs from an amino acid sequence of an HPPD of SEQ ID NO: 57 at position 237. Preferably, the amino acid at position 237 is other than serine. More preferably, the amino acid at position 237 is leucine.

It will be within the knowledge of the skilled artisan to identify conserved regions and motifs shared between the homologues, orthologues and paralogues of of SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, and respectively SEQ ID NO: 48 or 50, such as those depicted in Table 1. Having identified such conserved regions that may represent suitable binding motifs, amino acids corresponding to the amino acids listed in Table 4a and 4b, 4c, and 4d can be chosen to be substituted by any other amino acid, preferably by conserved amino acids as shown in table 3, and more preferably by the amino acids of tables 4a and 4b, 4c, and 4d.

The corresponding positions, i.e. preferred sites to be substituted are listed in the following Table 4e)

TABLE e

| SEQ- | | | | | | | | | | Pos | Pos | Pos | Pos | Pos | Pos | Pos | Pos | Pos | Pos |
|---|---|---|---|---|---|---|---|---|---|---|---|---|---|---|---|---|---|---|---|
| ID | Pos 1 | Pos 2 | Pos 3 | Pos 4 | Pos 5 | Pos 6 | Pos 7 | Pos 8 | Pos 9 | 10 | 11 | 12 | 13 | 14 | 15 | 16 | 17 | 18 | 19 |
| 53 | A227 | V228 | N230 | A251 | F253 | L265 | S267 | V269 | P280 | N282 | K291 | Q293 | I294 | R324 | M335 | P336 | S337 | P339 | P340 |
| 2 | V212 | V213 | N215 | A236 | F238 | L250 | S252 | V254 | P265 | N267 | R276 | Q278 | I279 | R309 | L320 | P321 | P322 | L324 | P325 |
| 5 | A270 | V271 | N273 | A294 | F296 | L308 | S310 | V312 | P323 | N325 | K333 | Q335 | I336 | R366 | M378 | K379 | R380 | S382 | E383 |
| 8 | A227 | V228 | N230 | A251 | F253 | L265 | S267 | V269 | P280 | N282 | K291 | Q293 | I294 | R324 | M335 | P336 | R337 | S339 | P340 |
| 11 | T192 | V193 | N195 | A216 | F218 | L230 | S232 | V234 | P245 | N247 | K255 | Q257 | I258 | R288 | M300 | K301 | R302 | S304 | D305 |
| 14 | V152 | V153 | N155 | Q178 | F180 | L189 | S191 | A193 | N204 | N206 | N212 | Q214 | I215 | R245 | L252 | S253 | V254 | N256 | S257 |
| 17 | G160 | V161 | N163 | R186 | F188 | L197 | S199 | V201 | P212 | N214 | N220 | Q222 | I223 | K253 | L260 | D261 | I262 | P264 | S265 |
| 20 | V145 | V146 | N148 | Q171 | F173 | L182 | S184 | A186 | N197 | N199 | S205 | Q207 | I208 | R238 | L245 | K246 | I247 | T249 | G250 |
| 22 | V218 | V219 | N221 | A242 | F244 | L256 | S258 | V260 | P271 | N273 | R282 | Q284 | I285 | R315 | M326 | A327 | P328 | Q330 | A331 |
| 24 | V218 | V219 | N221 | A242 | F244 | L256 | S258 | V260 | P271 | N273 | R282 | Q284 | I285 | R315 | M326 | A327 | P328 | Q330 | A331 |
| 26 | I218 | V219 | N221 | A242 | F244 | L256 | S258 | V260 | P271 | N273 | R282 | Q284 | I285 | Q315 | M326 | A327 | P328 | A330 | P331 |
| 28 | I218 | V219 | N221 | A242 | F244 | L256 | S258 | V260 | P271 | N273 | R282 | Q284 | I285 | Q315 | M326 | A327 | P328 | A330 | P331 |
| 30 | V212 | V213 | N215 | A236 | F238 | L250 | S252 | V254 | P265 | N267 | R276 | Q278 | I279 | Q309 | M320 | A321 | P322 | Q324 | P325 |
| 32 | V212 | V213 | N215 | A236 | F238 | L250 | S252 | V254 | P265 | N267 | R276 | Q278 | I279 | Q309 | M320 | A321 | P322 | Q324 | P325 |
| 34 | V218 | V219 | N221 | A242 | F244 | L256 | S258 | V260 | P271 | N273 | R282 | Q284 | I285 | R315 | M326 | A327 | P328 | Q330 | A331 |
| 36 | V144 | V145 | N147 | Y170 | Y172 | L181 | S183 | V185 | A196 | N198 | A204 | Q206 | I207 | R237 | L244 | Q245 | V246 | P248 | Q249 |
| 38 | V184 | V185 | N187 | W210 | A212 | L224 | S226 | V228 | P239 | N241 | K249 | Q251 | I252 | R282 | L289 | E290 | V291 | P293 | K294 |
| 40 | I176 | V177 | N179 | I202 | F204 | L216 | S218 | V220 | P230 | N232 | K240 | Q242 | I243 | E273 | L280 | K281 | T282 | G284 | S285 |
| 42 | M194 | V195 | N197 | I220 | F222 | L234 | S236 | V238 | P249 | N251 | K259 | Q261 | I262 | R292 | L299 | Y300 | V301 | D303 | T304 |
| 44 | A207 | V208 | N210 | A233 | F235 | L247 | S249 | V251 | P262 | N264 | K272 | Q274 | I275 | R305 | L312 | N313 | T314 | D316 | A317 |
| 46 | A207 | V208 | N210 | A233 | F235 | L247 | S249 | V251 | P262 | N264 | K272 | Q274 | I275 | R305 | L312 | N313 | T314 | D316 | A317 |
| 55 | A213 | V214 | N216 | S237 | F239 | L251 | S253 | V255 | P266 | N268 | K277 | Q279 | I280 | R310 | M321 | P322 | R323 | N325 | A326 |
| 57 | A213 | V214 | N216 | S237 | F239 | L251 | S253 | V255 | P266 | N268 | K277 | Q279 | I280 | R310 | M321 | P322 | R323 | N325 | A326 |
| 58 | A214 | V215 | N217 | A238 | F240 | L252 | S254 | V256 | P267 | N269 | K278 | Q280 | I281 | R311 | M322 | P323 | K324 | P326 | P327 |
| 59 | V224 | V225 | N227 | A248 | F250 | L262 | S264 | V266 | P277 | N279 | R288 | Q290 | I291 | R321 | L332 | A333 | P334 | P336 | P337 |
| 60 | V214 | V215 | N217 | A238 | F240 | L252 | S254 | V256 | P267 | N269 | R278 | Q280 | I281 | R311 | L322 | P323 | P324 | C326 | R327 |
| 61 | I219 | V220 | N222 | A243 | F245 | L257 | S259 | V261 | P272 | N274 | R283 | Q285 | I286 | Q316 | M327 | A328 | P329 | T331 | S332 |
| 62 | A226 | V227 | N229 | A250 | F252 | L264 | S266 | V268 | P279 | N281 | K290 | Q292 | I293 | R323 | M334 | P335 | S336 | P338 | P339 |
| 63 | T223 | V224 | N226 | A247 | F249 | L261 | S263 | V265 | P276 | N278 | K287 | Q289 | I290 | R320 | M331 | P332 | S333 | P335 | P336 |
| 64 | L163 | T164 | N166 | R189 | F191 | L200 | S202 | A204 | P215 | N217 | A224 | Q226 | I227 | K257 | M264 | T265 | A266 | P268 | D269 |
| 65 | L163 | T164 | N166 | R189 | F191 | L200 | S202 | A204 | P215 | N217 | A224 | Q226 | I227 | K257 | M264 | T265 | A266 | P268 | D269 |
| 66 | V218 | V219 | N221 | A242 | F244 | L256 | S258 | V260 | P271 | N273 | R282 | Q284 | I285 | R315 | M326 | A327 | P328 | Q330 | A331 |
| 67 | I219 | V220 | N222 | A243 | F245 | L257 | S259 | V261 | P272 | N274 | R283 | Q285 | I286 | Q316 | M327 | A328 | P329 | T331 | S332 |

| SEQ- | Pos | Pos | Pos | Pos | Pos | Pos | Pos | Pos | Pos | Pos | Pos | Pos | Pos | Pos | Pos | Pos | Pos | Pos |
|---|---|---|---|---|---|---|---|---|---|---|---|---|---|---|---|---|---|---|
| ID | 20 | 21 | 22 | 23 | 24 | 25 | 26 | 27 | 28 | 29 | 30 | 31 | 32 | 33 | 34 | 35 | 36 | 37 |
| 53 | R349 | E363 | L368 | F381 | L385 | G386 | T390 | F392 | I393 | G418 | F419 | K421 | G422 | F424 | S425 | E426 | L427 | I431 |
| 2 | L334 | Q348 | L353 | F366 | V370 | G371 | T375 | F377 | L378 | G403 | F404 | K406 | G407 | F409 | S410 | E411 | L412 | I416 |
| 5 | R392 | E406 | L411 | F424 | V428 | G429 | T433 | F435 | F436 | G467 | F468 | K470 | G471 | F473 | R474 | E475 | L476 | I480 |
| 8 | R349 | E363 | L368 | F381 | L385 | G386 | T390 | F392 | F393 | G423 | F424 | K426 | G427 | F429 | S430 | E431 | L432 | I436 |
| 11 | R314 | E328 | L333 | F346 | L350 | G351 | T355 | F357 | L358 | G396 | F397 | Q399 | G400 | F402 | R403 | E404 | L405 | I409 |
| 14 | R267 | E283 | L288 | F304 | I308 | F309 | T313 | F315 | F316 | G327 | F328 | Q330 | G331 | F333 | Q334 | A335 | L336 | I340 |
| 17 | E275 | E287 | L292 | F308 | I312 | F313 | T317 | F319 | F320 | G331 | F332 | Q334 | R335 | F337 | L338 | A339 | L340 | M344 |
| 20 | Y260 | Q272 | L277 | F293 | C297 | Y298 | T302 | F304 | W305 | G316 | F317 | Q319 | G320 | F322 | Q323 | A324 | L325 | V329 |
| 22 | L340 | Q354 | L359 | F372 | V376 | G377 | T381 | F383 | L384 | G409 | F410 | K412 | G413 | F415 | S416 | E417 | L418 | I422 |
| 24 | L340 | Q354 | L359 | F372 | V376 | G377 | T381 | F383 | L384 | G409 | F410 | K412 | G413 | F415 | S416 | E417 | L418 | I422 |
| 26 | R340 | Q354 | L359 | F372 | V376 | G377 | T381 | F383 | L384 | G409 | F410 | K412 | G413 | F415 | S416 | Q417 | L418 | I422 |
| 28 | R340 | Q354 | L359 | F372 | V376 | G377 | T381 | F383 | L384 | G409 | F410 | K412 | G413 | F415 | S416 | Q417 | L418 | I422 |
| 30 | I334 | Q348 | L353 | F366 | V370 | G371 | T375 | F377 | L378 | G403 | F404 | K406 | G407 | F409 | S410 | E411 | L412 | I416 |
| 32 | I334 | Q348 | L353 | F366 | V370 | G371 | T375 | F377 | L378 | G403 | F404 | K406 | G407 | F409 | S410 | E411 | L412 | I416 |
| 34 | I340 | Q354 | L359 | F372 | V376 | G377 | T381 | F383 | L384 | G409 | F410 | K412 | G413 | F415 | S416 | E417 | L418 | I422 |
| 36 | G259 | V276 | L281 | F301 | L305 | F306 | T310 | F312 | F313 | G324 | F325 | E327 | A328 | F330 | Q331 | A332 | L333 | L337 |
| 38 | R302 | E318 | L323 | F336 | V340 | E341 | T345 | F347 | Y348 | G358 | F359 | I361 | G362 | F364 | K365 | A366 | L367 | L371 |
| 40 | R293 | E305 | L310 | F323 | V327 | T328 | T332 | F334 | F335 | S345 | F346 | N348 | G349 | F351 | K352 | A353 | L354 | I358 |
| 42 | R312 | K324 | L329 | F342 | I346 | V347 | T351 | F353 | F354 | S364 | F365 | V367 | G368 | F370 | K371 | A372 | L373 | I377 |
| 44 | R327 | Q339 | L344 | F357 | L361 | G362 | T366 | F368 | F369 | G379 | F380 | A382 | G383 | F385 | Q386 | A387 | L388 | I392 |
| 46 | R327 | Q339 | L344 | F357 | L361 | G362 | T366 | F368 | F369 | G379 | F380 | A382 | G383 | F385 | Q386 | A387 | L388 | I392 |
| 55 | R335 | E349 | L354 | F367 | L371 | G372 | T376 | F378 | I379 | G410 | F411 | K413 | G414 | F416 | G417 | A418 | L419 | I423 |
| 57 | R335 | E349 | L354 | F367 | L371 | G372 | T376 | F378 | I379 | G410 | F411 | K413 | G414 | F416 | G417 | A418 | L419 | I423 |
| 58 | R336 | D350 | L355 | F368 | V372 | G373 | S377 | F379 | V380 | G406 | F407 | K409 | G410 | F412 | S413 | E414 | L415 | I419 |
| 59 | R346 | Q360 | L365 | F378 | V382 | G383 | T387 | F389 | L390 | G415 | F416 | K418 | G419 | F421 | S422 | E423 | L424 | I428 |
| 60 | I336 | Q350 | L355 | F368 | V372 | G373 | T377 | F379 | L380 | G405 | F406 | K408 | G409 | F411 | S412 | E413 | L414 | I418 |
| 61 | R341 | Q355 | L360 | F373 | V377 | G378 | T382 | F384 | L385 | G410 | F411 | K413 | G414 | F416 | S417 | Q418 | L419 | I423 |
| 62 | R348 | E362 | L367 | F380 | V384 | G385 | T389 | F391 | I392 | G417 | F418 | K420 | G421 | F423 | S424 | E425 | L426 | I430 |
| 63 | R345 | E359 | L364 | F377 | L381 | G382 | T386 | F388 | I389 | G414 | F415 | K417 | G418 | F420 | S421 | E422 | L423 | I427 |
| 64 | R278 | Q290 | L295 | F312 | L316 | M317 | — | F321 | F322 | G332 | F333 | E335 | G336 | F338 | K339 | A340 | L341 | I345 |
| 65 | R278 | Q290 | L295 | F312 | L316 | M317 | — | F321 | F322 | G332 | F333 | E335 | G336 | F338 | K339 | A340 | L341 | I345 |
| 66 | I340 | Q354 | L359 | F372 | V376 | G377 | T381 | F383 | L384 | G409 | F410 | K412 | G413 | F415 | S416 | E417 | L418 | I422 |
| 67 | R341 | Q355 | L360 | F373 | V377 | G378 | T382 | F384 | L385 | G410 | F411 | K413 | G414 | F416 | S417 | Q418 | L419 | I423 |

In addition, the present invention refers to a method for identifying a HPPD-inhibiting herbicide by using a mut-HPPD encoded by a nucleic acid which comprises the nucleotide sequence of SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, or a variant or derivative thereof, and/or by using a mut-HST encoded by a nucleic acid which comprises the nucleotide sequence of SEQ ID NO: 47 or 49, or a variant or derivative thereof.

Said method comprises the steps of:
a) generating a transgenic cell or plant comprising a nucleic acid encoding a mut-HPPD, wherein the mut-HPPD is expressed;
b) applying a HPPD-inhibiting herbicide to the transgenic cell or plant of a) and to a control cell or plant of the same variety;
c) determining the growth or the viability of the transgenic cell or plant and the control cell or plant after application of said HPPD-inhibiting herbicide, and
d) selecting “HPPD-inhibiting herbicides” which confer reduced growth to the control cell or plant as compared to the growth of the transgenic cell or plant.

By “control cell” or “similar, wild-type, plant, plant tissue, plant cell or host cell” is intended a plant, plant tissue, plant cell, or host cell, respectively, that lacks the herbicide-resistance characteristics and/or particular polynucleotide of the invention that are disclosed herein. The use of the term “wild-type” is not, therefore, intended to imply that a plant, plant tissue, plant cell, or other host cell lacks recombinant DNA in its genome, and/or does not possess herbicide-resistant characteristics that are different from those disclosed herein.

Another object refers to a method of identifying a nucleotide sequence encoding a mut-HPPD which is resistant or tolerant to a HPPD-inhibiting herbicide, the method comprising:
a) generating a library of mut-HPPD-encoding nucleic acids,
b) screening a population of the resulting mut-HPPD-encoding nucleic acids by expressing each of said nucleic acids in a cell or plant and treating said cell or plant with a HPPD-inhibiting herbicide,
c) comparing the HPPD-inhibiting herbicide-tolerance levels provided by said population of mut-HPPD encoding nucleic acids with the HPPD-inhibiting herbicide-tolerance level provided by a control HPPD-encoding nucleic acid,
d) selecting at least one mut-HPPD-encoding nucleic acid that provides a significantly increased level of tolerance to a HPPD-inhibiting herbicide as compared to that provided by the control HPPD-encoding nucleic acid.

In a preferred embodiment, the mut-HPPD-encoding nucleic acid selected in step d) provides at least 2-fold as much resistance or tolerance of a cell or plant to a HPPD-inhibiting herbicide as compared to that provided by the control HPPD-encoding nucleic acid.

In a further preferred embodiment, the mut-HPPD-encoding nucleic acid selected in step d) provides at least 2-fold, at least 5-fold, at least 10-fold, at least 20-fold, at least 50-fold, at least 100-fold, at least 500-fold, as much resistance or tolerance of a cell or plant to a HPPD-inhibiting herbicide as compared to that provided by the control HPPD-encoding nucleic acid.

The resistance or tolerance can be determined by generating a transgenic plant or host cell, preferably a plant cell, comprising a nucleic acid sequence of the library of step a) and comparing said transgenic plant with a control plant or host cell, preferably a plant cell.

Another object refers to a method of identifying a plant or algae containing a nucleic acid comprising a nucleotide sequence encoding a mut-HPPD or mut-HST which is resistant or tolerant to a HPPD-inhibiting herbicide, the method comprising:
a) identifying an effective amount of a HPPD-inhibiting herbicide in a culture of plant cells or green algae that leads to death of said cells.
b) treating said plant cells or green algae with a mutagenizing agent,
c) contacting said mutagenized cells population with an effective amount of HPPD-inhibiting herbicide, identified in a),
d) selecting at least one cell surviving these test conditions,
e) PCR-amplification and sequencing of HPPD and/or HST genes from cells selected in d) and comparing such sequences to wild-type HPPD or HST gene sequences, respectively.

In a preferred embodiment, said mutagenizing agent is ethylmethanesulfonate (EMS).

Many methods well known to the skilled artisan are available for obtaining suitable candidate nucleic acids for identifying a nucleotide sequence encoding a mut-HPPD from a variety of different potential source organisms including microbes, plants, fungi, algae, mixed cultures etc. as well as environmental sources of DNA such as soil. These methods include inter alia the preparation of cDNA or genomic DNA libraries, the use of suitably degenerate oligonucleotide primers, the use of probes based upon known sequences or complementation assays (for example, for growth upon tyrosine) as well as the use of mutagenesis and shuffling in order to provide recombined or shuffled mut-HPPD-encoding sequences.

Nucleic acids comprising candidate and control HPPD encoding sequences can be expressed in yeast, in a bacterial host strain, in an alga or in a higher plant such as tobacco or Arabidopsis and the relative levels of inherent tolerance of the HPPD encoding sequences screened according to a visible indicator phenotype of the transformed strain or plant in the presence of different concentrations of the selected HPPD-inhibiting herbicide. Dose responses and relative shifts in dose responses associated with these indicator phenotypes (formation of brown color, growth inhibition, herbicidal effect etc) are conveniently expressed in terms, for example, of GR50 (concentration for 50% reduction of growth) or MIC (minimum inhibitory concentration) values where increases in values correspond to increases in inherent tolerance of the expressed HPPD. For example, in a relatively rapid assay system based upon transformation of a bacterium such as E. coli, each mut-HPPD encoding sequence may be expressed, for example, as a DNA sequence under expression control of a controllable promoter such as the lacZ promoter and taking suitable account, for example by the use of synthetic DNA, of such issues as codon usage in order to obtain as comparable a level of expression as possible of different HPPD sequences. Such strains expressing nucleic acids comprising alternative candidate HPPD sequences may be plated out on different concentrations of the selected HPPD-inhibiting herbicide in, optionally, a tyrosine supplemented medium and the relative levels of inherent tolerance of the expressed HPPD enzymes estimated on the basis of the extent and MIC for inhibition of the formation of the brown, ochronotic pigment.

In another embodiment, candidate nucleic acids are transformed into plant material to generate a transgenic plant, regenerated into morphologically normal fertile plants which are then measured for differential tolerance to selected HPPD-inhibiting-herbicides. Many suitable methods for transformation using suitable selection markers such as kanamycin, binary vectors such as from Agrobacterium and plant regeneration as, for example, from tobacco leaf discs are well known in the art. Optionally, a control population of plants is likewise transformed with a nucleic acid expressing the control HPPD. Alternatively, an untransformed dicot plant such as Arabidopsis or Tobacco can be used as a control since this, in any case, expresses its own endogenous HPPD. The average, and distribution, of herbicide tolerance levels of a range of primary plant transformation events or their progeny to HPPD-inhibiting herbicides, as depicted, for example, in Table 2 are evaluated in the normal manner based upon plant damage, meristematic bleaching symptoms etc. at a range of different concentrations of herbicides. These data can be expressed in terms of, for example, GR50 values derived from dose/response curves having “dose” plotted on the x-axis and “percentage kill”, “herbicidal effect”, “numbers of emerging green plants” etc. plotted on the y-axis where increased GR50 values correspond to increased levels of inherent tolerance of the expressed HPPD. Herbicides can suitably be applied pre-emergence or post-emergence.

Another object refers to an isolated nucleic acid encoding a mut-HPPD, wherein the nucleic acid is identifiable by a method as defined above.

In another embodiment, the invention refers to a plant cell transformed by a wild-type or a mut-HPPD nucleic acid according to the present invention or or a plant cell which has been mutated to obtain a plant expressing a wild-type or a mut-HPPD nucleic acid, wherein expression of the nucleic acid in the plant cell results in increased resistance or tolerance to a HPPD-inhibiting herbicide as compared to a wild-type variety of the plant cell.

The term “expression/expressing” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

To obtain the desired effect, i.e. plants that are tolerant or resistant to the HPPD-inhibiting herbicide of the present invention, it will be understood that the at least one nucleic acid is “overexpressed” by methods and means known to the person skilled in the art.

The term “increased expression” or “overexpression” as used herein means any form of expression that is additional to the original wild-type expression level. Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art. For general information see: The Maize Handbook, Chapter 116, Freeling and Walbot, Eds., Springer, N.Y. (1994)

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds is harvested in both cases, and these seeds can be distinguished from non-transgenic seeds by growing under the above-described selective conditions. In addition the stable transformation of plastids is of advantages because plastids are inherited maternally in most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229). The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Preferably, the wild-type or mut-HPPD nucleic acid (a) or wild-type or mut-HST nucleic acid (b) comprises a polynucleotide sequence selected from the group consisting of: a) a polynucleotide as shown in SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, or a variant or derivative thereof; b) a polynucleotide as shown in SEQ ID NO: 47 or 49, or a variant or derivative thereof; c) a polynucleotide encoding a polypeptide as shown in SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, or a variant or derivative thereof; d) a polynucleotide comprising at least 60 consecutive nucleotides of any of a) through c); and e) a polynucleotide complementary to the polynucleotide of any of a) through d).

Preferably, the expression of the nucleic acid in the plant results in the plant's increased resistance to HPPD-inhibiting herbicide as compared to a wild-type variety of the plant.

In another embodiment, the invention refers to a plant, preferably a transgenic plant, comprising a plant cell according to the present invention, wherein expression of the nucleic acid in the plant results in the plant's increased resistance to HPPD-inhibiting herbicide as compared to a wild-type variety of the plant.

The plants described herein can be either transgenic crop plants or non-transgenic plants.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by nonnatural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein. Furthermore, the term “transgenic” refers to any plant, plant cell, callus, plant tissue, or plant part, that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

Plants containing mutations arising due to non-spontaneous mutagenesis and selective breeding are referred to herein as non-transgenic plants and are included in the present invention. In embodiments wherein the plant is transgenic and comprises multiple mut-HPPD nucleic acids, the nucleic acids can be derived from different genomes or from the same genome. Alternatively, in embodiments wherein the plant is non-transgenic and comprises multiple mut-HPPD nucleic acids, the nucleic acids are located on different genomes or on the same genome.

In certain embodiments, the present invention involves herbidicide-resistant plants that are produced by mutation breeding. Such plants comprise a polynucleotide encoding a mut-HPPD and/or a mut-HST and are tolerant to one or more “HPPD-inhibiting herbicides”. Such methods can involve, for example, exposing the plants or seeds to a mutagen, particularly a chemical mutagen such as, for example, ethyl methanesulfonate (EMS) and selecting for plants that have enhanced tolerance to at least one or more HPPD-inhibiting herbicide.

However, the present invention is not limited to herbicide-tolerant plants that are produced by a mutagenesis method involving the chemical mutagen EMS. Any mutagenesis method known in the art may be used to produce the herbicide-resistant plants of the present invention. Such mutagenesis methods can involve, for example, the use of any one or more of the following mutagens: radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 250 to 290 nm), and chemical mutagens such as base analogues (e.g., 5-bromo-uracil), related cornpounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, or acridines. Herbicide-resistant plants can also be produced by using tissue culture methods to select for plant cells comprising herbicide-resistance mutations and then regenerating herbicide-resistant plants therefrom. See, for example, U.S. Pat. Nos. 5,773,702 and 5,859,348, both of which are herein incorporated in their entirety by reference. Further details of mutation breeding can be found in “Principals of Cultivar Development” Fehr, 1993 Macmillan Publishing Company the disclosure of which is incorporated herein by reference

In addition to the definition above, the term “plant” is intended to encompass crop plants at any stage of maturity or development, as well as any tissues or organs (plant parts) taken or derived from any such plant unless otherwise clearly indicated by context. Plant parts include, but are not limited to, stems, roots, flowers, ovules, stamens, leaves, embryos, meristematic regions, callus tissue, anther cultures, gametophytes, sporophytes, pollen, microspores, protoplasts, and the like.

The plant of the present invention comprises at least one mut-HPPD nucleic acid or overexpressed wild-type HPPD nucleic acid, and has increased tolerance to a HPPD-inhibiting herbicide as compared to a wild-type variety of the plant. It is possible for the plants of the present invention to have multiple wild-type or mut-HPPD nucleic acids from different genomes since these plants can contain more than one genome. For example, a plant contains two genomes, usually referred to as the A and B genomes. Because HPPD is a required metabolic enzyme, it is assumed that each genome has at least one gene coding for the HPPD enzyme (i.e. at least one HPPD gene). As used herein, the term “HPPD gene locus” refers to the position of an HPPD gene on a genome, and the terms “HPPD gene” and “HPPD nucleic acid” refer to a nucleic acid encoding the HPPD enzyme. The HPPD nucleic acid on each genome differs in its nucleotide sequence from an HPPD nucleic acid on another genome. One of skill in the art can determine the genome of origin of each HPPD nucleic acid through genetic crossing and/or either sequencing methods or exonuclease digestion methods known to those of skill in the art.

The present invention includes plants comprising one, two, three, or more mut-HPPD alleles, wherein the plant has increased tolerance to a HPPD-inhibiting herbicide as compared to a wild-type variety of the plant. The mut-HPPD alleles can comprise a nucleotide sequence selected from the group consisting of a polynucleotide as defined in SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, or a variant or derivative thereof, a polynucleotide encoding a polypeptide as defined in SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, or a variant or derivative, homologue, orthologue, paralogue thereof, a polynucleotide comprising at least 60 consecutive nucleotides of any of the aforementioned polynucleotides; and a polynucleotide complementary to any of the aforementioned polynucleotides.

“Alleles” or “allelic variants” are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms

The term “variety” refers to a group of plants within a species defined by the sharing of a common set of characteristics or traits accepted by those skilled in the art as sufficient to distinguish one cultivar or variety from another cultivar or variety. There is no implication in either term that all plants of any given cultivar or variety will be genetically identical at either the whole gene or molecular level or that any given plant will be homozygous at all loci. A cultivar or variety is considered “true breeding” for a particular trait if, when the true-breeding cultivar or variety is self-pollinated, all of the progeny contain the trait. The terms “breeding line” or “line” refer to a group of plants within a cultivar defined by the sharing of a common set of characteristics or traits accepted by those skilled in the art as sufficient to distinguish one breeding line or line from another breeding line or line. There is no implication in either term that all plants of any given breeding line or line will be genetically identical at either the whole gene or molecular level or that any given plant will be homozygous at all loci. A breeding line or line is considered “true breeding” for a particular trait if, when the true-breeding line or breeding line is self-pollinated, all of the progeny contain the trait. In the present invention, the trait arises from a mutation in a HPPD gene of the plant or seed.

In some embodiments, traditional plant breeding is employed whereby the HPPD-inhibiting herbicides-tolerant trait is introduced in the progeny plant resulting therefrom. In one embodiment, the present invention provides a method for producing a HPPD-inhibiting herbicides-tolerant progeny plant, the method comprising: crossing a parent plant with a HPPD-inhibiting herbicides-tolerant plant to introduce the HPPD-inhibiting herbicides-tolerance characteristics of the HPPD-inhibiting herbicides-tolerant plant into the germplasm of the progeny plant, wherein the progeny plant has increased tolerance to the HPPD-inhibiting herbicides relative to the parent plant. In other embodiments, the method further comprises the step of introgressing the HPPD-inhibiting herbicides-tolerance characteristics through traditional plant breeding techniques to obtain a descendent plant having the HPPD-inhibiting herbicides-tolerance characteristics.

The herbicide-resistant plants of the invention that comprise polynucleotides encoding mut-HPPD and/or mut-HST polypeptides also find use in methods for increasing the herbicide-resistance of a plant through conventional plant breeding involving sexual reproduction. The methods comprise crossing a first plant that is a herbicide-resistant plant of the invention to a second plant that may or may not be resistant to the same herbicide or herbicides as the first plant or may be resistant to different herbicide or herbicides than the first plant. The second plant can be any plant that is capable of producing viable progeny plants (i.e., seeds) when crossed with the first plant. Typically, but not necessarily, the first and second plants are of the same species. The methods can optionally involve selecting for progeny plants that comprise the mut-HPPD and/or mut-HST polypeptides of the first plant and the herbicide resistance characteristics of the second plant. The progeny plants produced by this method of the present invention have increased resistance to a herbicide when compared to either the first or second plant or both. When the first and second plants are resistant to different herbicides, the progeny plants will have the combined herbicide tolerance characteristics of the first and second plants. The methods of the invention can further involve one or more generations of backcrossing the progeny plants of the first cross to a plant of the same line or genotype as either the first or second plant. Alternatively, the progeny of the first cross or any subsequent cross can be crossed to a third plant that is of a different line or genotype than either the first or second plant. The present invention also provides plants, plant organs, plant tissues, plant cells, seeds, and non-human host cells that are transformed with the at least one polynucleotide molecule, expression cassette, or transformation vector of the invention. Such transformed plants, plant organs, plant tissues, plant cells, seeds, and non-human host cells have enhanced tolerance or resistance to at least one herbicide, at levels of the herbicide that kill or inhibit the growth of an untransformed plant, plant tissue, plant cell, or non-human host cell, respectively. Preferably, the transformed plants, plant tissues, plant cells, and seeds of the invention are Arabidopsis thaliana and crop plants.

In other aspects, plants of the invention include those plants which, in addition to being HPPD-inhibiting herbicides-tolerant, have been subjected to further genetic modifications by breeding, mutagenesis or genetic engineering, e.g. have been rendered tolerant to applications of specific other classes of herbicides, such as AHAS inhibitors; auxinic herbicides; bleaching herbicides such as hydroxyphenylpyruvate dioxygenase (HPPD) inhibitors or phytoene desaturase (PDS) inhibitors; EPSPS inhibitors such as glyphosate; glutamine synthetase (GS) inhibitors such as glufosinate; lipid biosynthesis inhibitors such as acetyl CoA carboxylase (ACCase) inhibitors; or oxynil {i.e. bromoxynil or ioxynil) herbicides as a result of conventional methods of breeding or genetic engineering, Thus, HPPD-inhibiting herbicides-tolerant plants of the invention can be made resistant to multiple classes of herbicides through multiple genetic modifications, such as resistance to both glyphosate and glufosinate or to both glyphosate and a herbicide from another class such as HPPD inhibitors, AHAS inhibitors, or ACCase inhibitors. These herbicide resistance technologies are, for example, described in Pest Management Science (at volume, year, page): 61, 2005, 246; 61, 2005, 258; 61, 2005, 277; 61, 2005, 269; 61, 2005, 286; 64, 2008, 326; 64, 2008, 332; Weed Science 57, 2009, 108; Australian Journal of Agricultural Research 58, 2007, 708; Science 316, 2007, 1185; and references quoted therein. For example, HPPD-inhibiting herbicides-tolerant plants of the invention, in some embodiments, may be tolerant to ACCase inhibitors, such as “dims” {e.g., cycloxydim, sethoxydim, clethodim, or tepraloxydim), “fops” {e.g., clodinafop, diclofop, fluazifop, haloxyfop, or quizalofop), and “dens” (such as pinoxaden); to auxinic herbicides, such as dicamba; to EPSPS inhibitors, such as glyphosate; to other HPPD inhibitors; and to GS inhibitors, such as glufosinate.

In addition to these classes of inhibitors, HPPD-inhibiting herbicides-tolerant plants of the invention may also be tolerant to herbicides having other modes of action, for example, chlorophyll/carotenoid pigment inhibitors, cell membrane disrupters, photosynthesis inhibitors, cell division inhibitors, root inhibitors, shoot inhibitors, and combinations thereof.

Such tolerance traits may be expressed, e.g.: as mutant or wild-type HPPD proteins, as mutant AHASL proteins, mutant ACCase proteins, mutant EPSPS proteins, or mutant glutamine synthetase proteins; or as mutant native, inbred, or transgenic aryloxyalkanoate dioxygenase (AAD or DHT), haloarylnitrilase (BXN), 2,2-dichloropropionic acid dehalogenase (DEH), glyphosate-N-acetyltransferase (GAT), glyphosate decarboxylase (GDC), glyphosate oxidoreductase (GOX), glutathione-S-transferase (GST), phosphinothricin acetyltransferase (PAT or bar), or CYP450s proteins having an herbicide-degrading activity. HPPD-inhibiting herbicides-tolerant plants hereof can also be stacked with other traits including, but not limited to, pesticidal traits such as Bt Cry and other proteins having pesticidal activity toward coleopteran, lepidopteran, nematode, or other pests; nutrition or nutraceutical traits such as modified oil content or oil profile traits, high protein or high amino acid concentration traits, and other trait types known in the art.

Furthermore, in other embodiments, HPPD-inhibiting herbicides-tolerant plants are also covered which are, by the use of recombinant DNA techniques and/or by breeding and/or otherwise selected for such characteristics, rendered able to synthesize one or more insecticidal proteins, especially those known from the bacterial genus Bacillus, particularly from Bacillus thuringiensis, such as [delta]-endotoxins, e.g. CryIA(b), CryIA(c), CryIF, CryIF(a2), CryIIA(b), CryIIIA, CryIIIB(bI) or Cry9c; vegetative insecticidal proteins (VIP), e.g. VIP1, VIP2, VIP3 or VIP3A; insecticidal proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such streptomycete toxins; plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxy-steroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e.g. WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e.g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda).

In some embodiments, expression of one or more protein toxins (e.g., insecticidal proteins) in the HPPD-inhibiting herbicides-tolerant plants is effective for controlling organisms that include, for example, members of the classes and orders: Coleoptera such as the American bean weevil Acanthoscelides obtectus; the leaf beetle Agelastica alni; click beetles (Agriotes lineatus, Agriotes obscurus, Agriotes bicolor); the grain beetle Ahasverus advena; the summer schafer Amphimallon solstitialis; the furniture beetle Anobium punctatum; Anthonomus spp. (weevils); the Pygmy mangold beetle Atomaria linearis; carpet beetles (Anthrenus spp., Attagenus spp.); the cowpea weevil Callosobruchus maculates; the fried fruit beetle Carpophilus hemipterus; the cabbage seedpod weevil Ceutorhynchus assimilis; the rape winter stem weevil Ceutorhynchus picitarsis; the wireworms Conoderus vespertinus and Conoderus falli; the banana weevil Cosmopolites sordidus; the New Zealand grass grub Costelytra zealandica; the June beetle Cotinis nitida; the sunflower stem weevil Cylindrocopturus adspersus; the larder beetle Dermestes lardarius; the corn rootworms Diabrotica virgifera, Diabrotica virgifera virgifera, and Diabrotica barberi; the Mexican bean beetle Epilachna varivestis; the old house borer Hylotropes bajulus; the lucerne weevil Hypera postica; the shiny spider beetle Gibbium psylloides; the cigarette beetle Lasioderma serricorne; the Colorado potato beetle Leptinotarsa decemlineata; Lyctus beetles {Lyctus spp., the pollen beetle Meligethes aeneus; the common cockshafer Melolontha melolontha; the American spider beetle Mezium americanum; the golden spider beetle Niptus hololeuc s; the grain beetles Oryzaephilus surinamensis and Oryzaephilus Mercator; the black vine weevil Otiorhynchus sulcatus; the mustard beetle Phaedon cochleariae, the crucifer flea beetle Phyllotreta cruciferae; the striped flea beetle Phyllotreta striolata; the cabbage steam flea beetle Psylliodes chrysocephala; Ptinus spp. (spider beetles); the lesser grain borer Rhizopertha dominica; the pea and been weevil Sitona lineatus; the rice and granary beetles Sitophilus oryzae and Sitophilus granaries; the red sunflower seed weevil Smicronyx fulvus; the drugstore beetle Stegobium paniceum; the yellow mealworm beetle Tenebrio molitor, the flour beetles Tribolium castaneum and Tribolium confusum; warehouse and cabinet beetles {Trogoderma spp.); the sunflower beetle Zygogramma exclamationis; Dermaptera (earwigs) such as the European earwig Forficula auricularia and the striped earwig Labidura riparia; Dictyoptera such as the oriental cockroach Blatta orientalis; the greenhouse millipede Oxidus gracilis; the beet fly Pegomyia betae; the frit fly Oscinella frit; fruitflies (Dacus spp., Drosophila spp.); Isoptera (termites) including species from the familes Hodotermitidae, Kalotermitidae, Mastotermitidae, Rhinotermitidae, Serritermitidae, Termitidae, Termopsidae; the tarnished plant bug Lygus lineolaris; the black bean aphid Aphis fabae; the cotton or melon aphid Aphis gossypii; the green apple aphid Aphis pomi; the citrus spiny whitefly Aleurocanthus spiniferus; the sweet potato whitefly Bemesia tabaci; the cabbage aphid Brevicoryne brassicae; the pear psylla Cacopsylla pyricola; the currant aphid Cryptomyzus ribis; the grape phylloxera Daktulosphaira vitifoliae; the citrus psylla Diaphorina citri; the potato leafhopper Empoasca fabae; the bean leafhopper Empoasca Solana; the vine leafhopper Empoasca vitis; the woolly aphid Eriosoma lanigerum; the European fruit scale Eulecanium corni; the mealy plum aphid Hyalopterus arundinis; the small brown planthopper Laodelphax striatellus; the potato aphid Macrosiphum euphorbiae; the green peach aphid Myzus persicae; the green rice leafhopper Nephotettix cinticeps; the brown planthopper Nilaparvata lugens; the hop aphid Phorodon humuli; the bird-cherry aphid Rhopalosiphum padi; the grain aphid Sitobion avenae; Lepidoptera such as Adoxophyes orana (summer fruit tortrix moth); Archips podana (fruit tree tortrix moth); Bucculatrix pyrivorella (pear leafminer); Bucculatrix thurberiella (cotton leaf perforator); Bupalus piniarius (pine looper); Carpocapsa pomonella (codling moth); Chilo suppressalis (striped rice borer); Choristoneura fumiferana (eastern spruce budworm); Cochylis hospes (banded sunflower moth); Diatraea grandiosella (southwestern corn borer); Eupoecilia ambiguella (European grape berry moth); Helicoverpa armigera (cotton bollworm); Helicoverpa zea (cotton bollworm); Heliothis vires cens (tobacco budworm), Homeosoma electellum (sunflower moth); Homona magnanima (oriental tea tree tortrix moth); Lithocolletis blancardella (spotted tentiform leafminer); Lymantria dispar (gypsy moth); Malacosoma neustria (tent caterpillar); Mamestra brassicae (cabbage armyworm); Mamestra configurata (Bertha armyworm); Operophtera brumata (winter moth); Ostrinia nubilalis (European corn borer), Panolis flammea (pine beauty moth), Phyllocnistis citrella (citrus leafminer); Pieris brassicae (cabbage white butterfly); Rachiplusia ni (soybean looper); Spodoptera exigua (beet armywonn); Spodoptera littoralis (cotton leafworm); Sylepta derogata (cotton leaf roller); Trichoplusia ni (cabbage looper); Orthoptera such as the common cricket Acheta domesticus, tree locusts (Anacridium spp.), the migratory locust Locusta migratoria, the twostriped grasshopper Melanoplus bivittatus, the differential grasshopper Melanoplus differ entialis, the redlegged grasshopper Melanoplus femurrubrum, the migratory grasshopper Melanoplus sanguinipes, the northern mole cricket Neocurtilla hexadectyla, the red locust Nomadacris septemfasciata, the shortwinged mole cricket Scapteriscus abbreviatus, the southern mole cricket Scapteriscus borellii, the tawny mole cricket Scapteriscus vicinus, and the desert locust Schistocerca gregaria; Symphyla such as the garden symphylan Scutigerella immaculata; Thysanoptera such as the tobacco thrips Frankliniella fusca, the flower thrips Frankliniella intonsa, the western flower thrips Frankliniella occidentalism the cotton bud thrips Frankliniella schultzei, the banded greenhouse thrips Hercinothrips femoralis, the soybean thrips Neohydatothrips variabilis, Kelly's citrus thrips Pezothrips kellyanus, the avocado thrips Scirtothrips perseae, the melon thrips Thrips palmi, and the onion thrips Thrips tabaci; and the like, and combinations comprising one or more of the foregoing organisms.

In some embodiments, expression of one or more protein toxins (e.g., insecticidal proteins) in the HPPD-inhibiting herbicides-tolerant plants is effective for controlling flea beetles, i.e. members of the flea beetle tribe of family Chrysomelidae, preferably against Phyllotreta spp., such as Phyllotreta cruciferae and/or Phyllotreta triolata. In other embodiments, expression of one or more protein toxins {e.g., insecticidal proteins) in the HPPD-inhibiting herbicides-tolerant plants is effective for controlling cabbage seedpod weevil, the Bertha armyworm, Lygus bugs, or the diamondback moth.

It is to be understood that the plant of the present invention can comprise a wild-type HPPD nucleic acid in addition to a mut-HPPD nucleic acid. It is contemplated that the HPPD-inhibiting herbicide tolerant lines may contain a mutation in only one of multiple HPPD isoenzymes. Therefore, the present invention includes a plant comprising one or more mut-HPPD nucleic acids in addition to one or more wild-type HPPD nucleic acids.

In another embodiment, the invention refers to a seed produced by a transgenic plant comprising a plant cell of the present invention, wherein the seed is true breeding for an increased resistance to a HPPD-inhibiting herbicide as compared to a wild-type variety of the seed.

In another embodiment, the invention refers to a method of producing a transgenic plant cell with an increased resistance to a HPPD-inhibiting herbicide as compared to a wild-type variety of the plant cell comprising, transforming the plant cell with an expression cassette comprising a nucleic acid encoding a wild-type or a mut-HPPD as defined SUPRA.

In another embodiment, the invention refers to a method of producing a transgenic plant comprising, (a) transforming a plant cell with an expression cassette comprising a nucleic acid encoding a wild-type or a mut-HPPD, and (b) generating a plant with an increased resistance to HPPD-inhibiting herbicide from the plant cell.

Consequently, HPPD nucleic acids encoding a wild-type or a mut-HPPD useful for the invention are provided in expression cassettes for expression in the plant of interest. The cassette will include regulatory sequences operably linked to a HPPD nucleic acid sequence encoding a wild-type or a mut-HPPD of the invention. The term “regulatory element” as used herein refers to a polynucleotide that is capable of regulating the transcription of an operably linked polynucleotide. It includes, but not limited to, promoters, enhancers, introns, 5′ UTRs, and 3′ UTRs. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the HPPD nucleic acid sequence to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a mut-HPPD nucleic acid sequence of the invention, and a transcriptional and translational termination region (i.e., termination region) functional in plants. The promoter may be native or analogous, or foreign or heterologous, to the plant host and/or to the HPPD nucleic acid sequence of the invention. Additionally, the promoter may be the natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the HPPD nucleic acid sequence of the invention, it is intended that the promoter is not the native or naturally occurring promoter for the operably linked HPPD nucleic acid sequence of the invention. As used herein, a chimeric gene comprises a coding sequence operably linked to a transcription initiation region that is heterologous to the coding sequence.

While it may be preferable to express the HPPD nucleic acids of the invention using heterologous promoters, the native promoter sequences may be used. Such constructs would change expression levels of the HPPD protein in the plant or plant cell. Thus, the phenotype of the plant or plant cell is altered.

The termination region may be native with the transcriptional initiation region, may be native with the operably linked HPPD sequence of interest, may be native with the plant host, or may be derived from another source (i.e., foreign or heterologous to the promoter, the HPPD nucleic acid sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64:671-674; Sanfacon et al. (1991) Genes Dev. 5: 141-149; Mogen et al. (1990) Plant Cell 2: 1261-1272; Munroe et al. (1990) Gene 91: 151-158; Ballas t al. (1989) Nucleic Acids Res. 17:7891-7903; and Joshi et al. (1987) Nucleic Acid Res. 15:9627-9639. Where appropriate, the gene(s) may be optimized for increased expression in the transformed plant. That is, the genes can be synthesized using plant-preferred codons for improved expression. See, for example, Campbell and Gowri (1990) Plant Physiol. 92: 1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures. Nucleotide sequences for enhancing gene expression can also be used in the plant expression vectors. These include the introns of the maize AdhI, intronl gene (Callis et al. Genes and Development 1: 1183-1200, 1987), and leader sequences, (W-sequence) from the Tobacco Mosaic virus (TMV), Maize Chlorotic Mottle Virus and Alfalfa Mosaic Virus (Gallie et al. Nucleic Acid Res. 15:8693-8711, 1987 and Skuzeski et al. Plant Mol. Biol. 15:65-79, 1990). The first intron from the shrunken-1 locus of maize, has been shown to increase expression of genes in chimeric gene constructs. U.S. Pat. Nos. 5,424,412 and 5,593,874 disclose the use of specific introns in gene expression constructs, and Gallie et al. (Plant Physiol. 106:929-939, 1994) also have shown that introns are useful for regulating gene expression on a tissue specific basis. To further enhance or to optimize mut-HPPD gene expression, the plant expression vectors of the invention may also contain DNA sequences containing matrix attachment regions (MARs). Plant cells transformed with such modified expression systems, then, may exhibit overexpression or constitutive expression of a nucleotide sequence of the invention.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. ScL USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and trans versions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2: 163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced HPPD expression within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20: 181-196; Orozco et al. (1993) Plant Mol Biol. 23(6): 1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression. In one embodiment, the nucleic acids of interest are targeted to the chloroplast for expression. In this manner, where the nucleic acid of interest is not directly inserted into the chloroplast, the expression cassette will additionally contain a chloroplast-targeting sequence comprising a nucleotide sequence that encodes a chloroplast transit peptide to direct the gene product of interest to the chloroplasts. Such transit peptides are known in the art. With respect to chloroplast-targeting sequences, “operably linked” means that the nucleic acid sequence encoding a transit peptide (i.e., the chloroplast-targeting sequence) is linked to the HPPD nucleic acid of the invention such that the two sequences are contiguous and in the same reading frame. See, for example, Von Heijne et al. (1991) Plant Mol. Biol. Rep. 9: 104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481. Any chloroplast transit peptide known in the art can be fused to the amino acid sequence of a mature HPPD protein of the invention by operably linking a choloroplast-targeting sequence to the 5′-end of a nucleotide sequence encoding a mature mut-HPPD protein of the invention. Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Lamppa et al. (1988) J. Biol. Chem. 263: 14996-14999). See also Von Heijne et al. (1991) Plant Mol. Biol. Rep. 9: 104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196: 1414-1421; and Shah et al. (1986) Science 233:478-481.

Methods for transformation of chloroplasts are known in the art. See, for example, Svab et al. (1990) Proc. Natl. Acad. ScL USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305. The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

In a preferred embodiment, the HPPD nucleic acid encoding a wild-type or a mut-HPPD (a) or the HST nucleic acid (b) comprises a polynucleotide sequence selected from the group consisting of: a) a polynucleotide as shown in SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, or a variant or derivative thereof; b) a polynucleotide as shown in SEQ ID NO: 47 or 49, or a variant or derivative thereof; c) a polynucleotide encoding a polypeptide as shown in SEQ ID NO: 2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, or a variant or derivative thereof; d) a polynucleotide comprising at least 60 consecutive nucleotides of any of a) through c); and e) a polynucleotide complementary to the polynucleotide of any of a) through d)

Preferably, the expression cassette further comprises a transcription initiation regulatory region and a translation initiation regulatory region that are functional in the plant.

While the polynucleotides of the invention find use as selectable marker genes for plant transformation, the expression cassettes of the invention can include another selectable marker gene for the selection of transformed cells. Selectable marker genes, including those of the present invention, are utilized for the selection of transformed cells or tissues. Marker genes include, but are not limited to, genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christophers on et al (1992) Proc. Natl. Acad. ScL USA 89:6314-6318; Yao et al. (1992) Cell 71:63-72; Reznikoff (1992) Mol Microbiol 6:2419-2422; Barkley et al (1980) in The Operon, pp. 177-220; Hu et al (1987) Cell 48:555-566; Brown et al (1987) Cell 49:603-612; Figge et al (1988) Cell 52:713-722; Deuschle et al (1989) Proc. Natl Acad. AcL USA 86:5400-5404; Fuerst et al (1989) Proc. Natl Acad. ScL USA 86:2549-2553; Deuschle et al (1990) Science 248:480-483; Gossen (1993) Ph.D. Thesis, University of Heidelberg; Reines et al (1993) Proc. Natl Acad. ScL USA 90: 1917-1921; Labow et al (1990) Mol Cell Biol 10:3343-3356; Zambretti et al (1992) Proc. Natl Acad. ScL USA 89:3952-3956; Bairn et al (1991) Proc. Natl Acad. ScL USA 88:5072-5076; Wyborski et al (1991) Nucleic Acids Res. 19:4647-4653; Hillenand-Wissman (1989) Topics Mol Struc. Biol 10: 143-162; Degenkolb et al (1991) Antimicrob. Agents Chemother. 35: 1591-1595; Kleinschnidt et al (1988) Biochemistry 27: 1094-1104; Bonin (1993) Ph.D. Thesis, University of Heidelberg; Gossen et al (1992) Proc. Natl Acad. ScL USA 89:5547-5551; Oliva et al (1992) Antimicrob. Agents Chemother. 36:913-919; Hlavka et al (1985) Handbook of Experimental Pharmacology, Vol. 78 (Springer-Verlag, Berlin); Gill et al (1988) Nature 334:721-724. Such disclosures are herein incorporated by reference. The above list of selectable marker genes is not meant to be limiting. Any selectable marker gene can be used in the present invention.

The invention further provides an isolated recombinant expression vector comprising the expression cassette containing a HPPD nucleic acid as described above, wherein expression of the vector in a host cell results in increased tolerance to a HPPD-inhibiting herbicide as compared to a wild-type variety of the host cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operably linked to the nucleic acid sequence to be expressed. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein (e.g., mut-HPPD polypeptides, fusion polypeptides, etc.).

In a preferred embodiment of the present invention, the HPPD polypeptides are expressed in plants and plants cells such as unicellular plant cells (such as algae) (See Falciatore et al., 1999, Marine Biotechnology 1(3):239-251 and references therein) and plant cells from higher plants (e.g., the spermatophytes, such as crop plants). A HPPD polynucleotide may be “introduced” into a plant cell by any means, including transfection, transformation or transduction, electroporation, particle bombardment, agroinfection, biolistics, and the like.

Suitable methods for transforming or transfecting host cells including plant cells can be found in Sambrook et al. (Molecular Cloning: A Laboratory Manual. 2nd, ed., Cold Spring Harbor Laboratory, Cold Spring Harbor Laboratory Press, Cold Spring Harbor, N. Y., 1989) and other laboratory manuals such as Methods in Molecular Biology, 1995, Vol. 44, Agrobacterium protocols, ed: Gartland and Davey, Humana Press, Totowa, N.J. As increased tolerance to HPPD-inhibiting herbicides is a general trait wished to be inherited into a wide variety of plants like maize, wheat, rye, oat, triticale, rice, barley, soybean, peanut, cotton, rapeseed and canola, manihot, pepper, sunflower and tagetes, solanaceous plants like potato, tobacco, eggplant, and tomato, Vicia species, pea, alfalfa, bushy plants (coffee, cacao, tea), Salix species, trees (oil palm, coconut), perennial grasses, and forage crops, these crop plants are also preferred target plants for a genetic engineering as one further embodiment of the present invention. In a preferred embodiment, the plant is a crop plant. Forage crops include, but are not limited to, Wheatgrass, Canarygrass, Bromegrass, Wildrye Grass, Bluegrass, Orchardgrass, Alfalfa, Salfoin, Birdsfoot Trefoil, Alsike Clover, Red Clover, and Sweet Clover.

In one embodiment of the present invention, transfection of a mut-HPPD polynucleotide into a plant is achieved by Agrobacterium mediated gene transfer. One transformation method known to those of skill in the art is the dipping of a flowering plant into an Agrobacteria solution, wherein the Agrobacteria contains the mut-HPPD nucleic acid, followed by breeding of the transformed gametes. Agrobacterium mediated plant transformation can be performed using for example the GV3101(pMP90) (Koncz and Schell, 1986, Mol. Gen. Genet. 204:383-396) or LBA4404 (Clontech) Agrobacterium tumefaciens strain. Transformation can be performed by standard transformation and regeneration techniques (Deblaere et al., 1994, Nucl. Acids. Res. 13:4777-4788; Gelvin, Stanton B. and Schilperoort, Robert A, Plant Molecular Biology Manual, 2nd Ed.—Dordrecht: Kluwer Academic Publ., 1995.—in Sect., Ringbuc Zentrale Signatur: BT11-P ISBN 0-7923-2731-4; Glick, Bernard R. and Thompson, John E., Methods in Plant Molecular Biology and Biotechnology, Boca Raton: CRC Press, 1993 360 S., ISBN 0-8493-5164-2). For example, rapeseed can be transformed via cotyledon or hypocotyl transformation (Moloney et al., 1989, Plant Cell Report 8:238-242; De Block et al., 1989, Plant Physiol. 91:694-701). Use of antibiotics for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., 1994, Plant Cell Report 13:282-285. Additionally, transformation of soybean can be performed using for example a technique described in European Patent No. 0424 047, U.S. Pat. No. 5,322,783, European Patent No. 0397 687, U.S. Pat. No. 5,376,543, or U.S. Pat. No. 5,169,770. Transformation of maize can be achieved by particle bombardment, polyethylene glycol mediated DNA uptake, or via the silicon carbide fiber technique. (See, for example, Freeling and Walbot “The maize handbook” Springer Verlag: New York (1993) ISBN 3-540-97826-7). A specific example of maize transformation is found in U.S. Pat. No. 5,990,387, and a specific example of wheat transformation can be found in PCT Application No. WO 93/07256.

According to the present invention, the introduced HPPD polynucleotide may be maintained in the plant cell stably if it is incorporated into a non-chromosomal autonomous replicon or integrated into the plant chromosomes. Alternatively, the introduced mut-HPPD polynucleotide may be present on an extra-chromosomal non-replicating vector and be transiently expressed or transiently active. In one embodiment, a homologous recombinant microorganism can be created wherein the mut-HPPD polynucleotide is integrated into a chromosome, a vector is prepared which contains at least a portion of an HPPD gene into which a deletion, addition, or substitution has been introduced to thereby alter, e.g., functionally disrupt, the endogenous HPPD gene and to create a mut-HPPD gene. To create a point mutation via homologous recombination, DNA-RNA hybrids can be used in a technique known as chimeraplasty (Cole-Strauss et al., 1999, Nucleic Acids Research 27(5):1323-1330 and Kmiec, 1999, Gene therapy American Scientist 87(3):240-247). Other homologous recombination procedures in Triticum species are also well known in the art and are contemplated for use herein.

In the homologous recombination vector, the wild-type or mut-HPPD gene can be flanked at its 5′ and 3′ ends by an additional nucleic acid molecule of the HPPD gene to allow for homologous recombination to occur between the exogenous wild-type or mut-HPPD gene carried by the vector and an endogenous HPPD gene, in a microorganism or plant. The additional flanking HPPD nucleic acid molecule is of sufficient length for successful homologous recombination with the endogenous gene. Typically, several hundreds of base pairs up to kilobases of flanking DNA (both at the 5′ and 3′ ends) are included in the vector (see e.g., Thomas, K. R., and Capecchi, M. R., 1987, Cell 51:503 for a description of homologous recombination vectors or Strepp et al., 1998, PNAS, 95(8):4368-4373 for cDNA based recombination in Physcomitrella patens). However, since the mut-HPPD gene normally differs from the HPPD gene at very few amino acids, a flanking sequence is not always necessary. The homologous recombination vector is introduced into a microorganism or plant cell (e.g., via polyethylene glycol mediated DNA), and cells in which the introduced mut-HPPD gene has homologously recombined with the endogenous HPPD gene are selected using art-known techniques.

In another embodiment, recombinant microorganisms can be produced that contain selected systems that allow for regulated expression of the introduced gene. For example, inclusion of a mut-HPPD gene on a vector placing it under control of the lac operon permits expression of the mut-HPPD gene only in the presence of IPTG. Such regulatory systems are well known in the art.

Another aspect of the invention pertains to host cells into which a recombinant expression vector of the invention has been introduced. The terms “host cell” and “recombinant host cell” are used interchangeably herein. It is understood that such terms refer not only to the particular subject cell but they also apply to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a mut-HPPD polynucleotide can be expressed in bacterial cells such as C. glutamicum, insect cells, fungal cells, or mammalian cells (such as Chinese hamster ovary cells (CHO) or COS cells), algae, ciliates, plant cells, fungi or other microorganisms like C. glutamicum. Other suitable host cells are known to those skilled in the art.

A host cell of the invention, such as a prokaryotic or eukaryotic host cell in culture, can be used to produce (i.e., express) a mut-HPPD polynucleotide. Accordingly, the invention further provides methods for producing mut-HPPD polypeptides using the host cells of the invention. In one embodiment, the method comprises culturing the host cell of invention (into which a recombinant expression vector encoding a mut-HPPD polypeptide has been introduced, or into which genome has been introduced a gene encoding a wild-type or mut-HPPD polypeptide) in a suitable medium until mut-HPPD polypeptide is produced. In another embodiment, the method further comprises isolating mut-HPPD polypeptides from the medium or the host cell. Another aspect of the invention pertains to isolated mut-HPPD polypeptides, and biologically active portions thereof. An “isolated” or “purified” polypeptide or biologically active portion thereof is free of some of the cellular material when produced by recombinant DNA techniques, or chemical precursors or other chemicals when chemically synthesized. The language “substantially free of cellular material” includes preparations of mut-HPPD polypeptide in which the polypeptide is separated from some of the cellular components of the cells in which it is naturally or recombinantly produced. In one embodiment, the language “substantially free of cellular material” includes preparations of a mut-HPPD polypeptide having less than about 30% (by dry weight) of non-mut-HPPD material (also referred to herein as a “contaminating polypeptide”), more preferably less than about 20% of non-mut-HPPD material, still more preferably less than about 10% of non-mut-HPPD material, and most preferably less than about 5% non-mut-HPPD material.

When the mut-HPPD polypeptide, or biologically active portion thereof, is recombinantly produced, it is also preferably substantially free of culture medium, i.e., culture medium represents less than about 20%, more preferably less than about 10%, and most preferably less than about 5% of the volume of the polypeptide preparation. The language “substantially free of chemical precursors or other chemicals” includes preparations of mut-HPPD polypeptide in which the polypeptide is separated from chemical precursors or other chemicals that are involved in the synthesis of the polypeptide. In one embodiment, the language “substantially free of chemical precursors or other chemicals” includes preparations of a mut-HPPD polypeptide having less than about 30% (by dry weight) of chemical precursors or non-mut-HPPD chemicals, more preferably less than about 20% chemical precursors or non-mut-HPPD chemicals, still more preferably less than about 10% chemical precursors or non-mut-HPPD chemicals, and most preferably less than about 5% chemical precursors or non-mut-HPPD chemicals. In preferred embodiments, isolated polypeptides, or biologically active portions thereof, lack contaminating polypeptides from the same organism from which the mut-HPPD polypeptide is derived. Typically, such polypeptides are produced by recombinant expression of, for example, a mut-HPPD polypeptide in plants other than, or in microorganisms such as C. glutamicum, ciliates, algae, or fungi.

As described above, the present invention teaches compositions and methods for increasing the HPPD-inhibiting herbicide, particularly bicycloarylcarboxamide tolerance of a crop plant or seed as compared to a wild-type variety of the plant or seed. In a preferred embodiment, the HPPD-inhibiting herbicide, particularly bicycloarylcarboxamide tolerance of a crop plant or seed is increased such that the plant or seed can withstand a HPPD-inhibiting herbicide, particularly bicycloarylcarboxamide application of preferably approximately 1-1000 g ai ha⁻¹, more preferably 10-500 g ai ha⁻¹, still more preferably 20-200 g ai ha⁻¹ and most preferably 40-100 g ai ha⁻¹. As used herein, to “withstand” a HPPD-inhibiting herbicide, particularly bicycloarylcarboxamide application means that the plant is either not killed or not injured by such application.

Furthermore, the present invention provides methods that involve the use of at least one HPPD-inhibiting herbicide, particularly bicycloarylcarboxamide as depicted in Table 2.

In these methods, the HPPD-inhibiting herbicide, particularly bicycloarylcarboxamide can be applied by any method known in the art including, but not limited to, seed treatment, soil treatment, and foliar treatment. Prior to application, the HPPD-inhibiting herbicide, particularly bicycloarylcarboxamide can be converted into the customary formulations, for example solutions, emulsions, suspensions, dusts, powders, pastes and granules. The use form depends on the particular intended purpose; in each case, it should ensure a fine and even distribution of the compound according to the invention.

By providing plants having increased tolerance to HPPD-inhibiting herbicide, particularly bicycloarylcarboxamide, a wide variety of formulations can be employed for protecting plants from weeds, so as to enhance plant growth and reduce competition for nutrients. A HPPD-inhibiting herbicide, particularly bicycloarylcarboxamide, can be used by itself for pre-emergence, post-emergence, pre-planting, and at-planting control of weeds in areas surrounding the crop plants described herein, or a HPPD-inhibiting herbicide formulation can be used that contains other additives. The HPPD-inhibiting herbicide, particularly bicycloarylcarboxamide, can also be used as a seed treatment. Additives found in a HPPD-inhibiting herbicide formulation include other herbicides, detergents, adjuvants, spreading agents, sticking agents, stabilizing agents, or the like. The HPPD-inhibiting herbicide formulation can be a wet or dry preparation and can include, but is not limited to, flowable powders, emulsifiable concentrates, and liquid concentrates. The HPPD-inhibiting herbicide and herbicide formulations can be applied in accordance with conventional methods, for example, by spraying, irrigation, dusting, or the like.

Suitable formulations are described in detail in PCT/EP2009/063387 and PCT/EP2009/063386, which are incorporated herein by reference.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1

### Cloning of HPPD Encoding Genes

(A) Cloning of Arabidopsis thaliana HPPD

The partial Arabidopsis thaliana AtHPPD coding sequence (SEQ ID No: 52) is amplified by standard PCR techniques from Arabidopsis thaliana cDNA using primers HuJ101 and HuJ102 (Table 5).

TABLE 5
PCR primers for AtHPPD amplification (SEQ ID NO:
70, 71)

| Primer name | Primer sequence (5′ → 3′) |
|---|---|
| HuJ101 | GGCCACCAAAACGCCG |
| HuJ102 | TCATCCCACTAACTGTTTGGCTTC |

The PCR-product is cloned in vector pEXP5-NT/TOPO® (Invitrogen, Carlsbad, USA) according to the manufacturer's instructions. The resulting plasmid pEXP5-NT/TOPO®-AtHPPD is isolated from E. coli TOP10 by performing a plasmid minipreparation. The expression cassette encoding N-terminally Hiss-tagged AtHPPD is confirmed by DNA sequencing.

(B) Cloning of Chlamydomonas reinhardtii HPPD1

The C. reinhardtii HPPD1 (CrHPPD1) coding sequence (SEQ ID No: 54) is codon-optimized for expression in E. coli and provided as a synthetic gene (Entelechon, Regensburg, Germany). The partial synthetic gene is amplified by standard PCR techniques using primers Ta1-1 and Ta1-2 (Table 6).

TABLE 6
PCR primers for CrHPPD1 amplification (SEQ ID NO:
72, 73)

| Primer name | Primer sequence (5′ → 3′) |
|---|---|
| Ta1-1 | GGCGCTGGCGGTGCGTCCACTAC |
| Ta1-2 | TCAAACGTTCAGGGTACGCTCGTAGTCTTCGATG |

The PCR-product is cloned in vector pEXP5-NT/TOPO® (Invitrogen, Carlsbad, USA) according to the manufacturer's instructions. The resulting plasmid pEXP5-NT/TOPO®-CrHPPD1 is isolated from E. coli TOP10 by performing a plasmid minipreparation. The expression cassette encoding N-terminally His6-tagged CrHPPD1 is confirmed by DNA sequencing.

(C) Cloning of C. reinhardtii HPPD2

The C. reinhardtii HPPD2 (CrHPPD2) coding sequence (SEQ ID No: 56) is codon-optimized for expression in E. coli and provided as a synthetic gene (Entelechon, Regensburg, Germany). The partial synthetic gene is amplified by standard PCR techniques using primers Ta1-3 and Ta1-4 (Table 7).

TABLE 7
PCR primers for CrHPPD2 amplification (SEQ ID NO:
74, 75)

| Primer name | Primer sequence (5′ → 3′) |
|---|---|
| Ta1-3 | GGTGCGGGTGGCGCTGGCACC |
| Ta1-4 | TCAAACGTTCAGGGTACGTTCGTAGTCCTCGATGG |

The PCR-product is cloned in vector pEXP5-NT/TOPO® (Invitrogen, Carlsbad, USA) according to the manufacturer's instructions. The resulting plasmid pEXP5-NT/TOPO®-CrHPPD2 is isolated from E. coli TOP10 by performing a plasmid minipreparation. The expression cassette encoding N-terminally His6-tagged CrHPPD2 is confirmed by DNA sequencing.

### (D) Cloning of Glycine max HPPD

The Glycine max HPPD (GmHPPD; Glyma14g03410) coding sequence is codon-optimized for expression in E. coli and provided as a synthetic gene (Entelechon, Regensburg, Germany). The partial synthetic gene is amplified by standard PCR techniques using primers Ta2-65 and Ta2-66 (Table 8).

TABLE 8
PCR primers for GmHPPD amplification (SEQ ID NO:
76, 77)

| Primer name | Primer sequence (5′ → 3′) |
|---|---|
| Ta2-65 | CCAATCCCAATGTGCAACG |
| Ta2-66 | TTATGCGGTACGTTTAGCCTCC |

The PCR-product is cloned in vector pEXP5-NT/TOPO® (Invitrogen, Carlsbad, USA) according to the manufacturer's instructions. The resulting plasmid pEXP5-NT/TOPO®-GmHPPD is isolated from E. coli TOP10 by performing a plasmid minipreparation. The expression cassette encoding N-terminally His6-tagged GmHPPD is confirmed by DNA sequencing.

(E) Cloning of Zea mays HPPD

The Zea mays HPPD (ZmHPPD; GRMZM2G088396) coding sequence is codon-optimized for expression in E. coli and provided as a synthetic gene (Entelechon, Regensburg, Germany). The partial synthetic gene is amplified by standard PCR techniques using primers Ta2-45 and Ta2-46 (Table 9).

TABLE 9
PCR primer for ZmHPPD amplification (SEQ ID NO:
78, 79)

| Primer name | Primer sequence (5′ → 3′) |
|---|---|
| Ta2-45 | CCACCGACTCCGACCGCCGCAGC |
| Ta2-46 | TCAGGAACCCTGTGCAGCTGCCGCAG |

The PCR-product is cloned in vector pEXP5-NT/TOPO® (Invitrogen, Carlsbad, USA) according to the manufacturer's instructions. The resulting plasmid pEXP5-NT/TOPO®-ZmHPPD is isolated from E. coli TOP10 by performing a plasmid minipreparation. The expression cassette encoding N-terminally His6-tagged ZmHPPD is confirmed by DNA sequencing.

(F) Cloning of Oryza sativa HPPD

The Oryza sativa HPPD (OsHPPD; Os02g07160) coding sequence is codon-optimized for expression in E. coli and provided as a synthetic gene (Entelechon, Regensburg, Germany). The partial synthetic gene is amplified by standard PCR techniques using primers Ta2-63 and Ta2-64 (Table 10).

TABLE 10
PCR primer for OsHPPD amplification (SEQ ID NO:
80, 81)

| Primer name | Primer sequence (5′ → 3′) |
|---|---|
| Ta2-63 | CCGCCGACTCCAACCCC |
| Ta2-64 | TTAAGAACCCTGAACGGTCGG |

The PCR-product is cloned in vector pEXP5-NT/TOPO® (Invitrogen, Carlsbad, USA) according to the manufacturer's instructions. The resulting plasmid pEXP5-NT/TOPO®-OsHPPD is isolated from E. coli TOP10 by performing a plasmid minipreparation. The expression cassette encoding N-terminally His6-tagged OsHPPD is confirmed by DNA sequencing.

### (G) Gene Synthesis and Subcloning

Other wild-type HPPD encoding genes, such as Hordeum vulgare (SEQ ID NO:1/2) or Picrophilus torridus HPPD gene (Seq ID NO: 39/40) were synthesized by Geneart (Regensburg, Germany) or Entelechon (Regensburg, Germany) and subcloned into a modified pET24D (Novagen) expression vector resulting in N-terminally His-tagged expression constructs.

### Example 2

### Heterologous Expression and Purification of Recombinant HPPD Enzymes

Recombinant HPPD enzymes are produced and overexpressed in E. coli. Chemically competent BL21 (DE3) cells (Invitrogen, Carlsbad, USA) are transformed with pEXP5-NT/TOPO® (see EXAMPLE 1) or with other expression vectors according to the manufacturer's instructions. Transformed cells are grown in autoinduction medium (ZYM 5052 supplemented with 100 μg/ml ampicillin) for 6 h at 37° C. followed by 24 h at 25° C.

At an OD600 (optical density at 600 nm) of 8 to 12, cells are harvested by centrifugation (8000×g). The cell pellet is resuspended in a lysis buffer (50 mM sodium phosphate buffer, 0.5 M NaCl, 10 mM Imidazole, pH 7.0) supplemented with complete EDTA free protease inhibitor mix (Roche-Diagnostics) and homogenized using an Avestin Press. The homogenate is cleared by centrifugation (40,000×g). Hiss-tagged HPPD or mutant variants are purified by affinity chromatography on a Protino Ni-IDA 1000 Packed Column (Macherey-Nagel) according to the manufacturer's instructions. Purified HPPD or mutant variants are dialyzed against 100 mM sodium phosphate buffer pH 7.0, supplemented with 10% glycerin and stored at −86° C. Protein content is determined according to Bradford using the Bio-Rad protein assay (Bio-Rad Laboratories, Hercules, USA). The purity of the enzyme preparation is estimated by SDS-PAGE.

### Example 3

### Assay for HPPD Activity

HPPD produces homogentisic acid and CO₂ from 4-hydroxyphenylpyruvate (4-HPP) and 02. The activity assay for HPPD is based on the analysis of homogentisic acid by reversed phase HPLC. The assay mixture can contain 150 mM potassium phosphate buffer pH 7.0, 50 mM L-ascorbic acid, 100 μM Catalase (Sigma-Aldrich), 1 μM FeSO₄ and 0.2 units of purified HPPD enzyme in a total volume of 505 μl. 1 unit is defined as the amount of enzyme that is required to produce 1 nmol of HGA per minute at 20° C.

After a preincubation of 30 min the reaction is started by adding 4-HPP to a final concentration of 0.05 mM. The reaction is allowed to proceed for 45 min at room temperature. The reaction is stopped by the addition of 50 μl of 4.5 M phosphoric acid. The sample is filtered using a 0.2 μM pore size PVDF filtration device.

5 μl of the cleared sample is analyzed on an UPLC HSS T3 column (particle size 1.8 μm, dimensions 2.1×50 mm; Waters) by isocratic elution using 90% 20 mM NaH₂PO₄ pH 2.2, 10% methanol (v/v).

HGA is detected electrochemically at 750 mV (mode: DC; polarity: positive) and quantified by integrating peak areas (Empower software; Waters).

Inhibitors are dissolved in DMSO (dimethylsulfoxide) to a concentration of 0.5 mM. From this stock solution serial five-fold dilutions are prepared in DMSO, which are used in the assay. The respective inhibitor solution accounts for 1% of the assay volume. Thus, final inhibitor concentrations range from 5 μM to 320 μM, respectively. Activities are normalized by setting the uninhibited enzyme activity to 100%. IC₅₀ values are calculated using non-linear regression.

### Example 4

### In Vitro Characterization of Wild-Type HPPD Enzymes

Using methods which are described in the above examples or well known in the art, purified, recombinant wild-type HPPD enzymes are characterized with respect to their kinetic properties and sensitivity towards HPPD inhibiting herbicides. Apparent michaelis constants (Kₘ) and maximal reaction velocities (Vₘₐₓ) are calculated by non-linear regression with the software GraphPad Prism 5 (GraphPad Software, La Jolla, USA) using a substrate inhibition model. Apparent k_{cat} values are calculated from Vₘₐₓ assuming 100% purity of the enzyme preparation. Weighted means (by standard error) of Kₘ and IC₅₀ values are calculated from at least three independent experiments. The Cheng-Prusoff equation for competitive inhibition (Cheng, Y. C.; Prusoff, W. H. Biochem Pharmacol 1973, 22, 3099-3108) is used to calculate dissociation constants (Kᵢ).

Field performance of the HPPD enzyme, which is used as a herbicide tolerance trait, may depend not only on its lack of sensitivity towards HPPD inhibiting herbicides but also on its activity. To assess the potential performance of a herbicide tolerance trait a tolerance index (TI) is calculated using the following formula:

$$TI = \frac{{k}_{cat} \times {K}_{i}}{{K}_{m}}$$

Easy comparison and ranking of each trait is enabled by normalizing tolerance indexes on Arabidopsis wild-type HPPD.

Examples of the data obtained in an in vitro assay are depicted in Table 1 and in Table 12.

TABLE 11
Determination of michaelis constants (Kₘ) for 4-HPP, turnover
numbers (k_{cat}), catalytic efficiencies (k_{cat}/Kₘ) and dissociation
constants (Kᵢ) for various HPPD enzymes.

| | Kₘ | | | |
|---|---|---|---|---|
| | [μM] | | k_{cat}/Kₘ | Kᵢ [nM] |
| Enzyme | (4-HPP) | k_{cat} [S⁻¹] | [μM⁻¹ S⁻¹] | (Inhibitor 1)* |
| Arabidopsis | 13 | 12.9 | 1 | 19 |
| Hordeum | 26 | 11.5 | 0.44 | 38 |

*The HPPD-inhibiting herbicide, particularly the bicycloarylcarboxamide used in this example is (NE)-8-chloro-N-(4-methoxy-1,2,5-oxadiazol-3-ylidene)-4,4-dimethyl-1,1-dioxo-2,3-dihydrothiochromene-7-carboxamide

TABLE 12
Normalized tolerance indexes of various HPPD enzymes

| | TI |
|---|---|
| | Inhibitor |
| Enzyme | 1* |
| Arabidopsis | 1 |
| Hordeum | 0.9 |
| Rhodococcus | 1.2 |
| (Seq ID 46) | |
| Rhodococcus | 25.2 |
| (Seq ID 44) | |
| Picrophilus | 0.4 |
| Seq ID 67 | 107.3 |
| Seq ID 69 | 13.2 |
| Synechococcus | 11.9 |

*The HPPD-inhibiting herbicide, particularly the bicycloarylcarboxamide used in this example is (NE)-8-chloro-N-(4-methoxy-1,2,5-oxadiazol-3-ylidene)-4,4-dimethyl-1,1-dioxo-2,3-dihydrothiochromene-7-carboxamide

The reference SEQ ID NO:53 was included as a comparative control in a representative number of experiments and the values given in Table 12 are the average values from a number of experiments. The TI values given for various HPPD enzymes from different organisms are normalized to the value of the reference SEQ ID NO:53 in the above example.

A number of conclusions can be derived from the data in Table 12. For example, a polynucleotides comprising a region which encodes Rhodoccocus HPPD (SEQ ID NO:44) resulted in a 25-fold more resistant HPPD enzyme compared to the likewise tolerance index of the Arabidopsis HPPD. It can be further seen that a polynucleotide comprising a region which encodes Synechoccus HPPD is selected as one which encodes an inhibitor-resistant HPPD because it is found that the tolerance index against the tested HPPD inhibitor is increased 11.9-fold, compared to the reference SEQ ID NO:53. In addition, the HPPD enzyme deriving from SEQ ID NO:69 resulted in an 107.3-fold increased resistance to the HPPD inhibiting herbicide tested in the present invention.

It is evident that any HPPD enzyme that is resistant towards herbicides, even if this protein is not exemplified in this text, is part of the subject-matter of this invention.

### Example 5

### Rational Mutagenesis

By means of structural biology and sequence alignment it is possible to choose a certain number of amino acids which can either directly or indirectly be involved in the binding of HPPD-inhibiting herbicides and then to mutagenize them and obtain tolerant HPPD enzymes.

### (A) Site-Directed Mutagenesis

PCR-based site directed mutagenesis of pEXP5-NT/TOPO®-AtHPPD is done with the QuikChange II Site-Directed Mutagenesis Kit (Stratagene, Santa Clara, USA) according to the manufacturers instructions. This technique requires two chemically synthesized DNA primers (forward and reverse primer) for each mutation. Exemplified primers that can be used for site directed mutagenesis of AtHPPD (SEQ ID NO:52/53) are listed in Table 13.

TABLE 13
PCR primers for site directed mutagenesis of AtH PPD (SEQ ID NOs. 82 to 147)

| Primer | | Mutation |
|---|---|---|
| name | Primer sequence (5′→ 3′) | AtHPPD |
| HuJ141 | GAGGATTCGACTTCGCGCCTTCTCCTCC | Met335 → Ala |
| HuJ142 | GGAGGAGAAGGCGCGAAGTCGAATCCTC | Met335 → Ala |
| HuJ143 | GAGGATTCGACTTCTGGCCTTCTCCTCCG | Met335 → Trp |
| HuJ144 | CGGAGGAGAAGGCCAGAAGTCGAATCCTC | Met335 → Trp |
| HuJ145 | GGAGGATTCGACTTCTTTCCTTCTCCTCCGC | Met335 → Phe |
| HuJ146 | GCGGAGGAGAAGGAAAGAAGTCGAATCCTCC | Met335 → Phe |
| HuJ147 | GTGACAGGCCGACGATAGCTATAGAGATAATCCAG | Phe392 → Ala |
| HuJ148 | CTGGATTATCTCTATAGCTATCGTCGGCCTGTCAC | Phe392 → Ala |
| HuJ153 | GACTTCATGCCTCCTCCTCCGCCTACTTAC | Ser337 → Pro |
| HuJ154 | GTAAGTAGGCGGAGGAGGAGGCATGAAGTC | Ser337 → Pro |
| HuJ155 | GATTCGACTTCATGGCTTCTCCTCCGCCTAC | Pro336 → Ala |
| HuJ156 | GTAGGCGGAGGAGAAGCCATGAAGTCGAATC | Pro336 → Ala |
| HuJ157 | CAGATCAAGGAGTGTCAGGAATTAGGGATTCTTG | Glu363 → Gln |
| HuJ158 | CAAGAATCCCTAATTCCTGACACTCCTTGATCTG | Glu363 → Gln |
| HuJ159 | CGGAACAAAGAGGAAGAGTGAGATTCAGACGTATTTGG | Gln293 → Val |
| HuJ160 | CCAAATACGTCTGAATCTCACTCTTCCTCTTTGTTCCG | Gln293 → Val |
| HuJ169 | CGTTGCTTCAAATCTTCCCGAAACCACTAGGTGACAGGCC | Thr382 → Pro |
| HuJ170 | GGCCTGTCACCTAGTGGTTTCGGGAAGATTTGAAGCAACG | Thr382 → Pro |
| HuJ171 | CAAATCTTCACAAAACCAGTGGGTGACAGGCCGACGAT | Leu385 → Val |
| HuJ172 | ATCGTCGGCCTGTCACCCACTGGTTTTGTGAAGATTTG | Leu385 → Val |
| HuJ173 | TGACAGGCCGACGATATTTCTGGAGATAATCCAGAGAGTA | Ile393 → Leu |
| HuJ174 | TACTCTCTGGATTATCTCCAGAAATATCGTCGGCCTGTCA | Ile393 → Leu |
| HuJ175 | GACTTCATGCCTGCGCCTCCGCCTACTTAC | Ser337 → Ala |
| HuJ176 | GTAAGTAGGCGGAGGCGCAGGCATGAAGTC | Ser337 → Ala |
| HuJ177 | GGCAATTTCTCTGAGTTCTTCAAGTCCATTGAAG | Leu427 → Phe |
| HuJ178 | CTTCAATGGACTTGAAGAACTCAGAGAAATTGCC | Leu427 → Phe |
| HuJ185 | GGAACAAAGAGGAAGAGTGTGATTCAGACGTATTTGG | Gln293 → Val |
| HuJ186 | CCAAATACGTCTGAATCACACTCTTCCTCTTTGTTCC | Gln293 → Val |
| Ta2-55 | GAGGATTCGACTTCAACCCTTCTCCTCC | Met335 → Asn |
| Ta2-56 | GGAGGAGAAGGGTTGAAGTCGAATCCTC | Met335 → Asn |
| Ta2-57 | GAGGATTCGACTTCCAGCCTTCTCCTCC | Met335 → Gln |
| Ta2-58 | GGAGGAGAAGGCTGGAAGTCGAATCCTC | Met335 → Gln |
| Ta2-59 | GGAACAAAGAGGAAGAGTAACATTCAGACGTATTTGG | Gln293 → Asn |
| Ta2-60 | CCAAATACGTCTGAATGTTACTCTTCCTCTTTGTTCC | Gln293 → Asn |
| Ta2-61 | GGAACAAAGAGGAAGAGTCACATTCAGACGTATTTGG | Gln293 → His |
| Ta2-62 | CCAAATACGTCTGAATGTGACTCTTCCTCTTTGTTCC | Gln293 → His |
| Ta2-126 | GGAACAAAGAGGAAGAGTGCGATTCAGACGTATTTGG | Gln293 → Ala |
| Ta2-127 | CCAAATACGTCTGAATCGCACTCTTCCTCTTTGTTCC | Gln293 → Ala |
| Ta2-140 | GGAACAAAGAGGAAGAGTCTGATTCAGACGTATTTGG | Gln293 → Leu |
| Ta2-141 | CCAAATACGTCTGAATCAGACTCTTCCTCTTTGTTCC | Gln293 → Leu |
| Ta2-138 | GGAACAAAGAGGAAGAGTATAATTCAGACGTATTTGG | Gln293 → Ile |
| Ta2-139 | CCAAATACGTCTGAATTATACTCTTCCTCTTTGTTCC | Gln293 → Ile |
| Ta2-150 | GGAACAAAGAGGAAGAGTTCGATTCAGACGTATTTGG | Gln293 → Ser |
| Ta2-151 | CCAAATACGTCTGAATCGAACTCTTCCTCTTTGTTCC | Gln293 → Ser |
| Ta2-194 | GAGGATTCGACTTCCACCCTTCTCCTCC | Met335 → His |
| Ta2-195 | GGAGGAGAAGGGTGGAAGTCGAATCCTC | Met335 → His |
| Ta2-196 | GAGGATTCGACTTCTACCCTTCTCCTCC | Met335 → Tyr |
| Ta2-197 | GGAGGAGAAGGGTAGAAGTCGAATCCTC | Met335 → Tyr |
| Ta2-190 | GAGGATTCGACTTCAGCCCTTCTCCTCC | Met335 → Ser |
| Ta2-191 | GGAGGAGAAGGGCTGAAGTCGAATCCTC | Met335 → Ser |
| Ta2-192 | GAGGATTCGACTTCACACCTTCTCCTCC | Met335 → Thr |
| Ta2-193 | GGAGGAGAAGGTGTGAAGTCGAATCCTC | Met335 → Thr |
| Ta2-188 | GAGGATTCGACTTCTGTCCTTCTCCTCC | Met335 → Cys |
| Ta2-189 | GGAGGAGAAGGACAGAAGTCGAATCCTC | Met335 → Cys |
| Ta2-215 | GGATTCGACTTCATGCGTTCTCCTCCGCC | Pro336 → Arg |
| Ta2-216 | GGCGGAGGAGAACGCATGAAGTCGAATCC | Pro336 → Arg |
| Ta2-200 | GAGGAATTAGGGATTTGGGTAGACAGAGATG | Leu368 → Trp |
| Ta2-201 | CATCTCTGTCTACCCAAATCCCTAATTCCTC | Leu368 → Trp |
| Ta2-198 | GAGGAATTAGGGATTATGGTAGACAGAGATG | Leu368 → Met |
| Ta2-199 | CATCTCTGTCTACCATAATCCCTAATTCCTC | Leu368 → Met |
| Ta2-204 | GGTGGTTTTGGCAAACACAATTTCTCTGAG | Gly422 → His |
| Ta2-205 | CTCAGAGAAATTGTGTTTGCCAAAACCACC | Gly422 → His |
| Ta2-202 | GGTGGTTTTGGCAAATGCAATTTCTCTGAG | Gly422 → Cys |
| Ta2-203 | CTCAGAGAAATTGCATTTGCCAAAACCACC | Gly422 → Cys |
| Ta2-217 | GGTGGTTTTGGCACAGGCAATTTCTCTGAG | Lys421 → Thr |
| Ta2-218 | CTCAGAGAAATTGCCTGTGCCAAAACCACC | Lys421 → Thr |

Exemplified primers that can be used for site directed mutagenesis of HvHPPD (SEQ ID NO:1/2) are listed in Table 14.

TABLE 14
PCR primers for site directed mutagenesis of
HvHPPD (SEQ ID NOs: 148 to 155)

| Primer | | Mutation |
|---|---|---|
| name | Sequence (5′→ 3′) | HvHPPD |
| Ta2-279 | GGGAGGGTTTGACTTTCATCCACCTCCGCTG | Leu320 → |
| Ta2-280 | CAGCGGAGGTGGATGAAAGTCAAACCCTCCC | His |
| Ta2-246 | GGCTTCGACTTCTATCCACCCCCGCTG | Leu320 → |
| Ta2-247 | CAGCGGGGGTGGATAGAAGTCGAAGCC | Tyr |
| Ta2-248 | GGGTTCGGCAAATGCAACTTCTCCGAGCTG | Gly407 → |
| Ta2-249 | CAGCTCGGAGAAGTTGCATTTGCCGAACCC | Cys |
| Ta2-281 | GGAGGGTTTGACTTTCATGCACCTCCGCTG | Pro321 → |
| Ta2-282 | CAGCGGAGGTGCATGAAAGTCAAACCCTCC | Ala |

Mutant plasmids are isolated from E. coli TOP10 by performing a plasmid minipreparation and confirmed by DNA sequencing.

The combination of single amino acid substitutions is achieved by a stepwise mutagenesis approach.

### (B) In Vitro Characterization of HPPD Mutants

Purified, mutant HPPD enzymes are obtained by the methods described above. Dose response and kinetic measurements are carried out using the described HPPD activity assay. Apparent michaelis constants (Kₘ) and maximal reaction velocities (Vₘₐₓ) are calculated by non-linear regression with the software GraphPad Prism 5 (Graph Pad Software, La Jolla, USA) using a substrate inhibition model. Apparent k_{cat} values are calculated from Vₘₐₓ assuming 100% purity of the enzyme preparation. Weighted means (by standard error) of Kₘ and IC₅₀ values are calculated from at least three independent experiments. The Cheng-Prusoff equation for competitive inhibition (Cheng, Y. C.; Prusoff, W. H. Biochem Pharmacol 1973, 22, 3099-3108) is used to calculate dissociation constants (K).

Field performance of the optimized HPPD enzyme, which is used as a herbicide tolerance trait may depend not only on its lack of sensitivity towards HPPD inhibiting herbicides but also on its activity. To assess the potential performance of a herbicide tolerance trait a tolerance index (TI) is calculated using the following formula:

$$TI = \frac{{k}_{cat} \times {K}_{i}}{{K}_{m}}$$

Easy comparison and ranking of each trait is enabled by normalizing tolerance indexes on Arabidopsis wild-type HPPD.

Examples of the data obtained are depicted in Table 15 and in Table 16.

TABLE 15
Normalized tolerance indexes of various HPPD mutants generated in
the Arabidopsis HPPD (SEQ ID: 53).

| | TI |
|---|---|
| | Inhibitor |
| Arabidopsis HPPD variant | 1* |
| Wild-type | 1 |
| M335H, P336A, E363Q | 0.3 |

*The HPPD-inhibiting herbicide, particularly the bicycloarylcarboxamide, used in this example is (NE)-8-chloro-N-(4-methoxy-1,2,5-oxadiazol-3-ylidene)-4,4-dimethyl-1,1-dioxo-2,3-dihydrothiochromene-7-carboxamide.

TABLE 16
Normalized tolerance indexes of various HPPD mutants generated in the
Hordeum HPPD (SEQ ID: 2).

| | TI | TI |
|---|---|---|
| Hordeum HPPD variant | Inhibitor 1* | Inhibitor 2* |
| Wild-type | 1 | 1 |
| L320Q | 0.1 | 1.4 |
| L320H | 0.6 | 4.4 |
| L320H, P321A | 0.5 | 3.6 |
| L353M, P321R, V212I | 2.9 | n.d. |
| L353M, P321R, L320N | 0.2 | 3.5 |
| L353M, P321R, L320Q | 1.2 | 3.4 |
| G407C | 3.3 | n.d. |
| F404L | 2.1 | 3.1 |
| F377L | n.d. | 4.6 |
| S252T | 2.4 | 5.9 |
| R309K | 0.4 | 2.8 |

*The HPPD-inhibiting herbicide, particularly the bicycloarylcarboxamide used in this example is (NE)-8-chloro-N-(4-methoxy-1,2,5-oxadiazol-3-ylidene)-4,4-dimethyl-1,1-dioxo-2,3-dihydrothiochromene-7-carboxamide (Inhibitor 1) and 3,3,5-trimethyl-N-(1-methyltetrazol-5-yl)-1,1,4-trioxo-2H-thiochromene-6-carboxamide (Inhibitor 2),
n.d.—not determined.

A number of conclusions can be derived from the data in Table 15 and Table 16. The performance of various HPPD mutants in the in vitro assay indicated that certain amino acid substitutions within the coding sequence provided significant improvements relative to HPPD SEQ ID NO:2 in respect to the tolerance indexes against the HPPD-inhibiting herbicides of the present invention.

It can be seen from the results depicted in Table 16 that the substitution for leucine at position 320 to histidine provided an improvement of the Hordeum HPPD enzyme (SEQ ID NO:2) as the tolerance index for Inhibitor 2 increased 4.4-fold compared to the wild-type enzyme. In addition, the combined exchange of leucine 320 to histidine and proline 321 to alanine resulted in an HPPD inhibitor resistant enzyme because its tolerance index for Inhibitor 2 was increased 3.6-fold compared to the reference HPPD SEQ ID NO:2. The combined mutation of leucine 320 to asparagine or glutamine together with the exchange of proline 321 to alanine and leucine 353 to methionine relative to SEQ ID NO:2 resulted in a better tolerance index for Inhibitor 2 and an overall 3.5-fold increased resistance was observed.

In addition, a polynucleotide comprising a region which encodes Hordeum HPPD (SEQ ID NO:2) with phenylalanine 404 exchanged to leucine resulted in a 3-fold increased tolerance index compared to the benchmark enzyme. Furthermore, the exchange of phenylalanine 377 to leucine, or serine 252 to threonine resulted in HPPD enzymes that had a 4.6-fold and 5.9-fold, respectively, improved tolerance index compared to the control. Thus, those HPPD enzymes can be selected as transgenes that encode inhibitor-resistant HPPD enzymes because it was found that the tolerance index of the mutants was significantly improved against Inhibitor 2 tested in the present invention.

In addition, the exchange of glycine at position 407 to cysteine resulted in a significant improvement of the HPPD enzyme as the mutated HPPD enzyme had a better performance against the HPPD-inhibiting herbicide (Inhibitor 1) because its tolerance index was increased 3.3-fold compared to the reference HPPD SEQ ID NO:2.

It is evident that these examples indicate that a mutant HPPD enzyme can be selected as one which is resistant to HPPD-inhibiting herbicides because tolerance indexes of the mutants are greater than the tolerance index of the respective wild-type enzyme. It is evident that any mutation or combination of mutations which would make it possible to obtain an HPPD enzyme that is resistant to bicycloarylcarboxamide-derivative herbicides, even if this protein is not exemplified in this text, is part of the subject-matter of this invention.

### Example 6

### Preparation of Plants which Express Heterologous HPPD and/or HST Enzymes and which are Tolerant to “HPPD-Inhibiting Herbicides”

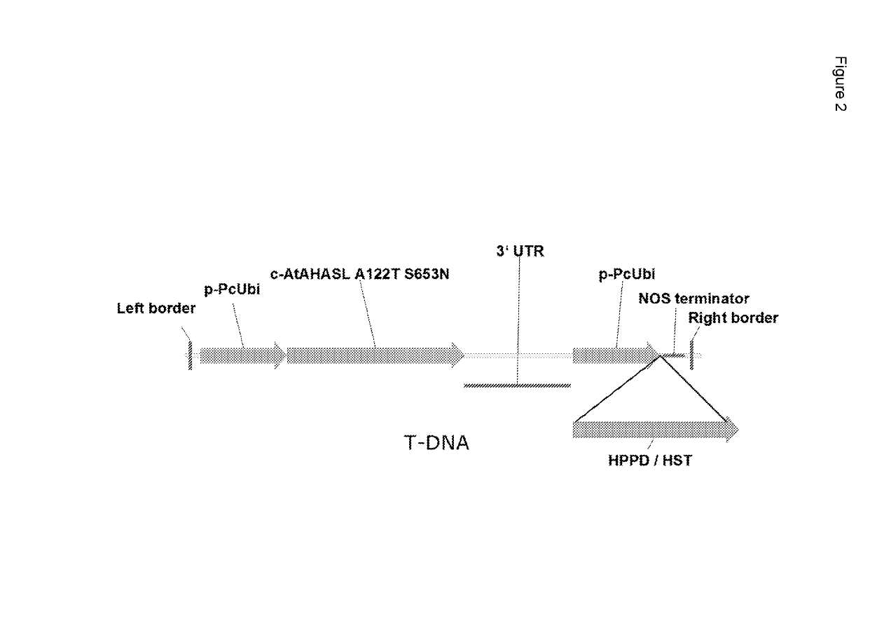

Various methods for the production of stably transformed plants are well known in the art. HPPD-inhibiting herbicide tolerant soybean (Glycine max) or corn (Zea mays) plants can be produced by a method described by Olhoft et al. (US patent 2009/0049567). Briefly, HPPD or HST encoding polynucleotides are cloned into a binary vector using standard cloning techniques as described by Sambrook et al. (Molecular cloning (2001) Cold Spring Harbor Laboratory Press). The final vector construct contains an HPPD or HST encoding sequence flanked by a promoter sequence (e.g. the ubiquitin promoter (PcUbi) sequence) and a terminator sequence (e.g. the nopaline synthase terminator (NOS) sequence) and a resistance marker gene cassette (e.g. AHAS) (FIG. 2). Optionally, the HPPD or HST gene can provide the means of selection. Agrobacterium-mediated transformation is used to introduce the DNA into soybean's axillary meristem cells at the primary node of seedling explants. After inoculation and co-cultivation with Agrobacteria, the explants are transferred to shoot induction medium without selection for one week. The explants are subsequently transferred to shoot induction medium with 1-3 μM imazapyr (Arsenal) for 3 weeks to select for transformed cells. Explants with healthy callus/shoot pads at the primary node are then transferred to shoot elongation medium containing 1-3 μM imazapyr until a shoot elongates or the explant dies. After regeneration, transformants are transplanted to soil in small pots, placed in growth chambers (16 hr day/8 hr night; 25° C. day/23° C. night; 65% relative humidity; 130-150 mE m-2 s-1) and subsequently tested for the presence of the T-DNA via Taqman analysis. After a few weeks, healthy, transgenic positive, single copy events are transplanted to larger pots and allowed to grow in the growth chamber. Transformation of corn plants is done by a method described by McElver and Singh (WO 2008/124495). Plant transformation vector constructs containing HPPD or HST sequences are introduced into maize immature embryos via Agrobacterium-mediated transformation. Transformed cells are selected in selection media supplemented with 0.5-1.5 μM imazethapyr for 3-4 weeks. Transgenic plantlets are regenerated on plant regeneration media and rooted afterwards. Transgenic plantlets are subjected to TaqMan analysis for the presence of the transgene before being transplanted to potting mixture and grown to maturity in greenhouse.

Arabidopsis thaliana is transformed with HPPD or HST sequences by floral dip method as described by McElver and Singh (WO 2008/124495). Transgenic Arabidopsis plants are subjected to TaqMan analysis for analysis of the number of integration loci.

Transformation of Oryza sativa (rice) are done by protoplast transformation as described by Peng et al. (U.S. Pat. No. 6,653,529)

T0 or T1 transgenic plant of soybean, corn, rice and Arabidopsis thaliana containing HPPD or HST sequences are tested for improved tolerance to HPPD-inhibiting herbicides in greenhouse studies.

### Example 7

### Greenhouse Experiments

Transgenic plants expressing heterologous HPPD or HST enzymes are tested for tolerance against HPPD-inhibiting herbicides in greenhouse experiments.

For the pre-emergence treatment, the herbicides are applied directly after sowing by means of finely distributing nozzles. The containers are irrigated gently to promote germination and growth and subsequently covered with transparent plastic hoods until the plants have rooted. This cover causes uniform germination of the test plants, unless this has been impaired by the herbicides.

For post emergence treatment, the test plants are first grown to a height of 3 to 15 cm, depending on the plant habit, and only then treated with the herbicides. For this purpose, the test plants are either shown directly and grown in the same containers, or they are first grown separately and transplanted into the test containers a few days prior to treatment.

For testing of T0 plants, cuttings can be used. In the case of soybean plants, an optimal shoot for cutting is about 7.5 to 10 cm tall, with at least two nodes present. Each cutting is taken from the original transformant (mother plant) and dipped into rooting hormone powder (indole-3-butyric acid, IBA). The cutting is then placed in oasis wedges inside a bio-dome. Wild-type cuttings are also taken simultaneously to serve as controls. The cuttings are kept in the bio-dome for 5-7 days and then transplanted to pots and then acclimated in the growth chamber for two more days.

Subsequently, the cuttings are transferred to the greenhouse, acclimated for approximately 4 days, and then subjected to spray tests as indicated.

Depending on the species, the plants are kept at 10-25° C. or 20-35° C. The test period extends over 3 weeks. During this time, the plants are tended and their response to the individual treatments is evaluated. Herbicide injury evaluations are taken at 2 and 3 weeks after treatment. Plant injury is rated on a scale of 0 to 9, 0 being no injury and 9 being complete death. Tolerance to HPPD-inhibiting herbicides can also be assessed in Arabidopsis. In this case transgenic Arabidopsis thaliana plants are assayed for improved tolerance to HPPD-inhibiting herbicides in 48-well plates. Seeds are surface sterilized by stirring for 5 min in ethanol+water (70+30 by volume), rinsing one time with ethanol+water (70+30 by volume) and two times with a sterile, deionized water. The seeds are resuspended in 0.1% agar dissolved in water (w/v). Four to five seeds per well are plated on solid nutrient medium consisting of half-strength Murashige Skoog nutrient solution, pH 5.8 (Murashige and Skoog (1962) Physiologia Plantarum 15: 473-497). Compounds are dissolved in dimethylsulfoxid (DMSO) and added to the medium prior solidification (final DMSO concentration 0.1%). Multi well plates are incubated in a growth chamber at 22° C., 75% relative humidity and 110 μmol Phot*m-2*s-1 with 14:10 h light:dark photoperiod. Seven to ten days after seeding growth inhibition is evaluated by comparison to wild-type plants. Tolerance factor is calculated by dividing the plant growth IC50 value of transgenic plants containing a HPPD and/or HST sequence by that of wild-type plants.

Additionally, T1 and T2 transgenic Arabidopsis plants can be tested for improved tolerance to HPPD-inhibiting herbicides in a greenhouse studies. Herbicide injury scoring is done 2-3 weeks after treatment and is rated on a scale of 0 to 100%, 0% being no injury and 100% being complete death.

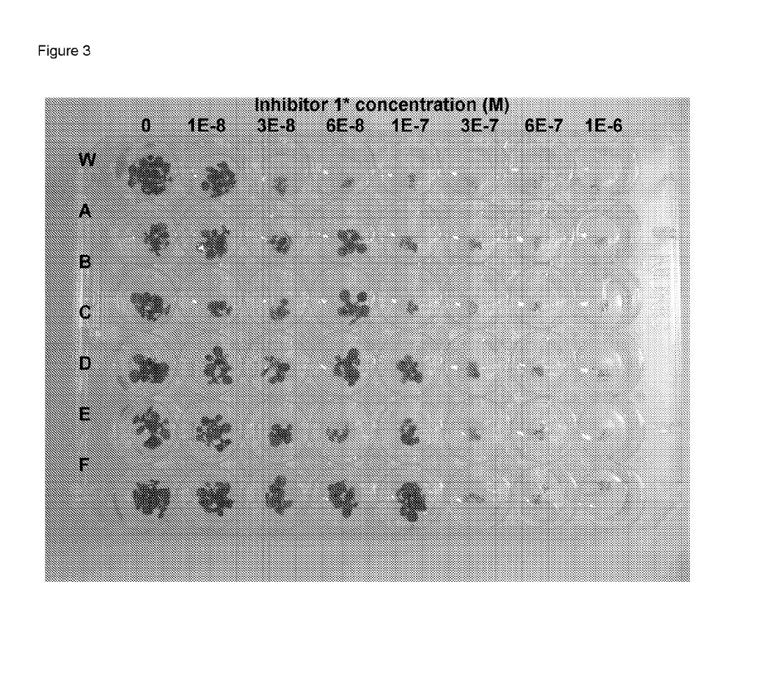

Examples of the data obtained are depicted in Table 17 and in FIG. 3.

TABLE 17
Tolerance factor observed for transgenic plants.

| | Tolerance | Tolerance |
|---|---|---|
| | factor | factor |
| Arabidopsis overexpression | towards | towards |
| line | Inhibitor 1* | Inhibitor 2* |
| Wild-type | 1 | 1 |
| AtHPPD (Seq ID: 53) | 6.4 | 3 |
| overexspression | | |
| AtHPPD (Seq ID: 53) | 37.5 | 60.8 |
| F381I | | |
| AtHPPD (Seq ID: 53) | 15.8 | 60.8 |
| M335H, P336G, E363Q | | |
| HvHPPD (SEQ ID: 2) over- | 76 | 10.2 |
| expression | | |
| HvHPPD (SEQ ID: 2) | 85 | 7.9 |
| L320H, P321A | | |
| Picrophilus HPPD | 4.5 | 2.6 |

*The HPPD-inhibiting herbicide, particularly the bicycloarylcarboxamide used in this example is (NE)-8-chloro-N-(4-methoxy-1,2,5-oxadiazol-3-ylidene)-4,4-dimethyl-1,1-dioxo-2,3-dihydrothiochromene-7-carboxamide (Inhibitor 1) and 3,3,5-trimethyl-N-(1-methyltetrazol-5-yl)-1,1,4-trioxo-2H-thiochromene-6-carboxamide (Inhibitor 2).

The results demonstrate that plants comprising a polynucleotide encoding Arabidopsis HPPD have an increased tolerance, which is 6.4-fold for Inhibitor 1 and 3-fold for Inhibitor 2, for HPPD-inhibiting herbicides compared to the untransformed control plants. Furthermore, it can be seen from the results depicted in Table 17 that the substitution of phenylalanine at position 381 to isoleucine in SEQ ID NO:53 provided a significantly improved tolerance to Inhibitor 1 and Inhibitor 2. This mutant showed a 37.5-fold and 60.8-fold, respectively, increased tolerance compared to the wild-type control. In addition, the combined mutation of methionine at position 335 to histidine together with the exchange of proline at position 336 to glycine and glutamate at position 363 to glutamin (SEQ ID NO:53) resulted in a significant improvement of the HPPD enzyme because it was found that the mutated HPPD enzyme had a 15.8-fold increased tolerance against Inhibitor 1 and a 60.8-fold increased tolerance against Inhibitor 2 compared to the untransformed wild-type control.

Furthermore, it can be seen from the results depicted above that transgenic plants overexpressing Hordeum HPPD (SEQ ID NO:2) were more resistant to Inhibitor 1 (76-fold) and Inhibitor 2 (10.2-fold). The substitution of leucine at position 320 to histidine together with proline 321 to alanine in SEQ ID NO:2 provided a significant improvement of the HPPD enzyme compared to the untransformed wild-type control with regard to the tolerance factor against Inhibitor 1 and Inhibitor 2 tested in the present invention. Those transgenic plants expressing the mutated version of SEQ ID NO:2 where leucine 320 was exchanged to histidine and proline 321 was substituted by alanine, can be selected as mutants which comprise an inhibitor-resistant HPPD because those plants had an 85-fold increased tolerance to Inhibitor 1 and 7.9-fold increased tolerance to Inhibitor 2 compared to untransformed wild-type plants and thus are useful for generating herbicide tolerant plants.

The results further demonstrate that plants comprising a polynucleotide encoding for HPPD from Picrophilus had a 4.5-fold increased tolerance factor for Inhibitor 1 and a 2.6-fold higher tolerance factor for Inhibitor 2.

1. A method for controlling undesired vegetation at a plant cultivation site, the method comprising the steps of:
a) providing, at said site, a plant that comprises at least one nucleic acid comprising
(i) a nucleotide sequence encoding a wild-type hydroxyphenyl pyruvate dioxygenase or a mutated hydroxyphenyl pyruvate dioxygenase (mut-HPPD) which is resistant or tolerant to a HPPD-inhibiting herbicide and/or
(ii) a nucleotide sequence encoding a wild-type homogentisate solanesyl transferase or a mutated homogentisate solanesyl transferase (mut-HST) which is resistant or tolerant to a HPPD-inhibiting herbicide
b) applying to said site an effective amount of said herbicide.
wherein the nucleotide sequence of (i) comprises the sequence of SEQ ID NO:1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, or a variant or derivative thereof, and wherein the mut-HPPD comprises a polypeptide which differs from an amino acid sequence of a wild-type HPPD at one or more positions corresponding to or at the following positions of SEQ ID NO:2:
the amino acid corresponding to or at position 236 is other than alanine;
the amino acid corresponding to or at position 411 is other than glutamic acid;
the amino acid corresponding to or at position 320 is other than leucine;
the amino acid corresponding to or at position 403 is other than glycine;
the amino acid corresponding to or at position 334 is other than leucine;
the amino acid corresponding to or at position 353 is other than leucine;
the amino acid corresponding to or at position 321 is other than proline;
the amino acid corresponding to or at position 212 is other than valine;
the amino acid corresponding to or at position 407 is other than glycine;
the amino acid corresponding to or at position 377 is other than phenylalanine;
the amino acid corresponding to or at position 412 is other than leucine;
the amino acid corresponding to or at position 278 is other than glutamine;
the amino acid corresponding to or at position 406 is other than lysine;
the amino acid corresponding to or at position 404 is other than phenylalanine;
the amino acid corresponding to or at position 409 is other than phenylalanine;
the amino acid corresponding to or at position 416 is other than isoleucine;
the amino acid corresponding to or at position 250 is other than leucine;
the amino acid corresponding to or at position 267 is other than asparagine;
the amino acid corresponding to or at position 252 is other than serine;
the amino acid corresponding to or at position 265 is other than proline;
the amino acid corresponding to or at position 371 is other than glycine;
the amino acid corresponding to or at position 375 is other than threonine;
the amino acid corresponding to or at position 309 is other than arginine;
the amino acid corresponding to or at position 279 is other than isoleucine;
the amino acid corresponding to or at position 366 is other than phenylalanine;
the amino acid corresponding to or at position 238 is other than phenylalanine;
the amino acid corresponding to or at position 213 is other than valine;
the amino acid corresponding to or at position 215 is other than asparagine;
the amino acid corresponding to or at position 410 is other than serine;
the amino acid corresponding to or at position 254 is other than valine.

2. The method according to claim 1, wherein the HPPD inhibiting herbicide is a bicycloarylcarboxamide.

3. The method according to claim 1 or 2, wherein the nucleotide sequence of (ii) comprises the sequence of SEQ ID NO:47 or 49, or a variant or derivative thereof.

4. (canceled)

5. (canceled)

6. A method for identifying a bicycloarylcarboxamide by using a mut-HPPD encoded by a nucleic acid which comprises the nucleotide sequence of SEQ ID NO:1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, or a variant or derivative thereof, and/or by using a mut-HST encoded by a nucleic acid which comprises the nucleotide sequence of SEQ ID NO:47 or 49, or a variant or derivative thereof.

7. The method according to claim 6, comprising the steps of:
a) generating a transgenic cell or plant comprising a nucleic acid encoding a mut-HPPD, wherein the mut-HPPD is expressed;
b) applying a bicycloarylcarboxamide to the transgenic cell or plant of a) and to a control cell or plant of the same variety;
c) determining the growth or the viability of the transgenic cell or plant and the control cell or plant after application of said test compound, and
d) selecting test compounds which confer reduced growth to the control cell or plant as compared to the growth of the transgenic cell or plant.

8. A method of identifying a nucleotide sequence encoding a mut-HPPD which is resistant or tolerant to a bicycloarylcarboxamide, the method comprising:
a) generating a library of mut-HPPD-encoding nucleic acids,
b) screening a population of the resulting mut-HPPD-encoding nucleic acids by expressing each of said nucleic acids in a cell or plant and treating said cell or plant with a bicycloarylcarboxamide
c) comparing the bicycloarylcarboxamide-tolerance levels provided by said population of mut-HPPD encoding nucleic acids with the bicycloarylcarboxamide-tolerance level provided by a control HPPD-encoding nucleic acid,
d) selecting at least one mut-HPPD-encoding nucleic acid that provides a significantly increased level of tolerance to a bicycloarylcarboxamide as compared to that provided by the control HPPD-encoding nucleic acid.

9. The method according to claim 8, wherein the mut-HPPD-encoding nucleic acid selected in step d) provides at least 2-fold as much tolerance to a bicycloarylcarboxamide as compared to that provided by the control HPPD-encoding nucleic acid.

10. The method according to claim 8 or 9, wherein the resistance or tolerance is determined by generating a transgenic plant comprising a nucleic acid sequence of the library of step a) and comparing said transgenic plant with a control plant.

11. An isolated nucleic acid encoding a mut-HPPD, wherein the mut-HPPD comprises a polypeptide which differs from an amino acid sequence of a wild-type HPPD at one or more positions corresponding to or at the following positions of SEQ ID NO:2:
the amino acid corresponding to or at position 236 is other than alanine;
the amino acid corresponding to or at position 411 is other than glutamic acid;
the amino acid corresponding to or at position 320 is other than leucine;
the amino acid corresponding to or at position 403 is other than glycine;
the amino acid corresponding to or at position 334 is other than leucine;
the amino acid corresponding to or at position 353 is other than leucine;
the amino acid corresponding to or at position 321 is other than proline;
the amino acid corresponding to or at position 212 is other than valine;
the amino acid corresponding to or at position 407 is other than glycine;
the amino acid corresponding to or at position 377 is other than phenylalanine;
the amino acid corresponding to or at position 412 is other than leucine;
the amino acid corresponding to or at position 278 is other than glutamine;
the amino acid corresponding to or at position 406 is other than lysine;
the amino acid corresponding to or at position 404 is other than phenylalanine;
the amino acid corresponding to or at position 409 is other than phenylalanine;
the amino acid corresponding to or at position 416 is other than isoleucine;
the amino acid corresponding to or at position 250 is other than leucine;
the amino acid corresponding to or at position 267 is other than asparagine;
the amino acid corresponding to or at position 252 is other than serine;
the amino acid corresponding to or at position 265 is other than proline;
the amino acid corresponding to or at position 371 is other than glycine;
the amino acid corresponding to or at position 375 is other than threonine;
the amino acid corresponding to or at position 309 is other than arginine;
the amino acid corresponding to or at position 279 is other than isoleucine;
the amino acid corresponding to or at position 366 is other than phenylalanine;
the amino acid corresponding to or at position 238 is other than phenylalanine;
the amino acid corresponding to or at position 213 is other than valine;
the amino acid corresponding to or at position 215 is other than asparagine;
the amino acid corresponding to or at position 410 is other than serine;
the amino acid corresponding to or at position 254 is other than valine.

12. An isolated nucleic acid encoding a mut-HPPD, wherein the mut-HPPD comprises a polypeptide which differs from an amino acid sequence of a wild-type HPPD at one or more positions corresponding to the following positions of SEQ ID NO:53:
the amino acid corresponding to or at position 228 is other than valine;
the amino acid corresponding to or at position 230 is other than asparagine;
the amino acid corresponding to or at position 251 is other than alanine;
the amino acid corresponding to or at position 253 is other than phenylalanine;
the amino acid corresponding to or at position 265 is other than leucine;
the amino acid corresponding to or at position 267 is other than serine;
the amino acid corresponding to or at position 280 is other than proline;
the amino acid corresponding to or at position 282 is other than asparagine;
the amino acid corresponding to or at position 291 is other than lysine;
the amino acid corresponding to or at position 293 is other than glutamine;
the amino acid corresponding to or at position 294 is other than isoleucine;
the amino acid corresponding to or at position 324 is other than arginine;
the amino acid corresponding to or at position 335 is other than methionine;
the amino acid corresponding to or at position 336 is other than proline;
the amino acid corresponding to or at position 337 is other than serine;
the amino acid corresponding to or at position 339 is other than proline;
the amino acid corresponding to or at position 340 is other than proline;
the amino acid corresponding to or at position 363 is other than glutamic acid;
the amino acid corresponding to or at position 368 is other than leucine;
the amino acid corresponding to or at position 381 is other than phenylalanine;
the amino acid corresponding to or at position 385 is other than leucine;
the amino acid corresponding to or at position 386 is other than glycine;
the amino acid corresponding to or at position 390 is other than threonine;
the amino acid corresponding to or at position 392 is other than phenylalanine;
the amino acid corresponding to or at position 393 is other than an isoleucine;
the amino acid corresponding to or at position 419 is other than phenylalanine;
the amino acid corresponding to or at position 421 is other than lysine;
the amino acid corresponding to or at position 422 is other than glycine;
the amino acid corresponding to or at position 424 is other than phenylalanine;
the amino acid corresponding to or at position 427 is other than leucine;
the amino acid corresponding to or at position 431 is other than isoleucine;
the amino acid corresponding to or at position 425 is other than serine;
the amino acid corresponding to or at position 269 is other than valine.

13. A transgenic plant cell transformed by a wild-type or mut-HPPD nucleic acid, wherein expression of the nucleic acid in the plant cell results in increased resistance or tolerance to a HPPD-inhibiting herbicide as compared to a wild type variety of the plant cell,
and wherein the wild-type or mut-HPPD nucleic acid comprises a polynucleotide sequence selected from the group consisting of: a) a polynucleotide as shown in SEQ ID NO:1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, or a variant or derivative thereof; b) a polynucleotide as shown in SEQ ID NO:47 or 49, or a variant or derivative thereof; c) a polynucleotide encoding a polypeptide as shown in SEQ ID NO:2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, 48, 50, or a variant or derivative thereof; d) a polynucleotide comprising at least 60 consecutive nucleotides of any of a) through c); and e) a polynucleotide complementary to the polynucleotide of any of a) through d).

14. A transgenic plant comprising a plant cell as defined in any of claim 13, wherein expression of the nucleic acid in the plant results in the plant's increased resistance to a HPPD-inhibiting herbicide as compared to a wild-type variety of the plant.

15. A plant that expresses a mutagenized or recombinant mut-HPPD comprising SEQ ID NO:2, a homologue, orthologue or paralogue thereof, in which the amino acid sequence differs from a HPPD amino acid sequence of a corresponding wild-type plant at one or more amino acid positions:
the amino acid corresponding to or at position 236 is other than alanine;
the amino acid corresponding to or at position 411 is other than glutamic acid;
the amino acid corresponding to or at position 320 is other than leucine;
the amino acid corresponding to or at position 403 is other than glycine;
the amino acid corresponding to or at position 334 is other than leucine;
the amino acid corresponding to or at position 353 is other than leucine;
the amino acid corresponding to or at position 321 is other than proline;
the amino acid corresponding to or at position 212 is other than valine;
the amino acid corresponding to or at position 407 is other than glycine;
the amino acid corresponding to or at position 377 is other than phenylalanine;
the amino acid corresponding to or at position 412 is other than leucine;
the amino acid corresponding to or at position 278 is other than glutamine;
the amino acid corresponding to or at position 406 is other than lysine;
the amino acid corresponding to or at position 404 is other than phenylalanine;
the amino acid corresponding to or at position 409 is other than phenylalanine;
the amino acid corresponding to or at position 416 is other than isoleucine;
the amino acid corresponding to or at position 250 is other than leucine;
the amino acid corresponding to or at position 267 is other than asparagine;
the amino acid corresponding to or at position 252 is other than serine;
the amino acid corresponding to or at position 265 is other than proline;
the amino acid corresponding to or at position 371 is other than glycine;
the amino acid corresponding to or at position 375 is other than threonine;
the amino acid corresponding to or at position 309 is other than arginine;
the amino acid corresponding to or at position 279 is other than isoleucine;
the amino acid corresponding to or at position 366 is other than phenylalanine;
the amino acid corresponding to or at position 238 is other than phenylalanine;
the amino acid corresponding to or at position 213 is other than valine;
the amino acid corresponding to or at position 215 is other than asparagine;
the amino acid corresponding to or at position 410 is other than serine;
the amino acid corresponding to or at position 254 is other than valine.
and wherein said mutagenized or recombinant mut-HPPD confers upon the plant increased herbicide tolerance, preferably bicycloarylcarboxamide herbicide tolerance, as compared to the corresponding wild-type variety of the plant when expressed therein.

16. A plant that expresses a mutagenized or recombinant mut-HPPD comprising SEQ ID NO:53, a homologue, orthologue or paralogue thereof, in which the amino acid sequence differs from a HPPD amino acid sequence of a corresponding wild-type plant at one or more amino acid positions:
the amino acid corresponding to or at position 228 is other than valine;
the amino acid corresponding to or at position 230 is other than asparagine;
the amino acid corresponding to or at position 251 is other than alanine;
the amino acid corresponding to or at position 253 is other than phenylalanine;
the amino acid corresponding to or at position 265 is other than leucine;
the amino acid corresponding to or at position 267 is other than serine;
the amino acid corresponding to or at position 280 is other than proline;
the amino acid corresponding to or at position 282 is other than asparagine;
the amino acid corresponding to or at position 291 is other than lysine;
the amino acid corresponding to or at position 293 is other than glutamine;
the amino acid corresponding to or at position 294 is other than isoleucine;
the amino acid corresponding to or at position 324 is other than arginine;
the amino acid corresponding to or at position 335 is other than methionine;
the amino acid corresponding to or at position 336 is other than proline;
the amino acid corresponding to or at position 337 is other than serine;
the amino acid corresponding to or at position 339 is other than proline;
the amino acid corresponding to or at position 340 is other than proline;
the amino acid corresponding to or at position 363 is other than glutamic acid;
the amino acid corresponding to or at position 368 is other than leucine;
the amino acid corresponding to or at position 381 is other than phenylalanine;
the amino acid corresponding to or at position 385 is other than leucine;
the amino acid corresponding to or at position 386 is other than glycine;
the amino acid corresponding to or at position 390 is other than threonine;
the amino acid corresponding to or at position 392 is other than phenylalanine;
the amino acid corresponding to or at position 393 is other than an isoleucine;
the amino acid corresponding to or at position 419 is other than phenylalanine;
the amino acid corresponding to or at position 421 is other than lysine;
the amino acid corresponding to or at position 422 is other than glycine;
the amino acid corresponding to or at position 424 is other than phenylalanine;
the amino acid corresponding to or at position 427 is other than leucine;
the amino acid corresponding to or at position 431 is other than isoleucine;
the amino acid corresponding to or at position 425 is other than serine;
the amino acid corresponding to or at position 269 is other than valine,
and wherein said mutagenized or recombinant mut-HPPD confers upon the plant increased herbicide tolerance, preferably bicycloarylcarboxamide herbicide tolerance, as compared to the corresponding wild-type variety of the plant when expressed therein.

17. A seed produced by a transgenic plant comprising a plant cell as defined in claim 13, wherein the seed is true breeding for an increased resistance to a HPPD-inhibiting herbicide as compared to a wild-type variety of the seed.

18. A method of producing a transgenic plant cell having an increased resistance to a HPPD-inhibiting herbicide, preferably bicycloarylcarboxamide, as compared to a wild-type variety of the plant cell comprising, transforming the plant cell with an expression cassette comprising an HPPD nucleic acid.

19. A method of producing a transgenic plant comprising: (a) transforming a plant cell with an expression cassette comprising an HPPD nucleic acid, and (b) generating a plant with an increased resistance to HPPD-inhibiting herbicide, preferably bicycloarylcarboxamide, from the plant cell.

20. The method of claim 18 or 19, wherein the HPPD nucleic acid comprises a polynucleotide sequence selected from the group consisting of: a) a polynucleotide as shown in SEQ ID NO:1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, or a variant or derivative thereof; b) a polynucleotide as shown in SEQ ID NO:47 or 49, or a variant or derivative thereof; c) a polynucleotide encoding a polypeptide as shown in SEQ ID NO:2, 5, 8, 11, 14, 17, 20, 22, 24, 26, 28, 30, 32, 34, 36, 38, 40, 42, 44, 46, 53, 55, 57, 58, 59, 60, 61, 62, 63, 64, 65, 66, 67, 48, 50, or a variant or derivative thereof d) a polynucleotide comprising at least 60 consecutive nucleotides of any of a) through c); and e) a polynucleotide complementary to the polynucleotide of any of a) through d).

21. The method of claim 18, wherein the expression cassette further comprises a transcription initiation regulatory region and a translation initiation regulatory region that are functional in the plant.

22. A method of identifying or selecting a transformed plant cell, plant tissue, plant or part thereof comprising: i) providing a transformed plant cell, plant tissue, plant or part thereof, wherein said transformed plant cell, plant tissue, plant or part thereof comprises a polynucleotide as shown in SEQ ID NO:1, 51, 3, 4, 6, 7, 9, 10, 12, 13, 15, 16, 18, 19, 21, 23, 25, 27, 29, 31, 33, 35, 37, 39, 41, 43, 45, 52, 54, 56, 68, 69, or a variant or derivative thereof, wherein the polynucleotide encodes an HPPD polypeptide that is used as a selection marker, and wherein said transformed plant cell, plant tissue, plant or part thereof may comprise a further isolated polynucleotide; ii) contacting the transformed plant cell, plant tissue, plant or part thereof with at least one HPPD-inhibiting compound, preferably bicycloarylcarboxamide; iii) determining whether the plant cell, plant tissue, plant or part thereof is affected by the inhibiting compound; and iv) identifying or selecting the transformed plant cell, plant tissue, plant or part thereof.

23. A seed produced by the plant of claim 14, wherein the seed is true breeding for an increased resistance to a HPPD-inhibiting herbicide as compared to a wild-type variety of the seed.

24. A seed produced by the plant of claim 15, wherein the seed is true breeding for an increased resistance to a HPPD-inhibiting herbicide as compared to a wild-type variety of the seed.

25. A seed produced by the plant of claim 16, wherein the seed is true breeding for an increased resistance to a HPPD-inhibiting herbicide as compared to a wild-type variety of the seed.


SEQUENCE LISTING (155 sequences)

SEQ ID NO: 1 - DNA, 1305 bp, Hordeum vulgare
atgccgcccacccccaccacccccgcggctaccggcgccgccgccgcggtgacgccggagcacgcgcgaccgcaccgaatggtccgcttcaacccgcgcagcgaccgcttccacacgctctccttccaccacgtcgagttctggtgcgcggacgccgcctccgccgccggccgcttcgcgttcgcgctcggcgcgccgctcgccgccaggtccgacctctccacggggaactccgcgcacgcctcccagctgctccgctcgggctccctcgccttcctcttcaccgcgccctacgccaacggctgcgacgccgccaccgcctccctgccctccttctccgccgacgccgcgcgccggttctccgccgaccacgggatcgcggtgcgctccgtagcgctgcgcgtcgcagacgccgccgaggccttccgcgccagcgtcgacgggggcgcgcgcccggccttcgcccccgtggacctcggccgcggcttcggcttcgcggaggtcgagctctacggcgacgtcgtgctccgcttcgtcagccacccggacggcacggacgtgcccttcttgccggggttcgagggcgtgaccaacccggacgccgtggactacggcctgacgcggttcgaccacgtcgtcggcaacgtcccggagcttgcccccgccgcagcctacatcgccgggttcacggggttccacgagttcgccgagttcacggcggaggacgtgggcacgaccgagagcgggctcaactcggtggtgctcgccaacaactcggagggcgtgctgctgccgctcaacgagccggtgcacggcaccaagcgccggagccagatacagacgttcctggaacaccacggcggcccgggcgtgcagcacatcgcggtggccagcagtgacgtgctcaggacgctcaggaagatgcgtgcgcgctccgccatgggcggcttcgacttcctgccacccccgctgccgaagtactacgaaggcgtgcgacgccttgccggggatgtcctctcggaggcgcagatcaaggaatgccaggagctgggtgtgctcgtcgatagggacgaccaaggggtgttgctccaaatcttcaccaagccagtaggggacaggccgaccttgttcctggagatgatccagaggatcgggtgcatggagaaggacgagagaggggaagagtaccagaagggtggctgcggcgggttcggcaaaggcaacttctccgagctgttcaagtccattgaagattacgagaagtcccttgaagccaagcaatctgctgcagttcagggatcatag

SEQ ID NO: 2 - protein, 434 aa, Hordeum vulgare
MPPTPTTPAATGAAAAVTPEHARPHRMVRFNPRSDRFHTLSFHHVEFWCADAASAAGRFAFALGAPLAARSDLSTGNSAHASQLLRSGSLAFLFTAPYANGCDAATASLPSFSADAARRFSADHGIAVRSVALRVADAAEAFRASVDGGARPAFAPVDLGRGFGFAEVELYGDVVLRFVSHPDGTDVPFLPGFEGVTNPDAVDYGLTRFDHVVGNVPELAPAAAYIAGFTGFHEFAEFTAEDVGTTESGLNSVVLANNSEGVLLPLNEPVHGTKRRSQIQTFLEHHGGPGVQHIAVASSDVLRTLRKMRARSAMGGFDFLPPPLPKYYEGVRRLAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 3 - DNA, 1470 bp, Fragilariopsis cylindrus
atgggtttctcttcagtatcgttatttccgctatcgtggtcggcgtcggcagtaacatcaacctatctcgagacgtccactagtacacaatcgaatgaagctgctactaccacttcgacaacggcgtgggaaccaaaattatcatcattagaagaaagaatagaaacggaaacgattgagtcgatcggtttccatcatatagaattttattgtggagatgctcgtagtatggcaaatcaattcgctgtctcattgggtatgtccgtcacgggtatcaccggccaatccacggggaatgatcaatgcatttcctatggattacaaagtggagagcagtttcgactattattaactgctccctattcacgagcgagagccactactcgcgatgacgacgacgacgacgacgacaacagtcctgatttggatgccgacgctccgatgccactccctaattataatgtagaagatgctcatactttcttccaaaatcatggcttagcagctcgagcggttggcatagaagtcatggatgccaaaaaagctttcgaggtatccgtggccaatggcgcaattccagtactggaaccaacctttcttcccaacggatgctacatctcagaagttgaattgtacggtgacgttgtgttgagatacgtgagtttcatcacatcgaatgaaaatcatacttataatgatgatgcatcacaaccatttctacctcatttagcaccaataattgatcaaagtaggaaagaggatgatgataataatgatgatggttttgggttatataaaatcgaccatgccgttgggaatgttcccaatttacaagaggtatactcacatatccaaaaatttacaggatttcatgaatttgctgaatttacatcagaagatgttggaactgtagactctggattaaattctgttgttttagccagtgacagtgaagcgattctgttacctataaatgaaccaactaatggacgacgaaaatcacaaattcaaacgtatctagaacagaacgagggccctggtctacaacatttagcagtcaaaacgaaagatatattttcaaccgtccgaaagatgcgaagaagtcaacaaggtatgtcgggatttgaattgatgaaacgaccgagtgaggaatattacaaagaacttcctgatcgacttggtgatcaattgacacccacgcagtatcaagaattagaggaacttggtatccttgcggattccgatgaggaaggaattttgatgcaaatttttaccaagcccgtcggtgatcgacctacattcttttttgaactaattcaacgaatcggttgcgtcattgagcatgacgatgacgacaggcaggagttatcagttgatcttgaacgaccaggatgtggtggttttggtaagggtaatttccgagaacttttcagatcaattgaagagcacgagaaaactttaaaggtatag

SEQ ID NO: 4 - DNA, 1467 bp, Fragilariopsis cylindrus
atgggttttagcagcgttagcctgtttccgctgagctggtcagcaagcgcagttaccagcacctatctggaaaccagcaccagcacccagagcaatgaagcagcaaccaccaccagtaccaccgcatgggaaccgaaactgagcagcctggaagaacgtattgaaaccgaaaccattgaaagcattggctttcatcacattgaattttattgcggtgatgcacgtagcatggcaaatcagtttgcagttagcctgggtatgagcgttaccggtattaccggtcagagcaccggtaatgatcagtgtattagctatggtctgcagagcggtgaacagtttcgtctgctgctgaccgcaccgtatagccgtgcacgtgcaaccacccgtgatgatgatgatgacgatgacgataatagtccggatctggatgcagatgcaccgatgccgctgccgaattataatgttgaagatgcccataccttttttcagaatcatggtctggcagcacgtgcagttggtattgaagttatggatgccaaaaaagcctttgaagttagcgttgcaaatggtgcaattccggttctggaaccgacctttctgccgaatggttgttatatttctgaagtggaactgtatggtgatgttgttctgcgttatgtgagctttattaccagcaatgaaaaccacacctacaatgatgatgccagccagccgtttctgccgcatctggcaccgattattgatcagagccgtaaagaagatgatgataataatgatgatggctttggcctgtataaaattgatcatgccgttggtaatgtgccgaatctgcaagaagtttatagccatattcagaaatttaccggctttcatgaatttgccgaatttaccagcgaagatgttggcaccgttgatagcggtctgaatagcgttgttctggcaagcgatagcgaagcaattctgctgccgattaatgaaccgaccaatggtcgtcgtaaaagccagattcagacatatctggaacagaatgaaggtccgggtctgcagcatctggccgttaaaaccaaagatatttttagcaccgtgcgtaaaatgcgtcgtagccagcagggtatgagcggttttgaactgatgaaacgtccgagcgaagaatattataaagaactgccggatcgtctgggtgatcagctgaccccgacccagtatcaagaattagaagaactgggtattctggcagatagtgatgaagaaggtattctgatgcagatttttaccaaaccggttggtgatcgtccgacctttttttttgaactgattcagcgtattggctgcgtgattgaacatgatgatgatgatcgtcaagaactgagcgttgatctggaacgtccgggttgtggtggttttggtaaaggtaattttcgtgaactgtttcgcagcattgaagaacatgaaaaaaccctgaaagtg

SEQ ID NO: 5 - protein, 489 aa, Fragilariopsis cylindrus
MGFSSVSLFPLSWSASAVTSTYLETSTSTQSNEAATTTSTTAWEPKLSSLEERIETETIESIGFHHIEFYCGDARSMANQFAVSLGMSVTGITGQSTGNDQCISYGLQSGEQFRLLLTAPYSRARATTRDDDDDDDDNSPDLDADAPMPLPNYNVEDAHTFFQNHGLAARAVGIEVMDAKKAFEVSVANGAIPVLEPTFLPNGCYISEVELYGDVVLRYVSFITSNENHTYNDDASQPFLPHLAPIIDQSRKEDDDNNDDGFGLYKIDHAVGNVPNLQEVYSHIQKFTGFHEFAEFTSEDVGTVDSGLNSVVLASDSEAILLPINEPTNGRRKSQIQTYLEQNEGPGLQHLAVKTKDIFSTVRKMRRSQQGMSGFELMKRPSEEYYKELPDRLGDQLTPTQYQELEELGILADSDEEGILMQIFTKPVGDRPTFFFELIQRIGCVIEHDDDDRQELSVDLERPGCGGFGKGNFRELFRSIEEHEKTLKV

SEQ ID NO: 6 - DNA, 1341 bp, Chlorella sp.
atggtggtcgaggctgcggccgcctccaacggcaatggcgcaggggaggaggtgttcagcaagaagctcgtgggatatgacggtttccagcgccacaacccacgctccgaccgcttccccatgcacaagttccaccacgtcgagttctggtgcggcgatgccaccaccaccagctgcaggcaagcgcgccccccagaagcacccatgtttgggtatgggctgggcctgactctggtggccaagagcgaccagtccacgggcaaccaccactacgcgtcgtacgtcatgcagtcgggcgatcttgtgatggcctttaccgcgccctacagcacccagacagacaagagcggcagcagcccgcccgcagcgtacgaccaggacgccgcctacgccttcctcaagaagcacggcatggcggtgcgcgcctttggaatcctggtggacgatgccgcggaggcgtaccgcatagccactgcccacggcggggtgggtgtggcgccacccaccacccgcacggacgcagccagcggcaccagcctgacgtggagcgaggtgcagctgtacggcgactgcgtgctgcgctttgtcagcggcgactacgagggcgccttcatccccggctaccagcccgtggaagacgcgccccaagtctcctacggcctgcagcgcctggaccatgcggtgggcaacgtgccagagctgatccctcaagtggagtacatggctcgcagcctgggctggcacgagtttgctgagttcactgccgaggatgtgggcactgtggactcgggcctcaactccatggtcatggccaacaacaacgagatgattctgctgccggtcaacgagcccacccacggcaccaagcgcaagagccagatccagaccttcctggagcagaatgaggggcccgggctgcagcacatggccctgaaaacagacgacatcgtagccaccatgcgacagctccgggccaggtctgcgtttggcggcttcgacttcatgcccaggccttcgcctgactactaccgcaagctgcctgcccgcatcggcagcctgctgacggcgcagcagtacaaggacgttgaggagctggggctgcttgtggacaaggatgaccagggcgtgctgctccagatcttcaccaagccgctgggcgaccgacccaccgtgtttttcgaaatcatccagcgcctgtgcgccctggagccgcaggcgcccaagagccagcgcggcgcggtgccttccgaggtcggcggctgcggcggctttggcaagggcaacttcagtgagctgttcaagagcatcgaggtgtacgagacggatctgggcatcaactaa

SEQ ID NO: 7 - DNA, 1341 bp, Chlorella sp
atggttgttgaagcagcagcagcaagcaatggtaatggtgccggtgaagaagtgtttagcaaaaaactggtgggctatgatggttttcagcgtcataatccgcgtagcgatcgttttccgatgcataaatttcatcatgtggaattttggtgtggtgatgcaaccaccaccagttgtcgtcaggcacgtcctccggaagcaccgatgtttggttatggtctgggtctgaccctggttgcaaaaagcgatcagagcaccggtaatcatcattatgcaagctatgttatgcagagcggtgatctggttatggcatttaccgcaccgtatagcacccagaccgataaaagcggtagcagtccgcctgcagcatacgatcaggatgcagcatacgcctttctgaaaaaacatggtatggcagttcgtgcatttggtattctggttgatgatgcagcagaagcatatcgtattgcaaccgcacatggtggtgttggtgttgcacctccgaccacccgtaccgatgcagcaagcggcaccagcctgacctggtctgaagttcagctgtatggtgattgtgttctgcgttttgttagcggtgattatgaaggtgcatttattccgggttatcagccggttgaagatgcaccgcaggttagctatggtctgcagcgtctggatcatgcagttggtaatgttccggaactgattccgcaggttgaatatatggcacgtagcctgggttggcatgaatttgcagaatttaccgcagaagatgttggcaccgttgatagcggtctgaatagcatggttatggccaataacaatgaaatgattctgctgccggtgaatgaaccgacccacggcaccaaacgtaaaagccagattcagacctttctggaacagaatgaaggtccgggtctgcagcacatggcactgaaaaccgatgatattgttgcaaccatgcgtcagctgcgtgcacgtagcgcatttggtggttttgattttatgcctcgtccgagtccggattattatcgtaaactgcctgcacgtattggtagcctgctgaccgcacagcagtataaagatgttgaagaactgggtctgctggttgataaagatgatcagggtgttctgctgcagatttttaccaaaccgctgggtgatcgtccgaccgttttttttgaaattattcagcgtctgtgtgcactggaaccgcaggcaccgaaaagccagcgtggtgcagttccgagcgaagttggtggttgtggtggttttggtaaaggtaattttagcgaactgtttaaaagcattgaagtgtatgaaaccgatctgggcatcaattaa

SEQ ID NO: 8 - protein, 446 aa, Chlorella sp
MVVEAAAASNGNGAGEEVFSKKLVGYDGFQRHNPRSDRFPMHKFHHVEFWCGDATTTSCRQARPPEAPMFGYGLGLTLVAKSDQSTGNHHYASYVMQSGDLVMAFTAPYSTQTDKSGSSPPAAYDQDAAYAFLKKHGMAVRAFGILVDDAAEAYRIATAHGGVGVAPPTTRTDAASGTSLTWSEVQLYGDCVLRFVSGDYEGAFIPGYQPVEDAPQVSYGLQRLDHAVGNVPELIPQVEYMARSLGWHEFAEFTAEDVGTVDSGLNSMVMANNNEMILLPVNEPTHGTKRKSQIQTFLEQNEGPGLQHMALKTDDIVATMRQLRARSAFGGFDFMPRPSPDYYRKLPARIGSLLTAQQYKDVEELGLLVDKDDQGVLLQIFTKPLGDRPTVFFEIIQRLCALEPQAPKSQRGAVPSEVGGCGGFGKGNFSELFKSIEVYETDLGIN

SEQ ID NO: 9 - DNA, 1257 bp, Thalassiosira
tttcatcatattgaattcttcgccagtgatgcgcttacgacagccaagcggtttgagctagcgttgggattgccaattacgtgttggagttcattggctacggggaacgatgtttgtgttacctacggattggaggggatgcaaactcgaaagattgaaaccgacaacgcaaacaagaacggaggggcaggatccaatgtacaaatgactgcacctcttcctcttccggggtatgatattgacaaagctcatgagttttactcgaagcacggtttggcagtacgagctgtgggagtggaagtgaaggatgcaactgtagcttatgcaaatgctgttgagaatggtgctacaggagtattggagcctacgattgttgaaaactttaacagcgatggagattcgcagaagtgtcatatggctgaggtggaattgtatggtgatgtggtgttgagattagtcagttttcatggagattgtagtgccgaacaatctacattccttcctcacttgtcgccgtatccatccaacagcaacaagaacaaaccaacttacggacttgctcgtctagatcacacggtgggcaatgttcccaacctcctcgcgacgcaacgatacattcaaacattcaccaactaccatcccttcgcagagttcactccggaagatgtgggaacagtcgactctggtcttaatagtgtagtacttgcatcagacaacgagaatgttttgctgcctctcaatgaacctaccgaaggtaaacgaaagagtcagattcaaacatatctagagcagaacgagggaccgggactgcagcatattgccatcaagacgaatgatatctttgataccattgcaaagatgagacacgcagaagagaactttggaggtttcgagttgatgaaacgtccatcggatgagtattacaaagagttgccttcgagattgggtgataagttgactgtcgagcaatacaaacagctggaagagttgggaatactggcagatgcagatgacgaaggtatattgcttcaaatattcacaaagccattgggagacaggcccaccttgttccttgagattattcagcgaattgggtgtgtgttaccggatgacgatgaagcaactgatgaaggggaagctaagaatgcacacaatagaatagtcagagaacgtcccggatgtggtggatttggtcagggcaacttccgtgaactcttcaaagcaattgaagaacatgagaagacactcaaggtctaa

SEQ ID NO: 10 - DNA, 1257 bp, Thalassiosira
tttcatcatatcgaattttttgcctcagatgcactgaccaccgcaaaacgctttgaactggcactgggtctgccgattacctgttggagcagcctggcaaccggtaatgatgtttgtgttacctatggtctggaaggtatgcagacccgtaaaattgaaaccgataacgccaataaaaacggtggtgcaggtagcaatgttcagatgaccgcaccgctgccgctgcctggttatgatattgataaagcccatgaattctatagcaaacatggtctggcagttcgtgcagttggtgttgaagttaaagatgcaaccgttgcctatgcaaatgcagttgaaaatggtgcaaccggtgttctggaaccgaccattgttgaaaactttaatagtgatggcgatagccagaaatgtcacatggccgaagttgaactgtatggtgatgttgttctgcgtctggttagctttcatggtgattgtagcgcagaacagagcacctttctgccgcatctgagcccgtatccgagcaatagcaataaaaacaaaccgacctatggcctggcacgtctggatcataccgttggtaatgttccgaatctgctggcaacccagcgttatattcagacctttaccaactatcatccgtttgcagaatttacaccggaagatgttggcaccgttgatagcggtctgaatagcgttgttctggcaagcgataatgaaaatgttctgctgccgctgaatgaaccgaccgaaggtaaacgtaaaagccagattcagacctatctggaacagaatgaaggtccgggtctgcagcatattgcaattaaaaccaatgatatttttgataccattgccaaaatgcgccatgccgaagaaaattttggtggttttgaactgatgaaacgtccgtccgatgaatactataaagaactgccgagccgtctgggtgataaactgaccgttgaacagtataaacagctggaagaactgggtattctggcagatgcagatgatgaaggtattctgctgcagatttttaccaaaccgctgggtgatcgtccgaccctgtttctggaaattattcagcgtattggttgtgttctgccggatgatgatgaagcaaccgatgaaggtgaagcaaaaaatgcccataatcgtattgttcgtgaacgtccgggttgtggtggttttggtcagggtaattttcgcgaactgtttaaagccattgaagaacatgaaaaaaccctgaaagtttaa

SEQ ID NO: 11 - protein, 418 aa, Thalassiosira pseudonana
FHHIEFFASDALTTAKRFELALGLPITCWSSLATGNDVCVTYGLEGMQTRKIETDNANKNGGAGSNVQMTAPLPLPGYDIDKAHEFYSKHGLAVRAVGVEVKDATVAYANAVENGATGVLEPTIVENFNSDGDSQKCHMAEVELYGDVVLRLVSFHGDCSAEQSTFLPHLSPYPSNSNKNKPTYGLARLDHTVGNVPNLLATQRYIQTFTNYHPFAEFTPEDVGTVDSGLNSVVLASDNENVLLPLNEPTEGKRKSQIQTYLEQNEGPGLQHIAIKTNDIFDTIAKMRHAEENFGGFELMKRPSDEYYKELPSRLGDKLTVEQYKQLEELGILADADDEGILLQIFTKPLGDRPTLFLEIIQRIGCVLPDDDEATDEGEAKNAHNRIVRERPGCGGFGQGNFRELFKAIEEHEKTLKV

SEQ ID NO: 12 - DNA, 1092 bp, Cyanothece
atggaaatcgatcatattcatttctacgttgaagatgcagcacatcaacgagattggtttattgataaaatggggtttcaatccatcagcaacagtatccatgatgacacttatagcgaagtagtagggaatcagtctgtttactttatcttatcttctcccctcaacgatgctagtccagtttcttattacttgaaatctcatcctccgggggttgctgatgttgcttttcgtgttgacaatcttaattttttattagacaaagtatcccgttttaaggtcgaaattattaatcaatctagtctaacagcttttcctctaaataaaccagtgaaattcgcgaaacttaaaggatggggttctgtcaatcataccttaattgatcaggcaagtcctaggacttttattagctcaaaaatgattgctaaaagcgatattattgggattgatcatgttgttttaaatgttcctcaaggtgaactccccttagccataaattggtacaaaaatgtatttgattttataagtcatcaacagttcaacatccaaacagaacattcggggttatctagtgaagccttagttgatagttcaggaaaagtacaatttaatattaatcaaccaagttctactaattctcagattcaggaatttttagaccataataacggttcaggcattcaacatattggtttaaaatcaagtaatattttacaaagtgttgcacaaatgcgtcaaaggggattaccctttttatccgttcctaattcctattaccaaaacctaaaagaattgattagaaaatcgacaatttcttgtttaagccaacaggaactagaacaaattgaaactgaacaaattctagtttgttggccagaagataacccgacttcaatcctgatgcaaattttcactcaacccatttttaagcagccgactttcttttttgaattaattcaaagacgcaaccaagcacagggatttggccaaggtaattttcaagcgttatttgaagccatagaatcagaacaaatcaagagaaatagggtatcctcacgagtcactttacaggctgtaacaccccaatcttga

SEQ ID NO: 13 - DNA, 1089 bp, Cyanothece
atggaaattgatcatattcatttttatgtggaagatgcagcccatcagcgtgattggtttattgataaaatgggctttcagagcattagcaatagcattcatgatgatacctatagcgaagttgtgggtaatcagagcgtgtatttcattctgagcagtccgctgaatgatgcaagtccggttagctattatctgaaaagccatcctccgggtgttgcagatgttgcatttcgtgttgataatctgaattttctgctggataaagtgagccgctttaaagtggaaatcattaatcagagcagcctgaccgcatttccgctgaataaaccggttaaatttgccaaactgaaaggttggggtagcgttaatcataccctgattgatcaggcaagtccgcgtacctttattagcagcaaaatgattgccaaaagcgatattattggcattgatcatgtggttctgaatgttccgcagggtgaactgccgctggcaattaattggtacaaaaatgtgtttgattttattagccatcagcagtttaatattcagaccgaacatagcggtctgagcagcgaagcactggttgatagcagcggtaaagttcagtttaatattaatcagccgagcagcaccaatagccagattcaagaatttctggatcataataatggcagcggcattcagcatattggtctgaaaagcagcaatattctgcagagcgttgcacagatgcgtcagcgtggtctgccgtttctgagcgttccgaatagctattatcagaatctgaaagaactgattcgcaaaagcaccattagctgtctgagccagcaagaactggaacaaattgaaaccgaacaaattctggtttgttggcctgaagataatccgaccagcattctgatgcagatttttacccagccgatttttaaacagccgacctttttttttgaactgattcagcgtcgtaatcaggcacagggttttggtcagggtaattttcaggcactgtttgaagcaattgaaagtgaacaaattaaacgtaatcgtgttagcagccgtgttaccctgcaggcagttacaccgcagagc

SEQ ID NO: 14 - protein, 363 aa, Cyanothece
MEIDHIHFYVEDAAHQRDWFIDKMGFQSISNSIHDDTYSEVVGNQSVYFILSSPLNDASPVSYYLKSHPPGVADVAFRVDNLNFLLDKVSRFKVEIINQSSLTAFPLNKPVKFAKLKGWGSVNHTLIDQASPRTFISSKMIAKSDIIGIDHVVLNVPQGELPLAINWYKNVFDFISHQQFNIQTEHSGLSSEALVDSSGKVQFNINQPSSTNSQIQEFLDHNNGSGIQHIGLKSSNILQSVAQMRQRGLPFLSVPNSYYQNLKELIRKSTISCLSQQELEQIETEQILVCWPEDNPTSILMQIFTQPIFKQPTFFFELIQRRNQAQGFGQGNFQALFEAIESEQIKRNRVSSRVTLQAVTPQS

SEQ ID NO: 15 - DNA, 1068 bp, Acaryochloris marina
atggattttgatcatattcatttttatgttcatgattccaagcaatgccagcgttggtttactaacgttttaggatttcaatatcttgggagcaatactacggctgatcggcagattgaagttgtctcttctggggcgattgtctgtatattttccagccctctaaaccgaaccagcccagttgcccagtatctacaacaacaccctcctggtgtcgttgatttggctttcttggtcccggacgttcaggctacgcttacctgcgctgtccagtcaggagcaacccttttacaacctttgaccgaagaaaaaaacgaccaaggaacgttaacttggggaaaagtacgagggtggggagcgttagaacataccttggtagagcgaagagggcaaacctccactctgccatccagcatttttcccatctcaattcatgggcatgatgcccatcagagtctatttacccagattgatcatggggttttgaatgtgggtaagcatcagctgcaagctgctgtgagttggtatcagcgcatatttggatttaaaactcaccgatattttgatattcaaacgcgtcgttcaggtctgcgcagtgaagtgttgacccatccccaaggccaaatcaagtttccgatcaatgaacccacctcagcgaattcccaaatccaagaatttctagaggtcaatcggggggcaggtattcaacatatcgcattgggaacttctaatattgttgaaacggttactcagcttaagcatcgagggctatccatcctagatattccacccagctactatcaacgcttacgacaccagtttgagcaagtctattcccacctcgattggcatgccctggaaacacaacatattctggctgattttgaggaagatgctggagccggaattctattgcaaaccttcacaaagcctatctttccacaacccacttttttctttgaaattattgagcgccaacggcaggcccaagggttcggacaacgaaactttttggccctttttcaagccatggagcgggaacaacagaaacggggagtattgctatag

SEQ ID NO: 16 - DNA, 1065 bp, Acaryochloris marina
atggattttgatcacatccacttttatgtgcatgacagcaaacagtgtcagcgttggtttaccaatgttctgggttttcagtatctgggtagcaataccaccgcagatcgtcagattgaagttgttagcagcggtgcaattgtttgcatttttagcagtccgctgaatcgtaccagtccggttgcacagtatctgcagcagcatccgcctggtgttgttgatctggcatttctggttccggatgttcaggcaaccctgacctgtgcagttcagagcggtgccaccctgctgcagccgctgaccgaagaaaaaaatgatcagggtacactgacctggggtaaagttcgtggttggggtgcactggaacacaccctggttgaacgtcgtggtcagaccagcaccctgccgagcagcatttttccgattagcattcatggtcatgatgcacatcagagcctgtttacccagattgatcatggtgttctgaatgttggtaaacatcagctgcaggcagcagttagctggtatcagcgtatttttggctttaaaacccaccgctattttgatattcagacccgtcgtagcggtctgcgtagcgaagttctgacccatccgcagggtcagatcaaatttccgatcaatgaaccgaccagcgcaaatagccagattcaagaatttctggaagttaatcgtggtgccggtattcagcatattgcactgggcaccagcaatattgttgaaaccgttacccagctgaaacatcgtggtctgagcattctggatattccgcctagctattatcagcgtctgcgtcatcagtttgaacaggtttatagccatctggattggcatgccctggaaacccagcatattctggcagattttgaagaagatgcaggcgcaggtattctgctgcaaacctttaccaaaccgatttttccgcagccgaccttttttttcgaaattattgaacgtcagcgtcaggcacagggttttggtcagcgcaattttctggcactgtttcaggcaatggaacgtgaacagcagaaacgtggtgtgctgctg

SEQ ID NO: 17 - protein, 355 aa, Acaryochloris marina
MDFDHIHFYVHDSKQCQRWFTNVLGFQYLGSNTTADRQIEVVSSGAIVCIFSSPLNRTSPVAQYLQQHPPGVVDLAFLVPDVQATLTCAVQSGATLLQPLTEEKNDQGTLTWGKVRGWGALEHTLVERRGQTSTLPSSIFPISIHGHDAHQSLFTQIDHGVLNVGKHQLQAAVSWYQRIFGFKTHRYFDIQTRRSGLRSEVLTHPQGQIKFPINEPTSANSQIQEFLEVNRGAGIQHIALGTSNIVETVTQLKHRGLSILDIPPSYYQRLRHQFEQVYSHLDWHALETQHILADFEEDAGAGILLQTFTKPIFPQPTFFFEIIERQRQAQGFGQRNFLALFQAMEREQQKRGVLL

SEQ ID NO: 18 - DNA, 1020 bp, Synechocystis sp. PCC 6803
atggaattcgactatcttcatttatacgttgacgattatcagtcagctcatcgttgttatcaacgtcaatggggtttcacttgcgtaaataaaattattactgaccaaggaattactggcatctaccaacaggggcaaatacttctgctaatttcggcatcggaatctagtttgagtagatatgccgactatctccagaaacatccccccggcgtaggtgaagtcgcttggcaggtggccaattggcaaaaaattcagcatcaattatcagaattacagatagaaaccacaccagttattcatcctctgactaaagcagaaggattaacttttttgctctggggagatgtgcaccatagcatttatcctgttcgttctgagctaaatcagaataaaacattgcatggtgttggtttaacgaccatcgaccatgtggtgctaaacattgccgccgatcaatttacccaggcttcccaatggtatcaacaggtgtttggctggtcggtgcagcagagttttactgtcaatacgccccattctggtctgtatagcgaagccctggccagtgccaatgggaaagtccaatttaacctcaattgtcccaccaataacagttcccaaattcaaacttttttagccaataaccatggggctggtattcaacatgtcgctttttccactacgagtattacgcgaactgtggctcatctgcgggaaaggggcgtaaattttttaaaaatccccactggctattatcaacagcaaagaaacagtagctattttaattatgcaagtttggattgggataccttacagtgcctagaaattttgctggatgatcaagataatacgggggagcgattactgctacaaatttttagtcagccttgctatggagtaggcactctattttgggaaattattgaacgccgccaccgggcaaaaggatttggtcaaggaaactttcaagctctctatgaagcggtggagactttagaaaaacagttagaagtgccataa

SEQ ID NO: 19 - DNA, 1020 bp, Synechocystis sp. PCC 6803
atggaattcgattacctgcatctgtatgttgatgactatcagagcgctcaccgttgttaccagcgtcagtggggctttacttgtgtgaacaaaatcatcaccgaccagggtatcactggtatttaccagcagggtcagatcctgctgctgatcagcgcttctgaatcttccctctctcgctatgccgattacctccagaaacatccgccaggtgtaggtgaagtcgcctggcaggtcgcaaactggcagaaaattcagcaccagctgtccgaactgcagattgaaactaccccggtgattcacccactgaccaaagcagaaggcctgactttcctgctgtggggtgacgttcaccactccatctacccagtacgtagcgagctgaaccagaacaaaaccctgcatggcgttggtctgaccactatcgatcacgtggttctgaacatcgcagcggaccagttcacccaggcgagccagtggtatcagcaggtattcggttggtccgttcagcagtctttcacggttaacaccccgcattccggtctgtactctgaagctctggcgtctgcgaacggcaaagttcagttcaacctgaactgcccgaccaacaacagctcccagattcagaccttcctggcgaacaaccacggtgctggtatccagcacgttgcattctctactacctctatcacccgtacggtcgctcacctgcgtgaacgtggcgtgaacttcctgaaaatcccgaccggttactatcagcagcagcgcaacagctcctacttcaactacgcgtctctggattgggataccctgcagtgcctggagattctgctggacgaccaggacaacactggcgaacgcctgctgctgcagatcttttctcagccgtgctatggcgtgggtacgctgttttgggaaattatcgagcgccgtcaccgtgctaaaggctttggccagggcaactttcaggcactgtacgaggcagtagaaaccctggaaaaacagctcgaagtgccataa

SEQ ID NO: 20 - protein, 339 aa, Synechocystis sp. PCC 6803
MEFDYLHLYVDDYQSAHRCYQRQWGFTCVNKIITDQGITGIYQQGQILLLISASESSLSRYADYLQKHPPGVGEVAWQVANWQKIQHQLSELQIETTPVIHPLTKAEGLTFLLWGDVHHSIYPVRSELNQNKTLHGVGLTTIDHVVLNIAADQFTQASQWYQQVFGWSVQQSFTVNTPHSGLYSEALASANGKVQFNLNCPTNNSSQIQTFLANNHGAGIQHVAFSTTSITRTVAHLRERGVNFLKIPTGYYQQQRNSSYFNYASLDWDTLQCLEILLDDQDNTGERLLLQIFSQPCYGVGTLFWEIIERRHRAKGFGQGNFQALYEAVETLEKQLEVP

SEQ ID NO: 21 - DNA, 1326 bp, Alopecurus myosuroides
atgcctccgaccaccgcaaccgcaaccggtgctgcagcagcagccgttacaccggaacatgcagcacgtcgttttccgcgtgttgttcgtgttaatccgcgtagcgatcgttttccggttctggcatttcatcatgttgaattttggtgtgccgatgcagcaagcgcagcaggtcgttttagctttgcactgggtgcaccgctggcagcacgtagcgatctgagcaccggtaatagcagccatgcaagccatctgctgcgtagtggtgcactggcatttctgtttaccgcaccgtatgcaccgcctccgcaggatgcagcagatgcagccgctaccgccagcattccgagctttagcaccgaagcagcacgtacctttagcagcgcacatggtctggcagttcgtagcgttgcaattcgtgttgcagatgccgcagaagcatttcataccagcgttgcgggtggtgcacgtccggcatttgcaccggcagatctgggtagcggttttggtctggccgaagttgaactgtatggtgatgttgttctgcgttttgttagtcatccggatggtgatgatgttccgtttctgccgggttttgaaggtgttagccgtccgggtgcaatggattatggtctgacccgttttgatcatgttgttggtaatgttccggaaatggcaccggttgcagcatatatgaaaggttttaccggctttcatgaatttgccgaatttaccgcagaagatgttggcaccgcagaaagcggtctgaatagcgttgttctggcaaataatagcgaagcagttctgctgccgctgaatgaaccggtgcatggcaccaaacgtcgtagccagattcagacctatctggattatcatggtggtccgggtgttcagcatattgcactggcaagcagtgatgttctgcgtaccctgcgtgaaatgcgtgcacgtagcgcaatgggtggttttgaatttatggcaccgccgcaggcaaaatattatgaaggtgttcgtcgtctggctggtgatgttctgagcgaagcacagattaaagaatgtcaggaactgggcgttctggttgatcgtgatgatcagggtgttctgctgcagatttttaccaaaccggttggtgatcgtcgtccgaccttttttctggaaatgattcagcgtattggctgcatggaaaaagatgaaattggccaggaatatcagaaaggcggctgtggtggttttggtaaaggtaattttagcgaactgtttaaaagcattgaagattatgaaaaaagcctggaagccaaacagagcgcagttgcacagcagagctaa

SEQ ID NO: 22 - protein, 441 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSTEAARTFSSAHGLAVRSVAIRVADAAEAFHTSVAGGARPAFAPADLGSGFGLAEVELYGDVVLRFVSHPDGDDVPFLPGFEGVSRPGAMDYGLTRFDHVVGNVPEMAPVAAYMKGFTGFHEFAEFTAEDVGTAESGLNSVVLANNSEAVLLPLNEPVHGTKRRSQIQTYLDYHGGPGVQHIALASSDVLRTLREMRARSAMGGFEFMAPPQAKYYEGVRRLAGDVLSEAQIKECQELGVLVDRDDQGVLLQIFTKPVGDRRPTFFLEMIQRIGCMEKDEIGQEYQKGGCGGFGKGNFSELFKSIEDYEKSLEAKQSAVAQQS

SEQ ID NO: 23 - DNA, 1323 bp, Alopecurus myosuroides
atgccaccaactactgctactgctacaggtgctgctgctgcagctgttactccagaacatgctgctagaaggttcccaagagttgttagagttaacccaaggtctgataggttcccagttcttgctttccatcatgttgagttttggtgtgctgatgctgcttctgctgctggaagattttcttttgctcttggtgctccacttgctgctagatctgatttgtctactggaaactcttctcacgcttctcaccttttgagatctggtgctcttgctttccttttcactgctccttatgctccaccaccacaagatgctgcagatgcagcagctactgcttctattccatctttttcaactgaggctgctaggactttctcttctgctcatggattggctgttagatctgtggctattagagttgcagatgctgcagaggctttccatacttctgttgctggtggtgctagaccagcttttgctccagctgatcttggatctggatttggacttgctgaggttgagctttacggtgatgttgttcttagattcgtgtctcacccagatggtgatgatgttccatttcttccaggattcgagggtgttagtagaccaggtgctatggattatggactcactaggttcgatcacgttgtgggaaatgttccagaaatggctccagttgctgcttacatgaagggattcactggatttcatgagttcgctgagttcactgctgaggatgttggaactgctgagtctggacttaactctgttgtgcttgctaacaactctgaggctgttcttttgccacttaatgagccagttcacggcactaagagaagatctcagattcagacttacctcgattaccatggtggaccaggtgttcaacatattgctcttgcttcatctgatgtgcttaggactcttagagagatgagagctagatctgctatgggaggatttgagtttatggctccaccacaagctaagtattacgaaggtgttagaaggcttgctggtgatgttctttctgaggctcaaatcaaagagtgccaagagcttggagttcttgtggatagagatgatcagggtgtgcttctccagattttcactaagccagttggagataggccaacattcttcttggagatgattcagaggatcggctgcatggaaaaggatgagattggacaagagtaccaaaagggcggatgtggtggatttggaaagggaaatttctccgagcttttcaagtccatcgaggattacgagaagtctcttgaggctaagcaatctgctgttgctcaacagtcttga

SEQ ID NO: 24 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSTEAARTFSSAHGLAVRSVAIRVADAAEAFHTSVAGGARPAFAPADLGSGFGLAEVELYGDVVLRFVSHPDGDDVPFLPGFEGVSRPGAMDYGLTRFDHVVGNVPEMAPVAAYMKGFTGFHEFAEFTAEDVGTAESGLNSVVLANNSEAVLLPLNEPVHGTKRRSQIQTYLDYHGGPGVQHIALASSDVLRTLREMRARSAMGGFEFMAPPQAKYYEGVRRLAGDVLSEAQIKECQELGVLVDRDDQGVLLQIFTKPVGDRPTFFLEMIQRIGCMEKDEIGQEYQKGGCGGFGKGNFSELFKSIEDYEKSLEAKQSAVAQQS

SEQ ID NO: 25 - DNA, 1323 bp, Sorghum halapense
atgcctccgaccccgaccaccgcagcagcaacaggtgccgcagttgcagcagcaagcgcagaacaggcagcatttcgtctggttggtcatcgtaattttgttcgtgttaatccgcgtagcgatcgttttcataccctggcatttcatcatgttgaactgtggtgtgccgatgcagccagcgcagcaggtcgttttagctttggtctgggtgcaccgctggcagcacgtagcgatctgagcaccggtaataccgcacatgcaagcctgctgctgcgttcaggtgcactggcatttctgtttaccgcaccgtatgcccatggtgctgatgcagcaaccgcaagcctgccgagctttagcgcagcagaagcacgtcgttttgcagcagatcatggtctggcagttcgtgccgttgcactgcgtgttgcagatgccgaagatgcatttcgtgcaagcgttgcagccggtgcacgtccggcatttgaaccggttgaactgggtctgggttttcgtctggccgaagttgaactgtatggtgatgttgttctgcgttatgttagctatccggatgatgcagatgcaagctttctgccgggttttgttggtgttagcagtccgggtgcggcagattatggcctgcgtcgttttgatcatattgtgggtaatgttccggaactggcaccggcagcggcatattttgcaggttttaccggctttcatgaatttgcagaatttaccgcagaagatgttggcaccaccgaaagcggtctgaatagcatggttctggcaaataatgccgaaaatgttctgctgccgctgaatgaaccggtgcatggcaccaaacgtcgtagccagattcagacctttctggatcatcatggtggtccgggtgttcagcacatggcactggcaagtgatgatgtgctgcgtaccctgcgtgaaatgcaggcatgtagtgcaatgggtggttttgaatttatggcaccgccggcaccggaatattatgatggtgttcgtcgtcgtgccggtgatgttctgaccgaagcacagattaaagaatgtcaggaactgggcgttctggttgatcgtgatgatcagggtgttctgctgcagatttttaccaaaccggttggtgatcgcccgaccttttttctggaaattattcagcgtattggttgcatggaaaaagatgaaaaaggccaggaatatcagaaaggcggttgtggtggttttggtaaaggtaattttagccagctgtttaaaagcattgaagattatgaaaaaagcctggaagcaaaacaggcagctgcagcacagggtccgtaa

SEQ ID NO: 26 - protein, 440 aa, Sorghum halapense
MPPTPTTAAATGAAVAAASAEQAAFRLVGHRNFVRVNPRSDRFHTLAFHHVELWCADAASAAGRFSFGLGAPLAARSDLSTGNTAHASLLLRSGALAFLFTAPYAHGADAATASLPSFSAAEARRFAADHGLAVRAVALRVADAEDAFRASVAAGARPAFEPVELGLGFRLAEVELYGDVVLRYVSYPDDADASFLPGFVGVSSPGAADYGLRRFDHIVGNVPELAPAAAYFAGFTGFHEFAEFTAEDVGTTESGLNSMVLANNAENVLLPLNEPVHGTKRRSQIQTFLDHHGGPGVQHMALASDDVLRTLREMQACSAMGGFEFMAPPAPEYYDGVRRRAGDVLTEAQIKECQELGVLVDRDDQGVLLQIFTKPVGDRPTFFLEIIQRIGCMEKDEKGQEYQKGGCGGFGKGNFSQLFKSIEDYEKSLEAKQAAAAQGP

SEQ ID NO: 27 - DNA, 1323 bp, Sorghum halapense
atgcctccgaccccgaccaccgcagcagcaacaggtgccgcagttgcagcagcaagcgcagaacaggcagcatttcgtctggttggtcatcgtaattttgttcgtgttaatccgcgtagcgatcgttttcataccctggcatttcatcatgttgaactgtggtgtgccgatgcagccagcgcagcaggtcgttttagctttggtctgggtgcaccgctggcagcacgtagcgatctgagcaccggtaataccgcacatgcaagcctgctgctgcgttcaggtgcactggcatttctgtttaccgcaccgtatgcccatggtgctgatgcagcaaccgcaagcctgccgagctttagcgcagcagaagcacgtcgttttgcagcagatcatggtctggcagttcgtgccgttgcactgcgtgttgcagatgccgaagatgcatttcgtgcaagcgttgcagccggtgcacgtccggcatttgaaccggttgaactgggtctgggttttcgtctggccgaagttgaactgtatggtgatgttgttctgcgttatgttagctatccggatgatgcagatgcaagctttctgccgggttttgttggtgttaccagtccgggtgcggcagattatggcctgaaacgttttgatcatattgtgggtaatgttccggaactggcaccggcagcggcatattttgcaggttttaccggctttcatgaatttgcagaatttaccgcagaagatgttggcaccaccgaaagcggtctgaatagcatggttctggcaaataatgccgaaaatgttctgctgccgctgaatgaaccggtgcatggcaccaaacgtcgtagccagattcagacctttctggatcatcatggtggtccgggtgttcagcacatggcactggcaagtgatgatgtgctgcgtaccctgcgtgaaatgcaggcacgtagtgcaatgggtggttttgaatttatggcaccgccggcaccggaatattatgatggtgttcgtcgtcgtgccggtgatgttctgaccgaagcacagattaaagaatgtcaggaactgggcgttctggttgatcgtgatgatcagggtgttctgctgcagatttttaccaaaccggttggtgatcgcccgaccttttttctggaaattattcagcgtattggttgcatggaaaaagatgaaaaaggccaggaatatcagaaaggcggttgtggtggttttggtaaaggtaattttagccagctgtttaaaagcattgaagattatgaaaaaagcctggaagcaaaacaggcagctgcagcacagggtccgtaa

SEQ ID NO: 28 - protein, 453 aa, Sorghum halapense
MPPTPTTAAATGAAVAAASAEQAAFRLVGHRNFVRVNPRSDRFHTLAFHHVELWCADAASAAGRFSFGLGAPLAARSDLSTGNTAHASLLLRSGALAFLFTAPYAHGADAATASLPSFSAAEARRFAADHGLAVRAVALRVADAEDAFRASVAAGARPAFEPVELGLGFRLAEVELYGDVVLRYVSYPTTRTRPSCRGSWADDADASFLPGFVGVTSPGAADYGLKRFDHIVGNVPELAPAAAYFAGFTGFHEFAEFTAEDVGTTESGLNSMVLANNAENVLLPLNEPVHGTKRRSQIQTFLDHHGGPGVQHMALASDDVLRTLREMQARSAMGGFEFMAPPAPEYYDGVRRRAGDVLTEAQIKECQELGVLVDRDDQGVLLQIFTKPVGDRPTFFLEIIQRIGCMEKDEKGQEYQKGGCGGFGKGNFSQLFKSIEDYEKSLEAKQAAAAQGP

SEQ ID NO: 29 - DNA, 1311 bp, Poa annua
atgcctccgaccaccgcaaccgccaccgcagcagcaaccgttacaccggaacatgcagcacgtcgttttccgcgtgttgttcgtgttaatccgcgtagcgatcgttttccggttctgagctttcatcatgttgaattttggtgtgccgatgcagcaagcgcagcaggtcgttttagctttgcactgggtgcaccgctggcagcacgtagcgatctgagcaccggtaatagcgcacatgcaagcctgctgctgcgttcaggtgcactggcatttctgtttaccgcaccgtatgcaccgcagccgcaggatgcagataccgcaagcattccgagctttagcgcagatgcagcacgtgcatttagcgcagcacatggtctggcagttcgtagcgttgcagttcgtgttgcagatgccgcagatgcatttcgtgcaagcattgcagccggtgcacgtccggcatttgcaccggcagatctgggtcgtggttttggtctggccgaagttgaactgtatggtgatgttgttctgcgttttgttagccatccggatgcagatgatgcaccgccgtttctgccgggttttgaagcagttagccgtcgtccgggtgccgttgattatggtctgacccgttttgatcatgttgttggtaatgttccggaaatgggtccggtgattgattatattaaaggctttatgggctttcatgaatttgccgaatttaccgcagaagatgttggcaccaccgaaagcggtctgaatagcgttgttctggcaaataatagcgaagcagttctgctgccgctgaatgaaccggtgcatggcaccaaacgtcgtagccagattcagacctatctggaatatcatggtggtccgggtgttcagcatattgcactggcaagcagtgatgttctgcgtaccctgcgtgaaatgcaggcacgttcagcaatgggtggttttgaatttatggcaccgccgcagccgaaatattatgaaggtgttcgtcgtattgccggtgatgttctgagcgaagcacagattaaagaatgtcaggaactgggcgttctggttgatcgtgatgatcagggtgttctgctgcagatttttaccaaaccggttggtgatcgtccgaccttttttctggaaatgattcagcgtattggctgcatggaaaaagatgaacgtggtcaggaatatcagaaaggcggttgtggcggttttggtaaaggtaattttagcgaactgtttaaaagcattgaagattatgaaaaaagcctggaagccaaacagagcgcagttgcacagcagagctaa

SEQ ID NO: 30 - protein, 436 aa, Poa annua
MPPTTATATAAATVTPEHAARRFPRVVRVNPRSDRFPVLSFHHVEFWCADAASAAGRFSFALGAPLAARSDLSTGNSAHASLLLRSGALAFLFTAPYAPQPQDADTASIPSFSADAARAFSAAHGLAVRSVAVRVADAADAFRASIAAGARPAFAPADLGRGFGLAEVELYGDVVLRFVSHPDADDAPPFLPGFEAVSRRPGAVDYGLTRFDHVVGNVPEMGPVIDYIKGFMGFHEFAEFTAEDVGTTESGLNSVVLANNSEAVLLPLNEPVHGTKRRSQIQTYLEYHGGPGVQHIALASSDVLRTLREMQARSAMGGFEFMAPPQPKYYEGVRRIAGDVLSEAQIKECQELGVLVDRDDQGVLLQIFTKPVGDRPTFFLEMIQRIGCMEKDERGQEYQKGGCGGFGKGNFSELFKSIEDYEKSLEAKQSAVAQQS

SEQ ID NO: 31 - DNA, 1305 bp, Poa annua
atgccaccaactactgctactgctacagctgctgctactgttactccagaacatgctgctagaaggttcccaagagttgttagagttaacccaaggtctgataggttcccagttctttctttccaccacgttgaattttggtgtgctgatgctgcttctgctgctggaagattttcttttgctcttggtgctccacttgctgctagatctgatttgtctactggaaattctgctcacgcttctttgcttttgaggtctggtgctcttgctttcctttttactgctccttatgctccacaaccacaggatgctgatactgcatcaattccatctttctcagctgatgctgcaagggctttttctgctgctcatggattggctgttagatctgttgctgttagagttgctgatgcagctgatgctttcagagcttctattgctgcaggtgctagaccagcttttgctccagctgatcttggaagaggatttggacttgctgaggttgagctttacggtgatgttgttcttagattcgtgtctcacccagatgctgatgatgctccatttcttccaggatttgaggctgtttctagaccaggtgctgttgattatggactcactaggttcgatcacgttgtgggaaatgttccagaaatgggaccagtgatcgattacatcaagggattcatgggattccatgagttcgctgagtttactgctgaggatgttggaactactgagtctggacttaactctgttgtgcttgctaacaactctgaggctgttcttttgccacttaatgagccagttcacggcactaagagaagatctcagattcagacttaccttgagtaccatggtggaccaggtgttcaacatattgctcttgcttcatctgatgtgcttaggactcttagagagatgcaagctagatctgctatgggaggatttgagtttatggctccaccacaacctaagtattacgagggtgttagaaggattgctggtgatgttctttccgaggctcaaatcaaagagtgtcaagagcttggagtgcttgtggatagagatgatcagggtgtgcttctccagattttcactaagccagttggagataggccaacattcttcttggagatgattcagaggatcggctgcatggaaaaggatgagagaggtcaagagtatcaaaagggcggatgtggtggatttggaaagggaaatttctccgagcttttcaagtccatcgaggattacgagaagtctcttgaggctaagcaatctgctgttgctcaacagtcttga

SEQ ID NO: 32 - protein, 434 aa, Poa annua
MPPTTATATAAATVTPEHAARRFPRVVRVNPRSDRFPVLSFHHVEFWCADAASAAGRFSFALGAPLAARSDLSTGNSAHASLLLRSGALAFLFTAPYAPQPQDADTASIPSFSADAARAFSAAHGLAVRSVAVRVADAADAFRASIAAGARPAFAPADLGRGFGLAEVELYGDVVLRFVSHPDADDAPFLPGFEAVSRPGAVDYGLTRFDHVVGNVPEMGPVIDYIKGFMGFHEFAEFTAEDVGTTESGLNSVVLANNSEAVLLPLNEPVHGTKRRSQIQTYLEYHGGPGVQHIALASSDVLRTLREMQARSAMGGFEFMAPPQPKYYEGVRRIAGDVLSEAQIKECQELGVLVDRDDQGVLLQIFTKPVGDRPTFFLEMIQRIGCMEKDERGQEYQKGGCGGFGKGNFSELFKSIEDYEKSLEAKQSAVAQQS

SEQ ID NO: 33 - DNA, 1323 bp, Lolium multiflorum
atgcctccgacaccggcaaccgcaaccggtgctgcagcagcagcagttacaccggaacatgcagcacgtagctttccgcgtgttgttcgtgttaatccgcgtagcgatcgttttccggttctgagctttcatcatgttgaactgtggtgtgccgatgcagcaagcgcagcaggtcgttttagctttgcactgggtgctccgctggcagcccgtagcgatctgagcaccggtaatagcgcacatgcaagcctgctgctgcgtagcggtgcactggcatttctgtttaccgcaccgtatgcaccgcctccgcaggaagcagcaaccgcagctgcaaccgcaagcattccgagctttagcgcagatgcagcccgtacctttgcagcagcacatggtctggcagttcgtagcgttggtgttcgtgttgccgatgcagcggaagcatttcgtgttagcgttgccggtggtgcacgtccggcatttgcaccggcagatctgggtcatggttttggtctggccgaagttgaactgtatggtgatgttgttctgcgttttgttagctatccggatgaaaccgatctgccgtttctgccgggttttgaacgtgttagcagtccgggtgccgttgattatggtctgacccgttttgatcatgttgttggtaatgttccggaaatggcaccggttattgattatatgaaaggctttctgggctttcatgaatttgcagaatttaccgcagaagatgttggcaccaccgaaagcggtctgaatagcgttgttctggcaaataatagcgaaaatgttctgctgccgctgaatgaaccggtgcatggcaccaaacgtcgtagccagattcagacctatctggattatcatggtggtccgggtgttcagcatattgcactggcaagcaccgatgttctgcgtaccctgcgtgaaatgcgtgcacgtaccccgatgggtggttttgaatttatggcaccgccgcaggcaaaatattatgaaggtgttcgtcgtattgccggtgatgttctgagcgaagaacaaattaaagaatgtcaggaactgggcgttctggttgatcgtgatgatcagggtgttctgctgcagatttttaccaaaccggttggtgatcgtccgaccttttttctggaaatgattcagcgtattggctgcatggaaaaagatgaagttggtcaggaatatcagaaaggcggttgtggtggttttggtaaaggtaattttagcgaactgtttaaaagcattgaagattatgaaaaaaccctggaagccaaacagagcgttgttgcacagaaaagctaa

SEQ ID NO: 34 - protein, 440 aa, Lolium multiflorum
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSENVLLPLNEPVHGTKRRSQIQTYLDYHGGPGVQHIALASTDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKTLEAKQSVVAQKS

SEQ ID NO: 35 - DNA, 1053 bp, Synechococcus sp
atgaacccgtccattcgaattgtccaagggatccaccacctgcacttctacctttgggatctgccccgttggcgggaacacttttgtcgggtttggggcttccgggtggcaagcgacgccggcaacaccctggagctggagcagggatccctgcgcttgcgcctgtctcagccggcacgggcgggggacgaggtggaccgccatttgcagcggcatgggccgggggtggtggatgtggccttggcggtgggagagcaggagctaccggccttggcggagctgttgcggggccgaggcgcccaactggcgtggatcccggcagcagcggcgctctgcctccacaccccctacgggatccggcattctctgatccctggccccttggatgccgcccctgccgaagcgggcctgttttcccactgggatcacgtggtgttgaacgtggagcagggatccctgcaggcggcagccgactggtatgggcgggtgctgggctggcggcggctgtaccgctacagcatcggcaccgccacctccggcctggaaagcgtggtggtgggggatccggaagcggggatccaatgggccatcaacgagcccacctgtgccgcttcccagattcaggagtttttgcatgcccatggcggcccgggcattcagcacgcggcgctgcacagctcagacattgttgccagcctgcgccggttgcggcaggggggagtggactttttgcaagtggcgccgcagtactacaccagcctggaaagggagctggggttggcgctccgttctgcccttgggcaggccatctcctggcaagacctggtggagcagcagatccttctggatgctaccctgcccgcttctgatggccaggatcgcccccttctgctgcagacctttacccagcccctctttggtcggcccacctttttctttgaagtcattcaacggctaggcggggccacgggctttggcgaggccaattttcaggctttgttcgaggccctggaacggcaacagcgacagcgacaccaggcgctgaccccttag

SEQ ID NO: 36 - protein, 350 aa, Synechococcus sp
MNPSIRIVQGIHHLHFYLWDLPRWREHFCRVWGFRVASDAGNTLELEQGSLRLRLSQPARAGDEVDRHLQRHGPGVVDVALAVGEQELPALAELLRGRGAQLAWIPAAAALCLHTPYGIRHSLIPGPLDAAPAEAGLFSHWDHVVLNVEQGSLQAAADWYGRVLGWRRLYRYSIGTATSGLESVVVGDPEAGIQWAINEPTCAASQIQEFLHAHGGPGIQHAALHSSDIVASLRRLRQGGVDFLQVAPQYYTSLERELGLALRSALGQAISWQDLVEQQILLDATLPASDGQDRPLLLQTFTQPLFGRPTFFFEVIQRLGGATGFGEANFQALFEALERQQRQRHQALTP

SEQ ID NO: 37 - DNA, 1149 bp, Blepharisma japonicum
atgacttattacgacaagcaagaaacgcgtccagatcttggcgaattctatggtttccatcacgttcgtttttacgtctccaactcagagcaagccgcttcgttctacacatctcgctttgggttttctccggttgcctatgaaggattggaaacaggaaaccaaaaattctgtaccaatgtcgtccgaagcaaccatgtagtcatcgcttttacctcagctctcactcctgaagacaatgaagtgaaccgtcacgttggcaagcatagtgatggagttcaagacattgcctttagtgtaagtgacgcaagagggatgtatgagaaagcgatagctaaaggctgtaaaagcttccgtgagccacaggttttacaagatcaatttggatctgttataatagcgtctctccagacttatggagacactgttcacacattagtccaaaatgtcgactatacaggaccctttttgcctggcttcagagcaatcacaaaagatgatccattaaactctgcctttcctcaggtaaattatgacattattgatcatgttgtaggaaatcagcctggtggcgatatgactcctacagtagaatggtatgagaaatatctagaatttcatcgatattggtctgctgatgagtctgtaatccataccgattattcagcattaaggtctgttgtggttgctgattgggatgaagtgatcaaaatgcctattaatgagcctgctgatggacttagaaaaagtcaaatccaagaatatgtcgaatattatggtggagcaggcgtacaacatattgccttaaaagtcaatgatattatttcagtaataagcaccttaagggctagaggtgtggaattcttagaagttcctcctaaatattatgatagcttaagaaaaagacttgcgcattctgcggtacaaattgaagaagacttaaaaagaattgaagaccttcatattttggttgactttgacgaccgtgggtatttacttcagattttcacaaaaccagtagaagacagacctactctgttttatgaaattattcaaagacataataacaatggattcggaattggaaattttaaagccctatttgaatcattggaacaagagcaagaaagaagaggtaatttgatctaa

SEQ ID NO: 38 - protein, 350 aa, Blephrisman japonicum; identical to SEQ ID NO: 36

SEQ ID NO: 39 - DNA, 1107 bp, Picrophilus torridus
atgtatggcaaaaatttaatctcagaactaagggaaaaggagatctttaaacgattacatcacgtggaattttacgttagcagtgccaaaacatggtcatatttcatgaacaggggtcttggatttaaaacagtggcatatgccggtccagaaaccgggataagggacaagatatcctatgttatgtcccagggcactgcaaggatatcttttacatcatcaatgaatgatgatagctatatatcgaatcatgttaaaaaacacggggatggcgtaaaggatatagcacttgaggtcgatgatctggacgaggcaaaaagcctgatagaaaagtatggaacaaaggtttcaaaaataaatgaaataaaggatggaaatggaaagataagaactgcagagataaaaacgtacggtgaaaccgttcatacattaatagaaaccggggattacaatggcgtattcatgcccggttatgaggaatctgaaataaattcaaaaaacactgggataaaaaagatcgatcatatagttggaaatgtctatgagggcgagatggatagctgggttaatttttacatagaaaaacttggctttgagcatttaataacctttgatgataaagatataagaactgattacagcgcattaagatcaaaggttgtaaaatacaatgacgatatcgtatttccaataaatgagcctgcaaagggcttaagaaaatcacagatagaggaatatcttgactattacaggtctgagggcgttcagcacatagcactgttaactgatgatataataaaaactgtatccatgatggaggaaaacggcatagaatttttaaaaacaccaggatcatactatgaatccctatcatcaaggataggctcaatagacgaggatttaaatgaaatagagaaacataacatacttgtggatcgtgatgagaacggatacctattacagatcttcacaaagcctgttactgacaggccaacgttcttctttgaggtcatacagagaaagggtgcaaggtcattcggcaacggtaactttaaggcactttttgaggcgatagaaagggagcaggcaaagagaggaaacctatga

SEQ ID NO: 40 - protein, 368 aa, Picrophilus torridus
MYGKNLISELREKEIFKRLHHVEFYVSSAKTWSYFMNRGLGFKTVAYAGPETGIRDKISYVMSQGTARISFTSSMNDDSYISNHVKKHGDGVKDIALEVDDLDEAKSLIEKYGTKVSKINEIKDGNGKIRTAEIKTYGETVHTLIETGDYNGVFMPGYEESEINSKNTGIKKIDHIVGNVYEGEMDSWVNFYIEKLGFEHLITFDDKDIRTDYSALRSKVVKYNDDIVFPINEPAKGLRKSQIEEYLDYYRSEGVQHIALLTDDIIKTVSMMEENGIEFLKTPGSYYESLSSRIGSIDEDLNEIEKHNILVDRDENGYLLQIFTKPVTDRPTFFFEVIQRKGARSFGNGNFKALFEAIEREQAKRGNL

SEQ ID NO: 41 - DNA, 1164 bp, Kordia algicida
atggcagcagaaataaaaaacttaaaagatttacaaaatacagaatacggactcaaaaaattatttgacgaagcagaagactttcttccacttttaggaacagactacgtagaattatacgtcgggaacgccaaacaatcggcacatttctacaaaacggcttttggttttcaatcagaagcttacgcaggattggaaacaggattaaccgacagagtttcatacgtattaaaacaagataaaattcgcttggtcttaacaacaccattaggaaaaggtggcgaaatcaatgagcatatcgatttacacggcgatggcgtaaaagtagtagcactttgggtagaagatgctacaaaagcctttgaagaaacgaccaaaagaggcgcaaaaccgtacatggaaccaacaaaagaagaagatgaaaacggatatgtaattcgctcaggaatctatacgtacggagaaacggttcatgtttttgtagaacgtaaaaactataacggagtctttttaccaggatatcaaagatgggaatctcactacaatccggagccagttggcttaaaattcatcgatcacatggtaggaaatgtaggttggggagaaatgaaagaatggtgtgaattctacgcgaaagtaatgggatttgcgcaaattatctcctttacagatgatgatatttctaccgattttactgcgttgatgagtaaagtaatgagtaatggaaatggtagaatcaaatttccaatcaatgaacccgcagaaggaaaaaagaaatcgcaaattgaagaatatctagacttttacaatggttcaggagtacaacatattgcggttgctacagacaatattattgatacggtttcgcaaatgcgcgaacgtggagtagaattcttatacgttccagatacatattatgatgacttgttagaacgtgttggcgacatcgatgaagatgtagaagaactcaaaaaacacggaatcttaattgatcgtgatgaagaaggatacttattgcagttatttaccaaaaccattgtagacagaccaacaatgttctttgaagtcattcagcgtaaaggcgcacaatcatttggagtaggaaactttaaagctttatttgaagcgatagaaagagaacaagctgctcgcggaacattgtaa

SEQ ID NO: 42 - protein, 368 aa, Kordia algicida; identical to SEQ ID NO: 40

SEQ ID NO: 43 - DNA, 1206 bp, Rhodococcus sp.
atgacgatcgagcagactctcaccgacaaggaacgcctggcaggtctcgacctcggccagctcgagcagttggtcgggctcgtcgagtacgacggcacccgcgacccgttcccggtcagcggctgggatgccgtcgtctgggtggtcggcaacgccacccagaccgcccactacttccagtccgcgttcgggatgaccctcgtcgcctactccggacccaccaccggcaaccgcgaccaccacagcttcgtcctcgaatccggggccgtccgcttcgtcatcaaaggcgccgtgaacccggacagccccctgatcgaccaccaccgcacccacggcgacggcgtcgtcgacatcgccctcgccgtccccgacgtcgacaagtgcatcgcccacgcccgcgcccagggcgccaccgtcctcgacgaaccccacgacgtgaccgacgaccacggcaccgtccgcctcgccgcgatcgccacctacggcgacacccgccacaccctcgtcgaccgcagccactacaccggcccctacctgcccggctacaccgcccgcacctccggccacaccaaacgggacggggcacccaagcgcctgttccaggccctcgaccacgtcgtcggcaacgtcgaactcggcaagatggaccactgggtcgacttctacaaccgggtcatgggctttacgaacatggccgagttcgtcggcgaggacatcgccaccgactactccgcgctgatgagcaaggtcgtctccaacggcaaccaccgggtcaagttccccctcaacgaacccgccctcgccaagaaacgctcgcagatcgacgaatacctcgacttctaccgcggccccggcgcccagcacctggccctggccaccaatgacatcctcaccgccgtcgaccagctgaccgccgagggcgtcgagttcctggccacccccgactcctactacgaggaccccgaactgcgggcccggatcggcaacgtccgcgcccccatcgccgaactgcagaaacgcggcatcctcgtcgaccgcgacgaagacggctacctgctgcagatcttcaccaaacccctcgtcgaccggcccaccgtgttcttcgaactcatcgaacgccacggctccctcggcttcggcatcggcaacttcaaagccctcttcgaggccatcgaacgcgaacaagccgcccgcggaaacttctga

SEQ ID NO: 44 - protein, 401 aa, Rhodococcus sp.
MTIEQTLTDKERLAGLDLGQLEQLVGLVEYDGTRDPFPVSGWDAVVWVVGNATQTAHYFQSAFGMTLVAYSGPTTGNRDHHSFVLESGAVRFVIKGAVNPDSPLIDHHRTHGDGVVDIALAVPDVDKCIAHARAQGATVLDEPHDVTDDHGTVRLAAIATYGDTRHTLVDRSHYTGPYLPGYTARTSGHTKRDGAPKRLFQALDHVVGNVELGKMDHWVDFYNRVMGFTNMAEFVGEDIATDYSALMSKVVSNGNHRVKFPLNEPALAKKRSQIDEYLDFYRGPGAQHLALATNDILTAVDQLTAEGVEFLATPDSYYEDPELRARIGNVRAPIAELQKRGILVDRDEDGYLLQIFTKPLVDRPTVFFELIERHGSLGFGIGNFKALFEAIEREQAARGNF

SEQ ID NO: 45 - DNA, 1209 bp, Rhodococcus sp
atgactaccgccgacattcgcctgacgccccgcgaggtggccgcacatctggagaccgacgagctccggcagttggtcgggctcgtcgaacacgacgacgcgtcggatccgtttcccgtggtcgcgatggatgccgtggtgttcgtgtgcggcaacgcgacgcagagcacgcagtacttcgtctccacgtggggcatgaccctcgtcgcctacgccgggccggagaccggtcagcgctcgcacaagtccttcgtcctcgagtcggggtcggcacggttcgtgctgcacggcgccgtcgatccgaagagcccgctcgcggaccatcaccgggcgcacggcgacggcgtggtggacctggcgatggaagttctcgacgtcgaccgctgcatcgcgcatgcacgctcgcagggggccaccattctcgaggagccgcgcgacgtcacggatcagttcggcaccgtgcggctcgcggcgatcgccacgtacggcagcacccggcacaccatcgtcgaccgaagccgatacgacggcccctacctccccggattcgtcgcgcgctccagcggtttcgcggcgcgaccgggtaaacccccgcgattgttccaggcgctcgaccacgccgtcggcaacgtcgagatgggccggatggatcactgggtccggttctacaaccgcgtcatgggcttcacgaacatggccgaattcgtcggcgacgacatcgccacggagtactcggcgctgatgtcgaaggtcgtggcgaacggcaatcaccgggtgaagttcccgctcaacgaacccgcggtgggaaagaagaagtcgcagatcgacgaatatctcgagttctacggtgagccgggctgccagcatctggccctcgcgaccggagacatcctcgcgacggtggacgcgttgcgggccgagggtgtcgaattcctgaacacacccgacgcgtactacgaggacccacagctgcgcgcccggatcggcagggtgcgggtgccggtggaggaactgcagaagcgcggaatcctcgtcgaccgcgacgaggacggatacctcctgcagatcttcaccaaaccgctcggcgaccggccgaccgtgttcttcgaggtgatcgaacggcacggttcgctcgggttcggggcgggtaacttccaggccctgttcgaatccatcgagcgtgagcaggcggcgcgcggcaatctgtga

SEQ ID NO: 46 - protein, 402 aa, Rhodococcus sp
MTTADIRLTPREVAAHLETDELRQLVGLVEHDDASDPFPVVAMDAVVFVCGNATQSTQYFVSTWGMTLVAYAGPETGQRSHKSFVLESGSARFVLHGAVDPKSPLADHHRAHGDGVVDLAMEVLDVDRCIAHARSQGATILEEPRDVTDQFGTVRLAAIATYGSTRHTIVDRSRYDGPYLPGFVARSSGFAARPGKPPRLFQALDHAVGNVEMGRMDHWVRFYNRVMGFTNMAEFVGDDIATEYSALMSKVVANGNHRVKFPLNEPAVGKKKSQIDEYLEFYGEPGCQHLALATGDILATVDALRAEGVEFLNTPDAYYEDPQLRARIGRVRVPVEELQKRGILVDRDEDGYLLQIFTKPLGDRPTVFFEVIERHGSLGFGAGNFQALFESIEREQAARGNL

SEQ ID NO: 47 - DNA, 1161 bp, Arabidopsis thaliana
atggagctctcgatctcacaatcaccgcgtgttcggttctcgtctctggcgcctcgtttcttagcagcttctcatcatcatcgtccttctgtgcatttagctgggaagtttataagcctccctcgagatgttcgcttcacgagcttatcaacttcaagaatgcggtccaaatttgtttcaaccaattatagaaaaatctcaatccgggcatgttctcaggttggtgctgctgagtctgatgatccagtgctggatagaattgcccggttccaaaatgcttgctggagatttcttagaccccatacaatccgcggaacagctttaggatccactgccttggtgacaagagctttgatagagaacactcatttgatcaaatggagtcttgtactaaaggcactttcaggtcttcttgctcttatttgtgggaatggttatatagtcggcatcaatcagatctacgacattggaatcgacaaagtgaacaaaccatacttgccaatagcagcaggagatctatcagtgcagtctgcttggttgttagtgatattttttgcgatagcagggcttttagttgtcggatttaactttggtccattcattacaagcctatactctcttggcctttttctgggaaccatctattctgttccacccctcagaatgaaaagattcccagttgcagcatttcttattattgccacggtacgaggtttccttcttaactttggtgtgtaccatgctacaagagctgctcttggacttccatttcagtggagtgcacctgtggcgttcatcacatcttttgtgacactgtttgcactggtcattgctattacaaaggaccttcctgatgttgaaggagatcgaaagttccaaatatcaaccctggcaacaaaacttggagtgagaaacattgcattcctcggttctggacttctgctagtaaattatgtttcagccatatcactagctttctacatgcctcaggtttttagaggtagcttgatgattcctgcacatgtgatcttggcttcaggcttaattttccagacatgggtactagaaaaagcaaactacaccaaggaagctatctcaggatattatcggtttatatggaatctcttctacgcagagtatctgttattccccttcctctag

SEQ ID NO: 48 - protein, 386 aa, Arabidopsis thaliana
MELSISQSPRVRFSSLAPRFLAASHHHRPSVHLAGKFISLPRDVRFTSLSTSRMRSKFVSTNYRKISIRACSQVGAAESDDPVLDRIARFQNACWRFLRPHTIRGTALGSTALVTRALIENTHLIKWSLVLKALSGLLALICGNGYIVGINQIYDIGIDKVNKPYLPIAAGDLSVQSAWLLVIFFAIAGLLVVGFNFGPFITSLYSLGLFLGTIYSVPPLRMKRFPVAAFLIIATVRGFLLNFGVYHATRAALGLPFQWSAPVAFITSFVTLFALVIAITKDLPDVEGDRKFQISTLATKLGVRNIAFLGSGLLLVNYVSAISLAFYMPQVFRGSLMIPAHVILASGLIFQTWVLEKANYTKEAISGYYRFIWNLFYAEYLLFPFL

SEQ ID NO: 49 - DNA, 1113 bp, Chlamydomonas
atggacctttgcagctcaactggaagaggagcatgcctttcgccggcatccacgtcgcggccgtgcccagcaccagtgcatttgcgcggccgacgcctggctttctctccggctcagcctgctggacggcgccacttgccggtgctctcatctgcagcggtccccgctcccctcccaaatggtggaaacgacgagagcttcgcacaaaaactggctaactttccaaacgccttctggaagttcctgcggccacacaccatccgggggactatcctgggcaccacagctgtgaccgccaaggtccttatggagaaccccggctgcatagactgggcactgctgccgaaggcgctgctcggcctggtggcgctgctgtgcggcaacggctacattgtgggcatcaaccaaatctacgacgtcgacattgacgtggtcaacaagccattcctccccgtggcgtcgggcgagctgtcgccggcgctggcgtggggcctgtgtctgtcgctggcggctgcgggcgcgggcatcgtagccgccaacttcggcaacctcatcaccagcctctacacctttggcctcttcctgggcaccgtgtacagtgtgcctcccctgcgcctgaagcagtacgcggtgccggccttcatgatcatcgccacggtgcgcggcttcctgctcaacttcggcgtgtacagcgccacgcgggcggcactgggactgcccttcgagtggagcccggccgtcagcttcatcacggtgtttgtgacgctgtttgccactgtgatcgccatcaccaaggacctgccggacgtggagggcgaccaggccaacaacatctccaccttcgccacgcgcatgggcgtgcgcaacgtggcactgctggccatcggccttctcatggccaactacctgggtgccatcgcgctggcactcacctactccaccgccttcaacgtgccgctcatggcgggcgcgcacgccatcctggccgccacgctggcgctgcgcacgctcaagctgcacgccgccagctacagccgggaggcggtggcgtccttctaccgctggatctggaacctgttctacgccgagtacgcgctgctgccgttcctgtag

SEQ ID NO: 50 - protein, 370 aa, Chlamydomonas
MDLCSSTGRGACLSPASTSRPCPAPVHLRGRRLAFSPAQPAGRRHLPVLSSAAVPAPLPNGGNDESFAQKLANFPNAFWKFLRPHTIRGTILGTTAVTAKVLMENPGCIDWALLPKALLGLVALLCGNGYIVGINQIYDVDIDVVNKPFLPVASGELSPALAWGLCLSLAAAGAGIVAANFGNLITSLYTFGLFLGTVYSVPPLRLKQYAVPAFMIIATVRGFLLNFGVYSATRAALGLPFEWSPAVSFITVFVTLFATVIAITKDLPDVEGDQANNISTFATRMGVRNVALLAIGLLMANYLGAIALALTYSTAFNVPLMAGAHAILAATLALRTLKLHAASYSREAVASFYRWIWNLFYAEYALLPFL

SEQ ID NO: 51 - DNA, 1305 bp, Hordeum vulgare
atggcacctccaactccaacaacacctgccgctactggtgcagccgcagctgtaactcctgaacatgcgaggccacatcggatggttcgattcaatccgagatctgatagattccatactctgagcttccatcatgtggaattctggtgtgctgatgcagcttctgcagctggacgtttcgcttttgcccttggagctcctttagcagcgagatcagacttgagcacaggaaacagtgcacacgcatctcaacttctgcgttcaggaagccttgcgttcctgtttactgcaccgtatgctaacggatgcgacgcagcaactgcctcacttccttctttcagtgcagatgcagctagacgattctcagccgatcatggaattgcagtcagatctgtggctttgcgagttgctgatgctgccgaagctttcagggcatcagttgatggaggtgctaggcctgcttttgctcctgtggacttgggtagaggatttggctttgccgaggtcgaactctatggtgatgtggttctccggtttgtctctcacccagatggaacagatgttcctttcttgccagggtttgagggagtgacaaacccagatgcggtagattacggtctcacgagattcgaccatgtagtgggcaatgtaccggaattggctcctgcggctgcttacatagctggctttacgggatttcacgaattcgcggaattcaccgctgaggatgtcggaaccacagaatcagggctgaattccgtcgtccttgccaacaattccgaaggggtattgctgcctcttaacgagcctgtgcatggcacgaaaagacgtagccagatacagaccttcctagaacatcacggtggaccaggtgttcaacacattgctgttgccagcagtgatgtactcaggacgcttcgtaagatgagagctaggagtgcgatgggagggtttgactttctaccacctccgctgccaaaatactatgagggtgtgaggagactggctggtgatgttttgtctgaagcgcagatcaaggagtgtcaggaattaggggtgctcgttgacagagatgatcaaggggtgcttctccagatctttactaagccggttggtgataggcctaccctctttctagagatgattcaacgtatcgggtgtatggaaaaggacgagagaggtgaggagtatcaaaagggtggatgcggcggttttgggaaaggtaatttctccgagctgttcaagtcgatcgaagattacgagaaatcccttgaggcgaaacaatctgcagctgttcaaggatcg

SEQ ID NO: 52 - DNA, 1338 bp, Arabidopsis
atgggccaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcttcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 53 - protein, 445 aa, Arabidopsis
MGHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 54 - DNA, 1299 bp, Chlamydomonas
atgggcgctggtggcgcttctaccacggtagcgaatggcgggatcaagttggtagggcacaagaattttgtgcgctataatccacaatccgaccggtttgctattaagaggttccatagcttcgagttctggtgcgcggatgcgaccaacacatacaagcggttctcttatggcctgggcatgccgctggtcgccaagtccgaccagtccaccaacaaccagctctttgcctcctacgtgctgcgctccaacgacctggtcttcaccttcaccgcgccctacagccgcaagtgcgcctcggtcagcgagggcgttccgctgcgtcactacaacatcgaccatgcgtatgagttcatcaactcgcacgggctggcggtgcgggcagtaggcctgctggtggatgacgccaagacggcgtacgaggtgtctgtggcgcacggggccaagggcgtgctgccgccggtggagttgcgggatgaggcgagcggcaccagccaggtcatctcggaggtcattgtttacggggacgtcgttttccgctacgtgtcgggctccttcgagggccctttcatggccggctacacgccagtcacagactcgccggtcgcgtcgattgggttacagcgcgtggaccacgcggtgggcaacacacacgacctgatcaaggccgtggagtacatcaccgggttctgtggcttccacgagttctcagagtttgttgcggaggacgtgggcactgtggacagcggcctgaacagcatggtgcttgccaacaacgaggagaccatattgatgcctgtgaacgagcccaccttcggcacgccgcgcaagagccaaatccagacctacctggagcagaacgaggggccggggctgcagcacctggcgctgctcagcaacgacatcttcaccaccctgcgggagatgcgcgcgcgcagcgagctgggtggcttcgagttcatgccgcgggcaaatgcgaagtactacaaagacatgtacgcccgcatcggcgactcgctcacgccgcagcagtacagggaggtggaggagctgggcatcctggtggacaaggacgaccagggcgtgctgctgcagatcttcaccaagccgctgggcgaccggcccacggtgtttattgagatcatccagcgtgtgggctgcatgcgggaggtgaaggagcctgctacgggcgctgtggtggggacggagcaggcggctggctgcggcggcttcgggaaaggcaacttcggcgccctcttcaagtccattgaggactatgagcgcaccctaaatgtgtag

SEQ ID NO: 55 - protein, 432 aa, Chamydomonas
MGAGGASTTVANGGIKLVGHKNFVRYNPQSDRFAIKRFHSFEFWCADATNTYKRFSYGLGMPLVAKSDQSTNNQLFASYVLRSNDLVFTFTAPYSRKCASVSEGVPLRHYNIDHAYEFINSHGLAVRAVGLLVDDAKTAYEVSVAHGAKGVLPPVELRDEASGTSQVISEVIVYGDVVFRYVSGSFEGPFMAGYTPVTDSPVASIGLQRVDHAVGNTHDLIKAVEYITGFCGFHEFSEFVAEDVGTVDSGLNSMVLANNEETILMPVNEPTFGTPRKSQIQTYLEQNEGPGLQHLALLSNDIFTTLREMRARSELGGFEFMPRANAKYYKDMYARIGDSLTPQQYREVEELGILVDKDDQGVLLQIFTKPLGDRPTVFIEIIQRVGCMREVKEPATGAVVGTEQAAGCGGFGKGNFGALFKSIEDYERTLNV

SEQ ID NO: 56 - DNA, 1299 bp, Chlamydomonas
atgggagcgggtggtgcaggcaccggagatcgggaggggggcattaagctcgtgggctacaagaatttcgtgcgccagaacccgctttcagacaaattcaccgtccacaagtttcatcacatcgatttctggtgcggagatgcaacaaacacatcgaagcggttctcctacggcctgggcatgccgctggtcgccaagtccgaccagtccaccaacaaccagctctttgcctcctacgtgctgcgctccaacgacctggtcttcaccttcaccgcgccctacagccgcaagtgcgcctcggtcagcgagggcgttccgctgcgtcactacaacatcgaccatgcgtatgagttcatcaactcgcacgggctggcggtgcgggcagtaggcctgctggtggatgacgccaagacggcgtacgaggtgtctgtggcgcacggggccaagggcgtgctgccgccggtggagctgcgggatgaggcgagcggcaccagccaggtcatctcggaggtgctgctgtacggcgaggtcgtgctgcgctacgtgtcgggctccttccagggccccttcctggccggctacacgcccgtcacagactcggccgtgacctccttcggcctgcaacgtctggaccacgcggtgggcaacacccatgacctgatcaaggccgtggagtacatcaccggcttcacaggtttccacgagttctcagagtttgttgcggaggacgtgggcactgtggacagcggcctgaacagcatggtgctggcctccaacaacgaggcagtgctgctgcctgtgaacgagcccacctttggcacgccgcgcaagagccaaatccagacctacctggagcagaacgaggggccggggctgcagcacctggcgctgctcagcaacgacatcttcaccaccctgcgggagatgcgcgcgcgcagcgagctgggtggcttcgagttcatgccacgggcaaatgccaagtactacaaagacatgtacgcccgcatcggcgactcgctcacgccgcagcagtacagggaggtggaggagctgggcatcctggtggacaaggacgaccagggcgtgctgctgcagatcttcaccaagccgctgggcgaccggcccacggtgtttattgagatcatccagcgtgtgggctgcatgcgggaggtgaaagagcctgctacgggcgctgtggtggggacggagcaggcggctggctgcggcggcttcgggaaaggcaacttcggtgccctcttcaagtccattgaggactatgagcgcaccttaaatgtttaa

SEQ ID NO: 57 - protein, 432 aa, Chlamydomonas
MGAGGAGTGDREGGIKLVGYKNFVRQNPLSDKFTVHKFHHIDFWCGDATNTSKRFSYGLGMPLVAKSDQSTNNQLFASYVLRSNDLVFTFTAPYSRKCASVSEGVPLRHYNIDHAYEFINSHGLAVRAVGLLVDDAKTAYEVSVAHGAKGVLPPVELRDEASGTSQVISEVLLYGEVVLRYVSGSFQGPFLAGYTPVTDSAVTSFGLQRLDHAVGNTHDLIKAVEYITGFTGFHEFSEFVAEDVGTVDSGLNSMVLASNNEAVLLPVNEPTFGTPRKSQIQTYLEQNEGPGLQHLALLSNDIFTTLREMRARSELGGFEFMPRANAKYYKDMYARIGDSLTPQQYREVEELGILVDKDDQGVLLQIFTKPLGDRPTVFIEIIQRVGCMREVKEPATGAVVGTEQAAGCGGFGKGNFGALFKSIEDYERTLNV

SEQ ID NO: 58 - protein, 433 aa, Physcomitrella
MGLDKSESEGSVVGPLHLVGCERFVRNNPKTDRFGVERFHHVEFWCGDASNTWRRFSWGLGMHLVAKSDQTTGNQTYCSYAIQSNELVFAFTAPYSSTIDQTNTKMPHPGYKSDEARSFTDSHGLAVRAVGILVDDADEAFRISVEHGAVSVLEPHVLSDDAKGGKMVMAEVKLYGDVVLRYVSEQGYKGSMLPNYEEVESLPLSYGLVRLDHAVGNVHNLAEAVNYIAKFTGFHEFAEFTAGDVGTTESGLNSMVVASNNEMVLLPINEPTFGTKRKSQIQTYLEHNEGPGLQHLALICDNIFSTLREMRTRTHIGGFDFMPKPPPTYYKNLANRVGDILTAEQIKECDELGILVDKDDQGVLLQIFTKPVGDRPSIFVEIIQRIGCMDKDESTGATVQKGGCGGFGKGNFSELFKSIEEYEKTLDGTLKVH

SEQ ID NO: 59 - protein, 446 aa, Oryza
MPPTPTPTATTGAVSAAAAAGENAGFRLVGHRRFVRANPRSDRFQALAFHHVELWCADAASAAGRFAFALGAPLAARSDLSTGNSAHASLLLRSASVAFLFTAPYGGDHGVGADAATTASIPSFSPGAARRFAADHGLAVHAVALRVADAADAFRASVAAGARPAFQPADLGGGFGLAEVELYGDVVLRFVSHPDGADAPFLPGFEGVSNPGAVDYGLRRFDHVVGNVPELAPVAAYISGFTGFHEFAEFTAEDVGTAESGLNSVVLANNAETVLLPLNEPVHGTKRRSQIQTYLDHHGGPGVQHIALASDDVLGTLREMRARSAMGGFEFLAPPPPNYYDGVRRRAGDVLSEEQINECQELGVLVDRDDQGVLLQIFTKPVGDRPTFFLEMIQRIGCMEKDESGQEYQKGGCGGFGKGNFSELFKSIEEYEKSLEAKQAPTVQGS

SEQ ID NO: 60 - protein, 436 aa, Triticum
MPPTPTTPAATGAGAAAAVTPEHARPRRMVRFNPRSDRFHTLSFHHVEFWCADAASAAGRFAFALGAPLAARSDLSTGNSVHASQLLRSGNLAFLFTAPYANGCDAATASLPSFSADAARRFSADHGLAVRSIALRVADAAEAFRASVDGGARPAFSPVDLGRGFGFAEVELYGDVVLRFVSHPDDTDVPFLPGFEGVSNPDAVDYGLTRFDHVVGNVPELAPAAAYVAGFAGFHEFAEFTTEDVGTAESGLNSMVLANNSEGVLLPLNEPVHGTKRRSQIQTFLEHHGGSGVQHIAVASSDVLRTLREMRARSAMGGFDFLPPRCRKYYEGVRRIAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 61 - protein, 444 aa, Zea
MPPTPTAAAAGAAVAAASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEEAFRTSVAAGARPAFGPVDLGRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFPKPVGDRPTLFLEIIQRIGCMERDEKGQEYQKGGCGGFGKGNFSQLFKSIEDYEKSLEAMQAAAAATAQGS

SEQ ID NO: 62 - protein, 443 aa, Glycine
  misc_feature (393)..(393): Xaa can be any naturally occurring amino acid
MCNEIQAQAQAQAQPGFKLVGFKNFVRTNPKSDRFQVNRFHHIEFWCTDATNASRRFSWGLGMPIVAKSDLSTGNQIHASYLLRSGDLSFLFSAPYSPSLSAGSSAASSASIPSFDAATCLAFAAKHGFGVRAIALEVADAEAAFSASVAKGAEPASPPVLVDDRTGFAEVRLYGDVVLRYVSYKDAAPQAPHADPSRWFLPGFEAAASSSSFPELDYGIRRLDHAVGNVPELAPAVRYLKGFSGFHEFAEFTAEDVGTSESGLNSVVLANNSETVLLPLNEPVYGTKRKSQIETYLEHNEGAGVQHLALVTHDIFTTLREMRKRSFLGGFEFMPSPPPTYYANLHNRAADVLTVDQIKQCEELGILVDRDDQGTLLQIFTKPVGDRPTIFIXIIQRIGCMVEDEEGKVYQKGACGGFGKGNFSELFKSIEEYEKTLEAKRTA

SEQ ID NO: 63 - protein, 445 aa, Vitis
MGKQNTTTNNPAPGFKLVGFSNFLRTNPMSDRFGVKRFHHIEFWSTDATNLARRFSWGLGMPIVAKSDLSTGNVIHASYLTRSGDLNFLFTAPYSPSIAGDLENAAATASIPSFDHSACHAFAASHGLGVRAIAIEVDDAEGAFHTSVAHGARPMSPPVTMGGSVVISEVHLYGDAVLRYVSYKNPNPNATSDPSSWFLPGFEAVDEGSSFPVDFGLRRVDHTVGNVPKLAPVVTYLKQFTGFHEFAEFTAEDVGTSESGLNSVVLASNNEMVLLPLNEPVFGTKRKSQIQTYLEHNEGPGVQHLALMSDDIFRTLREMRRRSGVGGFDFMPSPPPTYYRNVKKRAGDVLTDDQIKECEELGILVDKDDQGTLLQIFTKPLGDRPTIFIEIIQRLGCMVKDDEGKVSQKGGCGGFGKGNFSELFKSIEEYEKTLGAKRIVDPAPV

SEQ ID NO: 64 - protein, 358 aa, Pseudomonas fluorescens
MADQYENPMGLMGFEFIEFASPTPGTLEPIFEIMGFTKVATHRSKNVHLYRQGEINLILNNQPDSLASYFAAEHGPSVCGMAFRVKDSQQAYNRALELGAQPIHIETGPMELNLPAIKGIGGAPLYLIDRFGEGSSIYDIDFVYLEGVDRNPVGAGLKVIDHLTHNVYRGRMAYWANFYEKLFNFREARYFDIKGEYTGLTSKAMSAPDGMIRIPLNEESSKGAGQIEEFLMQFNGEGIQHVAFLTEDLVKTWDALKKIGMRFMTAPPDTYYEMLEGRLPNHGEPVDQLQARGILLDGSSIEGDKRLLLQIFSETLMGPVFFEFIQRKGDDGFGEGNFKALFESIERDQVRRGVLTTD

SEQ ID NO: 65 - protein, 358 aa, Pseudomonas fluorescens
MADLYENPMGLMGFEFIELASPTPNTLEPIFEIMGFTKVATHRSKDVHLYRQGAINLILNNEPHSVASYFAAEHGPSVCGMAFRVKDSQKAYKRALELGAQPIHIETGPMELNLPAIKGIGGAPLYLIDRFGEGSSIYDIDFVFLEGVDRHPVGAGLKIIDHLTHNVYRGRMAYWANFYEKLFNFREIRYFDIKGEYTGLTSKAMTAPDGMIRIPLNEESSKGAGQIEEFLMQFNGEGIQHVAFLSDDLIKTWDHLKSIGMRFMTAPPDTYYEMLEGRLPNHGEPVGELQARGILLDGSSESGDKRLLLQIFSETLMGPVFFEFIQRKGDDGFGEGNFKALFESIERDQVRRGVLSTD

SEQ ID NO: 66 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 67 - protein, 444 aa, Zea mays
MGPTPTAAAAGAAVAAASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEDAFRASVAAGARPAFGPVDLGRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAAAAAAQGS

SEQ ID NO: 68 - DNA, 1332 bp, Zea mays
atgggtccgaccccgaccgcaacagcagccggtgcagcagttgcagcagcaagcgcagcagaacaggcagcatttcgtctggttggtcatcgtaattttgttcgttttaatccgcgttccgatcgttttcataccctggcatttcatcatgttgaactgtggtgtgcagatgcagcatcagcagcaggtcgttttagctttgcactgggtgcaccgctggcagcacgtagcgatctgagcaccggtaatagcgcacatgcaagtctgctgctgcgtagcggtagcctgagctttctgtttaccgcaccgtatgcacatggtgccgatgcagccaccgcagcactgccgagctttagcgcagccgcagcccgtcgttttgcagcagatcatggtctggcagttcgtgcagttgcactgcgtgttgcagatgccgaagatgcatttcgtgcaagcgttgcagcgggtgcacgtccggcatttggtccggttgatctgggtcgtggttttcgcctggccgaagtggaactgtatggtgatgttgttctgcgttatgttagctatccggatggtgccgcaggcgaaccgtttctgcctggttttgaaggtgttgcaagtccgggtgcagccgattatggcctgagccgttttgatcatattgttggtaatgtgccggaactggcaccggcagcagcatattttgcaggttttaccggttttcatgaatttgccgaatttaccaccgaagatgttggcaccgcagaaagcggtctgaatagcatggcactggcaaataatagcgaaaatgtactgctgccgctgaatgaaccggtgcatggcaccaaacgtcgtagccagattcagacctttctggatcatcatggtggtccgggtgttcagcatattgcactggcatcagatgatgtgctgcgtaccctgcgtgaaatgcaggcacgtagtgcaatgggtggctttgaatttctgcctccgcctccgagcgattattatgatggtgttcgtcgtgaagccggtgatgttctgaccgaagcacagattaatgaatgtcaagaactgggtgttatggtggatcgtgatgatcagggtgtcctgctgcagatttttaccaaaccggttggtgatcgtccgaccttttttctggaaattattcagcgtattggctgcatggaaaaagatgaaaaaggccaagaatatcagaaaggtggttgtggtggttttggcaaagataactttagccagctgtttaaaagcatcgaggattatgaaaaaagcctggaagcaaaacaagccgcagcagccgcaaccgcacagggtagc

SEQ ID NO: 69 - DNA, 1332 bp, Zea mays
atgggtccgaccccgaccgcaacagcagccggtgcagcagttgcagcagcaagcgcagcagaacaggcagcatttcgtctggttggtcatcgtaattttgttcgttttaatccgcgtagcgatcgttttcagaccctggcatttcatcatgttgaactgtggtgtgcagatgcagcatcagcagcaggtcgttttagctttgcactgggtgttccgctggcagcacgtagtgatctgagcaccggtaatagcgcacatgcaagtctgctgctgcgtagcggtagcctgagcctgctgtttaccgcaccgtatgcacatggtgccgatgcagccaccgcagcactgccgagctttagcgcagccgcagcccgtcgttttgcagcagatcatggtctggcagttcgtgcagttgccctgcgtgttgcagatgccgaagatgcatttcgtgcaagcgttgcagcgggtgcacgtccggcatttggtccggttgatctgggtcgtggttttcgcctggccgaagtggaactgtatggtgatgttgttctgcgttatgttagctatccggatggtgccgcaggcgaaccgtttctgcctggttttgaaggtgttgcaagtccgggtgcagccgattatggcctgagccgttttgatcatattgttggtaatgtgccggaactggcaccggcagcagcatatatggcaggttttaccggttttcatgaatttgcagaatttaccaccgaagatgttggcaccaccgaaagcggtctgaatagcatggcactggcaaataatagcgaaaatgtactgctgccgctgaatgaaccggtgcatggcaccaaacgtcgtagccagattcagacctttctggatcatcatggtggtccgggtgttcagcatattgcactggcatcagatgatgtgctgcgtaccctgcgtgaaatgcgtgcacgtagcgcaatgggtggctttgaatttctgcctccgcctctgagcgattattatgatggtgttcgtcgttgtgccggtgatgttctgaccgaagcacagattaatgaatgtcaagaactgggtgtgatggtggatcgtgatgatcagggtgtcctgctgcagatttttaccaaaccggttggtgatcgtccgaccttttttctggaaattattcagcgtattggctgcatggaaaaagatgaaaaaggccaagaatatcagaaaggtggttgtggtggttttggcaaaggtaattttggtcagctgtttaaaagcatcgaggactatgaaaaaagcctggaagcaaaacaagccgcagcagccgcaaccgcacagggtagc

SEQ ID NO: 70 - DNA, 16 bp, Artificial
  Primer
ggccaccaaaacgccg

SEQ ID NO: 71 - DNA, 24 bp, Artificial
  Primer
tcatcccactaactgtttggcttc

SEQ ID NO: 72 - DNA, 23 bp, Artificial
  Primer
ggcgctggcggtgcgtccactac

SEQ ID NO: 73 - DNA, 34 bp, Artificial
  Primer
tcaaacgttcagggtacgctcgtagtcttcgatg

SEQ ID NO: 74 - DNA, 21 bp, Artificial
  Primer
ggtgcgggtggcgctggcacc

SEQ ID NO: 75 - DNA, 35 bp, Artificial
  Primer
tcaaacgttcagggtacgttcgtagtcctcgatgg

SEQ ID NO: 76 - DNA, 19 bp, Artificial
  Primer
ccaatcccaatgtgcaacg

SEQ ID NO: 77 - DNA, 22 bp, Artificial
  Primer
ttatgcggtacgtttagcctcc

SEQ ID NO: 78 - DNA, 23 bp, Artificial
  Primer
ccaccgactccgaccgccgcagc

SEQ ID NO: 79 - DNA, 26 bp, Artificial
  Primer
tcaggaaccctgtgcagctgccgcag

SEQ ID NO: 80 - DNA, 17 bp, Artificial
  Primer
ccgccgactccaacccc

SEQ ID NO: 81 - DNA, 21 bp, Artificial
  Primer
ttaagaaccctgaacggtcgg

SEQ ID NO: 82 - DNA, 28 bp, Artificial
  Primer
gaggattcgacttcgcgccttctcctcc

SEQ ID NO: 83 - DNA, 28 bp, Artificial
  Primer
ggaggagaaggcgcgaagtcgaatcctc

SEQ ID NO: 84 - DNA, 29 bp, Artificial
  Primer
gaggattcgacttctggccttctcctccg

SEQ ID NO: 85 - DNA, 29 bp, Artificial
  Primer
cggaggagaaggccagaagtcgaatcctc

SEQ ID NO: 86 - DNA, 31 bp, Artificial
  Primer
ggaggattcgacttctttccttctcctccgc

SEQ ID NO: 87 - DNA, 31 bp, Artificial
  Primer
gcggaggagaaggaaagaagtcgaatcctcc

SEQ ID NO: 88 - DNA, 35 bp, Artificial
  Primer
gtgacaggccgacgatagctatagagataatccag

SEQ ID NO: 89 - DNA, 35 bp, Artificial
  Primer
ctggattatctctatagctatcgtcggcctgtcac

SEQ ID NO: 90 - DNA, 30 bp, Artificial
  Primer
gacttcatgcctcctcctccgcctacttac

SEQ ID NO: 91 - DNA, 30 bp, Artificial
  Primer
gtaagtaggcggaggaggaggcatgaagtc

SEQ ID NO: 92 - DNA, 31 bp, Artificial
  Primer
gattcgacttcatggcttctcctccgcctac

SEQ ID NO: 93 - DNA, 31 bp, Artificial
  Primer
gtaggcggaggagaagccatgaagtcgaatc

SEQ ID NO: 94 - DNA, 34 bp, Artificial
  Primer
cagatcaaggagtgtcaggaattagggattcttg

SEQ ID NO: 95 - DNA, 34 bp, Artificial
  Primer
caagaatccctaattcctgacactccttgatctg

SEQ ID NO: 96 - DNA, 38 bp, Artificial
  Primer
cggaacaaagaggaagagtgagattcagacgtatttgg

SEQ ID NO: 97 - DNA, 38 bp, Artificial
  Primer
ccaaatacgtctgaatctcactcttcctctttgttccg

SEQ ID NO: 98 - DNA, 40 bp, Artificial
  Primer
cgttgcttcaaatcttcccgaaaccactaggtgacaggcc

SEQ ID NO: 99 - DNA, 40 bp, Artificial
  Primer
ggcctgtcacctagtggtttcgggaagatttgaagcaacg

SEQ ID NO: 100 - DNA, 38 bp, Artificial
  Primer
caaatcttcacaaaaccagtgggtgacaggccgacgat

SEQ ID NO: 101 - DNA, 38 bp, Artificial
  Primer
atcgtcggcctgtcacccactggttttgtgaagatttg

SEQ ID NO: 102 - DNA, 40 bp, Artificial
  Primer
tgacaggccgacgatatttctggagataatccagagagta

SEQ ID NO: 103 - DNA, 40 bp, Artificial
  Primer
tactctctggattatctccagaaatatcgtcggcctgtca

SEQ ID NO: 104 - DNA, 30 bp, Artificial
  Primer
gacttcatgcctgcgcctccgcctacttac

SEQ ID NO: 105 - DNA, 30 bp, Artificial
  Primer
gtaagtaggcggaggcgcaggcatgaagtc

SEQ ID NO: 106 - DNA, 34 bp, Artificial
  Primer
ggcaatttctctgagttcttcaagtccattgaag

SEQ ID NO: 107 - DNA, 34 bp, Artificial
  Primer
cttcaatggacttgaagaactcagagaaattgcc

SEQ ID NO: 108 - DNA, 37 bp, Artificial
  Primer
ggaacaaagaggaagagtgtgattcagacgtatttgg

SEQ ID NO: 109 - DNA, 37 bp, Artificial
  Primer
ccaaatacgtctgaatcacactcttcctctttgttcc

SEQ ID NO: 110 - DNA, 28 bp, Artificial
  Primer
gaggattcgacttcaacccttctcctcc

SEQ ID NO: 111 - DNA, 28 bp, Artificial
  Primer
ggaggagaagggttgaagtcgaatcctc

SEQ ID NO: 112 - DNA, 28 bp, Artificial
  Primer
gaggattcgacttccagccttctcctcc

SEQ ID NO: 113 - DNA, 28 bp, Artificial
  Primer
ggaggagaaggctggaagtcgaatcctc

SEQ ID NO: 114 - DNA, 37 bp, Artificial
  Primer
ggaacaaagaggaagagtaacattcagacgtatttgg

SEQ ID NO: 115 - DNA, 37 bp, Artificial
  Primer
ccaaatacgtctgaatgttactcttcctctttgttcc

SEQ ID NO: 116 - DNA, 37 bp, Artificial
  Primer
ggaacaaagaggaagagtcacattcagacgtatttgg

SEQ ID NO: 117 - DNA, 37 bp, Artificial
  Primer
ccaaatacgtctgaatgtgactcttcctctttgttcc

SEQ ID NO: 118 - DNA, 37 bp, Artificial
  Primer Ta2-126
ggaacaaagaggaagagtgcgattcagacgtatttgg

SEQ ID NO: 119 - DNA, 37 bp, Artificial
  Primer Ta2-127
ccaaatacgtctgaatcgcactcttcctctttgttcc

SEQ ID NO: 120 - DNA, 37 bp, Artificial
  Primer
ggaacaaagaggaagagtctgattcagacgtatttgg

SEQ ID NO: 121 - DNA, 37 bp, Artificial
  Primer
ccaaatacgtctgaatcagactcttcctctttgttcc

SEQ ID NO: 122 - DNA, 37 bp, Artificial
  Primer
ggaacaaagaggaagagtataattcagacgtatttgg

SEQ ID NO: 123 - DNA, 37 bp, Artificial
  Primer
ccaaatacgtctgaattatactcttcctctttgttcc

SEQ ID NO: 124 - DNA, 37 bp, Artificial
  Primer
ggaacaaagaggaagagttcgattcagacgtatttgg

SEQ ID NO: 125 - DNA, 37 bp, Artificial
  Primer
ccaaatacgtctgaatcgaactcttcctctttgttcc

SEQ ID NO: 126 - DNA, 28 bp, Artificial
  Primer
gaggattcgacttccacccttctcctcc

SEQ ID NO: 127 - DNA, 28 bp, Artificial
  Primer
ggaggagaagggtggaagtcgaatcctc

SEQ ID NO: 128 - DNA, 28 bp, Artificial
  Primer
gaggattcgacttctacccttctcctcc

SEQ ID NO: 129 - DNA, 28 bp, Artificial
  Primer
ggaggagaagggtagaagtcgaatcctc

SEQ ID NO: 130 - DNA, 28 bp, Artificial
  Primer
gaggattcgacttcagcccttctcctcc

SEQ ID NO: 131 - DNA, 28 bp, Artificial
  Primer
ggaggagaagggctgaagtcgaatcctc

SEQ ID NO: 132 - DNA, 28 bp, Artificial
  Primer
gaggattcgacttcacaccttctcctcc

SEQ ID NO: 133 - DNA, 28 bp, Artificial
  Primer
ggaggagaaggtgtgaagtcgaatcctc

SEQ ID NO: 134 - DNA, 28 bp, Artificial
  Primer
gaggattcgacttctgtccttctcctcc

SEQ ID NO: 135 - DNA, 28 bp, Artificial
  Primer
ggaggagaaggacagaagtcgaatcctc

SEQ ID NO: 136 - DNA, 29 bp, Artificial
  Primer
ggattcgacttcatgcgttctcctccgcc

SEQ ID NO: 137 - DNA, 29 bp, Artificial
  Primer
ggcggaggagaacgcatgaagtcgaatcc

SEQ ID NO: 138 - DNA, 31 bp, Artificial
  Primer
gaggaattagggatttgggtagacagagatg

SEQ ID NO: 139 - DNA, 31 bp, Artificial
  Primer
catctctgtctacccaaatccctaattcctc

SEQ ID NO: 140 - DNA, 31 bp, Artificial
  Primer
gaggaattagggattatggtagacagagatg

SEQ ID NO: 141 - DNA, 31 bp, Artificial
  Primer
catctctgtctaccataatccctaattcctc

SEQ ID NO: 142 - DNA, 30 bp, Artificial
  Primer
ggtggttttggcaaacacaatttctctgag

SEQ ID NO: 143 - DNA, 30 bp, Artificial
  Primer
ctcagagaaattgtgtttgccaaaaccacc

SEQ ID NO: 144 - DNA, 30 bp, Artificial
  Primer
ggtggttttggcaaatgcaatttctctgag

SEQ ID NO: 145 - DNA, 30 bp, Artificial
  Primer
ctcagagaaattgcatttgccaaaaccacc

SEQ ID NO: 146 - DNA, 30 bp, Artificial
  Primer
ggtggttttggcacaggcaatttctctgag

SEQ ID NO: 147 - DNA, 30 bp, Artificial
  Primer
ctcagagaaattgcctgtgccaaaaccacc

SEQ ID NO: 148 - DNA, 31 bp, Artificial
  Primer
gggagggtttgactttcatccacctccgctg

SEQ ID NO: 149 - DNA, 31 bp, Artificial
  Primer
cagcggaggtggatgaaagtcaaaccctccc

SEQ ID NO: 150 - DNA, 27 bp, Artificial
  Primer
ggcttcgacttctatccacccccgctg

SEQ ID NO: 151 - DNA, 27 bp, Artificial
  Primer
cagcgggggtggatagaagtcgaagcc

SEQ ID NO: 152 - DNA, 30 bp, Artificial
  Primer
gggttcggcaaatgcaacttctccgagctg

SEQ ID NO: 153 - DNA, 30 bp, Artificial
  Primer
cagctcggagaagttgcatttgccgaaccc

SEQ ID NO: 154 - DNA, 30 bp, Artificial
  Primer
ggagggtttgactttcatgcacctccgctg

SEQ ID NO: 155 - DNA, 30 bp, Artificial
  Primer
cagcggaggtgcatgaaagtcaaaccctcc